(19) United States
(12) Patent Application Publication
(10) Pub. No.: US 2023/0226040 A1
(43) Pub. Date: Jul. 20, 2023

(54) COMBINATION THERAPY COMPRISING AN FGFR INHIBITOR AND A KRAS INHIBITOR

(71) Applicant: Incyte Corporation, Wilmington, DE (US)

(72) Inventors: Alejandro Amador Arjona, Malvern, PA (US); Angela Abdollahi, Kennett Square, PA (US); Margaret Favata, North East, MD (US); Jonathan Rios-Doria, Wallingford, PA (US); Hui Wang, Hockessin, DE (US); Susan Wee, Moorestown, NJ (US); Pei Gan, Claymont, DE (US); Jeffrey C. Yang, Wilmington, DE (US); Yong Li, Newark, DE (US); Le Zhao, Wilmington, DE (US); Chunhong He, Boothwyn, PA (US); Chunyin Marshall Law, Wilmington, DE (US); Wenyu Zhu, Media, PA (US); Xiaozhao Wang, Moorestown, NJ (US); Wenqing Yao, Chadds Ford, PA (US); Peter Niels Carlsen, Claymont, DE (US); Fenglei Zhang, Berwyn, PA (US); Qinda Ye, Claymont, DE (US); Matthew S. McCammant, Newark, DE (US); Rocco Policarpo, Wynnewood, PA (US); Artem Shvartsbart, Kennett Square, PA (US); Jeremy Roach, Philadelphia, PA (US); Gia Hoang, Wilmington, DE (US); Bin Hu, Garnet Valley, PA (US); Gencheng Li, Claymont, DE (US); Robert Susick, Wilmington, DE (US); Padmaja Polam, Kennett Square, PA (US); Chao Qi, Newark, DE (US); Alexander Sokolsky, Philadelphia, PA (US); Haolin Yin, Wilmington, DE (US); Chang Min, Wuhan (CN)

(21) Appl. No.: 17/991,482

(22) Filed: Nov. 21, 2022

Related U.S. Application Data
(60) Provisional application No. 63/352,491, filed on Jun. 15, 2022.
(60) Provisional application No. 63/317,654, filed on Mar. 8, 2022.
(60) Provisional application No. 63/282,017, filed on Nov. 22, 2021.

(51) Int. Cl. A61K 31/4745; A61P 35/00; A61P 11/00; A61K 31/5377; A61K 31/519; A61K 31/498; A61K 31/506; A61K 45/06

(52) U.S. Cl. CPC A61K 31/4745; A61P 35/00; A61P 11/00; A61K 31/5377; A61K 31/519; A61K 31/498; A61K 31/506; A61K 45/06

(57) ABSTRACT

The present disclosure relates to methods of treating cancer by administering a compound, which is a Fibroblast Growth Factor Receptor (FGFR) inhibitor, in combination with a Kirsten rat sarcoma (KRAS) inhibitor.

### FIELD

The present disclosure relates to methods of treating cancer by administering a compound, which is a Fibroblast Growth Factor Receptor (FGFR) inhibitor, in combination with a Kirsten rat sarcoma (KRAS) inhibitor.

### BACKGROUND

Ras proteins are part of the family of small GTPases that are activated by growth factors and various extracellular stimuli. The Ras family regulates intracellular signaling pathways responsible for growth, migration, survival and differentiation of cells. Activation of RAS proteins at the cell membrane results in the binding of key effectors and initiation of a cascade of intracellular signaling pathways within the cell, including the RAF and PI3K kinase pathways. Somatic mutations in RAS may result in uncontrolled cell growth and malignant transformation while the activation of RAS proteins is tightly regulated in normal cells (Simanshu, D. et al. Cell 170.1 (2017):17-33). The Ras family is comprised of three members: KRAS, NRAS and HRAS. RAS mutant cancers account for about 25% of human cancers. KRAS is the most frequently mutated isoform accounting for 85% of all RAS mutations whereas NRAS and HRAS are found mutated in 12% and 3% of all Ras mutant cancers respectively (Simanshu, D. et al. Cell 170.1 (2017):17-33). KRAS mutations are prevalent amongst the top three most deadly cancer types: pancreatic (97%), colorectal (44%), and lung (30%) (Cox, A.D. et al. Nat Rev Drug Discov (2014) 13:828-51). The majority of RAS mutations occur at amino acid residue 12, 13, and 61. The frequency of specific mutations varies between RAS gene isoforms and while G12 and Q61 mutations are predominant in KRAS and NRAS respectively, G12, G13 and Q61 mutations are most frequent in HRAS. Furthermore, the spectrum of mutations in a RAS isoform differs between cancer types. For example, KRAS G12D mutations predominate in pancreatic cancers (51%), followed by colorectal adenocarcinomas (45%) and lung cancers (17%) while KRAS G12V mutations are associated with pancreatic cancers (30%), followed by colorectal adenocarcinomas (27%) and lung adenocarcinomas (23%) (Cox, A.D. et al. Nat Rev Drug Discov (2014) 13:828-51). In contrast, KRAS G12C mutations predominate in non-small cell lung cancer (NSCLC) comprising 11-16% of lung adenocarcinomas, and 2-5% of pancreatic and colorectal adenocarcinomas (Cox, A.D. et al. Nat. Rev. Drug Discov. (2014) 13:828-51). Genomic studies across hundreds of cancer cell lines have demonstrated that cancer cells harboring KRAS mutations are highly dependent on KRAS function for cell growth and survival (McDonald, R. et al. Cell 170 (2017): 577-592). The role of mutant KRAS as an oncogenic driver is further supported by extensive in vivo experimental evidence showing mutant KRAS is required for early tumour onset and maintenance in animal models (Cox, A.D. et al. Nat Rev Drug Discov (2014) 13:828-51).

The Fibroblast Growth Factor Receptors (FGFR) are receptor tyrosine kinases that bind to fibroblast growth factor (FGF) ligands. There are four FGFR proteins (FGFR1-4) that are capable of binding ligands and are involved in the regulation of many physiological processes including tissue development, angiogenesis, wound healing, and metabolic regulation. Upon ligand binding, the receptors undergo dimerization and phosphorylation leading to stimulation of the protein kinase activity and recruitment of many intracellular docking proteins. These interactions facilitate the activation of an array of intracellular signaling pathways including Ras-MAPK, AKT-PI3K, and phospholipase C that are important for cellular growth, proliferation and survival (Reviewed in Eswarakumar et al. Cytokine & Growth Factor Reviews (2005) 16(2): 139-149).

Aberrant activation of this pathway either through overexpression of FGF ligands or FGFR or activating mutations in the FGFRs can lead to tumor development, progression, and resistance to conventional cancer therapies. In human cancer, genetic alterations including gene amplification, chromosomal translocations and somatic mutations that lead to ligand-independent receptor activation have been described. Large scale DNA sequencing of thousands of tumor samples has revealed that components of the FGFR pathway are among the most frequently mutated in human cancer.

Recently, the FDA approved the KRAS G12C inhibitor sotorasib to treat KRAS G12C mutated non-small cell lung cancer (NSCLC). Potential resistant mechanisms of KRAS G12C mutated tumors to KRAS G12C inhibitors are under investigation. Recently, several literature articles reported that epithelial-mesenchymal transition (EMT) is a cause of intrinsic resistance to KRAS G12C inhibitors, and FGFR1 high expression is associated with epithelial or mesenchymal cancer lines. (See, e.g., Adachi et al. Clinical Cancer Research, (2020) 26(22):5962-5973; Solanki et al. Clinical Cancer Research, (2021) 27(9):2533-2548; Kitai et al. Cancer Discovery, (2016) 6(7):754-69).

Inhibitors of FGFR are currently being developed for the treatment of cancer. For example, pemigatinib, or 3-(2,6-difluoro-3,5-dimethoxyphenyl)-1-ethyl-8-(morpholin-4-ylmethyl)-1,3,4,7-tetrahydro-2H-pyrrolo[3′,2′:5,6]pyrido[4,3-d]pyrimidin-2-one, and other small molecule inhibitors of FGFR are reported in U.S. Pat. No. 9,611,267 and US Publication Nos.: 2012/0165305; 2014/0045814; 2013/0338134; 2014/0171405; 2014/0315902; 2016/0115164; 2016/0244448; 2016/0244449, 2016/0244450, 2019/0337948, and 2020/0002338.

There remains a need for new treatment regimens for cancer with KRAS mutations while addressing potential drug resistant mechanisms such as those including mesenchymal phenotypes and FGFR dysfunction, in particular FGFR1 dysfunction. The present disclosure is directed toward this need and others using FGFR inhibitors, in particular FGFR1 inhibitors, in combination with KRAS inhibitors.

### SUMMARY

The present application provides, inter alfa, methods of treating cancer in a patient, comprising administering to said patient:

(i) an FGFR1 inhibitor; and

(ii) a KRAS inhibitor.

The present application further provides methods of treating cancer in a patient, comprising administering to the patient an FGFR1 inhibitor and a KRAS inhibitor.

The present application also provides use of an FGFR1 inhibitor and a KRAS inhibitor, for preparation of a medicament for treatment of cancer.

The present application further provides an FGFR1 inhibitor and a KRAS inhibitor, for use in any of the methods described herein.

The present application further provides methods of treating cancer in a patient, comprising administering to said patient:

(i) pemigatinib, or a pharmaceutically acceptable salt thereof; and

(ii) sotorasib, or a pharmaceutically acceptable salt thereof.

The present application also provides use of pemigatinib, or a pharmaceutically acceptable salt thereof; and a KRAS inhibitor, for preparation of a medicament for treatment of cancer.

The present application further provides pemigatinib, or a pharmaceutically acceptable salt thereof; and a KRAS inhibitor, for use in any of the methods described herein.

The present application also provides use of pemigatinib, or a pharmaceutically acceptable salt thereof; and sotorasib, or a pharmaceutically acceptable salt thereof, for preparation of a medicament for treatment of cancer.

The present application further provides pemigatinib, or a pharmaceutically acceptable salt thereof; and sotorasib, or a pharmaceutically acceptable salt thereof, for use in any of the methods described herein.

### DESCRIPTION OF DRAWINGS

FIG. 1A displays Western blots of FGFR1, pFRS2a, E-cadherin, Vimentin and beta-actin in four indicated cell lines.

FIG. 1B displays Western blots of pFRS2a and beta-actin in LU99 cell lysate with the indicated treatment.

FIG. 2 displays Western blots of pERK, pFRS2a, and beta-actin in LU99 cells treated with a KRAS G12C inhibitor, Compound 2, with and without pemigatinib or Compound 1, for 24 hours.

FIG. 3 is a graph depicting the tumor volume of LU99 tumor bearing mice administered (i) vehicle; (ii) 20 mg/kg of AMG-510; (iii) 0.3 mg/kg of pemigatinib; or (iv) the combination of AMG-510 and pemigatinib at 20 mg/kg and 0.3 mg/kg, respectively.

FIG. 4 is a graph depicting the inhibition of pERK in LU99 tumors from mice receiving 20 mg/kg of AMG-510, 0.3 mg/kg of pemigatinib, or the combination of AMG-510 and pemigatinib at 20 mg/kg and 0.3 mg/kg, respectively.

FIG. 5 depicts Western blots of pFRS2a and beta-actin in LU99 cell lysate with MRTX849 treatment.

FIG. 6 depicts Western blots of pERK, pFRS2a, and beta-actin in LU99 cells treated with the KRAS G12C inhibitor, MRTX849 with and without pemigatinib or Compound 1 for 24 hours.

FIG. 7 is a graph depicting the tumor volume of LU99 tumor bearing mice administered (i) vehicle; (ii) 10 mg/kg of MRTX849; (iii) 0.3 mg/kg of pemigatinib; or (iv) the combination of MRTX849 and pemigatinib at 10 mg/kg and 0.3 mg/kg, respectively.

FIG. 8 is a graph depicting the inhibition of pERK in LU99 tumors from mice receiving 10 mg/kg of MRTX849, 0.3 mg/kg of pemigatinib, or the combination of MRTX849 and pemigatinib at 10 mg/kg and 0.3 mg/kg, respectively.

FIG. 9A depicts Western blots for FGFR1, FGFR2, FGFR3, and FGFR4 knockdown experiments.

FIG. 9B shows the inhibitory effect of FGFR1 and FGFR4 siRNA knockdown in combination with Compound 3 on LU99 cell proliferation after 120 h.

FIG. 9C shows the inhibitory effect of FGFR1 and FGFR4 siRNA knockdown in combination with AMG510 on LU99 cell proliferation after 120 h.

FIG. 10A depicts Western blots for FGFR2 and FGFR3 knockdown experiments. FIG. 10B shows the inhibitory effect of FGFR1, FGFR2 and FGFR3 siRNA knockdown in combination with Compound 2 on LU99 cell proliferation after 120 h.

FIG. 10C shows the inhibitory effect of FGFR1, FGFR2 and FGFR3 siRNA knockdown in combination with AMG510 on LU99 cell proliferation after 120 h.

FIG. 11A depicts Western blots for FGFR1, FGFR2, FGFR3, and FGFR4 after knockdown of FGR1 in order to evaluate possible compensation of FGFR2, FGFR3, and FGFR4.

FIG. 11B shows the inhibitory effect of FGFR1, FGFR2, FGFR3, and FGFR4 siRNA knockdown in combination with Compound 2 on LU99 cell proliferation after 120 h.

FIG. 12 shows the Western blot analysis of FGFR1, pERK, and B-Actin in MiaPaca2 KRAS G12C resistance clones.

FIG. 13A depicts Western blots for FGFR1, FGFR2, FGFR3, and FGFR4 knockdown experiments.

FIG. 13B shows the inhibitory effect of siRNA knockdown of single FGFR isoforms in combination with Compound 5 on A427 cell proliferation after 120 h.

FIG. 13C shows the inhibitory effect of siRNA knockdown of multiple FGFR isoforms in combination with Compound 5 on A427 cell proliferation after 120 h.

FIG. 13D shows the inhibitory effect of siRNA knockdown of single FGFR isoforms in combination with Compound 6 on A427 cell proliferation after 120 h.

FIG. 13E shows the inhibitory effect of siRNA knockdown of multiple FGFR isoforms in combination with Compound 6 on A427 cell proliferation after 120 h.

FIG. 13F shows the inhibitory effect of siRNA knockdown of single FGFR isoforms in combination with Compound 7 on A427 cell proliferation after 120 h.

FIG. 13G shows the inhibitory effect of siRNA knockdown of multiple FGFR isoforms in combination with Compound 7 on A427 cell proliferation after 120 h.

### DETAILED DESCRIPTION

The present application provides, inter alio, a method of treating cancer in a patient, comprising administering an FGFR1 inhibitor in combination with a KRAS inhibitor.

FGFR inhibitors

Pemigatinib, having the structure:

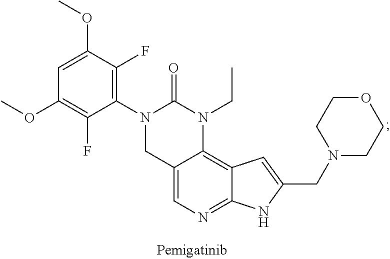 [SMILES: CCN1C(=O)N(c2c(F)c(OC)cc(OC)c2F)Cc2cnc3[nH]c(CN4CCOCC4)cc3c21]

is described in U.S. Pat. No. 9,611,267, the entirety of which is incorporated herein by reference. Pemigatinib is further described in US Publication Nos.: 2019/0337948 and 2020/0002338, the entireties of which are incorporated herein by reference. Pemigatinib as described herein can inhibit the activity of the FGFR1, FGFR2 and FGFR3 enzymes. For example, pemigatinib can be used to inhibit activity of an FGFR enzyme in a cell or in an individual or patient in need of inhibition of the enzyme by administering an inhibiting amount of pemigatinib to the cell, individual, or patient. As an FGFR inhibitor, pemigatinib is useful in the treatment of various diseases associated with abnormal expression or activity of the FGFR enzyme or FGFR ligands.

TAS-120 (futibatinib), having the structure:

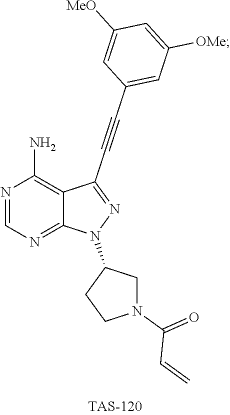 [SMILES: C=CC(=O)N1CC[C@H](n2nc(C#Cc3cc(OC)cc(OC)c3)c3c(N)ncnc32)C1]

is commercially available, i.e., Selleck Chemicals (https://www.selleckchem.com/products/tas-120.html). TAS-120 is further described in Chem. Med. Chem. 2019, 14, 494-500, which is incorporated herein by reference in its entirety. TAS-120 as described herein can inhibit the activity of the FGFR1, FGFR2, FGFR3 and FGFR4 enzymes.

Erdafitinib, having the structure:

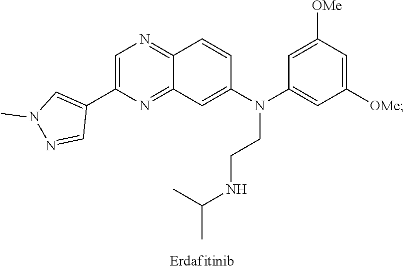 [SMILES: COc1cc(OC)cc(N(CCNC(C)C)c2ccc3ncc(-c4cnn(C)c4)nc3c2)c1]

is commercially available, i.e., Selleck Chemicals (https://www.selleckchem.com/products/jnj-42756493-erdafitinib.html). Erdafitinib is further described in Mol. Cancer Ther. 2017, 16(6), 1010-1020, which is incorporated herein by reference in its entirety. Erdafitinib as described herein can inhibit the activity of the FGFR1, FGFR2, FGFR3 and FGFR4 enzymes.

BGJ398 (infigratinib), having the structure:

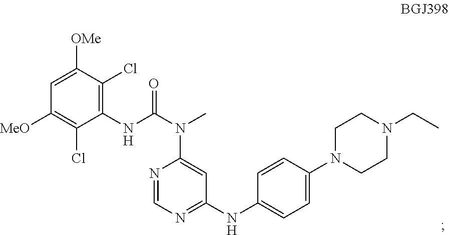 [SMILES: CCN1CCN(c2ccc(Nc3cc(N(C)C(=O)Nc4c(Cl)c(OC)cc(OC)c4Cl)ncn3)cc2)CC1]

is commercially available, i.e., Selleck Chemicals (https://www. sell eckchem com/products/bgj 398-nvp-bgj 398. html). BGJ398 is further described in J. Med. Chem. 2011, 54, 7066-7083, which is incorporated herein by reference in its entirety. BGJ398 as described herein can inhibit the activity of the FGFR1, FGFR2 and FGFR3 enzymes.

Compound 1 as described herein can inhibit the activity of the FGFR2 and FGFR3 enzymes, and is 40 times more selective for inhibition of FGFR2 and FGFR3 than for inhibition of FGFR1.

Compounds which inhibit FGFR will be useful in providing a means of preventing the growth or inducing apoptosis in tumors, particularly by inhibiting angiogenesis. The methods disclosed herein can be useful in treating or preventing proliferative disorders such as cancers. In particular, tumors with activating mutants of receptor tyrosine kinases or upregulation of receptor tyrosine kinases may be particularly sensitive to the methods described herein.

KRAS inhibitors

AMG-510 (sotorasib), having the structure:

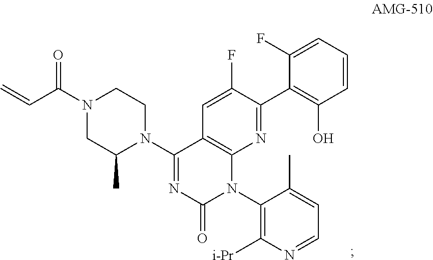 [SMILES: C=CC(=O)N1CCN(c2nc(=O)n(-c3c(C)ccnc3C(C)C)c3nc(-c4c(O)cccc4F)c(F)cc23)[C@@H](C)C1]

is commercially available, i.e., Selleck Chemicals (https://www.selleckchem.com/products/amg510.html). AMG-510 is further described in J. Med. Chem. 2020, 63, 52-65, which is incorporated herein by reference in its entirety. AMG-510 as described herein can inhibit the activity of the KRAS G12C mutated protein.

MRTX849 (adagrasib), having the structure:

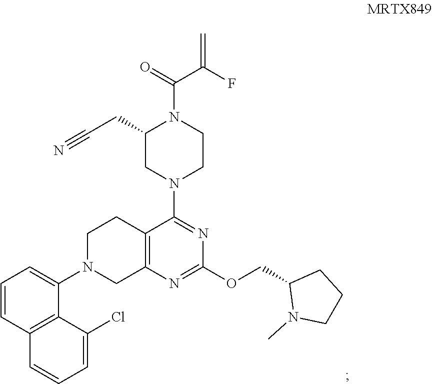 [SMILES: C=C(F)C(=O)N1CCN(c2nc(OC[C@@H]3CCCN3C)nc3c2CCN(c2cccc4cccc(Cl)c24)C3)C[C@@H]1CC#N]

is commercially available, i.e., Selleck Chemicals (https://www.selleckchem.com/products/mrtx849.html). MRTX849 is further described in J. Med. Chem. 2020, 63, 6679-6693, which is incorporated herein by reference in its entirety. MRTX849 as described herein can inhibit the activity of the KRAS G12C mutated protein.

2-((2S,4S)-4-(4-(3-(dimethylamino)-3-methylazetidin-1-yl)-6-fluoro-7-(5-fluoroquinolin-8-yl)-8-(trifluoromethyl)-1H-[1,2,3]triazolo[4,5-c]quinolin-1-yl)-1-((E)-4-methoxybut-2-enoyl)piperidin-2-yl)acetonitrile (Compound 2), having the structure:

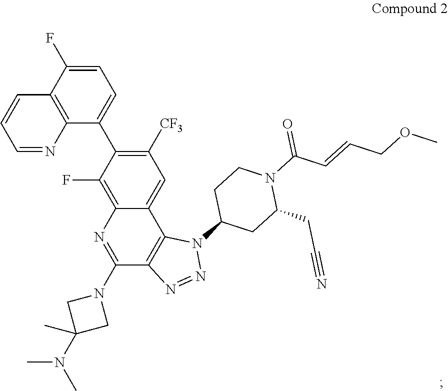 [SMILES: COC/C=C/C(=O)N1CC[C@H](n2nnc3c(N4CC(C)(N(C)C)C4)nc4c(F)c(-c5ccc(F)c6cccnc56)c(C(F)(F)F)cc4c32)C[C@H]1CC#N]

is disclosed in US Provisional Application No. US 63/219,274, which is incorporated herein by reference in its entirety. Compound 2 is also disclosed in US Provisional Application Nos. 63/292,774 and 63/310,811, each of which is incorporated herein by reference in its entirety. Compound 2 as described herein can inhibit the activity of the KRAS G12C mutated protein.

2-((2S,4S)-4-(4-(3-(dimethylamino)-3-methylazetidin-1-yl)-6-fluoro-7-(5-fluoroquinolin-8-yl)-8-(trifluoromethyl)-1H-[1,2,3]triazolo[4,5-c]quinolin-1-yl)-1-((E)-4-fluorobut enoyl)piperidin-2-yl)acetonitrile (Compound 3), having the structure:

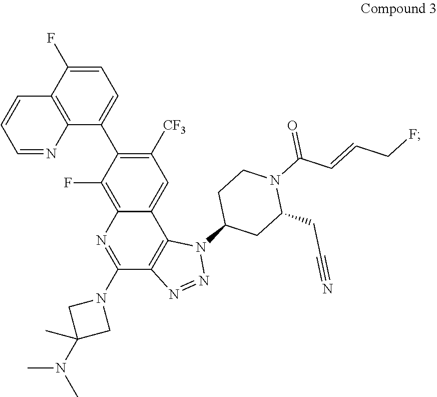 [SMILES: CN(C)C1(C)CN(c2nc3c(F)c(-c4ccc(F)c5cccnc45)c(C(F)(F)F)cc3c3c2nnn3[C@H]2CCN(C(=O)/C=C/CF)[C@H](CC#N)C2)C1]

is disclosed in US Provisional Application No. US 63/219,274, which is incorporated herein by reference in its entirety. Compound 3 as described herein can inhibit the activity of the KRAS G12C mutated protein.

2-((2S,4S)-1-acryloyl-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)piperidin-2-yl)acetonitrile (Compound 4), having the structure:

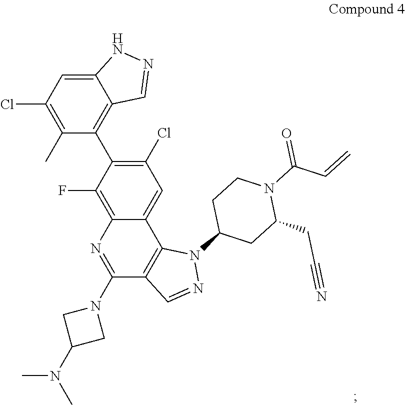 [SMILES: C=CC(=O)N1CC[C@H](n2ncc3c(N4CC(N(C)C)C4)nc4c(F)c(-c5c(C)c(Cl)cc6[nH]ncc56)c(Cl)cc4c32)C[C@H]1CC#N]

is disclosed in International Application Publication No. WO 2021/211864 A1, which is incorporated herein by reference in its entirety. Compound 4 as described herein can inhibit the activity of the KRAS G12C mutated protein.

3-(1-((1R,4R,5S)-2-Azabicyclo[2.1.1]hexan-5-yl)-6-fluoro-7-(7-fluoro-3-hydroxynaphthalen-1-yl)-2-methyl-4-(1H-1,2,4-triazol-1-yl)-1H-pyrrolo[3,2-c]quinolin-8-yl)propanenitrile (Compound 5), having the structure:

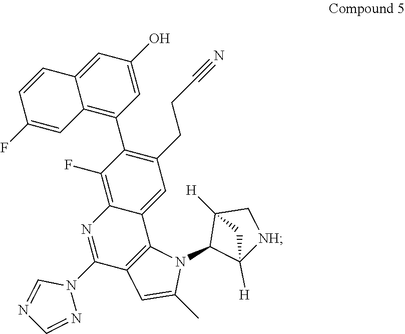 [SMILES: Cc1cc2c(-n3cncn3)nc3c(F)c(-c4cc(O)cc5ccc(F)cc45)c(CCC#N)cc3c2n1[C@H]1[C@H]2CN[C@@H]1C2]

is disclosed in International Application No. PCT/US2022/78048, which is incorporated herein by reference in its entirety. Compound 5 as described herein can inhibit the activity of the KRAS G12D mutated protein.

8-(24(R)-1-Acetylpyrrolidin-2-yl)-1-((1R,4R,5S)-2-azabicyclo[2.1.1]hexan-5-yl)-6-fluoro-8-methyl-4-(S)-1-((S)-1-methylpyrrolidin-2-yl)ethoxy)-1H-pyrrolo[3,2-c]quinolin-7-yl)-1,2,3,4-tetrahydronaphthalene-1-carbonitrile (Compound 6), having the structure:

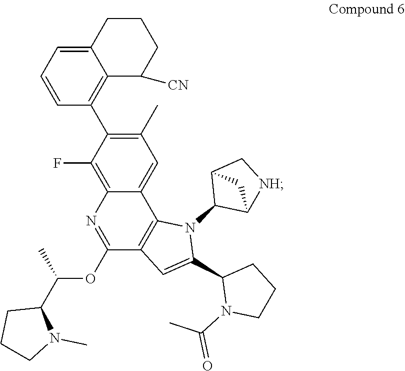 [SMILES: CC(=O)N1CCC[C@@H]1c1cc2c(O[C@@H](C)[C@@H]3CCCN3C)nc3c(F)c(-c4cccc5c4C(C#N)CCC5)c(C)cc3c2n1[C@H]1[C@H]2CN[C@@H]1C2]

is disclosed in Provisional Application No. 63/368,124, which is incorporated herein by reference in its entirety. Compound 6 as described herein can inhibit the activity of the KRAS G12D mutated protein.

3-(1-((1R,4R,5S)-2-azabicyclo[2.1.1]hexan-5-yl)-4-ethoxy-24(4-ethyl-2-oxopiperazin-1-yl)methyl)-6-fluoro-7-(3-hydroxynaphthalen-1-yl)-1H-pyrrolo[3,2-c]quinolin-8-yl)propanenitrile (Compound 7), having the structure:

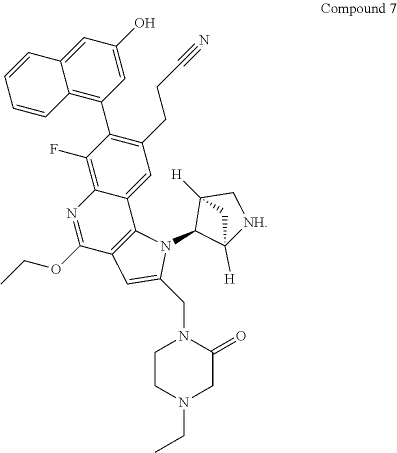 [SMILES: CCOc1nc2c(F)c(-c3cc(O)cc4ccccc34)c(CCC#N)cc2c2c1cc(CN1CCN(CC)CC1=O)n2[C@H]1[C@H]2CN[C@@H]1C2]

Compound 7 as described herein can inhibit the activity of the KRAS G12D mutated protein.

Compounds of Formula (I):

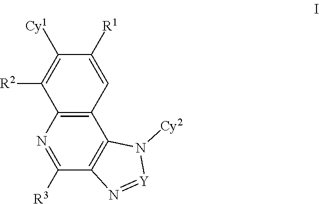

or a pharmaceutically acceptable salt thereof, wherein:

Y is N or CH;

R¹ is selected from Cl, CH₃, CH₂F, CHF₂, and CF₃;

Cy¹ is selected from

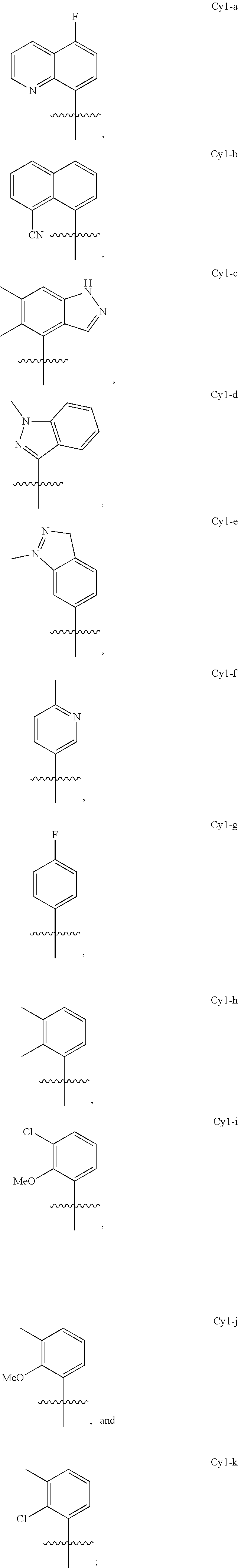

R² is selected from F and Cl;

R³ is selected from

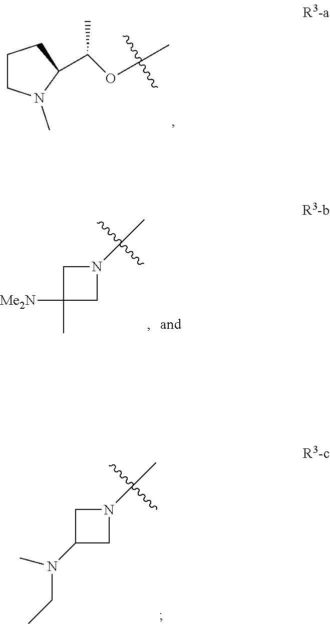

and,

Cy² is selected from

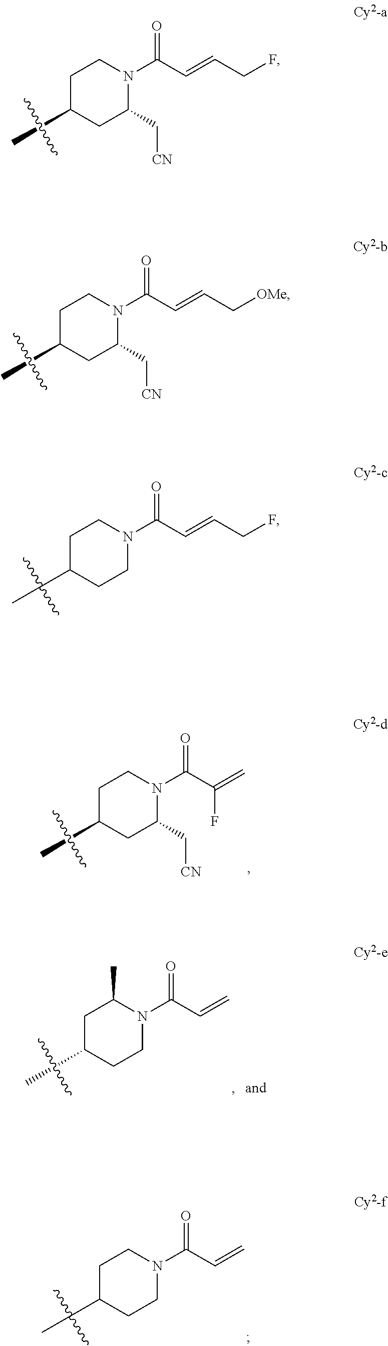

provided that the compound of Formula (I) is other than

2-((2S,4S)-4-(7-(2-chloro-3-methylphenyl)-4-(3-(dimethylamino)-3-
methylazetidin-1-yl)-6-fluoro-8-methyl-1H-[1,2,3]triazolo[4,5-c]quinolin-
1-yl)-1-((E)-4-fluorobut-2-enoyl)piperidin-2-yl)acetonitrile and
2-((2S,4S)-4-(4-(3-(dimethylamino)-3-methylazetidin-1-yl)-7-(2,3-
dimethylphenyl)-6-fluoro-8-methyl-1H-[1,2,3]triazolo[4,5-c]quinolin-
1-yl)-1-((E)-4-fluorobut-2-enoyl)piperidin-2-yl)acetonitrile

are disclosed in US Provisional Application No. US 63/219,274, which is incorporated herein by reference in its entirety. Compounds of Formula (I) as described herein can inhibit the activity of the KRAS G12C mutated protein.

Compounds of Formula (II):

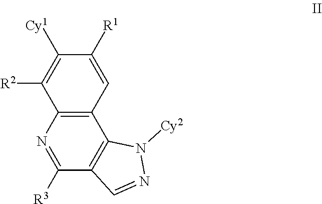

or a pharmaceutically acceptable salt thereof, wherein:

R¹ is selected from Cl, CH₃, CH₂F, CHF₂, and CF₃;

Cy¹ is selected from

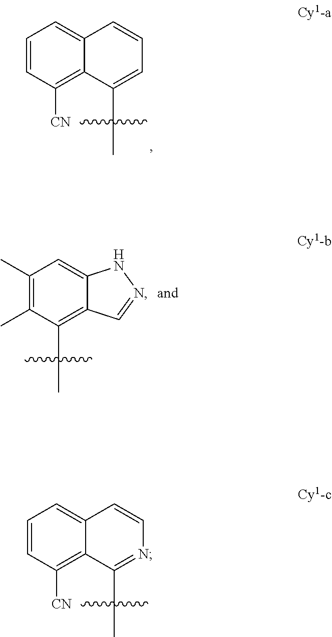 [SMILES: *c1cccc2cccc(C#N)c12 ; *c1c(C)c(C)cc2[nH]ncc12 ; *c1nccc2cccc(C#N)c12]

R² is selected from F and Cl;

R³ is selected from

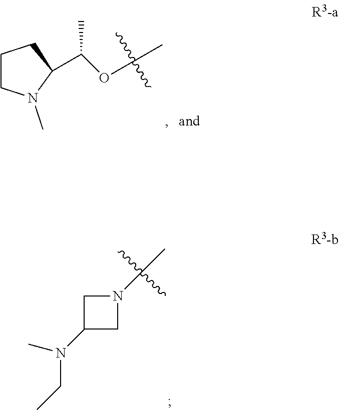

and,

Cy² is selected from

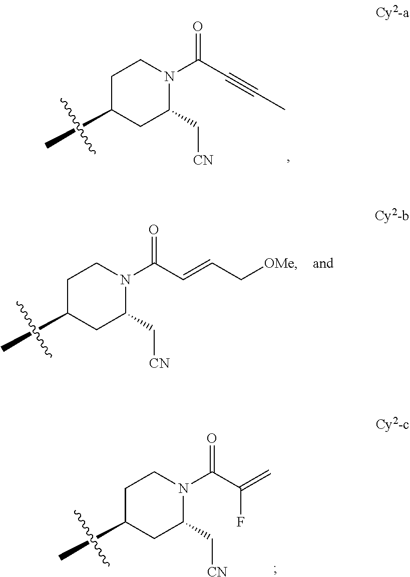

provided that the compound of Formula (II) is other than.

8-(1-((2S,4S)-2-(cyanomethyl)-1-(2-fluoroacryloyl)piperidin-4-yl)-6-fluoro-8-methyl-4-((S)-
1-((S)-1-methylpyrrolidin-2-yl)ethoxy)-1H-pyrazolo[4,3-c]quinolin-7-yl)-1-naphthonitrile,
8-(1-((2S,4S)-2-(cyanomethyl)-1-((E)-4-methoxybut-2-enoyl)piperidin-4-yl)-6-fluoro-8-
methyl-4-((S)-1-((S)-1-methylpyrrolidin-2-yl)ethoxy)-1H-pyrazolo[4,3-c]quinolin-7-yl)-1-
naphthonitrile,
2-((2S,4S)-4-(7-(5,6-dimethyl-1H-indazol-4-yl)-6-fluoro-8-methyl-4-((S)-1-((S)-1-
methylpyrrolidin-2-yl)ethoxy)-1H-pyrazolo[4,3-c]quinolin-1-yl)-1-(2-
fluoroacryloyl)piperidin-2-yl)acetonitrile,
2-((2S,4S)-1-(but-2-ynoyl)-4-(7-(5,6-dimethyl-1H-indazol-4-yl)-6-fluoro-8-methyl-4-((S)-1-
((S)-1-methylpyrrolidin-2-yl)ethoxy)-1H-pyrazolo[4,3-c]quinolin-1-yl)piperidin-2-
yl)acetonitrile, and
2-((2S,4S)-4-(7-(5,6-dimethyl-1H-indazol-4-yl)-6-fluoro-8-methyl-4-((S)-1-((S)-1-
methylpyrrolidin-2-yl)ethoxy)-1H-pyrazolo[4,3-c]quinolin-1-yl)-1-((E)-4-methoxybut-2-
enoyl)piperidin-2-yl)acetonitrile

are disclosed in US Provisional Application No. US 63/261,982, which is incorporated herein by reference in its entirety. Compounds of Formula (II) as described herein can inhibit the activity of the KRAS G12C mutated protein.

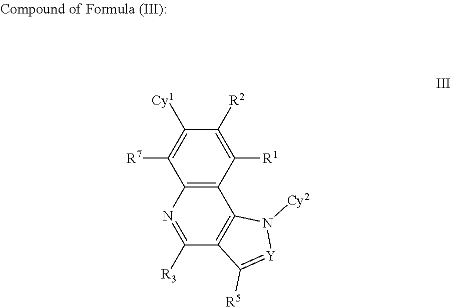

or a pharmaceutically acceptable salt thereof, wherein:

Y is N or CR⁶;

R¹ is selected from H, C₁₋₃ alkyl, C₁₋₃ haloalkyl, cyclopropyl, halo, D, CN, and OR^{a1}; wherein said C₁₋₃ alkyl and cyclopropyl are each optionally substituted with 1 or 2 substituents independently selected from Rg;

R² is selected from H, C₁₋₃ alkyl, C₁₋₃ haloalkyl, 4-6 membered heterocycloalkyl, phenyl, 5-6 membered heteroaryl, 4-6 membered heterocycloalkyl-C₁₋₃ alkylene, phenyl-C₁₋₃ alkylene, 5-6 membered heteroaryl-Cl-3 alkylene, halo, D, CN, and ORa²; wherein said C₁₋₃ alkyl, 4-6 membered heterocycloalkyl, phenyl, 5-6 membered heteroaryl, 4-6 membered heterocycloalkyl-C₁₋₃ alkylene, phenyl-C₁₋₃ alkylene, 5-6 membered heteroaryl-C₁₋₃ alkylene are each optionally substituted with 1 or 2 substituents independently selected from R^{g};

Cy¹ is selected from C3-lo cycloalkyl, 4-10 membered heterocycloalkyl, C₆₋₁₀ aryl and 6-10 membered heteroaryl; wherein the 4-10 membered heterocycloalkyl and 6-10 membered heteroaryl each has at least one ring-forming carbon atom and 1, 2, 3, or 4 ring-forming heteroatoms independently selected from N, O, and S; wherein a ring-forming carbon atom of 6-10 membered heteroaryl and 4-10 membered heterocycloalkyl is optionally substituted by oxo to form a carbonyl group; and wherein the C₃₋₁₀ cycloalkyl, 4-10 membered heterocycloalkyl, C₆₋₁₀ aryl and 6-10 membered heteroaryl are each optionally substituted with 1, 2, 3, or 4 substituents independently selected from R¹⁰;

R³ is selected from H, C₁₋₃ alkyl, C₁₋₃ haloalkyl, C₃₋₆ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, halo, D, CN, OR ^{f3}, C(O)NR^{c3}R^{d3}, NR^{c3}R^{j3}, and NR^{c3}C(O)R^{b3}; wherein said C₁₋₃ alkyl, C₃₋₆ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene are each optionally substituted with 1, 2, or 3 substituents independently selected from R³⁰;

R⁵ is selected from H, C₁₋₃ alkyl, C₁₋₃ haloalkyl, cyclopropyl, halo, D, CN, and OR^{a5}; wherein said C₁₋₃ alkyl and cyclopropyl are each optionally substituted with 1 or 2 substituents independently selected from Rg;

R⁶ is selected from H, C₁₋₃ alkyl, C₁₋₃ haloalkyl, C₃₋₆ cycloalkyl, 4-9 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, halo, D, CN, OR^{a6}, and C(P)NR^{c6}R^{d6}; wherein said C₁₋₃ alkyl, C₃₋₆ cycloalkyl, 4-9 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene are each optionally substituted with 1 or 2 substituents independently selected from R⁶⁰;

R⁷ is selected from H, C₁₋₃ alkyl, C₁₋₃ haloalkyl, cyclopropyl, halo, D, CN, and OR^{a7}; wherein said C₁₋₃ alkyl and cyclopropyl are each optionally substituted with 1 or 2 substituents independently selected from R^{g};

Cy² is selected from

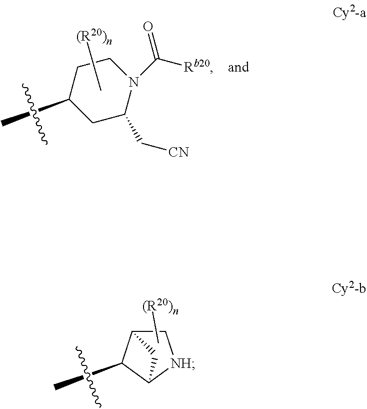

wherein n is 0, 1, or 2;

each R¹⁰ is independently selected from C₁₋₃ alkyl, C₁₋₃ haloalkyl, halo, D, CN, OR^{a10}, C(O)R^{b10}, C(O)NR^{c10}R^{d10}, C(O)OR^{a10}, NR^{c10}R^{d10}, and S(O)₂R^{b10};

each R²⁰ is independently selected from C₁₋₃ alkyl, C₁₋₃ haloalkyl, halo, D, CN, and OR^{a20};

each R³⁰ is independently selected from C₁₋₃ alkyl, C₁₋₃ haloalkyl, C₃₋₆ cycloalkyl, 4-6 membered heterocycloalkyl, phenyl, 5-6 membered heteroaryl, halo, D, CN, ORa³⁰, C(O)R^{b30}, C(O)NRc³⁰R^{d30}, C(O)ORa³⁰, NR^{c30}R^{d30} and S(O)₂R^{b30}; wherein said C₁₋₃ alkyl, C₃₋₆ cycloalkyl, 4-6 membered heterocycloalkyl, phenyl, and 5-6 membered heteroaryl are each optionally substituted with 1 or 2 substituents independently selected from R³¹;

each R³¹ is independently selected from C₁₋₃ alkyl, C₁₋₃ haloalkyl, halo, D, CN, ORa³¹, C(O)R^{b31}, C()NR^{c31}R^{d31}, C(O)NR^{a31}, NR^{c31}R^{d31}, and S(O)₂R^{b31};

each R³³ is independently selected from C₁₋₃ alkyl, C₁₋₃ haloalkyl, C₃₋₆ cycloalkyl, 4-membered heterocycloalkyl, 6-membered heterocycloalkyl, phenyl, 5-6 membered heteroaryl, halo, D, CN, OR^{a30}, C(O)NR^{c30}R^{d30}, and NR^{c30}R^{d30}; wherein said C₁₋₃ alkyl, C₃₋₆ cycloalkyl, 4-membered heterocycloalkyl, 6-membered heterocycloalkyl, phenyl, and 5-6 membered heteroaryl are each optionally substituted with 1 or 2 substituents independently selected from R³¹;

each R⁶⁰ is independently selected from C₁₋₃ alkyl, C₁₋₃ haloalkyl, 4-6 membered heterocycloalkyl, 5-6 membered heteroaryl, halo, D, CN, OR^{a60}, C(O)R^{b60}, C(O)NR^{c60}R^{d60}, NR^{c60}C(O)R^{b60}, C(O)OR^{a60}, NR^{c60}C(O)OR^{a60}, NR^{c60}R^{d60}, NR^{c60}S(O)₂R^{b60}; wherein said C₁₋₃ alkyl, 4-6 membered heterocycloalkyl, and 5-6 membered heteroaryl are each optionally substituted with 1 or 2 substituents independently selected from R⁶¹;

each R⁶¹ is independently selected from C₁₋₃ alkyl, C₁₋₃ haloalkyl, halo, D, CN, ORa⁶¹, and NR^{c61}R^{d61};

R^{a1} is selected from H, C₁₋₃ alkyl, and C₁₋₃ haloalkyl;

each R is independently selected from H, C₁₋₃ alkyl, and C₁₋₃ haloalkyl;

each R^{b3}, Rc³ and R^{d3} is independently selected from H, C₁₋₃ alkyl, C₁₋₃ haloalkyl, C₃₋₆ cycloalkyl, 4-6 membered heterocycloalkyl, phenyl and 5-6 membered heteroaryl; wherein said, C₁₋₃ alkyl, C₃₋₆ cycloalkyl, 4-6 membered heterocycloalkyl, phenyl and 5-6 membered heteroaryl are each optionally substituted with 1, 2, or 3 substituents independently selected from R³⁰;

or R^{c3} and R^{d3} attached to the same N atom, together with the N atom to which they are attached, form a 4-, 5-, or 6-membered heterocycloalkyl group optionally substituted with 1, 2, or 3 substituents independently selected from R³⁰;

R^{j3} is selected from C₁₋₃ alkyl, C₁₋₃ haloalkyl, C₃₋₆ cycloalkyl, 4-6 membered heterocycloalkyl, phenyl and 5-6 membered heteroaryl; wherein said, C₁₋₃ alkyl, C₃₋₆ cycloalkyl, 4-6 membered heterocycloalkyl, phenyl and 5-6 membered heteroaryl are each optionally substituted with 1, 2, or 3 sub stituents independently selected from R³⁰;

or R^{c3} and R^{j3} attached to the same N atom, together with the N atom to which they are attached, form a 4-, 5-, or 6-membered heterocycloalkyl group optionally substituted with 1, 2, or 3 substituents independently selected from R³⁰;

R^{f3} is selected from C₁₋₃ haloalkyl, C₃₋₆ cycloalkyl, 4-6 membered heterocycloalkyl, phenyl and 5-6 membered heteroaryl; wherein said C₁₋₃ haloalkyl, C₃₋₆ cycloalkyl, 4-6 membered heterocycloalkyl, phenyl, and 5-6 membered heteroaryl are each optionally substituted with 1, 2, or 3 substituents independently selected from R³⁰; or

R^{f3} is selected from

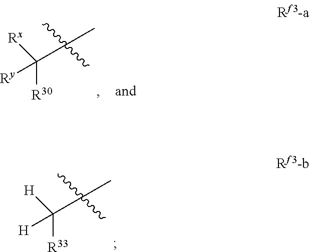

wherein IV is H or C₁₋₂ alkyl and Ry is C₁₋₂ alkyl;

or R^{x} and R^{y}, together with the C atom to which they are attached, form a 3-, or 4-membered cycloalkyl group;

R^{a5} is selected from H, C₁₋₃ alkyl, and C₁₋₃ haloalkyl;

each R^{a6}, R^{c6} and R^{d6} is independently selected from H, C₁₋₃ alkyl, C₁₋₃ haloalkyl, C₃₋₆ cycloalkyl, 4-6 membered heterocycloalkyl, phenyl and 5-6 membered heteroaryl; wherein said C₁₋₃ alkyl, C₃₋₆ cycloalkyl, 4-6 membered heterocycloalkyl, phenyl and 5-6 membered heteroaryl are each optionally substituted with 1 or 2 substituents independently selected from R⁶⁰;

R^{a7} is selected from H, C₁₋₃ alkyl, and C₁₋₃ haloalkyl;

each R^{a10}, R^{b10}, R^{c10} and R^{d10} independently selected from H, C₁₋₃ alkyl, and C₁₋₃ haloalkyl;

each R^{a20} is independently selected from H, C₁₋₃ alkyl, and C₁₋₃ haloalkyl;

R^{b20} is selected from NH₂, C₁₋₃ alkyl, and C₁₋₃ haloalkyl;

each R^{a30}, R^{b30}, R^{c30} and R^{d30} is independently selected from H, C₁₋₃ alkyl, and C₁₋₃ haloalkyl;

each R^{a31}, R^{b31}, R^{c31} and R^{d31} is independently selected from H, C₁₋₃ alkyl, and C₁₋₃ haloalkyl;

each R^{a60}, R^{b60}, R^{c60} and R^{d60} is independently selected from H, C₁₋₃ alkyl, C₁₋₃ haloalkyl, C₃₋₆ cycloalkyl, 4-6 membered heterocycloalkyl, and 5-6 membered heteroaryl; wherein said C₁₋₃ alkyl, C₃₋₆ cycloalkyl, 4-6 membered heterocycloalkyl, and 5-6 membered heteroaryl are each optionally substituted with 1 or 2 substituents independently selected from R⁶¹;

or any R^{c60} and R^{d60} attached to the same N atom, together with the N atom to which they are attached, form a 4-, 5-, or 6-membered heterocycloalkyl group optionally substituted with 1 or 2 substituents independently selected from R⁶¹; and

each R^{a61}, R^{c61}, and R^{d61}, is independently selected from H, C₁₋₃ alkyl, and C₁₋₃ haloalkyl; and

each R^{g} is independently selected from D, OH, CN, halo, C₁₋₃ alkyl, C₁₋₃ haloalkyl, C₁₋₃ alkoxy, C₁₋₃ haloalkoxy, amino, C₁₋₃ alkylamino, and di(C₁₋₃ alkyl)amino;

provided that the compound of Formula (III) is other than,

3-(1-((1R,4R,5S)-2-azabicyclo[2.1.1]hexan-5-yl)-8-(2-cyanoethyl)-4-ethoxy-6-fluoro-7-(3-hydroxynaphthalen-1-yl)-1H-pyrrolo[3,2-c]quinolin-2-yl)-N,N-dimethylpropanamide, are disclosed in PCT Application No. PCT/US22/78048, which is incorporated herein by reference in its entirety. Compounds of Formula (III) as described herein can inhibit the activity of the KRAS G12D mutated protein.

KRAS inhibitors are useful in the treatment of various diseases associated with abnormal expression or activity of KRAS. Compounds which inhibit KRAS will be useful in providing a means of preventing the growth or inducing apoptosis in tumors, or by inhibiting angiogenesis. It is therefore anticipated that compounds of the present disclosure will prove useful in treating or preventing proliferative disorders such as cancers. In particular, tumors with activating mutants of receptor tyrosine kinases or upregulation of receptor tyrosine kinases may be particularly sensitive to the inhibitors.

Provided herein is a method of treating cancer in a patient, comprising administering to said patient:

(i) an FGFR1 inhibitor; and

(ii) a KRAS inhibitor.

In some embodiments, the FGFR1 inhibitor is selected from pemigatinib, futibatinib, erdafitinib and infigratinib, or a pharmaceutically acceptable salt thereof. In some embodiments, the FGFR1 inhibitor is pemigatinib, or a pharmaceutically acceptable salt thereof In some embodiments, the FGFR1 inhibitor is futibatinib, or a pharmaceutically acceptable salt thereof.

In some embodiments, the FGFR1 inhibitor is erdafitinib, or a pharmaceutically acceptable salt thereof. In some embodiments, the FGFR1 inhibitor is infigratinib, or a pharmaceutically acceptable salt thereof. In some embodiments, the FGFR1 inhibitor further inhibits FGFR2, FGFR3, or a combination thereof.

In some embodiments, the KRAS inhibitor is a compound of Formula (I):

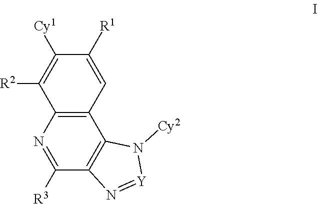

or a pharmaceutically acceptable salt thereof, wherein:

Y is N or CH;

R¹ is selected from Cl, CH₃, CH₂F, CHF₂, and CF₃;

Cy¹ is selected from

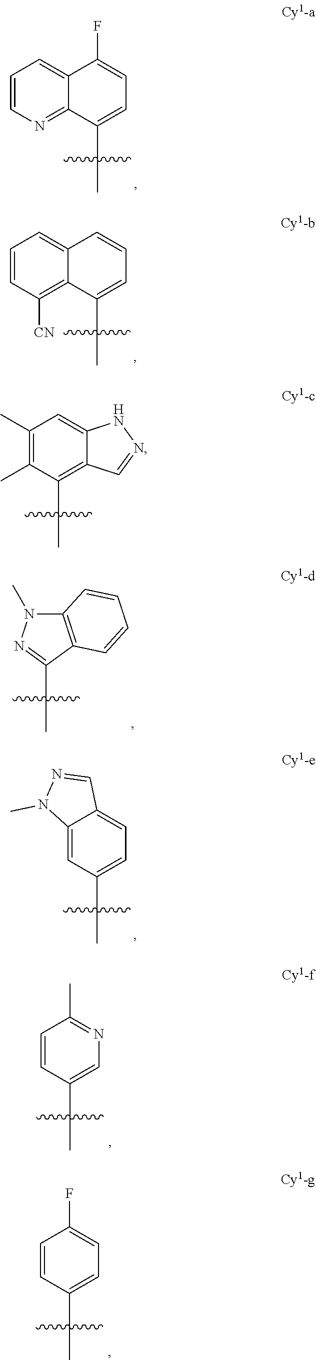

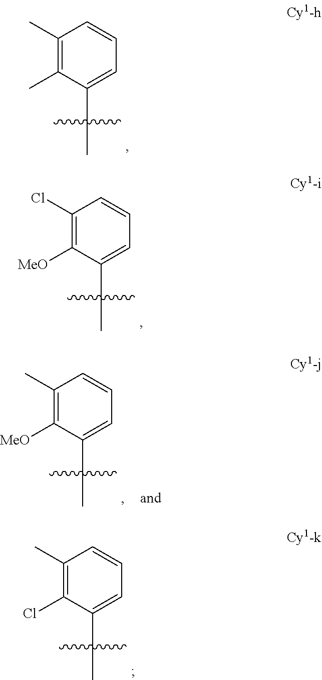

R² is selected from F and Cl;

R³ is selected from

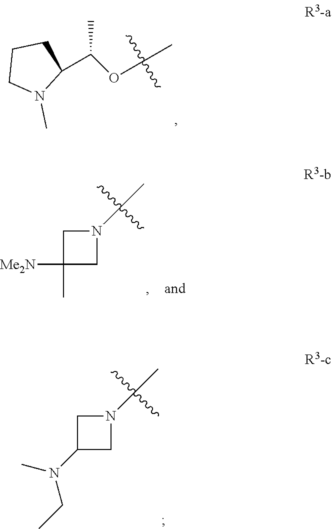

and,

Cy² is selected from

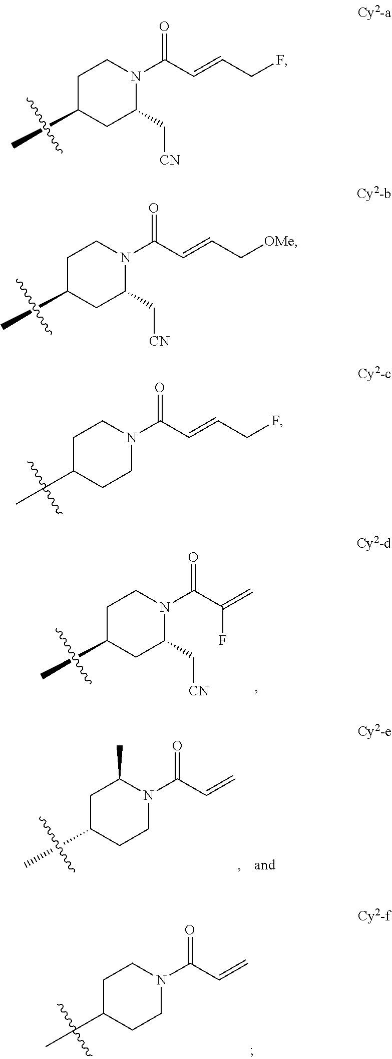

provided that the compound of Formula (I) is other than

2-((2S,4S)-4-(7-(2-chloro-3-methylphenyl)-4-(3-(dimethylamino)-3-
methylazetidin-1-yl)-6-fluoro-8-methyl-1H-[1,2,3]triazolo[4,5-c]quinolin-
1-yl)-1-((E)-4-fluorobut-2-enoyl)piperidin-2-yl)acetonitrile and
2-((2S,4S)-4-(4-(3-(dimethylamino)-3-methylazetidin-1-yl)-7-(2,3-
dimethylphenyl)-6-fluoro-8-methyl-1H-[1,2,3]triazolo[4,5-c]quinolin-
1-yl)-1-((E)-4-fluorobut-2-enoyl)piperidin-2-yl)acetonitrile.

In some embodiments, the compound of Formula (I) or the pharmaceutically acceptable salt thereof is selected from:

2-(2S,4S)-4-(4-(3-(dimethylamino)-3-methylazetidin-1-yl)-6-fluoro-7-(5-fluoroquinolin-8-yl)-8-(trifluoromethyl)-1H-[1,2,3]triazolo[4,5-c]quinolin-1-yl)-1-((E)-4-fluorobut-2-enoyl)piperidin-2-yl)acetonitrile;

2-((2S,4S)-4-(4-(3-(dimethylamino)-3-methylazetidin-1-yl)-6-fluoro-7-(2-methoxy-3-methylphenyl)-8-(trifluoromethyl)-1H-[1,2,3]triazolo[4,5-c]quinolin-1-yl)-14E)-4-fluorobut-2-enoyl)piperidin-2-yl)acetonitrile;

2-((2S,4S)-4-(7-(3-chloro-2-methoxyphenyl)-4-(3-(dimethylamino)-3-methylazetidin-1-yl)-6-fluoro-8-(trifluoromethyl)-1H-[1,2,3]triazolo[4,5-c]quinolin-1-yl)-1-((E)-4-fluorobut-2-enoyl)piperidin-2-yl)acetonitrile;

2-((2S,4S)-4-(6-fluoro-7-(5-fluoroquinolin-8-yl)-4-(S)-1-(S)-1-methylpyrrolidin-2-yl)ethoxy)-8-(trifluoromethyl)-1H-[1,2,3]triazolo[4,5-c]quinolin-1-yl)-1-((E)-4-fluorobut-2-enoyl)piperidin-2-yl)acetonitrile;

1-(4-(6-fluoro-7-(5-fluoroquinolin-8-yl)-4-((S)-1-((S)-1-methylpyrrolidin-2-yl)ethoxy)-8-(trifluoromethyl)-1H-[1,2,3]triazolo[4,5-c]quinolin-1-yl)piperidin-1-yl)prop-2-en-1-one;

2-((2S,4S)-4-(4-(3-(dimethylamino)-3-methylazetidin-1-yl)-'7-(2,3-dimethylphenyl)-6-fluoro-8-methyl-1H-[1,2,3]triazolo[4,5-c]quinolin-1-yl)-1-((E)-4-methoxybut-2-enoyl)piperidin-2-yl)acetonitrile;

2-((2S,4S)-4-(6-fluoro-8-methyl-7-(1-methyl-1H-indazol-6-yl)-4-((S)-1-(S)-1-methylpyrrolidin-2-yl)ethoxy)-1H-[1,2,3 ]triazolo[4, 5-c]quinolin-1-yl)-1-((E)-4-fluorobut-2-enoyl)piperidin-2-yl)acetonitrile;

2-((2S,4S)-4-(6-fluoro-8-methyl-7-(6-methylpyridin-3-yl)-4-((S)-1-((S)-1-methylpyrrolidin-2-yl)ethoxy)-1H-[1,2,3 ]triazolo[4, 5-c]quinolin-1-yl)-1-((E)-4-fluorobut-2-enoyl)piperidin-2-yl)acetonitrile;

2-((2S,4S)-4-(6-fluoro-8-methyl-7-(1-methyl-1H-indazol-3-yl)-4-((S)-1-((S)-1-methylpyrrolidin-2-yl)ethoxy)-1H-[1,2,3]triazolo[4,5-c]quinolin-1-yl)-1-(E)-4-methoxybut-2-enoyl)piperidin-2-yl)acetonitrile;

2-((2S,4S)-4-(6-fluoro-7-(4-fluorophenyl)-8-methyl-4-((S)-1-((S)-1-methylpyrrolidin-2-yl)ethoxy)-1H-[1,2,3 ]triazolo[4, 5-c]quinolin-1-yl)-1-((E)-4-methoxybut-2-enoyl)piperidin-2-yl)acetonitrile;

8-(1-(1-acryloylpiperidin-4-yl)-6-fluoro-8-methyl-4-(S)-1-(S)-1-methylpyrrolidin yl)ethoxy)-1H-[1,2,3]triazolo[4,5-c]quinolin-7-yl)-1-naphthonitrile;

2-((2S,4S)-4-(7-(2-chloro-3-methylphenyl)-4-(3-(dimethylamino)-3-methylazetidin-1-yl)-6-fluoro-8-methyl-1H-imidazo[4,5-c]quinolin-1-yl)-1-((E)-4-fluorobut-2-enoyl)piperidin yl)acetonitrile;

2-((2S,4S)-4-(7-(2-chloro-3-methylphenyl)-6-fluoro-8-methyl-4-((S)-1-((S)-1-methylpyrrolidin-2-yl)ethoxy)-1H-imidazo[4,5-c]quinolin-1-yl)-1-(2-fluoroacryloyl)piperidin-2-yl)acetonitrile;

8-(1-(2R,4S)-1-acryloyl-2-methylpiperidin-4-yl)-6-fluoro-8-methyl-4-(S)-1-(S)-1-methylpyrrolidin-2-yl)ethoxy)-1H-[1,2,3]triazolo[4,5-c]quinolin-7-yl)-1-naphthonitrile;

2-((2S,4S)-4-(7-(5, 6-dimethyl-1H-indazol-4-yl)-4-(3-(ethyl(methyl)amino)azetidin-1-yl)-6-fluoro-8-methyl-1H-[1,2,3]triazolo[4,5-c]quinolin-1-yl)-1-(2-fluoroacryloyl)piperidin-2-yl)acetonitrile;

8-(6-fluoro-1-(1-(E)-4-fluorobut-2-enoyl)piperidin-4-yl)-8-methyl-4-((S)-1-((S)-1-methylpyrrolidin-2-yl)ethoxy)-1H-[1,2,3]triazolo[4,5-c]quinolin-7-yl)-1-naphthonitrile;

8-(1-((2S,4S)-2-(cyanomethyl)-1-(2-fluoroacryloyl)piperidin-4-yl)-4-(3-(dimethylamino)-3-methylazetidin-1-yl)-6-fluoro-8-methyl-1H-[1,2,3]triazolo[4,5-c]quinolin-7-yl)-1-naphthonitrile;

2-((2S,4S)-4-(6, 8-dichloro-4-(3-(dimethylamino)-3-methylazetidin-1-yl)-7-(5-fluoroquinolin-8-yl)-1H-[1,2,3]triazolo[4,5-c]quinolin-1-yl)-1-((E)-4-methoxybut-2-enoyl)piperidin-2-yl)acetonitrile;

2-((2S,4S)-4-(6, 8-dichloro-4-(3-(dimethylamino)-3-methylazetidin-1-yl)-'7-(5-fluoroquinolin-8-yl)-1H-[1,2,3]triazolo[4,5-c]quinolin-1-yl)-1-((E)-4-fluorobut-2-enoyl)piperidin-2-yl)acetonitrile; and

2-((2S,4S)-4-(4-(3-(dimethylamino)-3-methylazetidin-1-yl)-6-fluoro-7-(5-fluoroquinolin-8-yl)-8-(trifluoromethyl)-1H-[1,2,3]triazolo[4,5-c]quinolin-1-yl)-1-(E)-4-methoxybut-2-enoyl)piperidin-2-yl)acetonitrile;

or a pharmaceutically acceptable salt thereof.

In an embodiment of Formula (I), or a pharmaceutically acceptable salt thereof,

Y is N or CH;

R¹ is selected from Cl, CH₃, CH₂F, CHF₂, and CF₃;

Cy¹ is selected from

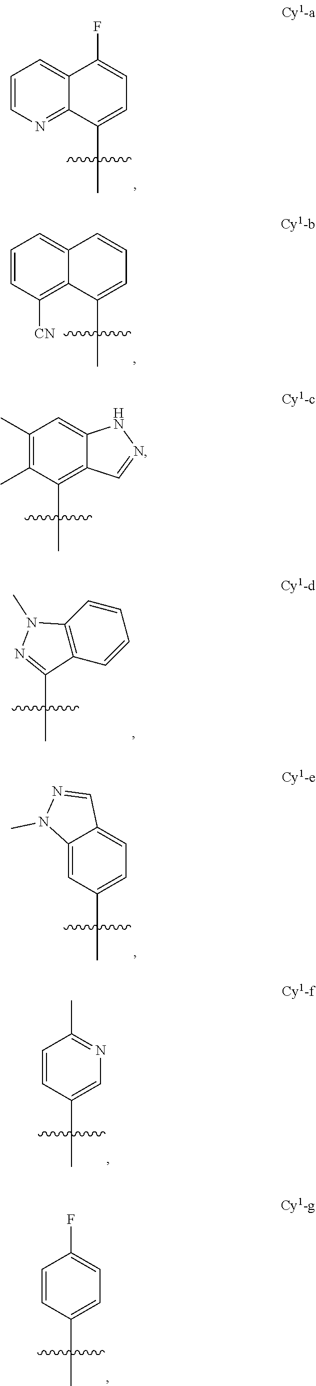

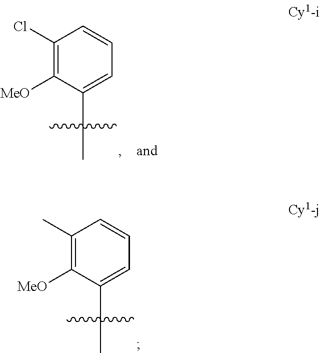

R² is selected from F and Cl;

R³ is selected from

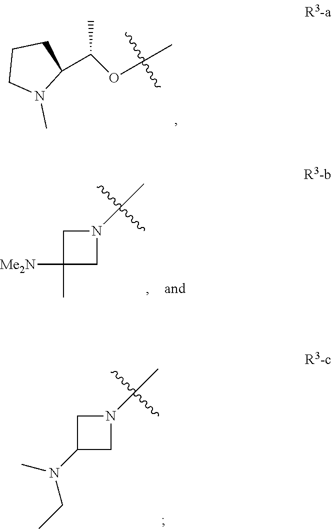

and,

Cy² is selected from

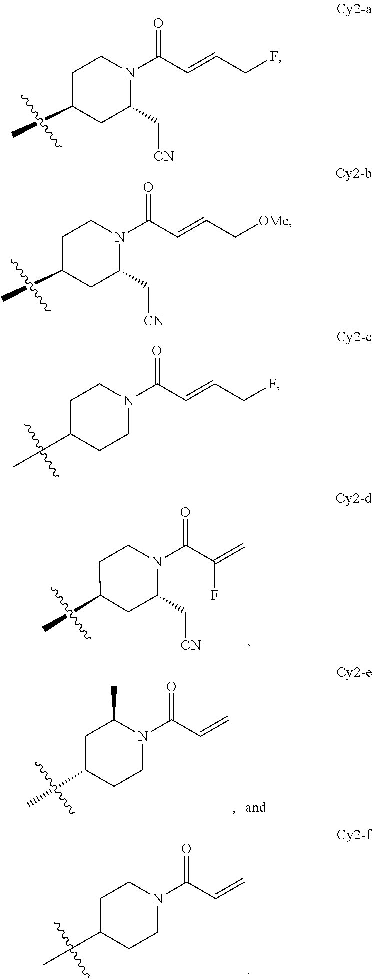

In some embodiments, the compound of Formula (I) or the pharmaceutically acceptable salt thereof is selected from:

2-((2S,4S)-4-(4-(3-(dimethylamino)-3-methylazetidin-1-yl)-6-fluoro-7-(5-fluoroquinolin-8-yl)-8-(trifluoromethyl)-1H-[1,2,3]triazolo[4,5-c]quinolin-1-yl)-1-((E)-4-fluorobut-2-enoyl)piperidin-2-yl)acetonitrile;

2-((2S,4S)-4-(4-(3-(dimethylamino)-3-methylazetidin-1-yl)-6-fluoro-7-(2-methoxy-3-methylphenyl)-8-(trifluoromethyl)-1H-[1,2,3]triazolo[4,5-c]quinolin-1-yl)-1-((E)-4-fluorobut-2-enoyl)piperidin-2-yl)acetonitrile;

2-((2S,4S)-4-(7-(3-chloro-2-methoxyphenyl)-4-(3-(dimethylamino)-3-methylazetidin-1-yl)-6-fluoro-8-(trifluoromethyl)-1H-[1,2,3]triazolo[4,5-c]quinolin-1-yl)-1-((E)-4-fluorobut-2-enoyl)piperidin-2-yl)acetonitrile;

2-((2S,4S)-4-(6-fluoro-7-(5-fluoroquinolin-8-yl)-4-(S)-1(S)-1-methylpyrrolidin-2-yl)ethoxy)-8-(trifluoromethyl)-1H-[1,2,3]triazolo[4,5-c]quinolin-1-yl)-1-((E)-4-fluorobut-2-enoyl)piperidin-2-yl)acetonitrile;

1-(4-(6-fluoro-7-(5-fluoroquinolin-8-yl)-4-((S)-1-((S)-1-methylpyrrolidin-2-yl)ethoxy)-8-(trifluoromethyl)-1H-[1,2,3]triazolo[4,5-c]quinolin-1-yl)piperidin-1-yl)prop-2-en-1-one;

2-((2S,4S)-4-(6-fluoro-8-methyl-7-(1-methyl-1H-indazol-6-yl)-4-((S)-1-(S)-1-methylpyrrolidin-2-yl)ethoxy)-1H-[1,2,3]triazolo[4,5-c]quinolin-1-yl)-1-((E)-4-fluorobut-2-enoyl)piperidin-2-yl)acetonitrile;

2-((2S,4S)-4-(6-fluoro-8-methyl-7-(6-methylpyridin-3-yl)-4-((S)-1-((S)-1-methylpyrrolidin-2-yl)ethoxy)-1H-[1,2,3]triazolo[4,5-c]quinolin-1-yl)-1-((E)-4-fluorobut-2-enoyl)piperidin-2-yl)acetonitrile;

2-((2S,4S)-4-(6-fluoro-8-methyl-7-(1-methyl-1H-indazol-3-yl)-4-((S)-1-((S) methylpyrrolidin-2-yl)ethoxy)-1H-[1,2,3]triazolo[4,5-c]quinolin-1-yl)-1-(E)-4-methoxybut enoyl)piperidin-2-yl)acetonitrile;

2-((2S,4S)-4-(6-fluoro-7-(4-fluorophenyl)-8-methyl-4-((S)-1-((S)-1-methylpyrrolidin yl)ethoxy)-1H-[1,2,3]triazolo[4,5-c]quinolin-1-yl)-1-((E)-4-methoxybut-2-enoyl)piperidin yl)acetonitrile;

8-(1-(1-acryloylpiperidin-4-yl)-6-fluoro-8-methyl-4-(S)-1-(S)-1-methylpyrrolidin-2-yl)ethoxy)-1H-[1,2,3]triazolo[4,5-c]quinolin-7-yl)-1-naphthonitrile;

8-(1-((2R,4S)-1-acryloyl-2-methylpiperidin-4-yl)-6-fluoro-8-methyl-4-(S)-1-(S)-1-methylpyrrolidin-2-yl)ethoxy)-1H-[1,2,3]triazolo[4,5-c]quinolin-7-yl)-1-naphthonitrile;

2-((2S,4S)-4-(7-(5, 6-dimethyl-1H-indazol-4-yl)-4-(3-(ethyl(methyl)amino)azetidin-1-yl)-6-fluoro-8-methyl-1H-[1,2,3]triazolo[4,5-c]quinolin-1-yl)-1-(2-fluoroacryloyl)piperidin-2-yl)acetonitrile;

8-(6-fluoro-1-(14(E)-4-fluorobut-2-enoyl)piperidin-4-yl)-8-methyl-4-(S)-1-((S)-1-methylpyrrolidin-2-yl)ethoxy)-1H-[1,2,3]triazolo[4,5-c]quinolin-7-yl)-1-naphthonitrile;

8-(1-(2S,4S)-2-(cyanomethyl)-1-(2-fluoroacryloyl)piperidin-4-yl)-4-(3-(dimethylamino)-3-methylazetidin-1-yl)-6-fluoro-8-methyl-1H-[1,2,3]triazolo[4,5-c]quinolin-7-yl)-1-naphthonitrile;

2-((2S,4S)-4-(6, 8-dichloro-4-(3-(dimethylamino)-3-methylazetidin-1-yl)-7-(5-fluoroquinolin-8-yl)-1H-[1,2,3 ]triazolo[4, 5-c]quinolin-1-yl)-1-((E)-4-methoxybut-2-enoyl)piperidin-2-yl)acetonitrile;

2-((2S,4S)-4-(6, 8-dichloro-4-(3-(dimethylamino)-3-methylazetidin-1-yl)-'7-(5-fluoroquinolin-8-yl)-1H-[1,2,3]triazolo[4,5-c]quinolin-1-yl)-1-((E)-4-fluorobut-2-enoyl)piperidin-2-yl)acetonitrile; and

2-((2S,4S)-4-(4-(3-(dimethylamino)-3-methylazetidin-1-yl)-6-fluoro-7-(5-fluoroquinolin-8-yl)-8-(trifluoromethyl)-1H-[1,2,3]triazolo[4,5-c]quinolin-1-yl)-1-((E)-4-methoxybut-2-enoyl)piperidin-2-yl)acetonitrile;

or a pharmaceutically acceptable salt thereof.

In some embodiments, the KRAS inhibitor is a compound of Formula (II):

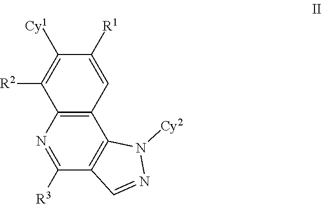

or a pharmaceutically acceptable salt thereof, wherein:

R¹ is selected from Cl, CH₃, CH₂F, CHF₂, and CF₃;

Cy¹ is selected from

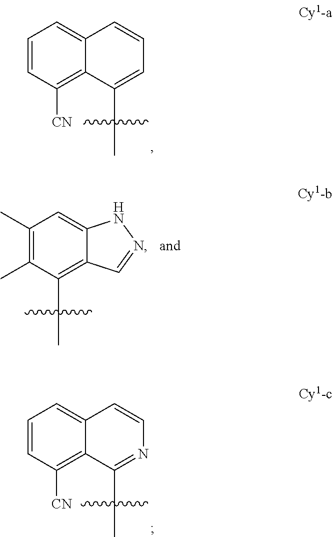 [SMILES: *c1cccc2cccc(C#N)c12 ; *c1c(C)c(C)cc2[nH]ncc12 ; *c1nccc2cccc(C#N)c12]

R² is selected from F and Cl;

R³ is selected from

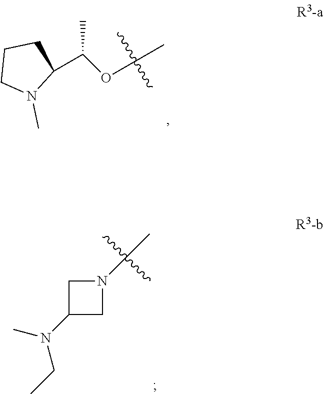

and,

Cy² is selected from

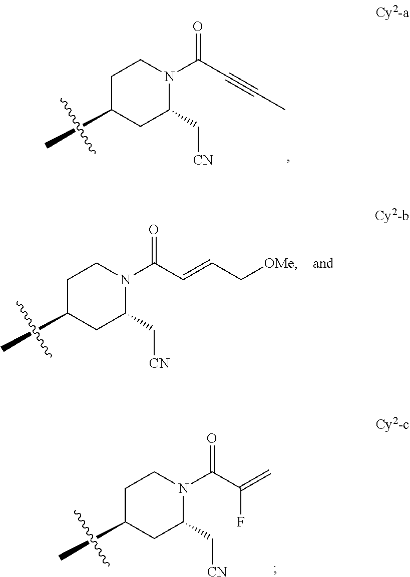

provided that the compound of Formula (II) is other than,

8-(1-((2S,4S)-2-(cyanomethyl)-1-(2-fluoroacryloyl)piperidin-4-yl)-6-fluoro-8-methyl-4-((S)-
1-((S)-1-methylpyrrolidin-2-yl)ethoxy)-1H-pyrazolo[4,3-c]quinolin-7-yl)-1-naphthonitrile,
8-(1-((2S,4S)-2-(cyanomethyl)-1-((E)-4-methoxybut-2-enoyl)piperidin-4-yl)-6-fluoro-8-
methyl-4-((S)-1-((S)-1-methylpyrrolidin-2-yl)ethoxy)-1H-pyrazolo[4,3-c]quinolin-7-yl)-1-
naphthonitrile,
2-((2S,4S)-4-(7-(5,6-dimethyl-1H-indazol-4-yl)-6-fluoro-8-methyl-4-((S)-1-((S)-1-
methylpyrrolidin-2-yl)ethoxy)-1H-pyrazolo[4,3-c]quinolin-1-yl)-1-(2-
fluoroacryloyl)piperidin-2-yl)acetonitrile,
2-((2S,4S)-1-(but-2-ynoyl)-4-(7-(5,6-dimethyl-1H-indazol-4-yl)-6-fluoro-8-methyl-4-((S)-1-
((S)-1-methylpyrrolidin-2-yl)ethoxy)-1H-pyrazolo[4,3-c]quinolin-1-yl)piperidin-2-
yl)acetonitrile, and
2-((2S,4S)-4-(7-(5,6-dimethyl-1H-indazol-4-yl)-6-fluoro-8-methyl-4-((S)-1-((S)-1-
methylpyrrolidin-2-yl)ethoxy)-1H-pyrazolo[4,3-c]quinolin-1-yl)-1-((E)-4-methoxybut-2-
enoyl)piperidin-2-yl)acetonitrile.

In some embodiments, the compound of Formula (II) or the pharmaceutically acceptable salt thereof is selected from:

1-(1-(2S,4S)-1-(but-2-ynoyl)-2-(cyanomethyl)piperidin-4-yl)-8-chloro-6-fluoro-4-(S)-1-((S)-1-methylpyrrolidin-2-yl)ethoxy)-1H-pyrazolo[4,3-c]quinolin-7-yl)isoquinoline-8-carbonitrile;

1-(8-chloro-1-(2S,4S)-2-(cyanomethyl)-1-((E)-4-methoxybut-2-enoyl)piperidin-4-yl)-6-fluoro-4-(S)-1-((S)-1-methylpyrrolidin-2-yl)ethoxy)-1H-pyrazolo[4,3-c]quinolin-7-yl)isoquinoline-8-carbonitrile;

1-(8-chloro-1-(2S,4S)-2-(cyanomethyl)-1-(2-fluoroacryloyl)piperidin-4-yl)-6-fluoro-4-((S)-1-(S)-1-methylpyrrolidin-2-yl)ethoxy)-1H-pyrazolo[4,3-c]quinolin-7-yl)isoquinoline-8-carbonitrile;

2-((2S,4S)-4-(8-chloro-7-(5,6-dimethyl-1H-indazol-4-yl)-4-(3-(ethyl(methyl)amino)azetidin-1-yl)-6-fluoro-1H-pyrazolo[4,3-c]quinolin-1-yl)-1-(2-fluoroacryloyl)piperidin-2-yl)acetonitrile;

8-(8-chloro-1-(2S,4S)-2-(cyanomethyl)-1-(2-fluoroacryloyl)piperidin-4-yl)-6-fluoro-4-((S)-1-(S)-1-methylpyrrolidin-2-yl)ethoxy)-1H-pyrazolo[4,3-c]quinolin-7-yl)-1-naphthonitrile;

8-(8-chloro-1-(2S,4S)-2-(cyanomethyl)-1-((E)-4-methoxybut-2-enoyl)piperidin-4-yl)-6-fluoro-4-(S)-1-((S)-1-methylpyrrolidin-2-yl)ethoxy)-1H-pyrazolo[4,3-c]quinolin-7-yl)-1-naphthonitrile; and

8-(1-(2S,4S)-1-(but-2-ynoyl)-2-(cyanomethyl)piperidin-4-yl)-6-fluoro-8-methyl-4-(S)-1-((S)-1-methylpyrrolidin-2-yl)ethoxy)-1H-pyrazolo[4,3-c]quinolin-7-yl)-1-naphthonitrile;

or a pharmaceutically acceptable salt thereof.

In an embodiment of Formula (II), or a pharmaceutically acceptable salt thereof,

R¹ is selected from Cl, CH₂F, CHF₂, and CF₃;

Cy¹ is selected from

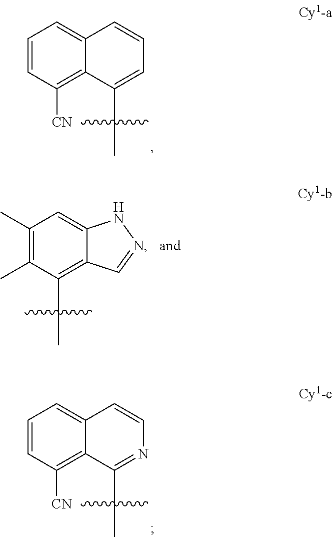 [SMILES: *c1cccc2cccc(C#N)c12 ; *c1c(C)c(C)cc2[nH]ncc12 ; *c1nccc2cccc(C#N)c12]

R² is selected from F and Cl;

R³ is selected from

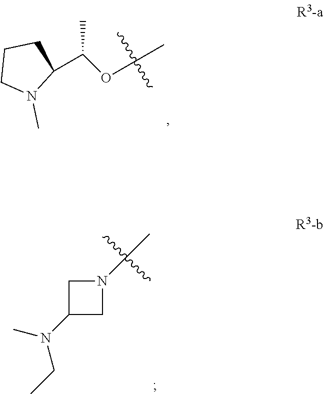

and,

Cy² is selected from

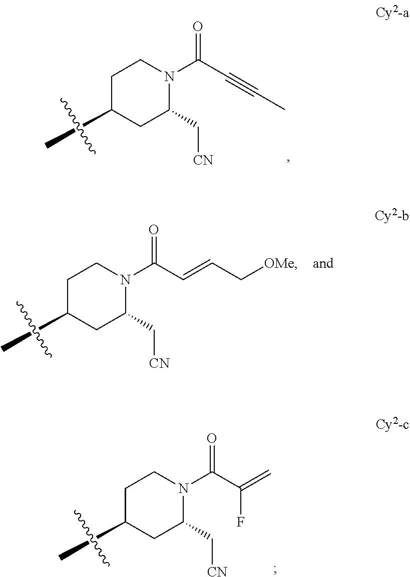

In another embodiment, the compound of Formula (II) or the pharmaceutically acceptable salt thereof is selected from:

1-(1-(2S,4S)-1-(but-2-ynoyl)-2-(cyanomethyl)piperidin-4-yl)-8-chloro-6-fluoro-4-(S)-1-((S)-1-methylpyrrolidin-2-yl)ethoxy)-1H-pyrazolo[4,3-c]quinolin-7-yl)isoquinoline-8-carbonitrile;

1-(8-chloro-1-(2S,4S)-2-(cyanomethyl)-1-(E)-4-methoxybut-2-enoyl)piperidin-4-yl)-6-fluoro-4-(S)-1-((S)-1-methylpyrrolidin-2-yl)ethoxy)-1H-pyrazolo[4,3-c]quinolin-7-yl)isoquinoline-8-carbonitrile;

1-(8-chloro-1-(2S,4S)-2-(cyanomethyl)-1-(2-fluoroacryloyl)piperidin-4-yl)-6-fluoro ((S)-1-(S)-1-methylpyrrolidin-2-yl)ethoxy)-1H-pyrazolo[4,3-c]quinolin-7-yl)isoquinoline carbonitrile;

2-((2S,4S)-4-(8-chloro-7-(5,6-dimethyl-1H-indazol-4-yl)-4-(3-(ethyl(methyl)amino)azetidin-1-yl)-6-fluoro-1H-pyrazolo[4,3-c]quinolin-1-yl)-1-(2-fluoroacryloyl)piperidin-2-yl)acetonitrile;

8-(8-chloro-1-(2S,4S)-2-(cyanomethyl)-1-(2-fluoroacryloyl)piperidin-4-yl)-6-fluoro-4-((S)-1-(S)-1-methylpyrrolidin-2-yl)ethoxy)-1H-pyrazolo[4,3-c]quinolin-7-yl)-1-naphthonitrile; and

8-(8-chloro-1-(2S,4S)-2-(cyanomethyl)-1-((E)-4-methoxybut-2-enoyl)piperidin-4-yl)-6-fluoro-4-(S)-1-((S)-1-methylpyrrolidin-2-yl)ethoxy)-1H-pyrazolo[4,3-c]quinolin-7-yl)-1-naphthonitrile;

or a pharmaceutically acceptable salt thereof.

In some embodiments, the KRAS inhibitor is a compound of Formula (III):

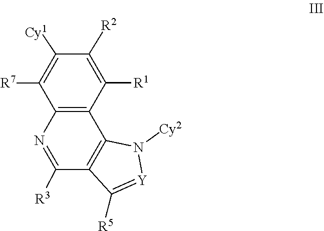

or a pharmaceutically acceptable salt thereof, wherein:

Y is N or CR⁶;

R¹ is selected from H, C₁₋₃ alkyl, C₁₋₃ haloalkyl, cyclopropyl, halo, D, CN, and OR^{a1}; wherein said C₁₋₃ alkyl and cyclopropyl are each optionally substituted with 1 or 2 substituents independently selected from R^{g};

R¹ is selected from H, C₁₋₃ alkyl, C₁₋₃ haloalkyl, 4-6 membered heterocycloalkyl, phenyl, 5-6 membered heteroaryl, 4-6 membered heterocycloalkyl-C₁₋₃ alkylene, phenyl-C₁₋₃ alkylene, 5-6 membered heteroaryl-Cl-3 alkylene, halo, D, CN, and ORa²; wherein said C₁₋₃ alkyl, 4-6 membered heterocycloalkyl, phenyl, 5-6 membered heteroaryl, 4-6 membered heterocycloalkyl-C₁₋₃ alkylene, phenyl-C₁₋₃ alkylene, 5-6 membered heteroaryl-C₁₋₃ alkylene are each optionally substituted with 1 or 2 substituents independently selected from R^{g};

Cy¹ is selected from C₃₋₁₀ cycloalkyl, 4-10 membered heterocycloalkyl, C₆₋₁₀ aryl and 6-10 membered heteroaryl; wherein the 4-10 membered heterocycloalkyl and 6-10 membered heteroaryl each has at least one ring-forming carbon atom and 1, 2, 3, or 4 ring-forming heteroatoms independently selected from N, O, and S; wherein a ring-forming carbon atom of 6-10 membered heteroaryl and 4-10 membered heterocycloalkyl is optionally substituted by oxo to form a carbonyl group; and wherein the C₃₋₁₀ cycloalkyl, 4-10 membered heterocycloalkyl, C₆₋₁₀ aryl and 6-10 membered heteroaryl are each optionally substituted with 1, 2, 3, or 4 substituents independently selected from R^{io};

R³ is selected from H, C₁₋₃ alkyl, C₁₋₃ haloalkyl, C₃₋₆ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, halo, D, CN, OR^{f3}, C(O)NR^{c3}R^{d3}, NR^{c3}R^{j3}, and NR^{c3}C(O)R^{b3}; wherein said C₁₋₃ alkyl, C₃₋₆ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene are each optionally substituted with 1, 2, or 3 substituents independently selected from R³⁰;

R⁵ is selected from H, C₁₋₃ alkyl, C₁₋₃ haloalkyl, cyclopropyl, halo, D, CN, and OR^{a5}; wherein said C₁₋₃ alkyl and cyclopropyl are each optionally substituted with 1 or 2 substituents independently selected from R^{g};

R⁶ is selected from H, C₁₋₃ alkyl, C₁₋₃ haloalkyl, C₃₋₆ cycloalkyl, 4-9 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, halo, D, CN, OR', and C(O)NRc⁶R^{d6}; wherein said C₁₋₃ alkyl, C₃₋₆ cycloalkyl, 4-9 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene are each optionally substituted with 1 or 2 substituents independently selected from R⁶⁰;

R⁷ is selected from H, C₁₋₃ alkyl, C₁₋₃ haloalkyl, cyclopropyl, halo, D, CN, and OR^{a7}; wherein said C₁₋₃ alkyl and cyclopropyl are each optionally substituted with 1 or 2 substituents independently selected from R^{g};

Cy² is selected from

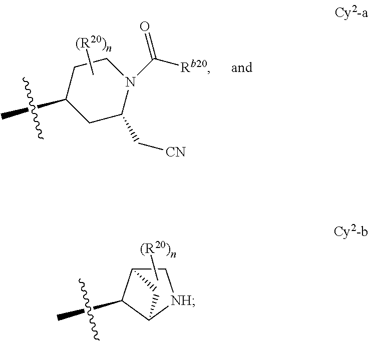

wherein n is 0, 1, or 2;

each R¹⁰ is independently selected from C₁₋₃ alkyl, C₁₋₃ haloalkyl, halo, D, CN, OR^{a10}, C(O)R^{b10}, C(O)NR^{c10}R^{d10}, C(O)OR^{a10}, NR^{c10}R^{d10}, and S(O)₂R^{b10};

each R²⁰ is independently selected from C₁₋₃ alkyl, C₁₋₃ haloalkyl, halo, D, CN, and OR^{a20};

each R³⁰ is independently selected from C₁₋₃ alkyl, C₁₋₃ haloalkyl, C₃₋₆ cycloalkyl, 4-6 membered heterocycloalkyl, phenyl, 5-6 membered heteroaryl, halo, D, CN, OR^{a30}, C(O)R^{b30}, C(O)NR^{c30}R^{d30}, C(O)OR^{a30}, NR^{c30}R^{d30}, and S(O)₂R^{b30}; wherein said C₁₋₃ alkyl, C₃₋₆ cycloalkyl, 4-6 membered heterocycloalkyl, phenyl, and 5-6 membered heteroaryl are each optionally substituted with 1 or 2 substituents independently selected from R³¹;

each R³¹ is independently selected from C₁₋₃ alkyl, C₁₋₃ haloalkyl, halo, D, CN, OR^{a31}, C(O)R^{b31}, C(O)NR^{c31}R^{d31}, C(O)OR^{a31}, NR^{c31}R^{d31}, and S(O)₂R^{b31};

each R³³ is independently selected from C₁₋₃ alkyl, C₁₋₃ haloalkyl, C₃₋₆ cycloalkyl, 4-membered heterocycloalkyl, 6-membered heterocycloalkyl, phenyl, 5-6 membered heteroaryl, halo, D, CN, OR^{a30}, C(O)NR^{c30}R^{d30}, and NR^{c30}R_{d30}, wherein said C₁₋₃ alkyl, C₃₋₆ cycloalkyl, 4-membered heterocycloalkyl, 6-membered heterocycloalkyl, phenyl, and 5-6 membered heteroaryl are each optionally substituted with 1 or 2 substituents independently selected from R³¹;

each R⁶⁰is independently selected from C₁₋₃ alkyl, C₁₋₃ haloalkyl, 4-6 membered heterocycloalkyl, 5-6 membered heteroaryl, halo, D, CN, OR^{a60}, C(O)R^{b60}, C(O)NR^{c60}R^{d60}, NR^{c60}C(O)R^{b60}, C(O)OR^{a60}, NR^{c60}C(O)OR^{a60}, NR^{c60}R^{d60}, NR^{c60}S(O)₂R^{b60}, and S(O)₂R^{b60}; wherein said C₁₋₃ alkyl, 4-6 membered heterocycloalkyl, and 5-6 membered heteroaryl are each optionally substituted with 1 or 2 substituents independently selected from R⁶¹;

each R⁶¹ is independently selected from C₁₋₃ alkyl, C₁₋₃ haloalkyl, halo, D, CN, OR^{a61}, and NR^{c61}R^{d61};

R^{a1} is selected from H, C₁₋₃ alkyl, and C₁₋₃ haloalkyl;

each R is independently selected from H, C₁₋₃ alkyl, and C₁₋₃ haloalkyl;

each R^{b3}, R^{c3} and R^{d3} is independently selected from H, C₁₋₃ alkyl, C₁₋₃ haloalkyl, C₃₋₆ cycloalkyl, 4-6 membered heterocycloalkyl, phenyl and 5-6 membered heteroaryl; wherein said , C₁₋₃ alkyl, C₃₋₆ cycloalkyl, 4-6 membered heterocycloalkyl, phenyl and 5-6 membered heteroaryl are each optionally substituted with 1, 2, or 3 substituents independently selected from R³⁰;

or R^{c3} and R^{d3} attached to the same N atom, together with the N atom to which they are attached, form a 4-, 5-, or 6-membered heterocycloalkyl group optionally substituted with 1, 2, or 3 substituents independently selected from R³⁰;

R^{j3} is selected from C₁₋₃ alkyl, C₁₋₃ haloalkyl, C₃₋₆ cycloalkyl, 4-6 membered heterocycloalkyl, phenyl and 5-6 membered heteroaryl; wherein said , C₁₋₃ alkyl, C₃₋₆ cycloalkyl, 4-6 membered heterocycloalkyl, phenyl and 5-6 membered heteroaryl are each optionally substituted with 1, 2, or 3 substituents independently selected from R³⁰;

or R^{c3} and R^{j3} attached to the same N atom, together with the N atom to which they are attached, form a 4-, 5-, or 6-membered heterocycloalkyl group optionally substituted with 1, 2, or 3 substituents independently selected from R³⁰;

R^{f3} is selected from C₁₋₃ haloalkyl, C₃₋₆ cycloalkyl, 4-6 membered heterocycloalkyl, phenyl and 5-6 membered heteroaryl; wherein said C₁₋₃ haloalkyl, C₃₋₆ cycloalkyl, 4-6 membered heterocycloalkyl, phenyl, and 5-6 membered heteroaryl are each optionally substituted with 1, 2, or 3 substituents independently selected from R³⁰; or

R^{f3} is selected from

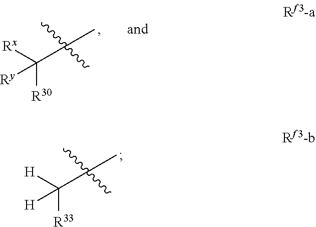

wherein R^{x} is H or C₁₋₂ alkyl and R^{y} is C₁₋₂ alkyl;

or R^{x} and R^{y}, together with the C atom to which they are attached, form a 3-, or 4-membered cycloalkyl group;

R^{a5} is selected from H, C₁₋₃ alkyl, and C₁₋₃ haloalkyl;

each R^{a5}, R^{c6} and R^{d6} is independently selected from H, C₁₋₃ alkyl, C₁₋₃ haloalkyl, C₃₋₆ cycloalkyl, 4-6 membered heterocycloalkyl, phenyl and 5-6 membered heteroaryl; wherein said C₁₋₃ alkyl, C₃₋₆ cycloalkyl, 4-6 membered heterocycloalkyl, phenyl and 5-6 membered heteroaryl are each optionally substituted with 1 or 2 substituents independently selected from R⁶⁰;

R^{a7} is selected from H, C₁₋₃ alkyl, and C₁₋₃ haloalkyl;

each R^{a10}, R^{b10}, R^{c10} and R^{d30} is independently selected from H, C₁₋₃ alkyl, and C₁₋₃ haloalkyl;

each R^{a20} is independently selected from H, C₁₋₃ alkyl, and C₁₋₃ haloalkyl;

R^{b20} is selected from NH₂, C₁₋₃ alkyl, and C₁₋₃ haloalkyl;

each R^{a30}, R^{b30}, R^{c30} and R^{d30} is independently selected from H, C₁₋₃ alkyl, and C₁₋₃ haloalkyl;

each R^{a31}, R^{b31}, R^{c31} and R^{d31} is independently selected from H, C₁₋₃ alkyl, and C₁₋₃ haloalkyl;

each R^{a60}, R^{b60}, R^{c60} and R^{d60} is independently selected from H, C₁₋₃ alkyl, and C₁₋₃ C₃₋₆ cycloalkyl, 4-6 membered heterocycloalkyl, and 5-6 membered heteroaryl; wherein said C₁₋₃ alkyl, C₃₋₆ cycloalkyl, 4-6 membered heterocycloalkyl, and 5-6 membered heteroaryl are each optionally substituted with 1 or 2 substituents independently selected from R⁶¹;

or any R^{c60} and R^{d60} attached to the same N atom, together with the N atom to which they are attached, form a 4-, 5-, or 6-membered heterocycloalkyl group optionally substituted with 1 or 2 substituents independently selected from R⁶¹; and

each R^{a61}, R^{c61}, and R^{d61}, is independently selected from H, C₁₋₃ alkyl, and C₁₋₃ haloalkyl; and

each R^{g} is independently selected from D, OH, CN, halo, C₁₋₃ alkyl, C₁₋₃ haloalkyl, C₁₋₃ alkoxy, C₁₋₃ haloalkoxy, amino, C₁₋₃ alkylamino, and di(C₁₋₃ alkyl)amino;

provided that the compound of Formula (III) is other than:

3-(1-((1R,4R,5S)-2-azabicyclo[2.1.1]hexan-5-yl)-8-(2-cyanoethyl)-4-ethoxy-6-fluoro-7-(3-hydroxynaphthalen-1-yl)-1H-pyrrolo[3,2-c]quinolin-2-yl)-N,N-dimethylpropanamide.

In some embodiments, the compound of Formula (III) is selected from:

3-(1-((1R,4R,5S)-2-azabicyclo[2.1.1]hexan-5-yl)-7-(7-chloro-3-hydroxynaphthalen-1-yl)-6-fluoro-2-methyl-4-(1H-1,2,4-triazol-1-yl)-1H-pyrrolo[3,2-c]quinolin-8-yl)propanenitrile;

3-(1-((1R,4R,5S)-2-azabicyclo[2.1.1]hexan-5-yl)-7-(5,7-difluoro-1H-indol-3-yl)-6-fluoro-2-methyl-4-(S)-1-((S)-1-methylpyrrolidin-2-yl)ethoxy)-1H-pyrrolo[3,2-c]quinolin-8-yl)propanenitrile;

3-(1-((1R,4R,5S)-2-Azabicyclo[2.1.1]hexan-5-yl)-6-fluoro-7-(6-fluoro-5-methyl-1H-indol-3-yl)-2-methyl-4-(S)-1-((S)-1-methylpyrrolidin-2-yl)ethoxy)-1H-pyrrolo[3,2-c]quinolin yl)propanenitrile;

3-(2-(3-(Azetidin-1-yl)-3-oxopropyl)-1-((1R, 4R, 5S)-2-azabicyclo[2.1.1]hexan-5-yl)-7-(2,3-dichlorophenyl)-6-fluoro-4-((S)-1-((S)-1-methylpyrrolidin-2-yl)ethoxy)-1H-pyrrolo [3,2-c]quinolin-8-yl)propanenitrile;

3-((1-(1R,4R,5S)-2-Azabicyclo[2.1.1]hexan-5-yl)-6-fluoro-7-(3-hydroxynaphthalen-1-yl)-8-methyl-4-((S)-1-((S)-1-methylpyrrolidin-2-yl)ethoxy)-1H-pyrrolo[3,2-c]quinolin-2-yl)methyl)oxazolidin-2-one;

8-(1-((1R,4R,5S)-2-Azabicyclo[2.1.1]hexan-5-yl)-6-fluoro-2,8-dimethyl-4-(5)-1-((S)-1-methylpyrrolidin-2-yl)ethoxy)-1H-pyrrolo[3,2-c]quinolin-7-yl)-1-naphthonitrile;

1-((2S,4S)-1-Acetyl-2-(cyanomethyl)piperidin-4-yl)-7-(8-cyanonaphthalen-1-yl)-6-fluoro-4-((S)-1-((S)-1-methylpyrrolidin-2-yl)ethoxy)-1H-pyrazolo[4,3-c]quinoline-8-carbonitrile;

8-(1-((1R,4R,5S)-2-Azabicyclo[2.1.1]hexan-5-yl)-6-fluoro-8-methyl-4-((5)-1-((S)-1-methylpyrrolidin-2-yl)ethoxy)-2-((3-oxomorpholino)methyl)-1H-pyrrolo[3,2-c]quinolin-7-yl)-1-naphthonitrile;

3-(7-(Benzo[b]thiophen-3-yl)-((1R, 4R,5S)-2-azabicyclo[2.1.1]hexan-5-yl)-6-fluoro-4-((S)-1-((S)-1-methylpyrrolidin-2-yl)ethoxy)-2-((2-oxopyrrolidin-1-yl)methyl)-1H-pyrrolo[3,2-c]quinolin-8-yl)propanenitrile;

3-(1-((1R,4R,5S)-2-Azabicyclo[2.1.1]hexan-5-yl)-4-(S)-1-(dimethylamino)propan-2-yl)oxy)-6-fluoro-7-(7-fluoronaphthalen-1-yl)-2-(2-oxopyrrolidin-1-yl)methyl)-1H-pyrrolo[3,2-c]quinolin-8-yl)propanenitrile;

8-(1-((1R,4R,5S)-2-Azabicyclo[2.1.1]hexan-5-yl)-8-(2-cyanoethyl)-6-fluoro-2-methyl-4-((S)-1-((S)-1-methylpyrrolidin-2-yl)ethoxy)-1H-pyrrolo[3,2-c]quinolin-7-yl)-1-naphthonitrile;

3-(1-((1R,4R,5S)-2-Azabicyclo[2.1.1]hexan-5-yl)-7-(2,3-dichloro-5-hydroxyphenyl)-6-fluoro-2-methyl-4-(S)-1-((S)-1-methylpyrrolidin-2-yl)ethoxy)-1H-pyrrolo[3,2-c]quinolin-8-yl)propanenitrile;

3-(1-((1R,4R,5S)-2-Azabicyclo[2.1.1]hexan-5-yl)-8-(2-cyanoethyl)-6-fluoro-4-(3-fluoro-1-methylazetidin-3-yl)methoxy)-7-(3-hydroxynaphthalen-1-yl)-1H-pyrrolo[3,2-c]quinolin-2-yl)-N,N-dimethylpropanamide;

3-(1-((1R,4R,5S)-2-Azabicyclo[2.1.1]hexan-5-yl)-6-fluoro-7-(3-hydroxynaphthalen-1-yl)-2-methyl-4-(5-methylpyrazin-2-yl)-1H-pyrrolo[3,2-c]quinolin-8-yl)propanenitrile;

3-(1-((1R,4R,5S)-2-Azabicyclo[2.1.1]hexan-5-yl)-6-fluoro-7-(7-fluoronaphthalen-1-yl) methyl-2-((4-methyl-2-oxopiperazin-1-yl)methyl)-1H-pyrrolo[3,2-c]quinolin-8-yl)propanenitrile;

3-(1-((1R,4R,5S)-2-azabicyclo[2.1.1]hexan-5-yl)-7-(2,3-dichloro-5-hydroxyphenyl)-4-ethoxy-6-fluoro-24(4-isopropyl-2-oxopiperazin-1-yl)methyl)-1H-pyrrolo[3,2-c]quinolin-8-yl)propanenitrile;

3-(1-((1R,4R,5S)-2-Azabicyclo[2.1.1]hexan-5-yl)-4-(3-(dimethylamino)-3-methylazetidin-1-yl)-6-fluoro-7-(7-fluoronaphthalen-1-yl)-2-((3-oxomorpholino)methyl)-1H-pyrrolo[3,2-c]quinolin-8-yl)propanenitrile;

3-(1-((1R,4R,5S)-2-azabicyclo[2.1.1]hexan-5-yl)-4-ethoxy-6-fluoro-7-(3-hydroxynaphthalen-1-yl)-2-(1-(3-oxomorpholino)ethyl)-1H-pyrrolo[3,2-c]quinolin-8-yl)propanenitrile;

3-(1-((endo)-2-Azabicyclo[2.1.1]hexan-5-yl)-6-fluoro-7-(3-hydroxynaphthalen-1-yl)-4-((S)-1-((S)-1-methylpyrrolidin-2-yl)ethoxy)-2-(pyridin-3-yl)-1H-pyrrolo[3,2-c]quinolin-8-yl)propanenitrile;

3-(2-(3-(azetidin-1-yl)-3-oxopropyl)-1-(1R,4R,5S)-2-azabicyclo[2.1.1]hexan-5-yl)-7-(7,8-difluoronaphthalen-1-yl)-6-fluoro-4-((S)-1-((S)-1-methylpyrrolidin-2-yl)ethoxy)-1H-pyrrolo[3,2-c]quinolin-8-yl)propanenitrile;

3-(2-(3-(azetidin-1-yl)-3-oxopropyl)-1-(1R,4R,5S)-2-azabicyclo[2.1.1]hexan-5-yl)-7-(6,7-difluoronaphthalen-1-yl)-6-fluoro-4-((S)-1-((S)-1-methylpyrrolidin-2-yl)ethoxy)-1H-pyrrolo[3,2-c]quinolin-8-yl)propanenitrile;

3-(1-((1R,4R,5S)-2-azabicyclo[2.1.1]hexan-5-yl)-6-fluoro-7-(7-fluoro-3-hydroxynaphthalen-1-yl)-2-methyl-4-((S)-1-((S)-1-methylpyrrolidin-2-yl)ethoxy)-1H-pyrrolo[3,2-c]quinolin-8-yl)propanenitrile;

1-(1-(2S,4S)-1-Acetyl-2-(cyanomethyl)piperidin-4-yl)-8-chloro-6-fluoro-4-((S)-1-((S)-1-methylpyrrolidin-2-yl)ethoxy)-1H-pyrazolo[4,3-c]quinolin-7-yl)isoquinoline-8-carbonitrile;

8-(1-((2S,4S)-1-acetyl-2-(cyanomethyl)piperidin-4-yl)-8-chloro-6-fluoro-4-((S)-1-((S) methylpyrrolidin-2-yl)ethoxy)-1H-pyrrolo[3,2-c]quinolin-7-yl)-1-naphthonitrile;

8-(1-(2S,4S)-1-acetyl-2-(cyanomethyl)piperidin-4-yl)-8-chloro-6-fluoro-4-((S)-1-((S) methylpyrrolidin-2-yl)ethoxy)-1H-pyrazolo[4,3-c]quinolin-7-yl)-1-naphthonitrile;

3-(1-((1R,4R,5S)-2-Azabicyclo[2.1.1]hexan-5-yl)-6-fluoro-7-(7-fluoro hydroxynaphthalen-1-yl)-2-methyl-4-(1H-1,2,4-triazol-1-yl)-1H-pyrrolo[3,2-c]quinolin-8-yl)propanenitrile;

3-(1-((1R,4R,5S)-2-Azabicyclo[2.1.1]hexan-5-yl)-6-fluoro-7-(7-fluoronaphthalen-1-yl)-2-methyl-4-(S)-1-((S)-1-methylpyrrolidin-2-yl)ethoxy)-1H-pyrrolo[3,2-c]quinolin-8-yl)propanenitrile;

(2R)-2-(1-((1R,4R,5S)-2-azabicyclo[2.1.1]hexan-5-yl)-8-(2-cyanoethyl)-7-(2,3-dichlorophenyl)-6-fluoro-4-(1H-1,2,4-triazol-1-yl)-1H-pyrrolo[3,2-c]quinolin-2-yl)-N,N-dimethylpyrrolidine-1-carboxamide;

methyl (2R)-2-(1-((1R,4R,5S)-2-azabicyclo[2.1.1]hexan-5-yl)-7-(2-chloro-3-methylphenyl)-8-(2-cyanoethyl)-6-fluoro-4-(1H-1,2,4-triazol-1-yl)-1H-pyrrolo[3,2-c]quinolin-2-yl)pyrrolidine-1-carboxylate;

Methyl (1S,3R,5S)-3-(1-((1R,4R,5S)-2-azabicyclo[2.1.1]hexan-5-yl)-8-(2-cyanoethyl)-7-(2,3-dichlorophenyl)-4-(6-(dimethylcarbamoyl)pyridin-3-yl)-6-fluoro-1H-pyrrolo[3,2-c]quinolin-2-yl)-2-azabicyclo[3.1.0]hexane-2-carboxylate;

3-(1-((1R,4R,5S)-2-Azabicyclo[2.1.1]hexan-5-yl)-7-(2,3-dichlorophenyl)-6-fluoro-4-methyl-2-(5-oxo-1,2,3,5-tetrahydroindolizin-3-yl)-1H-pyrrolo[3,2-c]quinolin-8-yl)propanenitrile;

Methyl (2R)-2-(1-((1R,4R,5S)-2-azabicyclo[2.1.1]hexan-5-yl)-8-(2-cyanoethyl)-7-(2,3-dichlorophenyl)-4-(6-(dimethylcarbamoyl)pyridin-3-yl)-6-fluoro-1H-pyrrolo[3,2-c]quinolin-2-yl)pyrrolidine-1-carboxylate;

Methyl (2R)-2-(1-((1R,4R,5S)-2-azabicyclo[2.1.1]hexan-5-yl)-8-(2-cyanoethyl)-7-(2,3-dichlorophenyl)-6-fluoro-4-(6-(methylcarbamoyl)pyridin-3-yl)-1H-pyrrolo[3,2-c]quinolin-2-yl)pyrrolidine-1-carboxylate;

3-(1-((1R,4R,5S)-2-Azabicyclo[2.1.1]hexan-5-yl)-7-(2-chloro-3-fluorophenyl)-2-((R)-1-(cyclopropanecarbonyl)pyrrolidin-2-yl)-6-fluoro-4-methyl-1H-pyrrolo[3,2-c]quinolin-8-yl)propanenitrile;

8-(2-((R)-1-Acetylpyrrolidin-2-yl)-1-(1R,4R,5S)-2-azabicyclo[2.1.1]hexan-5-yl) fluoro-8-methyl-4-(2-methylpyridin-4-yl)-1H-pyrrolo[3,2-c]quinolin-7-yl)-1,2,3,4-tetrahydronaphthalene-1-carbonitrile;

5-(1-((1R,4R,5S)-2-Azabicyclo[2.1.1]hexan-5-yl)-7-(3-chloro-2-methylphenyl)-8-(2-cyanoethyl)-6-fluoro-2-((R)-1-(2-oxopyrazin-1(2H)-yl)ethyl)-1H-pyrrolo[3,2-c]quinolin-4-yl)-N-methylpicolinamide;

3-(1-((1R,4R,5S)-2-Azabicyclo[2.1.1]hexan-5-yl)-6-fluoro-7-(7-fluoronaphthalen-1-yl)-4-(6-(2-hydroxypropan-2-yl)pyridin-3-yl)-2-((R)-1-(2-oxopyrazin-1(2H)-yl)ethyl)-1H-pyrrolo[3,2-c]quinolin-8-yl)propanenitrile;

3-(1-((1R,4R,5S)-2-Azabicyclo[2.1.1]hexan-5-yl)-7-(3-chloro-2-methylphenyl)-6-fluoro-4-(5-methylpyrazin-2-yl)-2-((R)-1-(2-oxopyrazin-1(2H)-yl)ethyl)-1H-pyrrolo[3,2-c]quinolin-8-yl)propanenitrile;

Methyl (2R)-2-(1-((1R,4R,5S)-2-azabicyclo[2.1.1]hexan-5-yl)-8-(2-cyanoethyl)-7-(2,3-dichlorophenyl)-6-fluoro-4-(5-fluoro-6-(methylcarbamoyl)pyridin-3-yl)-1H-pyrrolo[3,2-c]quinolin-2-yl)pyrrolidine-1-carboxylate;

Methyl (2R)-2-(1-((1R,4R,5S)-2-azabicyclo[2.1.1]hexan-5-yl)-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)pyrrolidine-1-carboxylate;

Ethyl (2R)-2-(1-((1R,4R,5S)-2-azabicyclo[2.1.1]hexan-5-yl)-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)pyrrolidine-1-carboxylate;

3-(1-((1R,4R,5S)-2-Azabicyclo[2.1.1]hexan-5-yl)-7-(2,3-dichlorophenyl)-2-((R)-1-(3,3-difluoroazetidine-1-carbonyl)pyrrolidin-2-yl)-6-fluoro-4-(methyl-d3)-1H-pyrrolo[3,2-c]quinolin-8-yl)propanenitrile;

3-(1-((1R,4R,5S)-2-Azabicyclo[2.1.1]hexan-5-yl)-7-(2,3-dichlorophenyl)-2-((R)-1-(3,3-difluoroazetidine-1-carbonyl)pyrrolidin-2-yl)-6-fluoro-1H-pyrrolo[3,2-c]quinolin-8-yl)propanenitrile;

3-(1-((1R,4R,5S)-2-Azabicyclo[2.1.1]hexan-5-yl)-7-(3-chloro-2-methylphenyl)-6-fluoro-4-(5-methylpyrazin-2-yl)-2-((R)-1-(3-oxomorpholino)ethyl)-1H-pyrrolo[3,2-c]quinolin-8-yl)propanenitrile;

5-(1-((1R,4R,5S)-2-Azabicyclo[2.1.1]hexan-5-yl)-8-(2-cyanoethyl)-6-fluoro-7-(7-fluoronaphthalen-1-yl)-24(R)-1-(3-oxomorpholino)ethyl)-1H-pyrrolo[3,2-c]quinolin-4-yl)-N-methylpicolinamide;

3-(1-((1R,4R,5S)-2-Azabicyclo[2.1.1]hexan-5-yl)-6-fluoro-7-(7-fluoronaphthalen-1-yl)-4-(6-(2-hydroxypropan-2-yl)pyridin-3-yl)-24(R)-1-(3-oxomorpholino)ethyl)-1H-pyrrolo[3,2-c]quinolin-8-yl)propanenitrile;

3-(1-((1R,4R,5S)-2-Azabicyclo[2.1.1]hexan-5-yl)-6-fluoro-7-(7-fluoronaphthalen-1-yl)-4-(5-methylpyrazin-2-yl)-2-((R)-1-(3-oxomorpholino)ethyl)-1H-pyrrolo[3,2-c]quinolin-8-yl)propanenitrile;

Methyl (1R,3R,5R)-3-(1-((1R,4R,5S)-2-azabicyclo[2.1.1]hexan-5-yl)-8-(2-cyanoethyl)-7-(2,3-dichlorophenyl)-4-(6-(dimethylcarbamoyl)pyridin-3-yl)-6-fluoro-1H-pyrrolo[3,2-c]quinolin-2-yl)-2-azabicyclo[3.1.0]hexane-2-carboxylate;

3-(1-((1R,4R,5S)-2-Azabicyclo[2.1.1]hexan-5-yl)-24(1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile;

3-(1-((1R,4R,5S)-2-Azabicyclo[2.1.1]hexan-5-yl)-7-(2,3-dichlorophenyl)-6-fluoro-4-(6-(2-hydroxypropan-2-yl)pyridin-3-yl)-2-((R)-1-(2-oxopyrazin-1(1R)-yl)ethyl)-1H-pyrrolo[3,2-c]quinolin-8-yl)propanenitrile;

Methyl (2R,4S)-2-(1-((1R,4R,5S)-2-azabicyclo[2.1.1]hexan-5-yl)-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)-4-fluoropyrrolidine-1-carboxylate;

Methyl (2R,5R)-2-(1-((1R,4R,5S)-2-azabicyclo[2.1.1]hexan-5-yl)-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)-5-methylpyrrolidine-1-carboxylate;

Methyl (2R)-2-(1-((1R,4R,5S)-2-azabicyclo[2.1.1]hexan-5-yl)-3-chloro-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)pyrrolidine-1-carboxylate;

4-(1-((1R,4R,5S)-2-Azabicyclo[2.1.1]hexan-5-yl)-8-(2-cyanoethyl)-7-(2,3-dichlorophenyl)-6-fluoro-2-((R)-1-(2-oxopyrazin-1(2H)-yl)ethyl)-1H-pyrrolo[3,2-c]quinolin-4-yl)-2-fluoro-N-methylbenzamide;

Methyl ((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)carbamate;

N-((1R)-1-(1-((1R,4R,5S)-2-Azabicyclo[2.1.1]hexan-5-yl)-8-(2-cyanoethyl)-7-(2,3-dichlorophenyl)-6-fluoro-4-methyl-1H-pyrrolo[3 ,2-c]quinolin-2-yl)ethyl)-2,2-difluoroacetamide;

N-((1R)-1-(1-((1R,4R,5S)-2-Azabicyclo[2.1.1]hexan-5-yl)-8-(2-cyanoethyl)-7-(2,3-dichlorophenyl)-6-fluoro-4-methyl-1H-pyrrolo[3 ,2-c]quinolin-2-yl)ethyl)-2,2-difluoroacetamide;

(2S)-N-((1R)-1-(1-((1R,4R,5S)-2-Azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)tetrahydrofuran-2-carboxamide;

N-((1R)-1-(1-((1R,4R,5S)-2-Azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)cyclopropanesulfonamide;

N-((1R)-1-(1-((1R,4R,5S)-2-Azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)thiazole-4-carboxamide;

N-((1R)-1-(1-((1R,4R,5S)-2-Azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)-N-methylcyclopropanecarboxamide;

N-((1R)-1-(1-((1R,4R,5S)-2-Azabicyclo[2.1.1]hexan-5-yl)-8-(2-cyanoethyl)-7-(2,3-dichlorophenyl)-6-fluoro-4-(1-hydroxyethyl)-1H-pyrrolo[3,2-c]quinolin-2-yl)ethyl)-1-methylcyclopropane-1-carboxamide;

3-(1-((1R,4R,5S)-2-Azabicyclo[2.1.1]hexan-5-yl)-7-(2,3-dichlorophenyl)-6-fluoro-4-(1-hydroxyethyl)-2-(1R,3R,5R)-2-(1-methylcyclopropane-1-carbonyl)-2-azabicyclo[3.1.0]hexan-3-yl)-1H-pyrrolo[3,2-c]quinolin-8-yl)propanenitrile;

3-(1-((1R,4R,5S)-2-Azabicyclo[2.1.1]hexan-5-yl)-7-(2,3-dichlorophenyl)-6-fluoro-2-((1R,3R,5R)-2-(1-fluorocyclopropane-1-carbonyl)-2-azabicyclo[3.1.0]hexan-3-yl)-4-(1-hydroxyethyl)-1H-pyrrolo[3,2-c]quinolin-8-yl)propanenitrile;

3-(1-((1R,4R,5S)-2-Azabicyclo[2.1.1]hexan-5-yl)-7-(2,3-dichlorophenyl)-6-fluoro-2-((1R,3R,5R)-2-(1-fluorocyclopropane-1-carbonyl)-2-azabicyclo[3.1.0]hexan-3-yl)-4-methyl-1H-pyrrolo[3,2-c]quinolin-8-yl)propanenitrile;

N-((1R)-1-(1-((1R,4R,5S)-2-Azabicyclo[2.1.1]hexan-5-yl)-8-(2-cyanoethyl)-7-(2,3-dichlorophenyl)-6-fluoro-4-(1-hydroxyethyl)-1H-pyrrolo[3,2-c]quinolin-2-yl)ethyl)-1-fluorocyclopropane-1-carboxamide;

N-((1R) - 1- (1-((1R , 4R ,5S)-2-azabicyclo[2.1.1]hexan-5-yl)-8-(2-cyanoethyl)-7-(2,3-dichlorophenyl)-6-fluoro-4-(1-hydroxyethyl)-1H-pyrrolo[3,2-c]quinolin-2-yl)ethyl) fluorocyclobutane-1-carboxamide;

3-(1-((1R,4R,5S)-2-azabicyclo[2.1.1]hexan-5-yl)-7-(3-chloro-2-methylphenyl)-2-(1-(2,6-dimethyl-3-oxo-2,3-dihydropyridazin-4-yl)ethyl)-6-fluoro-4-methyl-1H-pyrrolo[3,2-c]quinolin-8-yl)propanenitrile;

N-((1R)-1-(1-((1R , 4R , 5S) - 2- azabicyclo [2.1.1]hexan-5-yl) -8-(2- cyanoethyl)-7-(2,3-dichlorophenyl)-6-fluoro-4-methyl-1H- pyrrolo[3,2- c]quinolin-2- yl) ethyl)pyrimidine-4-carboxamide;

N-((1R)-1-(1-((1R, 4R, 5S)-2- azabicyclo [2.1.1]hexan-5-yl)-8-(2- cyanoethyl)-7- (2,3-dichlorophenyl)-6-fluoro-4-methyl-1H- pyrrolo[3,2-c]quinolin-2- yl) ethyl) pyridazine-3-carboxamide;

N-((1R)-1-(1-((1R, 4R, 5S)-2- azabicyclo[2.1.1]hexan-5-yl)-8-(2- cyanoethyl)-7-(2,3-dichlorophenyl)-6-fluoro-4-methyl- 1H- pyrrolo[3,2- c]quinolin-2-yl) ethyl)-3,3- difluoroazetidine-1-carboxamide;

3-(1-((1R,4R,5S)-2-azabicyclo[2.1.1]hexan-5-yl)-7-(2,3-dichlorophenyl)-6-fluoro-4-methyl-2-((R)-1-(1-methyl-1H-pyrazol-4-yl)amino)ethyl)-1H-pyrrolo[3,2-c]quinolin-8-yl)propanenitrile;

5-(1-((1R,4R,5S)-2-Azabicyclo[2.1.1]hexan-5-yl)-8-(2-cyanoethyl)-7-(2,3-dichlorophenyl)-6-fluoro-2-((R)-1-(1-fluorocyclopropane-1-carbonyl)pyrrolidin-2-yl)-1H-pyrrolo[3,2-c]quinolin-4-yl)-N,N-dimethylpicolinamide;

methyl (2R)-2-(1-((1R,4R,5S)-2-Azabicyclo[2.1.1]hexan-5-yl)-8-(2-cyanoethyl)-7-(2,3-dichlorophenyl)-4-(4-((dimethylamino)methyl)-2,3-difluorophenyl)-6-fluoro-1H-pyrrolo[3,2-c]quinolin-2-yl)pyrrolidine-1-carboxylate, or a pharmaceutically acceptable salt thereof.

In some embodiments, the compound of Formula (III) is selected from:

3-(2-((R)-1-Acetylpyrrolidin-2-yl)-1-((1R,4R,5S)-2-azabicyclo[2.1.1]hexan-5-yl)-7-(2,3-dichlorophenyl)-6-fluoro-4-((S)-1-((S)-1-methylpyrrolidin-2-yl)ethoxy)-1H-pyrrolo[3,2-c]quinolin-8-yl)propanenitrile;

4-(1-((1R,4R,5S)-2-Azabicyclo[2.1.1]hexan-5-yl)-8-(2-cyanoethyl)-6-fluoro-7-(7-fluoronaphthalen-1-yl)-4-(S)-1-((S)-1-methylpyrrolidin-2-yl)ethoxy)-1H-pyrrolo[3 ,2-c]quinolin-2-yl)-N,N,1-trimethyl-1H-pyrazole-5-carboxamide;

3-(2-((R)-1-Acetylpyrrolidin-2-yl)-1-((1R,4R,5S)-2-azabicyclo[2.1.1]hexan-5-yl)-3-chloro-7-(2,3-dichlorophenyl)-6-fluoro-4-((S)-1-((S)-1-methylpyrrolidin-2-yl)ethoxy)-1H-pyrrolo[3,2-c]quinolin-8-yl)propanenitrile; and

8-(2-((R)-1-Acetylpyrrolidin-2-yl)-1-((1R,4R,5S)-2-azabicyclo[2.1.1]hexan-5-yl)-6-fluoro-8-methyl-4-(S)-1-((S)-1-methylpyrrolidin-2-yl)ethoxy)-1H-pyrrolo[3,2-c]quinolin-7-yl)-1,2,3,4-tetrahydronaphthalene-1-carbonitrile,

or a pharmaceutically acceptable salt thereof.

In an embodiment of a compound Formula (III), or a pharmaceutically acceptable salt thereof:

Y is N or CR⁶;

R¹ is H;

R² is selected from C₁₋₃ alkyl, C₁₋₃ haloalkyl, halo, CN, and —CH₂CH₂CN;

Cy¹ is selected from C₆₋₁₀ aryl and 6-10 membered heteroaryl; wherein the 6-10 membered heteroaryl has at least one ring-forming carbon atom and 1 ring-forming heteroatom independently selected from N and S; and wherein the C₆₋₁₀ aryl and 6-10 membered heteroaryl are each optionally substituted with 1, 2, or 3 substituents independently selected from R^{io};

R³ is selected from C₁₋₃ alkyl, C₁₋₃ haloalkyl, 4-6 membered heterocycloalkyl, 5-6 membered heteroaryl, and OR^{f3}; wherein said C₁₋₃ alkyl, 4-6 membered heterocycloalkyl, and 5-6 membered heteroaryl are each optionally substituted with 1 or 2 substituents independently selected from R³⁰;

R⁵ is H;

R⁶ is selected from H, C₁₋₃ alkyl, C₁₋₃ haloalkyl, and 5-6 membered heteroaryl; wherein said C₁₋₃ alkyl and 5-6 membered heteroaryl are each optionally substituted with 1 or 2 substituents independently selected from R⁶⁰;

R⁷ is halo;

Cy² is selected from

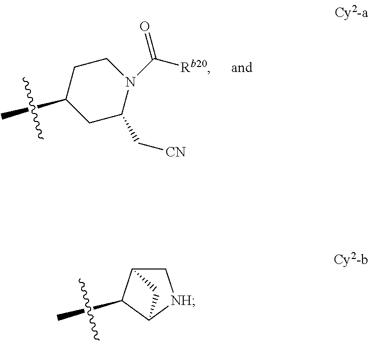

each R¹⁰ is independently selected from C₁₋₃ alkyl, C₁₋₃ haloalkyl, halo, CN, and OR^{a10};

each R³⁰ is independently selected from C₁₋₃ alkyl, C₁₋₃ haloalkyl, 4-6 membered heterocycloalkyl, halo, and NR^{c30}R^{d30}; wherein said C₁₋₃ alkyl and 4-6 membered heterocycloalkyl are each optionally substituted with 1 or 2 substituents independently selected from R³¹;

each R³¹ is independently selected from C₁₋₃ alkyl, C₁₋₃ haloalkyl, halo, and NR^{c31}R^{d31};

R³³ is selected from C₂₋₃ alkyl, C₁₋₃ haloalkyl, 4-membered heterocycloalkyl, 6-membered heterocycloalkyl, halo, and CN; wherein said C₂₋₃ alkyl, 4-membered heterocycloalkyl, and 6-membered heterocycloalkyl are each optionally substituted with 1 or 2 substituents independently selected from R³¹;

each R⁶⁰ is independently selected from C₁₋₃ alkyl, C₁₋₃ haloalkyl, 4-6 membered heterocycloalkyl, halo, and C(O)NR^{c60}R^{d60}; wherein said C₁₋₃ alkyl and 4-6 membered heterocycloalkyl are each optionally substituted with 1 or 2 substituents independently selected from R⁶¹;

each R⁶¹ is independently selected from C₁₋₃ alkyl, C₁₋₃ haloalkyl, and halo;

R^{f3} is C₁₋₃ haloalkyl; or

R^{f3} is selected from

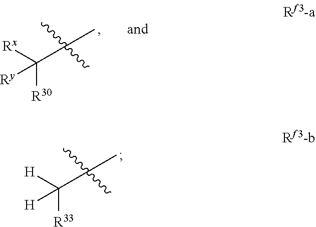

wherein R^{x} is H or C₁₋₂ alkyl and Ry is C₁₋₂ alkyl;

each Ra^{m} is independently selected from H, C₁₋₃ alkyl, and C₁₋₃ haloalkyl;

R^{b20} is selected from C₁₋₃ alkyl, and C₁₋₃ haloalkyl;

each R^{c30} and R^{d30} is independently selected from H, C₁₋₃ alkyl, and C₁₋₃ haloalkyl;

each Rc³¹ and R^{d31} is independently selected from H, C₁₋₃ alkyl, and C₁₋₃ haloalkyl; and

each R^{c60} and R^{d60} is independently selected from H, C₁₋₃ alkyl, C₁₋₃ haloalkyl, and 4-6 membered heterocycloalkyl; wherein said C₁₋₃ alkyl, and 4-6 membered heterocycloalkyl are each optionally substituted with 1 or 2 substituents independently selected from R⁶¹;

or any R^{c60} and R^{d60} attached to the same N atom, together with the N atom to which they are attached, form a 4- or 5-membered heterocycloalkyl group optionally substituted with 1 or 2 substituents independently selected from R⁶¹.

In some embodiments, the KRAS inhibitor is selected from sotorasib, adagrasib, Compound 2, Compound 3 and Compound 4, or a pharmaceutically acceptable salt thereof. In some embodiments, the KRAS inhibitor is selected from sotorasib, Compound 2, Compound 3 and Compound 4, or a pharmaceutically acceptable salt thereof. In some embodiments, the KRAS inhibitor is sotorasib, or a pharmaceutically acceptable salt thereof. In some embodiments, the KRAS inhibitor is adagrasib, or a pharmaceutically acceptable salt thereof. In some embodiments, the KRAS inhibitor is Compound 2, or a pharmaceutically acceptable salt thereof. In some embodiments, the KRAS inhibitor is Compound 3, or a pharmaceutically acceptable salt thereof. In some embodiments, the KRAS inhibitor is Compound 4, or a pharmaceutically acceptable salt thereof. In some embodiments, the KRAS inhibitor is a KRAS G12C inhibitor.

In some embodiments, the KRAS inhibitor is selected from sotorasib, adagrasib, Compound 2, Compound 3, Compound 4, Compound 5, Compound 6, and Compound 7, or a pharmaceutically acceptable salt thereof. In some embodiments, the KRAS inhibitor is selected from sotorasib, Compound 2, Compound 3, Compound 4, Compound 5, Compound 6, and Compound 7, or a pharmaceutically acceptable salt thereof. In some embodiments, the KRAS inhibitor is selected from Compound 5, Compound 6, and Compound 7, or a pharmaceutically acceptable salt thereof. In some embodiments, the KRAS inhibitor is Compound 5, or a pharmaceutically acceptable salt thereof. In some embodiments, the KRAS inhibitor is Compound 6, or a pharmaceutically acceptable salt thereof. In some embodiments, the KRAS inhibitor is Compound 7, or a pharmaceutically acceptable salt thereof In some embodiments, the KRAS inhibitor is a KRAS G12D inhibitor.

Provided herein is a method of treating cancer in a patient, comprising administering to said patient:

(i) pemigatinib, or a pharmaceutically acceptable salt thereof and

(ii) a KRAS inhibitor, which is a compound of Formula (I):

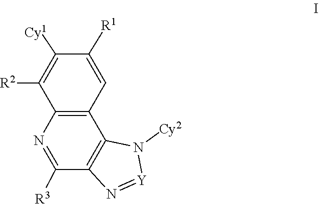

or a pharmaceutically acceptable salt thereof, wherein:

Y is N or CH;

R¹ is selected from Cl, CH₃, CH₂F, CHF₂, and CF₃;

Cy¹ is selected from

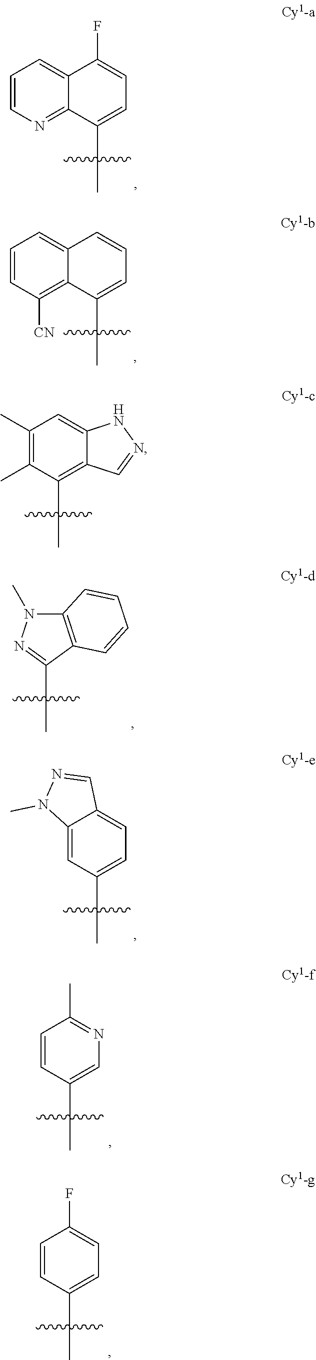

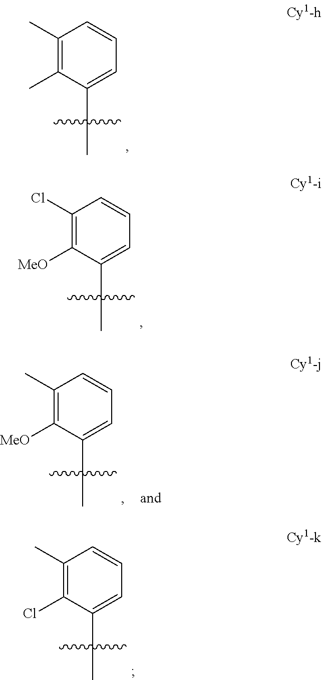

R² is selected from F and Cl;

R³ is selected from

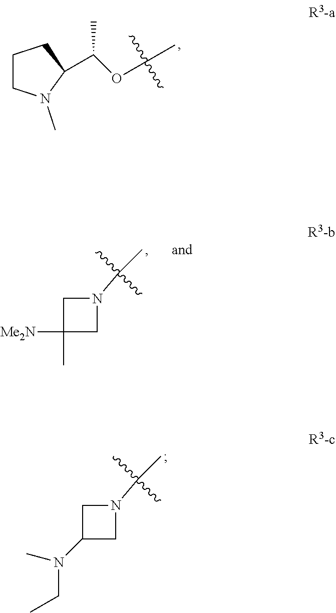

and,

Cy² is selected from

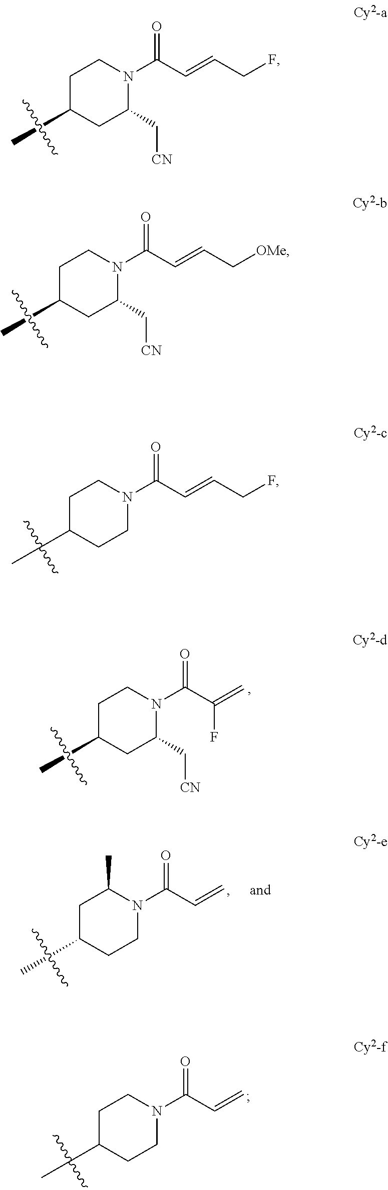

provided that the compound of Formula (I) is other than

2-((2S,4S)-4-(7-(2-chloro-3-methylphenyl)-4-(3-(dimethylamino)-3-
methylazetidin-1-yl)-6-fluoro-8-methyl-1H-[1,2,3]triazolo[4,5-c]quinolin-
1-yl)-1-((E)-4-fluorobut-2-enoyl)piperidin-2-yl)acetonitrile and
2-((2S,4S)-4-(4-(3-(dimethylamino)-3-methylazetidin-1-yl)-7-(2,3-
dimethylphenyl)-6-fluoro-8-methyl-1H-[1,2,3]triazolo[4,5-c]quinolin-
1-yl)-1-((E)-4-fluorobut-2-enoyl)piperidin-2-yl)acetonitrile.

In some embodiments, the compound of Formula (I) or the pharmaceutically acceptable salt thereof is selected from:

2-((2S,4S)-4-(4-(3-(dimethylamino)-3-methylazetidin-1-yl)-6-fluoro-7-(5-fluoroquinolin-8-yl)-8-(trifluoromethyl)-1H-[1,2,3]triazolo[4,5-c]quinolin-1-yl)-1-((E)-4-fluorobut enoyl)piperidin-2-yl)acetonitrile;

2-((2S,4S)-4-(4-(3-(dimethylamino)-3-methylazetidin-1-yl)-6-fluoro-7-(2-methoxy-3-methylphenyl)-8-(trifluoromethyl)-1H-[1,2,3]triazolo[4,5-c]quinolin-1-yl)-((E)-4-fluorobut-2-enoyl)piperidin-2-yl)acetonitrile;

2-((2S,4S)-4-(7-(3-chloro-2-methoxyphenyl)-4-(3-(dimethylamino)-3-methylazetidin-1-yl)-6-fluoro-8-(trifluoromethyl)-1H-[1,2,3]triazolo[4,5-c]quinolin-1-yl)-1-((E)-4-fluorobut-2-enoyl)piperidin-2-yl)acetonitrile;

2-((2S,4S)-4-(6-fluoro-7-(5-fluoroquinolin-8-yl)-4-(S)-1-(S)-1-methylpyrrolidin-2-yl)ethoxy)-8-(trifluoromethyl)-1H-[1,2,3]triazolo[4,5-c]quinolin-1-yl)-1-((E)-4-fluorobut-2-enoyl)piperidin-2-yl)acetonitrile;

1-(4-(6-fluoro-7-(5-fluoroquinolin-8-yl)-4-((S)-1-((S)-1-methylpyrrolidin-2-yl)ethoxy)-8-(trifluoromethyl)-1H-[1,2,3]triazolo[4,5-c]quinolin-1-yl)piperidin-1-yl)prop-2-en-1-one;

2-((2S,4S)-4-(4-(3-(dimethylamino)-3-methylazetidin-1-yl)-'7-(2,3-dimethylphenyl)-6-fluoro-8-methyl-1H-[1,2,3]triazolo[4,5-c]quinolin-1-yl)-1-((E)-4-methoxybut-2-enoyl)piperidin-2-yl)acetonitrile;

2-((2S,4S)-4-(6-fluoro-8-methyl-7-(1-methyl-1H-indazol-6-yl)-4-((S)-1-(S)-1-methylpyrrolidin-2-yl)ethoxy)-1H-[1,2,3]triazolo[4,5-c]quinolin-1-yl)-1-((E)-4-fluorobut-2-enoyl)piperidin-2-yl)acetonitrile;

2-((2S,4S)-4-(6-fluoro-8-methyl-7-(6-methylpyridin-3-yl)-4-((S)-1-((S)-1-methylpyrrolidin-2-yl)ethoxy)-1H-[1,2,3]triazolo[4,5-c]quinolin-1-yl)-1-((E)-4-fluorobut-2-enoyl)piperidin-2-yl)acetonitrile;

2-((2S,4S)-4-(6-fluoro-8-methyl-7-(1-methyl-1H-indazol-3-yl)-4-((S)-1-((S)-1-methylpyrrolidin-2-yl)ethoxy)-1H-[1,2,3]triazolo[4,5-c]quinolin-1-yl)-1-(E)-4-methoxybut-2-enoyl)piperidin-2-yl)acetonitrile;

2-((2S,4S)-4-(6-fluoro-7-(4-fluorophenyl)-8-methyl-4-((S)-1-((S)-1-methylpyrrolidin-2-yl)ethoxy)-1H-[1,2,3]triazolo[4,5-c]quinolin-1-yl)-1-((E)-4-methoxybut-2-enoyl)piperidin-2-yl)acetonitrile;

8-(1-(1-acryloylpiperidin-4-yl)-6-fluoro-8-methyl-4-(S)-1-(S)-1-methylpyrrolidin yl)ethoxy)-1H-[1,2,3]triazolo[4,5-c]quinolin-7-yl)-1-naphthonitrile;

2-((2S,4S)-4-(7-(2-chloro-3-methylphenyl)-4-(3-(dimethylamino)-3-methylazetidin-1-yl)-6-fluoro-8-methyl-1H-imidazo[4,5-c]quinolin-1-yl)-1-((E)-4-fluorobut-2-enoyl)piperidin yl)acetonitrile;

2-((2S,4S)-4-(7-(2-chloro-3-methylphenyl)-6-fluoro-8-methyl-4-((S)-1-((S)-1-methylpyrrolidin-2-yl)ethoxy)-1H-imidazo[4,5-c]quinolin-1-yl)-1-(2-fluoroacryloyl)piperidin-2-yl)acetonitrile;

8-(1-((2R,4S)-1-acryloyl-2-methylpiperidin-4-yl)-6-fluoro-8-methyl-4-(S)-1-(S)-1-methylpyrrolidin-2-yl)ethoxy)-1H-[1,2,3]triazolo[4,5-c]quinolin-7-yl)-1-naphthonitrile;

2-((2S,4S)-4-(7-(5, 6-dimethyl-1H-indazol-4-yl)-4-(3-(ethyl(methyl)amino)azetidin-1-yl)-6-fluoro-8-methyl-1H-[1,2,3]triazolo[4,5-c]quinolin-1-yl)-1-(2-fluoroacryloyl)piperidin-2-yl)acetonitrile;

8-(6-fluoro-1-(14(E)-4-fluorobut-2-enoyl)piperidin-4-yl)-8-methyl-4-(S)-1-((S)-1-methylpyrrolidin-2-yl)ethoxy)-1H-[1,2,3]triazolo[4,5-c]quinolin-7-yl)-1-naphthonitrile;

8-(1-((2S,4S)-2-(cyanomethyl)-1-(2-fluoroacryloyl)piperidin-4-yl)-4-(3-(dimethylamino)-3-methylazetidin-1-yl)-6-fluoro-8-methyl-1H-[1,2,3]triazolo[4,5-c]quinolin-7-yl)-1-naphthonitrile;

2-((2S,4S)-4-(6, 8-dichloro-4-(3-(dimethylamino)-3-methylazetidin-1-yl)-'7-(5-fluoroquinolin-8-yl)-1H-[1,2,3 ]triazolo[4, 5-c]quinolin-1-yl)-1-((E)-4-methoxybut-2-enoyl)piperidin-2-yl)acetonitrile;

2-((2S,4S)-4-(6, 8-dichloro-4-(3-(dimethylamino)-3-methylazetidin-1-yl)-'7-(5-fluoroquinolin-8-yl)-1H-[1,2,3 ]triazolo[4, 5-c]quinolin-1-yl)-1-((E)-4-fluorobut-2-enoyl)piperidin-2-yl)acetonitrile; and

2-((2S,4S)-4-(4-(3-(dimethylamino)-3-methylazetidin-1-yl)-6-fluoro-7-(5-fluoroquinolin-8-yl)-8-(trifluoromethyl)-1H-[1,2,3]triazolo[4,5-c]quinolin-1-yl)-1-(E)-4-methoxybut-2-enoyl)piperidin-2-yl)acetonitrile;

or a pharmaceutically acceptable salt thereof.

Provided herein is a method of treating cancer in a patient, comprising administering to said patient:

(i) pemigatinib, or a pharmaceutically acceptable salt thereof; and

(ii) a KRAS inhibitor, which is a compound of Formula (II):

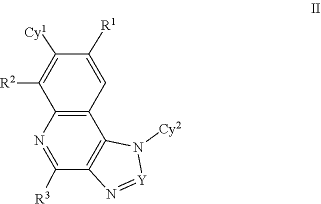

or a pharmaceutically acceptable salt thereof, wherein:

R¹ is selected from Cl, CH₃, CH₂F, CHF₂, and CF₃;

Cy¹ is selected from

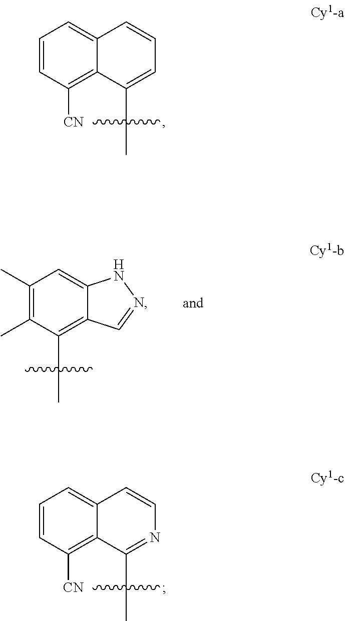 [SMILES: *c1cccc2cccc(C#N)c12 ; *c1c(C)c(C)cc2[nH]ncc12 ; "and" ; *c1nccc2cccc(C#N)c12]

R² is selected from F and Cl;

R³ is selected from

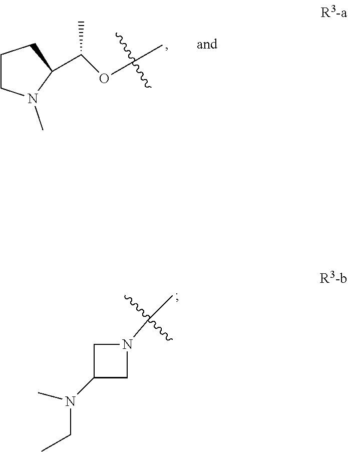

and,

Cy² is selected from

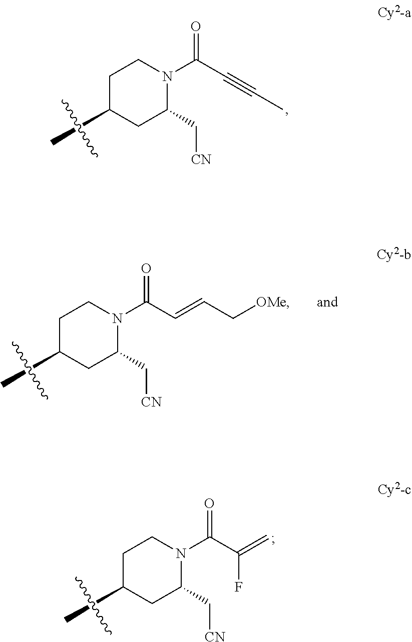

provided that the compound of Formula (II) is other than,

8-(1-((2S,4S)-2-(cyanomethyl)-1-(2-fluoroacryloyl)piperidin-4-yl)-6-fluoro-8-methyl-4-((S)-
1-((S)-1-methylpyrrolidin-2-yl)ethoxy)-1H-pyrazolo[4,3-c]quinolin-7-yl)-1-naphthonitrile,
8-(1-((2S,4S)-2-(cyanomethyl)-1-((E)-4-methoxybut-2-enoyl)piperidin-4-yl)-6-fluoro-8-
methyl-4-((S)-1-((S)-1-methylpyrrolidin-2-yl)ethoxy)-1H-pyrazolo[4,3-c]quinolin-7-yl)-1-
naphthonitrile,
2-((2S,4S)-4-(7-(5,6-dimethyl-1H-indazol-4-yl)-6-fluoro-8-methyl-4-((S)-1-((S)-1-
methylpyrrolidin-2-yl)ethoxy)-1H-pyrazolo[4,3-c]quinolin-1-yl)-1-(2-
fluoroacryloyl)piperidin-2-yl)acetonitrile,
2-((2S,4S)-1-(but-2-ynoyl)-4-(7-(5,6-dimethyl-1H-indazol-4-yl)-6-fluoro-8-methyl-4-((S)-1-
((S)-1-methylpyrrolidin-2-yl)ethoxy)-1H-pyrazolo[4,3-c]quinolin-1-yl)piperidin-2-
yl)acetonitrile, and
2-((2S,4S)-4-(7-(5,6-dimethyl-1H-indazol-4-yl)-6-fluoro-8-methyl-4-((S)-1-((S)-1-
methylpyrrolidin-2-yl)ethoxy)-1H-pyrazolo[4,3-c]quinolin-1-yl)-1-((E)-4-methoxybut-2-
enoyl)piperidin-2-yl)acetonitrile.

In some embodiments, the compound of Formula (II) or the pharmaceutically acceptable salt thereof is selected from:

1-(1-(2S,4S)-1-(but-2-ynoyl)-2-(cyanomethyl)piperidin-4-yl)-8-chloro-6-fluoro-4-(S)-1-((S)-1-methylpyrrolidin-2-yl)ethoxy)-1H-pyrazolo[4,3-c]quinolin-7-yl)isoquinoline-8-carbonitrile;

1-(8-chloro-1-(2S,4S)-2-(cyanomethyl)-1-(E)-4-methoxybut-2-enoyl)piperidin-4-yl)-6-fluoro-4-(S)-1-((S)-1-methylpyrrolidin-2-yl)ethoxy)-1H-pyrazolo[4,3-c]quinolin-7-yl)isoquinoline-8-carbonitrile;

1-(8-chloro-1-(2S,4S)-2-(cyanomethyl)-1-(2-fluoroacryloyl)piperidin-4-yl)-6-fluoro-4-((S)-1-(S)-1-methylpyrrolidin-2-yl)ethoxy)-1H-pyrazolo[4,3-c]quinolin-7-yl)isoquinoline-8-carbonitrile;

2-((2S,4S)-4-(8-chloro-7-(5,6-dimethyl-1H-indazol-4-yl)-4-(3-(ethyl(methyl)amino)azetidin-1-yl)-6-fluoro-1H-pyrazolo[4,3-c]quinolin-1-yl)-1-(2-fluoroacryloyl)piperidin-2-yl)acetonitrile;

8-(8-chloro-1-(2S,4S)-2-(cyanomethyl)-1-(2-fluoroacryloyl)piperidin-4-yl)-6-fluoro-4-((S)-1-(S)-1-methylpyrrolidin-2-yl)ethoxy)-1H-pyrazolo[4,3-c]quinolin-7-yl)-1-naphthonitrile;

8-(8-chloro-1-(2S,4S)-2-(cyanomethyl)-1-(E)-4-methoxybut-2-enoyl)piperidin-4-yl)-6-fluoro-4-(S)-1-((S)-1-methylpyrrolidin-2-yl)ethoxy)-1H-pyrazolo[4,3-c]quinolin-7-yl)-1-naphthonitrile; and

8-(1-(2S,4S)-1-(but-2-ynoyl)-2-(cyanomethyl)piperidin-4-yl)-6-fluoro-8-methyl-4-(S)-1-(S)-1-methylpyrrolidin-2-yl)ethoxy)-1H-pyrazolo[4,3-c]quinolin-7-yl)-1-naphthonitrile;

or a pharmaceutically acceptable salt thereof.

Provided herein is a method of treating cancer in a patient, comprising administering to said patient:

pemigatinib, or a pharmaceutically acceptable salt thereof; and

(ii) a KRAS inhibitor, which is a compound of Formula (III):

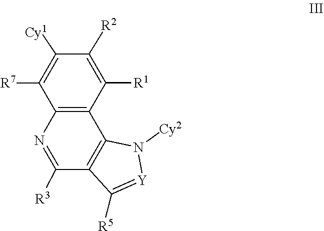

or a pharmaceutically acceptable salt thereof, wherein:

Y is N or CR⁶;

R¹ is selected from H, C₁₋₃ alkyl, C₁₋₃ haloalkyl, cyclopropyl, halo, D, CN, and OR^{a1}; wherein said C₁₋₃ alkyl and cyclopropyl are each optionally substituted with 1 or 2 substituents independently selected from R^{g};

R² is selected from H, C₁₋₃ alkyl, C₁₋₃ haloalkyl, 4-6 membered heterocycloalkyl, phenyl, 5-6 membered heteroaryl, 4-6 membered heterocycloalkyl-C₁₋₃ alkylene, phenyl-C₁₋₃ alkylene, 5-6 membered heteroaryl-C₁₋₃ alkylene, halo, D, CN, and OR^{a2}; wherein said C₁₋₃ alkyl, 4-6 membered heterocycloalkyl, phenyl, 5-6 membered heteroaryl, 4-6 membered heterocycloalkyl-C₁₋₃ alkylene, phenyl-C₁₋₃ alkylene, 5-6 membered heteroaryl-Cl-3 alkylene are each optionally substituted with 1 or 2 substituents independently selected from R^{g};

Cy¹ is selected from C₃₋₁₀ cycloalkyl, 4-10 membered heterocycloalkyl, C₆₋₁₀ aryl and 6-10 membered heteroaryl; wherein the 4-10 membered heterocycloalkyl and 6-10 membered heteroaryl each has at least one ring-forming carbon atom and 1, 2, 3, or 4 ring-forming heteroatoms independently selected from N, 0, and S; wherein a ring-forming carbon atom of 6-10 membered heteroaryl and 4-10 membered heterocycloalkyl is optionally substituted by oxo to form a carbonyl group; and wherein the C₃₋₁₀ cycloalkyl, 4-10 membered heterocycloalkyl, C₆₋₁₀ aryl and 6-10 membered heteroaryl are each optionally substituted with 1, 2, 3, or 4 substituents independently selected from R¹⁰;

R³ is selected from H, C₁₋₃ alkyl, C₁₋₃ haloalkyl, C₃₋₆ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, halo, D, CN, OR^{f3}, C(O)NR^{c3}R^{d3}, NR^{c3}R^{j3}, and NR^{c3}C(O)R^{b3}; wherein said C₁₋₃ alkyl, C₃₋₆ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene are each optionally substituted with 1, 2, or 3 substituents independently selected from R³⁰;

R⁵ is selected from H, C₁₋₃ alkyl, C₁₋₃ haloalkyl, cyclopropyl, halo, D, CN, and ORa⁵; wherein said C₁₋₃ alkyl and cyclopropyl are each optionally substituted with 1 or 2 substituents independently selected from Rg;

R⁶ is selected from H, C₁₋₃ alkyl, C₁₋₃ haloalkyl, C₃₋₆ cycloalkyl, 4-9 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, halo, D, CN, OR^{f3}, and C(O)NR^{c6}R^{d6}; wherein said C₁₋₃ alkyl, C₃₋₆ cycloalkyl, 4-9 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene are each optionally substituted with 1 or 2 substituents independently selected from R⁶⁰;

R⁷ is selected from H, C₁₋₃ alkyl, C₁₋₃ haloalkyl, cyclopropyl, halo, D, CN, and OR^{a7}; wherein said C₁₋₃ alkyl and cyclopropyl are each optionally substituted with 1 or 2 substituents independently selected from R^{g};

Cy² is selected from

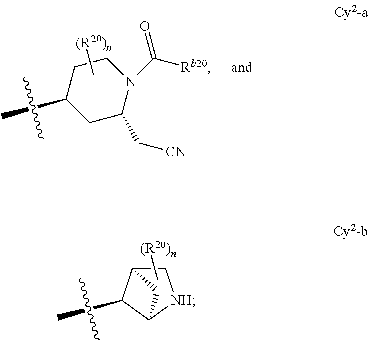

wherein n is 0, 1, or 2;

each R¹⁰ is independently selected from C₁₋₃ alkyl, C₁₋₃ haloalkyl, halo, D, CN, OR^{a10}, C(O)R^{b10}, C(O)NR^{c10}R^{d10}, C(O)OR^{a10}, NR^{c10}, R^{d10}, and S(O)₂R^{b10};

each R²⁰ is independently selected from C₁₋₃ alkyl, C₁₋₃ haloalkyl, halo, D, CN, and OR^{a20};

each R³⁰ is independently selected from C₁₋₃ alkyl, C₁₋₃ haloalkyl, C₃₋₆ cycloalkyl, 4-6 membered heterocycloalkyl, phenyl, 5-6 membered heteroaryl, halo, D, CN, OR^{a30}, C(O)R^{b30}, C(O)NR^{c30}R^{d30}, C(O)OR^{a30}, NR^{c30}R^{d30}, S(O)₂R^{b30}; wherein said C₁₋₃ alkyl, C₃₋₆ cycloalkyl, 4-6 membered heterocycloalkyl, phenyl, and 5-6 membered heteroaryl are each optionally substituted with 1 or 2 substituents independently selected from R³¹;

each R³¹ is independently selected from C₁₋₃ alkyl, C₁₋₃ haloalkyl, halo, D, CN, OR^{a31}, C(O)R^{b31}; C(O)NR^{c31}R^{d31}; C(O)ORₐ₃₁, NR_{c31}R_{d31} and S(O)₂R^{b31};

each R³³ is independently selected from C₁₋₃ alkyl, C₁₋₃ haloalkyl, C₃₋₆ cycloalkyl, 4-membered heterocycloalkyl, 6-membered heterocycloalkyl, phenyl, 5-6 membered heteroaryl, halo, D, CN, OR^{a30}, C(O)NR^{a30}R^{d30}, and NR^{c30}R^{d30}, wherein said C₁₋₃ alkyl, C₃₋₆ cycloalkyl, 4-membered heterocycloalkyl, 6-membered heterocycloalkyl, phenyl, and 5-6 membered heteroaryl are each optionally substituted with 1 or 2 substituents independently selected from R³¹; each R⁶⁰ is independently selected from C₁₋₃ alkyl, C₁₋₃ haloalkyl, 4-6 membered heterocycloalkyl, 5-6 membered heteroaryl, halo, D, CN, OR^{a60}, C(O)R^{b60}, C(O)NR^{c60}R^{d60}, NR^{c60}C(O)R^{b60}C(O)OR^{a60}, NR^{c60}C(O)OR^{a60}NR^{c60}R^{d60}NR^{c60}S(O)₂R^{b60} and S(O)₂R^{b60}; wherein said C₁₋₃ alkyl, 4-6 membered heterocycloalkyl, and 5-6 membered heteroaryl are each optionally substituted with 1 or 2 substituents independently selected from R⁶¹;

each R⁶¹ is independently selected from C₁₋₃ alkyl, C₁₋₃ haloalkyl, halo, D, CN, OR^{a61}, and NR^{c61}R^{d61};

R^{a1} is selected from H, C₁₋₃ alkyl, and C₁₋₃ haloalkyl;

each R^{a2} is independently selected from H, C₁₋₃ alkyl, and C₁₋₃ haloalkyl;

each R^{b3}, R^{c3} and R^{d3} is independently selected from H, C₁₋₃ alkyl, C₁₋₃ haloalkyl, C₃₋₆ cycloalkyl, 4-6 membered heterocycloalkyl, phenyl and 5-6 membered heteroaryl; wherein said , C₁₋₃ alkyl, C₃₋₆ cycloalkyl, 4-6 membered heterocycloalkyl, phenyl and 5-6 membered heteroaryl are each optionally substituted with 1, 2, or 3 substituents independently selected from R³⁰;

or R^{c30} and R^{d3} attached to the same N atom, together with the N atom to which they are attached, form a 4-, 5-, or 6-membered heterocycloalkyl group optionally substituted with 1, 2, or 3 substituents independently selected from R³⁰;

R^{j3} is selected from C₁₋₃ alkyl, C₁₋₃ haloalkyl, C₃₋₆ cycloalkyl, 4-6 membered heterocycloalkyl, phenyl and 5-6 membered heteroaryl; wherein said , C₁₋₃ alkyl, C₃₋₆ cycloalkyl, 4-6 membered heterocycloalkyl, phenyl and 5-6 membered heteroaryl are each optionally substituted with 1, 2, or 3 sub stituents independently selected from R³⁰;

or R^{c3} and R^{j3} attached to the same N atom, together with the N atom to which they are attached, form a 4-, 5-, or 6-membered heterocycloalkyl group optionally substituted with 1, 2, or 3 substituents independently selected from R³⁰;

R^{f3} is selected from C₁₋₃ haloalkyl, C₃₋₆ cycloalkyl, 4-6 membered heterocycloalkyl, phenyl and 5-6 membered heteroaryl; wherein said C₁₋₃ haloalkyl, C₃₋₆ cycloalkyl, 4-6 membered heterocycloalkyl, phenyl, and 5-6 membered heteroaryl are each optionally substituted with 1, 2, or 3 substituents independently selected from R³⁰; or

R^{f3} is selected from

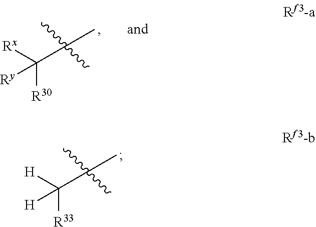

wherein IV is H or C₁₋₂ alkyl and R^{y} is C₁₋₂ alkyl;

or R^{x} and R^{y}, together with the C atom to which they are attached, form a 3-, or 4-membered cycloalkyl group;

R^{a5} is selected from H, C₁₋₃ alkyl, and C₁₋₃ haloalkyl;

each R^{a60}, R^{cb} and R^{d6} is independently selected from H, C₁₋₃ alkyl, C₁₋₃ haloalkyl, C₃₋₆ cycloalkyl, 4-6 membered heterocycloalkyl, phenyl and 5-6 membered heteroaryl; wherein said C₁₋₃ alkyl, C₃₋₆ cycloalkyl, 4-6 membered heterocycloalkyl, phenyl and 5-6 membered heteroaryl are each optionally substituted with 1 or 2 substituents independently selected from R⁶⁰;

R^{a5} is selected from H, C₁₋₃ alkyl, and C₁₋₃ haloalkyl;

each Rₐ₁₀, R^{b10}, R^{cb} and R_{d6} is independently selected from H, C₁₋₃ alkyl, and C₁₋₃ haloalkyl;

each R^{a5} is independently selected from H, C₁₋₃ alkyl, and C₁₋₃ haloalkyl; R^{b20} is selected from NH2, C₁₋₃ alkyl, and C₁₋₃ haloalkyl;

each R^{a20}, R^{b30}, R^{c30} and R^{d30} is independently selected from H, C₁₋₃ alkyl, and C₁₋₃ haloalkyl;

each R^{a31}, R^{b31}, R^{c31} and R^{d31} is independently selected from H, C₁₋₃ alkyl, and C₁₋₃ haloalkyl;

each R^{a60}, R^{b60}, R^{c60} and R^{d60} is independently selected from H, C₁₋₃ alkyl, C₁₋₃ haloalkyl, is independently selected from H, C₁₋₃ alkyl, C₁₋₃ haloalkyl,

C₃₋₆ cycloalkyl, 4-6 membered heterocycloalkyl, and 5-6 membered heteroaryl; wherein said C₁₋₃ alkyl, C₃₋₆ cycloalkyl, 4-6 membered heterocycloalkyl, and 5-6 membered heteroaryl are each optionally substituted with 1 or 2 substituents independently selected from R⁶¹;

or any R^{c60} and R^{d60} attached to the same N atom, together with the N atom to which they are attached, form a 4-, 5-, or 6-membered heterocycloalkyl group optionally substituted with 1 or 2 substituents independently selected from R⁶¹; and

each R^{a61}, R^{c61}, and R^{d61}, is independently selected from H, C₁₋₃ alkyl, and C₁₋₃ haloalkyl; and

each R^{g} is independently selected from D, OH, CN, halo, C₁₋₃ alkyl, C₁₋₃ haloalkyl, C₁₋₃ alkoxy, C₁₋₃ haloalkoxy, amino, C₁₋₃ alkylamino, and di(C₁₋₃ alkyl)amino;

provided that the compound of Formula (III) is other than,

3-(1-((1R,4R,5S)-2-azabicyclo[2.1.1]hexan-5-yl)-8-(2-cyanoethyl)-4-ethoxy-6-fluoro-7-(3-hydroxynaphthalen-1-yl)-1H-pyrrolo[3,2-c]quinolin-2-yl)-N,N-dimethylpropanamide.

In some embodiments, the compound of Formula (III) is selected from:

3-(1-((1R,4R,5S)-2-Azabicyclo[2.1.1]hexan-5-yl)-7-(7-chloro-3-hydroxynaphthalen-1-yl)-6-fluoro-2-methyl-4-(1H-1,2,4-triazol-1-yl)-1H-pyrrolo[3,2-c]quinolin-8-yl)propanenitrile;

3-(1-((1R,4R,5S)-2-Azabicyclo[2.1.1]hexan-5-yl)-7-(5,7-difluoro-1H-indol-3-yl)-6-fluoro-2-methyl-4-(S)-1-((S)-1-methylpyrrolidin-2-yl)ethoxy)-1H-pyrrolo[3,2-c]quinolin-8-yl)propanenitrile;

3-(1-((1R,4R,5S)-2-Azabicyclo[2.1.1]hexan-5-yl)-6-fluoro-7-(6-fluoro-5-methyl-1H-indol-3-yl)-2-methyl-4-(S)-1-((S)-1-methylpyrrolidin-2-yl)ethoxy)-1H-pyrrolo[3,2-c]quinolin-8-yl)propanenitrile;

3-(2-(3-(Azetidin-1-yl)-3-oxopropyl)-1-((1R, 4R, 5S)-2-azabicyclo[2.1.1]hexan-5-yl)-7-(2,3-dichlorophenyl)-6-fluoro-4-((S)-1-((S)-1-methylpyrrolidin-2-yl)ethoxy)-1H-pyrrolo[3,2-c]quinolin-8-yl)propanenitrile;

3-(1-((1R,4R,5S)-2-Azabicyclo[2.1.1]hexan-5-yl)-6-fluoro-7-(3-hydroxynaphthalen yl)-8-methyl-4-((S)-1-((S)- 1-methylpyrrolidin-2-yl)ethoxy)-1H-pyrrolo[3,2-c]quinolin yl)methyl)oxazolidin-2-one;

5-(1-((1R,4R,5S)-2-Azabicyclo[2.1.1]hexan-5-yl)-6-fluoro-2,8-dimethyl-4-(5)-1-((S) methylpyrrolidin-2-yl)ethoxy)-1H-pyrrolo[3,2-c]quinolin-7-yl)-1-naphthonitrile;

1-((2S,4S)-1-Acetyl-2-(cyanomethyl)piperidin-4-yl)-7-(8-cyanonaphthalen-1-yl) fluoro-4-((S)-14S)-1-methylpyrrolidin-2-yl)ethoxy)-1H-pyrazolo[4,3-c]quinoline-8-carbonitrile;

8-(1-((1R,4R,5S)-2-Azabicyclo[2.1.1]hexan-5-yl)-6-fluoro-8-methyl-4-((S)-1-((S)-1-methylpyrrolidin-2-yl)ethoxy)-2-((3-oxomorpholino)methyl)-1H-pyrrolo[3,2-c]quinolin-7-yl)-1-naphthonitrile;

3-(7-(Benzo[b]thiophen-3-yl)-1-((1R,4R,5S)-2-azabicyclo[2.1.1]hexan-5-yl)-6-fluoro-4-((S)-1-((S)-1-methylpyrrolidin-2-yl)ethoxy)-2-((2-oxopyrrolidin-1-yl)methyl)-1H-pyrrolo[3,2-c]quinolin-8-yl)propanenitrile;

3-(1-((1R,4R,5S)-2-Azabicyclo[2.1.1]hexan-5-yl)-4-(S)-1-(dimethylamino)propan-2-yl)oxy)-6-fluoro-7-(7-fluoronaphthalen-1-yl)-2-(2-oxopyrrolidin-1-yl)methyl)-1H-pyrrolo[3,2-c]quinolin-8-yl)propanenitrile;

8-(1-((1R,4R,5S)-2-Azabicyclo[2.1.1]hexan-5-yl)-8-(2-cyanoethyl)-6-fluoro-2-methyl-4-((S)-1-((S)-1-methylpyrrolidin-2-yl)ethoxy)-1H-pyrrolo[3,2-c]quinolin-7-yl)-1-naphthonitrile;

3-(1-(1R,4R,5S)-2-Azabicyclo[2.1.1]hexan-5-yl)-7-(2,3-dichloro-5-hydroxyphenyl)-6-fluoro-2-methyl-4-(S)-1-((S)-1-methylpyrrolidin-2-yl)ethoxy)-1H-pyrrolo[3,2-c]quinolin-8-yl)propanenitrile;

3-(1-((1R,4R,5S)-2-Azabicyclo[2.1.1]hexan-5-yl)-8-(2-cyanoethyl)-6-fluoro-4-(3-fluoro-1-methylazetidin-3-yl)methoxy)-7-(3-hydroxynaphthalen-1-yl)-1H-pyrrolo[3,2-c]quinolin-2-yl)-N,N-dimethylpropanamide;

3-(1-((1R,4R,5S)-2-Azabicyclo[2.1.1]hexan-5-yl)-6-fluoro-7-(3-hydroxynaphthalen-1-yl)-2-methyl-4-(5-methylpyrazin-2-yl)-1H-pyrrolo[3,2-c]quinolin-8-yl)propanenitrile;

3-(1-((1R,4R,5S)-2-Azabicyclo[2.1.1]hexan-5-yl)-6-fluoro-7-(7-fluoronaphthalen-1-yl)-4-methyl-2-((4-methyl-2-oxopiperazin-1-yl)methyl)-1H-pyrrolo[3,2-c]quinolin-8-yl)propanenitrile;

3-(1-((1R,4R,5S)-2-azabicyclo[2.1.1]hexan-5-yl)-7-(2,3-dichloro-5-hydroxyphenyl)-4-ethoxy-6-fluoro-24(4-isopropyl-2-oxopiperazin-1-yl)methyl)-1H-pyrrolo[3,2-c]quinolin-8-yl)propanenitrile;

3-(1-((1R,4R,5S)-2-Azabicyclo[2.1.1]hexan-5-yl)-4-(3-(dimethylamino) methylazetidin-1-yl)-6-fluoro-7-(7-fluoronaphthalen-1-yl)-2-((3-oxomorpholino)methyl)-1H-pyrrolo[3,2-c]quinolin-8-yl)propanenitrile;

3-(1-((1R,4R,5S)-2-azabicyclo[2.1.1]hexan-5-yl)-4-ethoxy-6-fluoro-7-(3-hydroxynaphthalen-1-yl)-2-(1-(3-oxomorpholino)ethyl)-1H-pyrrolo[3,2-c]quinolin-8-yl)propanenitrile;

3-(1-((endo)-2-Azabicyclo[2.1.1]hexan-5-yl)-6-fluoro-7-(3-hydroxynaphthalen-1-yl)-4-((S)-1-((S)-1-methylpyrrolidin-2-yl)ethoxy)-2-(pyridin-3-yl)-1H-pyrrolo[3,2-c]quinolin-8-yl)propanenitrile;

3-(2-(3-(azetidin-1-yl)-3-oxopropyl)-1-(1R,4R,5S)-2-azabicyclo[2.1.1]hexan-5-yl)-7-(7,8-difluoronaphthalen-1-yl)-6-fluoro-4-((S)-1-((S)-1-methylpyrrolidin-2-yl)ethoxy)-1H-pyrrolo[3,2-c]quinolin-8-yl)propanenitrile;

3-(2-(3-(azetidin-1-yl)-3-oxopropyl)-1-(1R,4R,5S)-2-azabicyclo[2.1.1]hexan-5-yl)-7-(6,7-difluoronaphthalen-1-yl)-6-fluoro-4-((S)-1-((S)-1-methylpyrrolidin-2-yl)ethoxy)-1H-pyrrolo[3,2-c]quinolin-8-yl)propanenitrile;

3-(1-((1R,4R,5S)-2-azabicyclo[2.1.1]hexan-5-yl)-6-fluoro-7-(7-fluoro-3-hydroxynaphthalen-1-yl)-2-methyl-4-((S)-1-((S)-1-methylpyrrolidin-2-yl)ethoxy)-1H-pyrrolo[3,2-c]quinolin-8-yl)propanenitrile;

1-(1-(2S,4S)-1-Acetyl-2-(cyanomethyl)piperi din-4-yl)-8-chloro-6-fluoro-4-((S)-1-((S)-1-methylpyrrolidin-2-yl)ethoxy)-1H-pyrazolo[4,3-c]quinolin-7-yl)isoquinoline-8-carbonitrile;

8-(1-(2S,4S)-1-acetyl-2-(cyanomethyl)piperidin-4-yl)-8-chloro-6-fluoro-4-((S)-1-((S)-1-methylpyrrolidin-2-yl)ethoxy)-1H-pyrrolo[3,2-c]quinolin-7-yl)-1-naphthonitrile;

8-(1-(2S,4S)-1-acetyl-2-(cyanomethyl)piperidin-4-yl)-8-chloro-6-fluoro-4-((S)-1-((S)-1-methylpyrrolidin-2-yl)ethoxy)-1H-pyrazolo[4,3-c]quinolin-7-yl)-1-naphthonitrile;

3-(1-((1R,4R,5S)-2-Azabicyclo[2.1.1]hexan-5-yl)-6-fluoro-7-(7-fluoro-3-hydroxynaphthalen-1-yl)-2-methyl-4-(1H-1,2,4-triazol-1-yl)-1H-pyrrolo[3,2-c]quinolin-8-yl)propanenitrile;

3-(1-((1R,4R,5S)-2-Azabicyclo[2.1.1]hexan-5-yl)-6-fluoro-7-(7-fluoronaphthalen-1-yl)-2-methyl-4-(S)-1-((S)-1-methylpyrrolidin-2-yl)ethoxy)-1H-pyrrolo[3,2-c]quinolin yl)propanenitrile;

(2R)-2-(1-((1R,4R,5S)-2-azabicyclo[2.1.1]hexan-5-yl)-8-(2-cyanoethyl)-7-(2,3-dichlorophenyl)-6-fluoro-4-(1H-1,2,4-triazol-1-yl)-1H-pyrrolo[3,2-c]quinolin-2-yl)-N,N-dimethylpyrrolidine-1-carboxamide;

methyl (2R)-2-(1-((1R,4R,5S)-2-azabicyclo[2.1.1]hexan-5-yl)-7-(2-chloro-3-methylphenyl)-8-(2-cyanoethyl)-6-fluoro-4-(1H-1,2,4-triazol-1-yl)-1H-pyrrolo[3,2-c]quinolin-2-yl)pyrrolidine-1-carboxylate;

Methyl (1S,3R,5S)-3-(1-((1R,4R,5S)-2-azabicyclo[2.1.1]hexan-5-yl)-8-(2-cyanoethyl)-7-(2,3-dichlorophenyl)-4-(6-(dimethylcarbamoyl)pyridin-3-yl)-6-fluoro-1H-pyrrolo[3,2-c]quinolin-2-yl)-2-azabicyclo[3.1.0]hexane-2-carboxylate;

3-(1-((1R,4R,5S)-2-Azabicyclo[2.1.1]hexan-5-yl)-7-(2,3-dichlorophenyl)-6-fluoro-4-methyl-2-(5-oxo-1,2,3,5-tetrahydroindolizin-3-yl)-1H-pyrrolo[3,2-c]quinolin-8-yl)propanenitrile;

Methyl (2R)-2-(1-((1R,4R,5S)-2-azabicyclo[2.1.1]hexan-5-yl)-8-(2-cyanoethyl)-7-(2,3-dichlorophenyl)-4-(6-(dimethylcarbamoyl)pyridin-3-yl)-6-fluoro-1H-pyrrolo[3,2-c]quinolin-2-yl)pyrrolidine-1-carboxylate;

Methyl (2R)-2-(1-((1R,4R,5S)-2-azabicyclo[2.1.1]hexan-5-yl)-8-(2-cyanoethyl)-7-(2,3-dichlorophenyl)-6-fluoro-4-(6-(methylcarbamoyl)pyridin-3-yl)-1H-pyrrolo[3,2-c]quinolin-2-yl)pyrrolidine-1-carboxylate;

3-(1-((1R,4R,5S)-2-Azabicyclo[2.1.1]hexan-5-yl)-7-(2-chloro-3-fluorophenyl)-2-((R)-1-(cyclopropanecarbonyl)pyrrolidin-2-yl)-6-fluoro-4-methyl-1H-pyrrolo[3,2-c]quinolin-8-yl)propanenitrile;

8-(24(R)-1-Acetylpyrrolidin-2-yl)-1-(1R,4R,5S)-2-azabicyclo[2.1.1]hexan-5-yl)-6-fluoro-8-methyl-4-(2-methylpyridin-4-yl)-1H-pyrrolo[3,2-c]quinolin-7-yl)-1,2,3,4-tetrahydronaphthalene-1-carbonitrile;

5-(1-((1R,4R,5S)-2-Azabicyclo[2.1.1]hexan-5-yl)-7-(3-chloro-2-methylphenyl)-8-(2-cyanoethyl)-6-fluoro-2-((R)-1-(2-oxopyrazin-1(2H)-yl)ethyl)-1H-pyrrolo[3,2-c]quinolin-4-yl)-N-methylpicolinamide;

3-(1-((1R,4R,5S)-2-Azabicyclo[2.1.1]hexan-5-yl)-6-fluoro-7-(7-fluoronaphthalen-1-yl)-4-(6-(2-hydroxypropan-2-yl)pyridin-3-yl)-2-((R)-1-(2-oxopyrazin-1(2H)-yl)ethyl)-1H-pyrrolo[3,2-c]quinolin-8-yl)propanenitrile;

3-(1-((1R,4R,5S)-2-Azabicyclo[2 .1. 1]hexan-5-yl)-7-(3-chloro-2-methylphenyl)-6-fluoro-4-(5-methylpyrazin-2-yl)-2-((R)-1-(2-oxopyrazin-1(2H)-yl)ethyl)-1H-pyrrol o [3 ,2-c]quinolin yl)propanenitrile;

Methyl (2R)-2-(1-((1R,4R,5S)-2-azabicyclo[2.1.1]hexan-5-yl)-8-(2-cyanoethyl)-7-(2,3-dichlorophenyl)-6-fluoro-4-(5-fluoro-6-(methylcarbamoyl)pyridin-3-yl)-1H-pyrrolo[3,2-c]quinolin-2-yl)pyrrolidine-1-carboxylate;

Methyl (2R)-2-(1-((1R,4R,5S)-2-azabicyclo [2.1.1]hexan-5-yl)-8-(2-cyanoethyl)-7-(2,3-dichlorophenyl)-6-fluoro-4-(6-(2-hydroxypropan-2-yl)pyridin-3-yl)-1H-pyrrolo[3 ,2-c]quinolin-2-yl)pyrrolidine-1-carboxylate;

Ethyl (2R)-2-(1-((1R,4R,5S)-2-azabicyclo[2 1.1]hexan-5-yl)-8-(2-cyanoethyl)-7-(2,3-dichlorophenyl)-6-fluoro-4-(6-(2-hydroxypropan-2-yl)pyridin-3-yl)-1H-pyrrolo[3 ,2-c]quinolin-2-yl)pyrrolidine-1-carboxylate;

3-(1-((1R,4R,5S)-2-Azabicyclo[2 1.1]hexan-5-yl)-7-(2,3-dichlorophenyl)-2-((R)-1-(3,3-difluoroazetidine-1-carbonyl)pyrrolidin-2-yl)-6-fluoro-4-(methyl-d3)-1H-pyrrolo[3 ,2-c]quinolin-8-yl)propanenitrile;

3-(1-((1R,4R,5S)-2-Azabicyclo[2 1.1]hexan-5-yl)-7-(2,3-dichlorophenyl)-2-((R)-1-(3 ,3-difluoroazetidine-1-carbonyl)pyrrolidin-2-yl)-6-fluoro-1H-pyrrolo[3,2-c]quinolin-8-yl)propanenitrile;

3-(1-((1R,4R,5S)-2-Azabicyclo[2 .1. 1]hexan-5-yl)-7-(3-chloro-2-methylphenyl)-6-fluoro-4-(5-methylpyrazin-2-yl)-2-((R)-1-(3-oxomorpholino)ethyl)-1H-pyrrolo[3 ,2-c]quinolin-8-yl)propanenitrile;

5-(1-((1R,4R,5S)-2-Azabicyclo[2.1.1]hexan-5-yl)-8-(2-cyanoethyl)-6-fluoro-7-(7-fluoronaphthalen-1-yl)-24(R)-1-(3-oxomorpholino)ethyl)-1H-pyrrolo[3,2-c]quinolin-4-yl)-N-methylpicolinamide;

3-(1-((1R,4R,5S)-2-Azabicyclo[2 .1. 1]hexan-5-yl)-6-fluoro-7-(7-fluoronaphthalen-1-yl)-4-(6-(2-hydroxypropan-2-yl)pyri din-3-yl)-2-((R)-1-(3-oxomorpholino)ethyl)-1H-pyrrolo [3,2-c]quinolin-8-yl)propanenitrile;

3-(1-((1R,4R,5S)-2-Azabicyclo[2.1.1]hexan-5-yl)-6-fluoro-7-(7-fluoronaphthalen-1-yl)-4-(5-methylpyrazin-2-yl)-2-((R)-1-(3-oxomorpholino)ethyl)-1H-pyrrolo[3,2-c]quinolin yl)propanenitrile;

Methyl (1R,3R,5R)-3-(1-((1R,4R,5S)-2-azabicyclo[2.1.1]hexan-5-yl)-8-(2-cyanoethyl)-7-(2,3-dichlorophenyl)-4-(6-(dimethylcarbamoyl)pyridin-3-yl)-6-fluoro-1H-pyrrolo[3,2-c]quinolin-2-yl)-2-azabicyclo[3.1.0]hexane-2-carboxylate;

3-(1-((1R,4R,5S)-2-Azabicyclo[2.1.1]hexan-5-yl)-2-(1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile;

3-(1-((1R,4R,5S)-2-Azabicyclo[2.1.1]hexan-5-yl)-7-(2,3-dichlorophenyl)-6-fluoro-4-(6-(2-hydroxypropan-2-yl)pyridin-3-yl)-2-((R)-1-(2-oxopyrazin-1(2H)-yl)ethyl)-1H-pyrrolo[3,2-c]quinolin-8-yl)propanenitrile;

Methyl (2R,4S)-2-(1-((1R,4R,5S)-2-azabicyclo[2.1.1]hexan-5-yl)-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)-4-fluoropyrrolidine-1-carboxylate;

Methyl (2R,5R)-2-(1-((1R,4R,5S)-2-azabicyclo[2.1.1]hexan-5-yl)-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)-5-methylpyrrolidine-1-carboxylate;

Methyl (2R)-2-(1-((1R,4R,5S)-2-azabicyclo[2.1.1]hexan-5-yl)-3-chloro-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)pyrrolidine-1-carboxylate;

4-(1-((1R,4R,5S)-2-Azabicyclo[2.1.1]hexan-5-yl)-8-(2-cyanoethyl)-7-(2,3-dichlorophenyl)-6-fluoro-2-((R)-1-(2-oxopyrazin-1(2H)-yl)ethyl)-1H-pyrrolo[3,2-c]quinolin-4-yl)-2-fluoro-N-methylbenzamide;

Methyl ((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)carbamate;

N-((1R)-1-(1-((1R,4R,5S)-2-Azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)-2,2-difluoroacetamide;

N-((1R)-1-(1-((1R,4R,5S)-2-Azabicyclo[2.1.1]hexan-5-yl)-8-(2-cyanoethyl)-7-(2,3-dichlorophenyl)-6-fluoro-4-methyl-1H-pyrrolo[3 ,2-c]quinolin-2-yl)ethyl)-2,2-difluoroacetamide;

(2S)-N-((1R)-1-(1-((1R,4R,5S)-2-Azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)tetrahydrofuran carboxamide;

N-(1R)-1-(1-((1R,4R,5S)-2-Azabicyclo[2.1.1]hexan-5-yl)-8-(2-cyanoethyl)-7-(2,3-dichlorophenyl)-6-fluoro-4-methyl-1H-pyrrolo[3,2-c]quinolin yl)ethyl)cyclopropanesulfonamide;

N-((1R)-1-(1-((1R,4R, 5S)-2-Azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)thiazole-4-carboxamide;

N-((1R)-1-(1-((1R,4R,5S)-2-Azabicyclo[2.1.1]hexan-5-yl)-8-(2-cyanoethyl)-7-(2 ,3-dichlorophenyl)-6-fluoro-4-methyl-1H -pyrrolo[3,2-c]quinolin-2-yl)ethyl)-N-methylcyclopropanecarboxamide;

N-((1R)-1-(1-((1R,4R,5S)-2-Azabicyclo[2.1.1]hexan-5-yl)-8-(2-cyanoethyl)-7-(2,3-dichlorophenyl)-6-fluoro-4-(1-hydroxyethyl)-1H-pyrrolo[3,2-c]quinolin-2-yl)ethyl)-1-methylcyclopropane-1-carboxamide;

3-(1-((1R,4R,5S)-2-Azabicyclo[2.1.1]hexan-5-yl)-7-(2,3-dichlorophenyl)-6-fluoro-4-(1-hydroxyethyl)-2-((1R,3R,5R)-2-(1-methylcyclopropane-1-carbonyl)-2-azabicyclo[3.1.0]hexan-3-yl)-1H-pyrrolo[3,2-c]quinolin-8-yl)propanenitrile;

3-(1-((1R,4R,5S)-2-Azabicyclo[2.1.1]hexan-5-yl)-7-(2,3-dichlorophenyl)-6-fluoro-2-((1R,3R,5R)-2-(1-fluorocyclopropane-1-carbonyl)-2-azabicyclo[3.1.0]hexan-3-yl)-4-(1-hydroxyethyl)-1H-pyrrolo[3,2-c]quinolin-8-yl)propanenitrile;

3-(1-((1R,4R,5S)-2-Azabicyclo[2.1.1]hexan-5-yl)-7-(2,3-dichlorophenyl)-6-fluoro-2-((1R,3R,5R)-2-(1-fluorocyclopropane-1-carbonyl)-2-azabicyclo[3.1.0]hexan-3-yl)-4-methyl-1H-pyrrolo[3,2-c]quinolin-8-yl)propanenitrile;

N-((1R)-1-(1-((1R,4R,5S)-2-Azabicyclo[2.1.1]hexan-5-yl)-8-(2-cyanoethyl)-7-(2,3-dichlorophenyl)-6-fluoro-4-(1-hydroxyethyl)-1H-pyrrolo[3,2-c]quinolin-2-yl)ethyl)-1-fluorocyclopropane-1-carboxamide;

N-((1R)-1-(1-((1R,4R,5S)-2-azabicyclo[2.1.1]hexan-5-yl)-8-(2-cyanoethyl)-7-(2,3-dichlorophenyl)-6-fluoro-4-(1-hydroxyethyl)-1H-pyrrolo[3,2-c]quinolin-2-yl)ethyl)-1-fluorocyclobutane-1-carboxamide;

3-(1-((1R,4R,5S)-2-azabicyclo[2.1.1]hexan-5-yl)-7-(3-chloro-2-methylphenyl)-2-(1-(2,6-dimethyl-3-oxo-2,3-dihydropyridazin-4-yl)ethyl)-6-fluoro-4-methyl-1H-pyrrolo[3,2-c]quinolin-8-yl)propanenitrile;

N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H -pyrrolo[3,2-c]quinolin-2-yl)ethyl)pyrimidine carboxamide;

N-((1R)-1-(1-((1R,4R, 5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)pyridazine-3-carboxamide;

N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)-3,3-difluoroazetidine-1-carboxamide;

3-(1-((1R,4R,5S)-2-azabicyclo[2.1.1]hexan-5-yl)-7-(2,3-dichlorophenyl)-6-fluoro-4-methyl-2-((R)-1-(1-methyl-1H-pyrazol-4-yl)amino)ethyl)-1H-pyrrolo[3,2-c]quinolin-8-yl)propanenitrile;

5-(1-((1R,4R,5S)-2-Azabicyclo[2.1.1]hexan-5-yl)-8-(2-cyanoethyl)-7-(2,3-dichlorophenyl)-6-fluoro-2-(R)-1-(1-fluorocyclopropane-1-carbonyl)pyrrolidin-2-yl)-1H-pyrrolo[3,2-c]quinolin-4-yl)-N,N-dimethylpicolinamide; and

methyl (2R)-2-(1-((1R,4R,5S)-2-Azabicyclo[2.1.1]hexan-5-yl)-8-(2-cyanoethyl)-7-(2,3-dichlorophenyl)-4-(4-((dimethylamino)methyl)-2,3-difluorophenyl)-6-fluoro-1H-pyrrolo[3,2-c]quinolin-2-yl)pyrrolidine-1-carboxylate;

or a pharmaceutically acceptable salt thereof.

In some embodiments, the compound of Formula (III) is selected from:

3-(2-(R)-1-Acetylpyrrolidin-2-yl)-1-((1R,4R,5S)-2-azabicyclo[2.1.1]hexan-5-yl)-7-(2,3-dichlorophenyl)-6-fluoro-4-((S)-1-((S)-1-methylpyrrolidin-2-yl)ethoxy)-1H-pyrrolo[3,2-c]quinolin-8-yl)propanenitrile;

4-(1-((1R,4R,5S)-2-Azabicyclo[2.1.1]hexan-5-yl)-8-(2-cyanoethyl)-6-fluoro-7-(7-fluoronaphthalen-1-yl)-4-(S)-1-(S)-1-methylpyrrolidin-2-yl)ethoxy)-1H-pyrrolo[3,2-c]quinolin-2-yl)-N,N,1-trimethyl-1H-pyrazole-5-carboxamide;

3-(2-*(R)-1-Acetylpyrrolidin-2-yl)-1-((1R,4R,5S)-2-azabicyclo[2.1.1]hexan-5-yl)-3-chloro-7-(2,3-dichlorophenyl)-6-fluoro-4-((S)-1-((S)-1-methylpyrrolidin-2-yl)ethoxy)-1H-pyrrolo[3,2-c]quinolin-8-yl)propanenitrile; and

8-(2-((R)-1-Acetylpyrrolidin-2-yl)-1-((1R,4R,5S)-2-azabicyclo[2.1.1]hexan-5-yl) fluoro-8-methyl-4-(S)-1-((S)-1-methylpyrrolidin-2-yl)ethoxy)-1H-pyrrolo[3,2-c]quinolin-7-yl)-1,2,3,4-tetrahydronaphthalene-1-carbonitrile,

or a pharmaceutically acceptable salt thereof.

Provided herein is a method of treating cancer in a patient, comprising administering to said patient:

(i) an FGFR1 inhibitor selected from pemigatinib, futibatinib, erdafitinib and infigratinib, or a pharmaceutically acceptable salt thereof; and

(ii) a KRAS inhibitor selected from sotorasib, adagrasib, Compound 2, Compound 3 and Compound 4, or a pharmaceutically acceptable salt thereof.

Provided herein is a method of treating cancer in a patient, comprising administering to said patient:

(i) an FGFR1 inhibitor selected from pemigatinib, futibatinib, erdafitinib and infigratinib, or a pharmaceutically acceptable salt thereof; and

(ii) a KRAS inhibitor selected from sotorasib, Compound 2, Compound 3 and Compound 4, or a pharmaceutically acceptable salt thereof.

Provided herein is a method of treating cancer in a patient, comprising administering to said patient:

an FGFR1 inhibitor selected from pemigatinib, futibatinib, erdafitinib and infigratinib, or a pharmaceutically acceptable salt thereof; and

(ii) a KRAS inhibitor selected from sotorasib, Compound 2, Compound 3, Compound 4, Compound 5, Compound 6, and Compound 7, or a pharmaceutically acceptable salt thereof.

Provided herein is a method of treating cancer in a patient, comprising administering to said patient:

an FGFR1 inhibitor selected from pemigatinib, futibatinib, erdafitinib and infigratinib, or a pharmaceutically acceptable salt thereof; and

(ii) a KRAS inhibitor selected from Compound 5, Compound 6, and Compound 7, or a pharmaceutically acceptable salt thereof.

In some embodiments, the FGFR1 inhibitor is pemigatinib, or a pharmaceutically acceptable salt thereof. In some embodiments, the FGFR1 inhibitor is futibatinib, or a pharmaceutically acceptable salt thereof. In some embodiments, the FGFR1 inhibitor is erdafitinib, or a pharmaceutically acceptable salt thereof. In some embodiments, the FGFR1 inhibitor is infigratinib, or a pharmaceutically acceptable salt thereof.

In some embodiments, the KRAS inhibitor is sotorasib, or a pharmaceutically acceptable salt thereof. In some embodiments, the KRAS inhibitor is adagrasib, or a pharmaceutically acceptable salt thereof. In some embodiments, the KRAS inhibitor is Compound 2, or a pharmaceutically acceptable salt thereof. In some embodiments, the KRAS inhibitor is Compound 3, or a pharmaceutically acceptable salt thereof. In some embodiments, the KRAS inhibitor is Compound 4, or a pharmaceutically acceptable salt thereof. In some embodiments, the KRAS inhibitor is Compound 5, or a pharmaceutically acceptable salt thereof In some embodiments, the KRAS inhibitor is Compound 6, or a pharmaceutically acceptable salt thereof. In some embodiments, the KRAS inhibitor is Compound 7, or a pharmaceutically acceptable salt thereof.

Provided herein is a method of treating cancer in a patient, comprising administering to said patient:

pemigatinib, or a pharmaceutically acceptable salt thereof; and

(ii) a KRAS inhibitor selected from sotorasib, adagrasib, Compound 2, Compound 3 and Compound 4, or a pharmaceutically acceptable salt thereof.

Provided herein is a method of treating cancer in a patient, comprising administering to said patient:

(i) pemigatinib, or a pharmaceutically acceptable salt thereof; and

(ii) a KRAS inhibitor selected from sotorasib, adagrasib, Compound 2, Compound 3 Compound 4, Compound 5, Compound 6, and Compound 7, or a pharmaceutically acceptable salt thereof.

Provided herein is a method of treating cancer in a patient, comprising administering to said patient:

(i) pemigatinib, or a pharmaceutically acceptable salt thereof; and

(ii) a KRAS inhibitor selected from Compound 5, Compound 6, and Compound 7, or

a pharmaceutically acceptable salt thereof.

Provided herein is a method of treating cancer in a patient, comprising administering to said patient:

(i) pemigatinib, or a pharmaceutically acceptable salt thereof; and

(ii) a KRAS inhibitor selected from sotorasib, Compound 2, Compound 3 and Compound 4, or a pharmaceutically acceptable salt thereof.

In some embodiments, the KRAS inhibitor is sotorasib, or a pharmaceutically acceptable salt thereof. In some embodiments, the KRAS inhibitor is adagrasib, or a pharmaceutically acceptable salt thereof. In some embodiments, the KRAS inhibitor is Compound 2, or a pharmaceutically acceptable salt thereof. In some embodiments, the KRAS inhibitor is Compound 3, or a pharmaceutically acceptable salt thereof. In some embodiments, the KRAS inhibitor is Compound 4, or a pharmaceutically acceptable salt thereof. In some embodiments, the KRAS inhibitor is Compound 5, or a pharmaceutically acceptable salt thereof In some embodiments, the KRAS inhibitor is Compound 6, or a pharmaceutically acceptable salt thereof. In some embodiments, the KRAS inhibitor is Compound 7, or a pharmaceutically acceptable salt thereof.

Provided herein is a method of treating cancer in a patient, comprising administering to said patient:

pemigatinib, or a pharmaceutically acceptable salt thereof; and

(ii) sotorasib, or a pharmaceutically acceptable salt thereof.

Provided herein is a method of treating cancer in a patient, comprising administering to said patient:

pemigatinib, or a pharmaceutically acceptable salt thereof; and

(ii) adagrasib, or a pharmaceutically acceptable salt thereof.

In vivo studies demonstrated that the combination of an FGFR1 inhibitor, i.e., pemigatinib, and a KRAS inhibitor, i.e., sotorasib, had synergistic effects in the treatment of lung cancer at certain dosages (See, e.g., Examples E and F).

In some embodiments, pemigatinib, or a pharmaceutically acceptable salt thereof, and sotorasib or a pharmaceutically acceptable salt thereof, are administered to the patient simultaneously. In some embodiments, pemigatinib, or a pharmaceutically acceptable salt thereof, and sotorasib or a pharmaceutically acceptable salt thereof, are administered to the patient sequentially.

In some embodiments, pemigatinib, or a pharmaceutically acceptable salt thereof, and adagrasib or a pharmaceutically acceptable salt thereof, are administered to the patient simultaneously. In some embodiments, pemigatinib, or a pharmaceutically acceptable salt thereof, and adagrasib or a pharmaceutically acceptable salt thereof, are administered to the patient sequentially.

Pemigatinib and its pharmaceutically acceptable salts can be administered to a subject, e.g., a subject in need thereof, for example, a human subject, by a variety of methods. For many applications, the route of administration is oral. In some embodiments, pemigatinib, or a pharmaceutically acceptable salt thereof, is administered as a pharmaceutical composition. In some embodiments, pemigatinib is administered orally. In some embodiments, pemigatinib is administered once daily.

In some embodiments, pemigatinib is administered in a daily dose of about 1 mg to about 50 mg. In some embodiments, pemigatinib is administered in a daily dose of about 1 mg to about 20 mg. In some embodiments, pemigatinib is administered in a daily dose of about 1 mg to about 15 mg. In some embodiments, pemigatinib is administered in a daily dose of about 1 mg to about 10 mg. In some embodiments, pemigatinib is administered in a daily dose of about 1 mg to about 5 mg. In some embodiments, pemigatinib is administered in a daily dose of about 5 mg to about 20 mg. In some embodiments, pemigatinib is administered in a daily dose of about 5 mg to about 10 mg. In some embodiments, pemigatinib is administered in a daily dose of about 10 mg to about 15 mg. In some embodiments, pemigatinib is administered in a daily dose of about 10 mg. In some embodiments, pemigatinib is administered in a daily dose of about 2 mg. In some embodiments, pemigatinib is administered in a daily dose of about 4.5 mg. In some embodiments, pemigatinib is administered in a daily dose of about 9 mg. In some embodiments, pemigatinib is administered in a daily dose of about 13.5 mg. In some embodiments, pemigatinib is administered in a daily dose of about 20 mg or less.

In some embodiments, pemigatinib is administered in a daily dose of about 15 mg or less. In some embodiments, pemigatinib is administered in a daily dose of about 10 mg or less. In some embodiments, pemigatinib is administered in a daily dose of about 9 mg or less. In some embodiments, pemigatinib is administered in a daily dose of about 8 mg or less. In some embodiments, pemigatinib is administered in a daily dose of about 7 mg or less. In some embodiments, pemigatinib is administered in a daily dose of about 6 mg or less. In some embodiments, pemigatinib is administered in a daily dose of about 5 mg or less. In some embodiments, pemigatinib is administered in a daily dose of about 4 mg or less. In some embodiments, pemigatinib is administered in a daily dose of about 3 mg or less. In some embodiments, pemigatinib is administered in a daily dose of about 2 mg or less. In some embodiments, pemigatinib is administered in a daily dose of about 1 mg or less.

In some embodiments, pemigatinib is administered as a tablet. In some embodiments, the tablet comprises about 0.5 mg to about 10 mg of pemigatinib. In some embodiments, the tablet comprises about 0.5 mg to about 5 mg pemigatinib. In some embodiments, the tablet comprises about 2 mg, about 4.5 mg, about 9 mg, about 13.5 mg, or about 18 mg of pemigatinib. In some embodiments, the tablet comprises about 0.5 mg of pemigatinib. In some embodiments, the tablet comprises about 2 mg of pemigatinib. In some embodiments, the tablet comprises about 4.5 mg of pemigatinib. In some embodiments, the tablet comprises about 9 mg of pemigatinib. In some embodiments, the tablet comprises about 13.5 mg of pemigatinib. In some embodiments, the tablet comprises about 18 mg of pemigatinib.

Sotorasib and its pharmaceutically acceptable salts can be administered to a subject, e.g., a subject in need thereof, for example, a human subject, by a variety of methods. For many applications, the route of administration is oral. In some embodiments, sotorasib, or a pharmaceutically acceptable salt thereof, is administered as a pharmaceutical composition.

In some embodiments, sotorasib is administered orally. In some embodiments, sotorasib is administered once daily.

In some embodiments, sotorasib is administered in a daily dose of about 10 mg to about 2000 mg. In some embodiments, sotorasib is administered in a daily dose of about 10 mg to about 1500 mg. In some embodiments, sotorasib is administered in a daily dose of about 50 mg to about 1000 mg. In some embodiments, sotorasib is administered in a daily dose of about 50 mg to about 300 mg. In some embodiments, sotorasib is administered in a daily dose of about 100 mg to about 200 mg. In some embodiments, sotorasib is administered in a daily dose of about 100 mg to about 150 mg. In some embodiments, sotorasib is administered in a daily dose of about 110 mg to about 140 mg. In some embodiments, sotorasib is administered in a daily dose of about 120 mg to about 135 mg. In some embodiments, sotorasib is administered in a daily dose of about 133 mg. In some embodiments, sotorasib is administered in a daily dose of about 120 mg.

In some embodiments, sotorasib is administered in a daily dose of about 2000 mg or less. In some embodiments, sotorasib is administered in a daily dose of about 1200 mg or less. In some embodiments, sotorasib is administered in a daily dose of about 1080 mg or less. In some embodiments, sotorasib is administered in a daily dose of about 960 mg or less. In some embodiments, sotorasib is administered in a daily dose of about 840 mg or less. In some embodiments, sotorasib is administered in a daily dose of about 720 mg or less. In some embodiments, sotorasib is administered in a daily dose of about 600 mg or less. In some embodiments, sotorasib is administered in a daily dose of about 500 mg or less. In some embodiments, sotorasib is administered in a daily dose of about 480 mg or less. In some embodiments, sotorasib is administered in a daily dose of about 360 mg or less. In some embodiments, sotorasib is administered in a daily dose of about 240 mg or less. In some embodiments, sotorasib is administered in a daily dose of about 120 mg or less.

In some embodiments, sotorasib is administered as a tablet. In some embodiments, the tablet comprises about 50 mg to about 1000 mg of sotorasib. In some embodiments, the tablet comprises about 50 mg to about 150 mg sotorasib. In some embodiments, the tablet comprises about 60 mg, about 120 mg, about 240 mg, about 360 mg, or about 480 mg of sotorasib. In some embodiments, the tablet comprises about 60 mg of sotorasib. In some embodiments, the tablet comprises about 120 mg of sotorasib. In some embodiments, the tablet comprises about 240 mg of sotorasib.

In some embodiments, pemigatinib, or a pharmaceutically acceptable salt thereof, and sotorasib, or a pharmaceutically acceptable salt thereof, are orally administered simultaneously in a daily dose of about 2 mg and 120 mg, respectively. In some embodiments, pemigatinib, or a pharmaceutically acceptable salt thereof, and sotorasib, or a pharmaceutically acceptable salt thereof, are orally administered sequentially in a daily dose of about 2 mg and 120 mg, respectively. In some embodiments, pemigatinib, or a pharmaceutically acceptable salt thereof, and sotorasib, or a pharmaceutically acceptable salt thereof, are each administered in the form of a tablet.

Adagrasib and its pharmaceutically acceptable salts can be administered to a subject, e.g., a subject in need thereof, for example, a human subject, by a variety of methods. For many applications, the route of administration is oral. In some embodiments, adagrasib, or a pharmaceutically acceptable salt thereof, is administered as a pharmaceutical composition.

In some embodiments, adagrasib is administered orally. In some embodiments, adagrasib is administered twice daily. In some embodiments, adagrasib is administered once daily.

In some embodiments, adagrasib is administered in a daily dose of about 10 mg to about 2000 mg. In some embodiments, adagrasib is administered in a daily dose of about 10 mg to about 1500 mg. In some embodiments, adagrasib is administered in a daily dose of about 50 mg to about 1500 mg. In some embodiments, adagrasib is administered in a daily dose of about 100 mg to about 1500 mg. In some embodiments, adagrasib is administered in a daily dose of about 500 mg to about 500 mg. In some embodiments, adagrasib is administered in a daily dose of about 1000 mg to about 1500 mg. In some embodiments, adagrasib is administered in a daily dose of about 50 mg to about 1200 mg. In some embodiments, adagrasib is administered in a daily dose of about 50 mg to about 1000 mg. In some embodiments, adagrasib is administered in a daily dose of about 50 mg to about 800 mg. In some embodiments, adagrasib is administered in a daily dose of about 100 mg to about 800 mg. In some embodiments, adagrasib is administered in a daily dose of about 200 mg to about 800 mg. In some embodiments, adagrasib is administered in a daily dose of about 500 mg to about 700 mg. In some embodiments, adagrasib is administered in a daily dose of about 50 mg to about 300 mg. In some embodiments, adagrasib is administered in a daily dose of about 100 mg to about 200 mg. In some embodiments, adagrasib is administered in a daily dose of about 100 mg to about 300 mg. In some embodiments, adagrasib is administered in a daily dose of about 150 mg to about 200 mg. In some embodiments, adagrasib is administered in a daily dose of about 150 mg. In some embodiments, adagrasib is administered in a daily dose of about 300 mg. In some embodiments, adagrasib is administered in a daily dose of about 450 mg. In some embodiments, adagrasib is administered in a daily dose of about 600 mg. In some embodiments, adagrasib is administered in a daily dose of about 750 mg. In some embodiments, adagrasib is administered in a daily dose of about 900 mg. In some embodiments, adagrasib is administered in a daily dose of about 1050 mg. In some embodiments, adagrasib is administered in a daily dose of about 1200 mg.

In some embodiments, adagrasib is administered in a daily dose of about 2000 mg or less. In some embodiments, adagrasib is administered in a daily dose of about 1500 mg or less. In some embodiments, adagrasib is administered in a daily dose of about 1350 mg or less. In some embodiments, adagrasib is administered in a daily dose of about 1200 mg or less. In some embodiments, adagrasib is administered in a daily dose of about 1050 mg or less. In some embodiments, adagrasib is administered in a daily dose of about 900 mg or less. In some embodiments, adagrasib is administered in a daily dose of about 750 mg or less. In some embodiments, adagrasib is administered in a daily dose of about 600 mg or less. In some embodiments, adagrasib is administered in a daily dose of about 450 mg or less. In some embodiments, adagrasib is administered in a daily dose of about 300 mg or less. In some embodiments, adagrasib is administered in a daily dose of about 150 mg or less.

In some embodiments, adagrasib is administered as a tablet. In some embodiments, the tablet comprises about 50 mg to about 1200 mg of adagrasib. In some embodiments, the tablet comprises about 50 mg to about 200 mg adagrasib. In some embodiments, the tablet comprises about 150 mg, about 300 mg, about 450 mg, or about 600 mg of adagrasib.

In some embodiments, pemigatinib, or a pharmaceutically acceptable salt thereof, and adagrasib, or a pharmaceutically acceptable salt thereof, are orally administered simultaneously in a daily dose of about 2 mg and 150 mg, respectively. In some embodiments, pemigatinib, or a pharmaceutically acceptable salt thereof, and adagrasib, or a pharmaceutically acceptable salt thereof, are orally administered simultaneously in a daily dose of about 2 mg and 300 mg, respectively. In some embodiments, pemigatinib, or a pharmaceutically acceptable salt thereof, and adagrasib, or a pharmaceutically acceptable salt thereof, are orally administered simultaneously in a daily dose of about 2 mg and 450 mg, respectively. In some embodiments, pemigatinib, or a pharmaceutically acceptable salt thereof, and adagrasib, or a pharmaceutically acceptable salt thereof, are orally administered simultaneously in a daily dose of about 2 mg and 600 mg, respectively. In some embodiments, pemigatinib, or a pharmaceutically acceptable salt thereof, and adagrasib, or a pharmaceutically acceptable salt thereof, are orally administered sequentially in a daily dose of about 2 mg and 1200 mg, respectively. In some embodiments, pemigatinib, or a pharmaceutically acceptable salt thereof, and adagrasib, or a pharmaceutically acceptable salt thereof, are each administered in the form of a tablet.

### Methods of Use

Cancer types for which KRAS harbors G12C, G12D and G12V mutations are implicated include, but are not limited to: carcinomas (e.g., pancreatic, colorectal, lung (i.e., non-small cell lung), ovarian, bladder, gastric, esophageal, breast, head and neck, cervical, skin, thyroid); hematopoietic malignancies (e.g., multiple myeloma, acute myelogenous leukemia, and myeloproliferative neoplasms); and other neoplasms (e.g., glioblastoma and sarcomas).

The methods disclosed herein are useful in the treatment of cancer.

In some embodiments, the cancer comprises one or more KRAS mutations. In some embodiments, the one or more KRAS mutations comprise mutations selected from G12C, G12D, C12V and combinations thereof In some embodiments, the one or more KRAS mutations is a G12C mutation. In some embodiments, the cancer further comprises high FGFR1 expression. In some embodiments, the cancer is implicated in the alteration of the MAPK signaling pathway. In some embodiments, the cancer is implicated in KRAS pathway dysregulation. In some embodiments, the cancer is implicated in FGFR pathway dysregulation. In some embodiments, the cancer comprises mesenchymal-like cells.

In some embodiments, the cancer is selected from carcinomas, pancreatic cancer, colorectal cancer, lung cancer, non-small cell lung cancer, ovarian cancer, bladder cancer, gastric cancer, esophageal cancer, breast cancer, head and neck cancer, cervical cancer, skin cancer, thyroid cancer, hematopoietic malignancies, multiple myeloma, acute myelogenous leukemia, myeloproliferative neoplasms, neoplasms, glioblastoma and sarcomas.

In some embodiments, the cancer is lung cancer. In some embodiments, the lung cancer is non-small cell lung cancer. In some embodiments, the cancer is colorectal cancer. In some embodiments, the cancer is pancreatic cancer. In some embodiments, the cancer is ovarian cancer.

In some embodiments, the cancer is bladder cancer, breast cancer (e.g., hormone R positive, triple negative), cervical cancer, colorectal cancer, cancer of the small intestine, colon cancer, rectal cancer, cancer of the anus, endometrial cancer, gastric cancer (e.g., gastrointestinal stromal tumors), head and neck cancer (e.g., cancers of the larynx, hypopharynx, nasopharynx, oropharynx, lips, and mouth, squamous head and neck cancers), kidney cancer (e.g., renal cell carcinoma, urothelial carcinoma, sarcoma, Wilms tumor), liver cancer (e.g., hepatocellular carcinoma, cholangiocellular carcinoma, liver angiosarcoma, hepatoblastoma), lung cancer (e.g., adenocarcinoma, small cell lung cancer and non-small cell lung carcinomas, parvicellular and non-parvicellular carcinoma, bronchial carcinoma, bronchial adenoma, pleuropulmonary blastoma), ovarian cancer, prostate cancer, testicular cancer, uterine cancer, vulvar cancer, esophageal cancer, gall bladder cancer, pancreatic cancer (e.g., exocrine pancreatic carcinoma), stomach cancer, thyroid cancer, parathyroid cancer, neuroendocrine cancer (e.g., pheochromocytoma, Merkel cell cancer, neuroendocrine carcinoma), skin cancer (e.g., squamous cell carcinoma, Kaposi sarcoma, Merkel cell skin cancer), or brain cancer (e.g., astrocytoma, medulloblastoma, ependymoma, neuro-ectodermal tumors, pineal tumors). In some embodiments, the cancer is a hematopoietic malignancy such as leukemia or lymphoma, multiple myeloma, chronic lymphocytic lymphoma, adult T cell leukemia, B-cell lymphoma, cutaneous T-cell lymphoma, acute myelogenous leukemia, Hodgkin's or non-Hodgkin's lymphoma, myeloproliferative neoplasms (e.g., 8p11 myeloproliferative syndrome, polycythemia vera, essential thrombocythemia, and primary myelofibrosis), myelodysplastic syndrome, chronic eosinophilic leukemia, Waldenstrom's Macroglubulinemia, hairy cell lymphoma, chronic myelogenic lymphoma, acute lymphoblastic lymphoma, AIDS-related lymphomas, or Burkitt's lymphoma.

In certain embodiments, provided herein is a method of treating myeloid/lymphoid neoplasms in a patient in need thereof In certain embodiments, the myeloid/lymphoid neoplasms are 8p11 myeloproliferative syndrome. As used herein, the term “8p11 myeloproliferative syndrome” (EMS) is meant to refer to myeloid/lymphoid neoplasms associated with eosinophilia and abnormalities of FGFR1 or myeloid/lymphoid neoplasms (MLN) with FGFR1 rearrangement. Eight P eleven myeloproliferative syndrome is reviewed in Jackson, Courtney C., et.al. Human Pathology, 2010, 41, 461-476. In certain embodiments, the myeloid/lymphoid neoplasm exhibits an 8p11 translocation. In certain embodiments, the 8p11 translocation is associated with activation of FGFR1. In certain embodiments, the patient has failed at least one previous treatment for myeloid/lymphoid neoplasms (e.g., 8p11 myeloproliferative syndrome). In some embodiments, the previous treatment is surgery or radiation therapy. In some embodiments, the patient has a history of hepatitis. In some embodiments, the hepatitis is chronic hepatitis B or hepatitis C. In some embodiments, the patient does not have a history of hepatitis.

In certain embodiments, the cancer is bladder cancer (e.g., urothelial carcinoma, squamous cell carcinoma, adenocarcinoma). In certain embodiments, the bladder cancer is the luminal papillary subtype of bladder cancer.

In certain embodiments, the cancer is glioblastoma or lung cancer.

In certain embodiments, the liver cancer is cholangiocellular carcinoma (e.g., intrahepatic, hilar or perihilar, distal extrahepatic). As used herein, cholangiocellular carcinoma is the same as cholangiocarcinoma or bile duct cancer. In certain embodiments, the cholangiocarcinoma is advanced or metastatic cholangiocarcinoma. In certain embodiments, the cholangiocarcinoma is surgically unresectable. In certain embodiments, the cholangiocarcinoma is intrahepatic. In certain embodiments, the cholangiocarcinoma is extrahepatic.

Other cancers treatable with the methods provided herein include tumors of the eye, glioblastoma, melanoma, rhabdosarcoma, lymphosarcoma, leiomyosarcoma, urothelial carcinoma (e.g., ureter, urethra, bladder, urachus), and osteosarcoma.

The methods of the present disclosure are also useful for the treatment of metastatic cancers, especially metastatic cancers that express PD-L1.

In some embodiments, diseases and indications that are treatable using the methods of the present disclosure include, but are not limited to hematological cancers, head and neck cancers, sarcomas, lung cancers, gastrointestinal cancers, genitourinary tract cancers, liver cancers, bone cancers, nervous system cancers, gynecological cancers, and skin cancers.

Exemplary hematological cancers treatable using the methods of the present disclosure include lymphomas and leukemias such as acute lymphoblastic leukemia (ALL), acute myelogenous leukemia (AML), acute promyelocytic leukemia (APL), chronic lymphocytic leukemia (CLL), small lymphocytic lymphoma (SLL), chronic myelogenous leukemia (CIVIL), diffuse large B-cell lymphoma (DLBCL), mantle cell lymphoma (MCL), marginal zone lymphoma (MZL), Non-Hodgkin lymphoma (including relapsed or refractory NHL), follicular lymphoma (FL), Hodgkin lymphoma, lymphoblastic lymphoma, myeloproliferative diseases (e.g., primary myelofibrosis (PMF), polycythemia vera (PV), essential thrombocytosis (ET)), myelodysplasia syndrome (MDS), T-cell acute lymphoblastic lymphoma (T-ALL), multiple myeloma, cutaneous T-cell lymphoma, peripheral T-cell lymphoma, Waldenstrom's Macroglubulinemia, hairy cell lymphoma, chronic myelogenic lymphoma and Burkitt's lymphoma.

Exemplary sarcomas treatable using the methods of the present disclosure include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, and teratoma.

Exemplary lung cancers treatable using the methods of the present disclosure include non-small cell lung cancer (NSCLC), small cell lung cancer, bronchogenic carcinoma (squamous cell, undifferentiated small cell, undifferentiated large cell, adenocarcinoma), alveolar (bronchiolar) carcinoma, bronchial adenoma, chondromatous hamartoma, and mesothelioma.

Exemplary gastrointestinal cancers treatable using the methods of the present disclosure include cancers of the esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), colorectal cancer, bile duct cancer (cholangiocarcinoma).

Exemplary genitourinary tract cancers treatable using the methods of the present disclosure include cancers of the kidney (adenocarcinoma, Wilm's tumor [nephroblastoma], renal cell carcinoma), bladder and urethra (squamous cell carcinoma, transitional cell carcinoma, adenocarcinoma, urothelial carcinoma), prostate (adenocarcinoma, sarcoma), and testis (seminoma, teratoma, embryonal carcinoma, teratocarcinoma, choriocarcinoma, sarcoma, interstitial cell carcinoma, fibroma, fibroadenoma, adenomatoid tumors, lipoma).

Exemplary liver cancers treatable using the methods of the present disclosure include hepatoma (hepatocellular carcinoma), cholangiocarcinoma, hepatoblastoma, angiosarcoma, hepatocellular adenoma, and hemangioma.

Exemplary bone cancers treatable using the methods of the present disclosure include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors

Exemplary nervous system cancers treatable using the methods of the present disclosure include cancers of the skull (osteoma, hemangioma, granuloma, xanthoma, osteitis deformans), meninges (meningioma, meningiosarcoma, gliomatosis), brain (astrocytoma, meduoblastoma, glioma, ependymoma, germinoma (pinealoma), glioblastoma, glioblastoma multiform, oligodendroglioma, schwannoma, retinoblastoma, congenital tumors), and spinal cord (neurofibroma, meningioma, glioma, sarcoma), as well as neuroblastoma, Lhermitte-Duclos disease, neoplasm of the central nervous system (CNS), primary CNS lymphoma and spinal axis tumor.

Exemplary gynecological cancers treatable using the methods of the present disclosure include cancers of the uterus (endometrial carcinoma), cervix (cervical carcinoma, pre -tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers treatable using the methods of the present disclosure include melanoma, basal cell carcinoma, squamous cell carcinoma, Kaposi's sarcoma, Merkel cell skin cancer, moles dysplastic nevi, lipoma, angioma, dermatofibroma, and keloids.

Exemplary head and neck cancers treatable using the methods of the present disclosure include glioblastoma, melanoma, rhabdosarcoma, lymphosarcoma, osteosarcoma, squamous cell carcinomas, adenocarcinomas, oral cancer, laryngeal cancer, nasopharyngeal cancer, nasal and paranasal cancers, thyroid and parathyroid cancers.

In some embodiments, the present disclosure provides a method for treating hepatocellular carcinoma in a patient in need thereof. In some embodiments, the present disclosure provides a method for treating Rhabdomyosarcoma, esophageal cancer, breast cancer, or cancer of a head or neck, in a patient in need thereof.

The methods described herein involve the treatment of cancers, for example solid tumors.

In some embodiments, the solid tumor is selected from skin cancer, lung cancer, lymphoma, sarcoma, bladder cancer, cancer of the ureter, urethra, and urachus, gastric cancer, cervical cancer, liver cancer, breast cancer, renal cancer, squamous cell carcinoma, colorectal cancer, endometrial cancer, anal cancer, and a tumor with microsatellite instability-high (MSI-H), mismatch repair deficient (dMMR) and/or DNA polymerase c exonuclease domain mutation positive disease.

In some embodiments, the solid tumor is selected from cholangiocarcinoma, melanoma, non-small cell lung cancer, small cell lung cancer, Hodgkin's lymphoma, urothelial carcinomagastric cancer, hepatocellular carcinoma, Merkel cell carcinoma, triple-negative breast cancer, renal cell carcinoma, squamous cell carcinoma of the head and neck, and colorectal cancer.

In some embodiments, the solid tumor is selected from sarcomas, head and neck cancer, melanoma, and non-small cell lung cancer. In some embodiments, the solid tumor is sarcoma. In some embodiments, the solid tumor is head and neck cancer. In some embodiments, the solid tumor is melanoma. In some embodiments, the solid tumor is non-small cell lung cancer. As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

As used herein, the term “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); and (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease. In some embodiments, the term “treating” or “treatment” refers to inhibiting or ameliorating the disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease, condition, or disorder.

As used herein, the term “coadministering” or “concomitant administering” refers to administering pemigatinib and one or more additional drugs (e.g., sotorasib) at or almost at the same time. In some embodiments, the one or more additional drug is adagrasib. For example, pemigatinib may be administered, e.g., on the same day, within a week, or within a month as the one or more additional drugs. In some embodiments, the one or more additional drugs is administered between administrations of pemigatinib.

As used herein, the term “therapy” refers to administration of a compound that is suitable for treating cancer. For example, therapy can refer to the administration of pemigatinib for treating cancer. As used herein, and unless otherwise specified, the term “about”, when used in connection with a numeric value or range of values, indicate that the value or range of values may deviate to an extent deemed reasonable by one of ordinary skill in the art. Specifically, the term “about”, when used in this context, indicates that the numeric value or range of values may vary by 5%, 4%, 3%, 2%, 1%, 0.9%, 0.8%, 0.7%, 0.6%, 0.5%, 0.4%, 0.3%, 0.2% or 0.1% of the recited value or range of values.

As used herein, the terms “cancer” and “carcinoma” are synonymous. As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” an FGFR enzyme with an FGFR1 inhibitor, e.g., pemigatinib, includes the administration of an FGFR1 inhibitor described herein to an individual or patient, such as a human, having FGFR, as well as, for example, introducing the FGFR1 inhibitor into a sample containing a cellular or purified preparation containing the FGFR enzyme. As a further example, “contacting” KRAS with a KRAS inhibitor described herein includes the administration of a KRAS inhibitor described herein to an individual or patient, such as a human, having KRAS, as well as, for example, introducing the KRAS inhibitor into a sample containing a cellular or purified preparation containing KRAS.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

As used herein, the phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

In some embodiments, a pharmaceutically acceptable salt of an FGFR1 inhibitor disclosed herein is used in the methods and combination therapies described herein. In some embodiments, a pharmaceutically acceptable salt of pemigatinib is used in the methods and combination therapies described herein. Salt forms of pemigatinib are described in US Publication No. 2019/0337948. In some embodiments, a pharmaceutically acceptable salt of a KRAS inhibitor disclosed herein is used in the methods and combination therapies described herein. In some embodiments, a pharmaceutically acceptable salt of sotorasib is used in the methods and combination therapies described herein. In some embodiments, a pharmaceutically acceptable salt of adagrasib is used in the methods and combination therapies described herein.

In some embodiments, solid forms (e.g., crystalline forms) of an FGFR1 inhibitor disclosed herein is used in the methods and combination therapies described herein. In some embodiments, solid forms (e.g., crystalline forms) of pemigatinib can also be used in the methods and combination therapies described herein. Solid forms of pemigatinib, and methods of preparing solid forms of pemigatinib, are described in U.S. Publication No. 2020/0002338. In some embodiments, solid forms (e.g., crystalline forms) of a KRAS inhibitor disclosed herein is used in the methods and combination therapies described herein. In some embodiments, solid forms (e.g., crystalline forms) of sotorasib can also be used in the methods and combination therapies described herein. In some embodiments, solid forms (e.g., crystalline forms) of adagrasib can also be used in the methods and combination therapies described herein.

It is appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination.

Combination Therapy with Additional Agents

Also provided herein is a method of treating cancer in a patient, comprising administering to said patient:

(i) pemigatinib, or a pharmaceutically acceptable salt thereof;

(ii) a KRAS inhibitor as described herein; and

(iii) one or more additional therapeutic agents.

Also provided herein is a method of treating cancer in a patient, comprising administering to said patient:

(i) pemigatinib, or a pharmaceutically acceptable salt thereof;

(ii) sotorasib, or a pharmaceutically acceptable salt thereof; and

(iii) one or more additional therapeutic agents.

Also provided herein is a method of treating cancer in a patient, comprising administering to said patient:

(i) pemigatinib, or a pharmaceutically acceptable salt thereof;

(ii) adagrasib, or a pharmaceutically acceptable salt thereof; and

(iii) one or more additional therapeutic agents.

Exemplary additional therapeutic agents are set forth below.

### I. Cancer Therapies

Cancer cell growth and survival can be impacted by dysfunction in multiple signaling pathways. Thus, it is useful to combine different enzyme/protein/receptor inhibitors, exhibiting different preferences in the targets which they modulate the activities of, to treat such conditions.

Targeting more than one signaling pathway (or more than one biological molecule involved in a given signaling pathway) may reduce the likelihood of drug-resistance arising in a cell population, and/or reduce the toxicity of treatment.

One or more additional pharmaceutical agents such as, for example, chemotherapeutics, anti-inflammatory agents, steroids, immunosuppressants, immune-oncology agents, metabolic enzyme inhibitors, chemokine receptor inhibitors, and phosphatase inhibitors, as well as targeted therapies such as Bcr-Ab1, Flt-3, EGFR, HER2, JAK, c-MET, VEGFR, PDGFR, c-Kit, IGF-1R, RAF, FAK, CDK2, and CDK4/6 kinase inhibitors such as, for example, those described in WO 2006/056399 can be used in combination with the treatment methods and regimens of the present disclosure for treatment of cancers and solid tumors. Other agents such as therapeutic antibodies can be used in combination with the treatment methods and regimens of the present disclosure for treatment of cancers and solid tumors. The one or more additional pharmaceutical agents can be administered to a patient simultaneously or sequentially.

The treatment methods as disclosed herein can be used in combination with one or more other enzyme/protein/receptor inhibitors therapies for the treatment of diseases, such as cancer and other diseases or disorders described herein. For example, the treatment methods and regimens of the present disclosure can be combined with one or more inhibitors of the following kinases for the treatment of cancer: Akt1, Akt2, Akt3, BCL2, CDK2, CDK4/6, TGF-βR, PKA, PKG, PKC, CaM-kinase, phosphorylase kinase, MEKK, ERK, MAPK, mTOR, EGFR, HER2, HER3, HER4, INS-R, IDH2, IGF-1R, IR-R, KRAS, PDGFaR, PDGFI3R, PI3K (alpha, beta, gamma, delta, and multiple or selective), CSF1R, KIT, FLK-II, KDR/FLK-1, FLK-4, flt-1, FGFR1, FGFR2, FGFR3, FGFR4, c-Met, PARP, Ron, Sea, TRKA, TRKB, TRKC, TAM kinases (Ax1, Mer, Tyro3), FLT3, VEGFR/F1t2, Flt4, EphA1, EphA2, EphA3, EphB2, EphB4, Tie2, Src, Fyn, Lck, Fgr, Btk, Fak, SYK, FRK, JAK, ABL, ALK and B-Raf. Non-limiting examples of inhibitors that can be combined with the treatment methods and regimens of the present disclosure for treatment of cancer include an FGFR inhibitor (FGFR1, FGFR2, FGFR3 or FGFR4, e.g., INCB62079), an EGFR inhibitor (also known as ErB-1 or HER-1; e.g., erlotinib, gefitinib, vandetanib, orsimertinib, cetuximab, necitumumab, or panitumumab), a VEGFR inhibitor or pathway blocker (e.g., bevacizumab, pazopanib, sunitinib, sorafenib, axitinib, regorafenib, ponatinib, cabozantinib, vandetanib, ramucirumab, lenvatinib, ziv-aflibercept), a PARP inhibitor (e.g., olaparib, rucaparib, veliparib or niraparib), a JAK inhibitor (JAK1 and/or JAK2, e.g., ruxolitinib, baricitinib, itacitinib (INCB39110), an LSD1 inhibitor (e.g., INCB59872 and INCB60003), a TDO inhibitor, a PI3K-delta inhibitor (e.g., INCB50465 and INCB50797), a PI3K-gamma inhibitor such as PI3K-gamma selective inhibitor, a Pim inhibitor (e.g., INCB53914), a CSF1R inhibitor, a TAM receptor tyrosine kinases (Tyro-3, Ax1, and Mer), an adenosine receptor antagonist (e.g., A2a/A2b receptor antagonist), an HPK1 inhibitor, a chemokine receptor inhibitor (e.g., CCR2 or CCRS inhibitor), a SHP1/2 phosphatase inhibitor, a histone deacetylase inhibitor (HDAC) such as an HDAC8 inhibitor, an angiogenesis inhibitor, an interleukin receptor inhibitor, bromo and extra terminal family members inhibitors (for example, bromodomain inhibitors or BET inhibitors such as INCB54329 and INCB57643), c-MET inhibitors (e.g., capmatinib), an anti-CD19 antibody (e.g., tafasitamab), an ALK2 inhibitor (e.g., INCB00928); or combinations thereof.

In some embodiments, the treatment methods described herein are combined with administration of a PI3K6 inhibitor. In some embodiments, the treatment methods described herein are combined with administration of a JAK inhibitor. In some embodiments, the treatment methods described herein are combined with administration of a JAK1 or JAK2 inhibitor (e.g., baricitinib or ruxolitinib). In some embodiments, the treatment methods described herein are combined with administration of a JAK1 inhibitor. In some embodiments, the treatment methods described herein are combined with administration of a JAK1 inhibitor, which is selective over JAK2.

Example antibodies that can be administered in combination therapy include, but are not limited to, trastuzumab (e.g., anti-HER2), ranibizumab (e.g., anti-VEGF-A), bevacizumab (AVASTIN™, e.g., anti-VEGF), panitumumab (e.g., anti-EGFR), cetuximab (e.g., anti-EGFR), rituxan (e.g., anti-CD20), and antibodies directed to c-MET.

One or more of the following agents may be administered to a patient in combination with the treatment methods of the present disclosure and are presented as a non-limiting list: a cytostatic agent, cisplatin, doxorubicin, taxotere, taxol, etoposide, irinotecan, camptostar, topotecan, paclitaxel, docetaxel, epothilones, tamoxifen, 5-fluorouracil, methoxtrexate, temozolomide, cyclophosphamide, SCH 66336, R115777, L778,123, BMS 214662, IRESSA™ (gefitinib), TARCEVA™ (erlotinib), antibodies to EGFR, intron, ara-C, adriamycin, cytoxan, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, oxaliplatin, leucovirin, ELOXATIN™ (oxaliplatin), pentostatine, vinblastine, vincristine, vindesine, bleomycin, dactinomycin, daunorubicin, doxorubicin, epirubicin, idarubicin, mithramycin, deoxycoformycin, mitomycin-C, L-asparaginase, teniposide 17.alpha.-ethinylestradiol, diethylstilbestrol, testosterone, Prednisone, Fluoxymesterone, Dromostanolone propionate, testolactone, megestrolacetate, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, avastin, HERCEPTIN™ (trastuzumab), BEXXAR™ (tositumomab), VELCADE™ (bortezomib), ZEVALIN™ (ibritumomab tiuxetan), TRISENOX™ (arsenic trioxide), XELODA™ (capecitabine), vinorelbine, porfimer, ERBITUX™ (cetuximab), thiotepa, altretamine, melphalan, trastuzumab, lerozole, fulvestrant, exemestane, ifosfomide, rituximab, C225 (cetuximab), Campath (alemtuzumab), clofarabine, cladribine, aphidicolon, rituxan, sunitinib, dasatinib, tezacitabine, Sm11, fludarabine, pentostatin, triapine, didox, trimidox, amidox, 3-AP, and MDL-101,731.

The treatment methods and regimens of the present disclosure can further be used in combination with other methods of treating cancers, for example by chemotherapy, irradiation therapy, tumor-targeted therapy, adjuvant therapy, immunotherapy or surgery. Examples of immunotherapy include cytokine treatment (e.g., interferons, GM-CSF, G-CSF, IL-2), CRS-207 immunotherapy, cancer vaccine, monoclonal antibody, bispecific or multi-specific antibody, antibody drug conjugate, adoptive T cell transfer, Toll receptor agonists, RIG-I agonists, oncolytic virotherapy and immunomodulating small molecules, including thalidomide or JAK1/2 inhibitor, PI3K6 inhibitor and the like. The compounds can be administered in combination with one or more anti-cancer drugs, such as a chemotherapeutic agent. Examples of chemotherapeutics include any of: abarelix, aldesleukin, alemtuzumab, alitretinoin, allopurinol, altretamine, anastrozole, arsenic trioxide, asparaginase, azacitidine, bevacizumab, bexarotene, baricitinib, bleomycin, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epacadostat, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, and zoledronate.

Additional examples of chemotherapeutics include proteosome inhibitors (e.g., bortezomib), thalidomide, revlimid, and DNA-damaging agents such as melphalan, doxorubicin, cyclophosphamide, vincristine, etoposide, carmustine, and the like.

Example steroids include corticosteroids such as dexamethasone or prednisone.

Example Bcr-Abl inhibitors include imatinib mesylate (GLEEVAC™), nilotinib, dasatinib, bosutinib, and ponatinib, and pharmaceutically acceptable salts. Other example suitable Bcr-Abl inhibitors include the compounds, and pharmaceutically acceptable salts thereof, of the genera and species disclosed in U.S. Pat. No. 5,521,184, WO 04/005281, and U.S. Ser. No. 60/578,491.

Example suitable Flt-3 inhibitors include midostaurin, lestaurtinib, linifanib, sunitinib, sunitinib, maleate, sorafenib, quizartinib, crenolanib, pacritinib, tandutinib, PLX3397 and ASP2215, and their pharmaceutically acceptable salts. Other example suitable Flt-3 inhibitors include compounds, and their pharmaceutically acceptable salts, as disclosed in WO 03/037347, WO 03/099771, and WO 04/046120.

Example suitable RAF inhibitors include dabrafenib, sorafenib, and vemurafenib, and their pharmaceutically acceptable salts. Other example suitable RAF inhibitors include compounds, and their pharmaceutically acceptable salts, as disclosed in WO 00/09495 and WO 05/028444.

Example suitable FAK inhibitors include VS-4718, VS-5095, VS-6062, VS-6063, BI853520, and GSK2256098, and their pharmaceutically acceptable salts. Other example suitable FAK inhibitors include compounds, and their pharmaceutically acceptable salts, as disclosed in WO 04/080980, WO 04/056786, WO 03/024967, WO 01/064655, WO 00/053595, and WO 01/014402.

Example suitable CDK4/6 inhibitors include palbociclib, ribociclib, trilaciclib, lerociclib, and abemaciclib, and their pharmaceutically acceptable salts. Other example suitable CDK4/6 inhibitors include compounds, and their pharmaceutically acceptable salts, as disclosed in WO 09/085185, WO 12/129344, WO 11/101409, WO 03/062236, WO 10/075074, and WO 12/061156.

The treatment methods and regimens of the present disclosure can further be used in combination with one or more other kinase inhibitors including imatinib, particularly for treating patients resistant to imatinib or other kinase inhibitors.

In some embodiments, the treatment methods of the disclosure can be used in combination with a chemotherapeutic in the treatment of cancer, and may improve the treatment response as compared to the response to the chemotherapeutic agent alone, without exacerbation of its toxic effects. In some embodiments, the treatment methods of the disclosure can be used in combination with a chemotherapeutic provided herein. For example, additional pharmaceutical agents used in the treatment of multiple myeloma, can include, without limitation, melphalan, melphalan plus prednisone [MP], doxorubicin, dexamethasone, and Velcade (bortezomib). Further additional agents used in the treatment of multiple myeloma include Bcr-Ab1, Flt-3, RAF and FAK kinase inhibitors. In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM). Additive or synergistic effects are desirable outcomes of combining treatment methods of the present disclosure with an additional agent.

The treatment methods of the disclosure can be combined with an antibody that binds to human PD-1 or human PD-L1, or antigen-binding fragment thereof.

In some embodiments, a corticosteroid such as dexamethasone is administered to a patient in combination with the treatment methods of the disclosure where the dexamethasone is administered intermittently as opposed to continuously.

The treatment methods described herein can be combined with another immunogenic agent, such as cancerous cells, purified tumor antigens (including recombinant proteins, peptides, and carbohydrate molecules), cells, and cells transfected with genes encoding immune stimulating cytokines. Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The treatment methods described herein can be used in combination with a vaccination protocol for the treatment of cancer. In some embodiments, the tumor cells are transduced to express GM-CSF. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). In some embodiments, the treatment methods and regimens of the present disclosure can be used in combination with tumor specific antigen such as heat shock proteins isolated from tumor tissue itself. In some embodiments, the treatment methods described herein can be combined with dendritic cells immunization to activate potent anti-tumor responses.

The treatment methods and regimens of the present disclosure can be used in combination with bispecific macrocyclic peptides that target Fe alpha or Fe gamma receptor-expressing effectors cells to tumor cells. The treatment methods and regimens of the present disclosure can also be combined with macrocyclic peptides that activate host immune responsiveness.

In some further embodiments, the treatment methods of the disclosure are combined with administration of other therapeutic agents to a patient prior to, during, and/or after a bone marrow transplant or stem cell transplant. The treatment methods and regimens of the present disclosure can be used in combination with bone marrow transplant for the treatment of a variety of tumors of hematopoietic origin.

When more than one pharmaceutical agents is administered to a patient, as discussed in any of the above embodiments, they can be administered simultaneously, separately, sequentially, or in combination (e.g., for more than two agents).

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

### II. Immune-Checkpoint Therapies

The treatment methods described herein can be used in combination with one or more immune checkpoint inhibitors for the treatment of diseases, such as cancer or infections. Exemplary immune checkpoint inhibitors include inhibitors against immune checkpoint molecules such as CBL-B, CD20, CD28, CD40, CD70, CD122, CD96, CD73, CD47, CDK2, GITR, CSF1R, JAK, PI3K delta, PI3K gamma, TAM, arginase, HPK1, CD137 (also known as 4-1BB), ICOS, A2AR, B7-H3, B7-H4, BTLA, CTLA-4, LAG3, TIM3, TLR (TLR7/8), TIGIT, CD112R, VISTA, PD-1, PD-L1 and PD-L2. In some embodiments, the immune checkpoint molecule is a stimulatory checkpoint molecule selected from CD27, CD28, CD40, ICOS, OX40, GITR and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, TIGIT, and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the treatment methods provided herein can be used in combination with one or more agonists of immune checkpoint molecules, e.g., OX40, CD27, GITR, and CD137 (also known as 4-1BB).

In some embodiments, the inhibitor of an immune checkpoint molecule is anti-PD1 antibody, anti-PD-L1 antibody, or anti-CTLA-4 antibody.

In some embodiments, the inhibitor of an immune checkpoint molecule is an inhibitor of PD-1 or PD-L1, e.g., an anti-PD-1 or anti-PD-L1 monoclonal antibody. In some embodiments, the anti-PD-1 or anti-PD-L1 antibody is nivolumab, pembrolizumab, atezolizumab, durvalumab, avelumab, cemiplimab, atezolizumab, avelumab, tislelizumab, spartalizumab (PDR001), cetrelimab (JNJ-63723283), toripalimab (JS001), camrelizumab (SHR-1210), sintilimab (IBI308), AB122 (GLS-010), AMP-224, AMP-514/MEDI-0680, BMS936559, JTX-4014, BGB-108, SHR-1210, MEDI4736, FAZ053, BCD-100, KN035, CS1001, BAT1306, LZMO09, AK105, HLX10, SHR-1316, CBT-502 (TQB2450), A167 (KL-A167), STI-A101 (ZKAB001), CK-301, BGB-A333, MSB-2311, HLX20, TSR-042, or LY3300054. In some embodiments, the inhibitor of PD-1 or PD-L1 is one disclosed in U.S. Pat. Nos. 7,488,802, 7,943,743, 8,008,449, 8,168,757, 8,217, 149, or 10,308,644; U.S. Publ. Nos. 2017/0145025, 2017/0174671, 2017/0174679, 2017/0320875, 2017/0342060, 2017/0362253, 2018/0016260, 2018/0057486, 2018/0177784, 2018/0177870, 2018/0179179, 2018/0179201, 2018/0179202, 2018/0273519, 2019/0040082, 2019/0062345, 2019/0071439, 2019/0127467, 2019/0144439, 2019/0202824, 2019/0225601, 2019/0300524, or 2019/0345170; or PCT Pub. Nos. WO 03042402, WO 2008156712, WO 2010089411, WO 2010036959, WO 2011066342, WO 2011159877, WO 2011082400, or WO 2011161699, which are each incorporated herein by reference in their entirety. In some embodiments, the inhibitor of PD-L1 is INCB086550.

In some embodiments, the antibody is an anti-PD-1 antibody, e.g., an anti-PD-1 monoclonal antibody. In some embodiments, the anti-PD-1 antibody is nivolumab, retifanlimab pembrolizumab, cemiplimab, spartalizumab, camrelizumab, cetrelimab, toripalimab, sintilimab, AB122, AMP-224, JTX-4014, BGB-108, BCD-100, BAT1306, LZMO09, AK105, HLX10, or TSR-042. In some embodiments, the anti-PD-1 antibody is nivolumab, pembrolizumab, cemiplimab, spartalizumab, camrelizumab, cetrelimab, toripalimab, or sintilimab. In some embodiments, the anti-PD-1 antibody is pembrolizumab. In some embodiments, the anti-PD-1 antibody is nivolumab. In some embodiments, the anti-PD-1 monoclonal antibody is retifanlimab. In some embodiments, the anti-PD-1 antibody is cemiplimab. In some embodiments, the anti-PD-1 antibody is spartalizumab. In some embodiments, the anti-PD-1 antibody is camrelizumab. In some embodiments, the anti-PD-1 antibody is cetrelimab. In some embodiments, the anti-PD-1 antibody is toripalimab. In some embodiments, the anti-PD-1 antibody is sintilimab. In some embodiments, the anti-PD-1 antibody is AB122. In some embodiments, the anti-PD-1 antibody is AMP-224. In some embodiments, the anti-PD-1 antibody is JTX-4014. In some embodiments, the anti-PD-1 antibody is BGB-108. In some embodiments, the anti-PD-1 antibody is BCD-100. In some embodiments, the anti-PD-1 antibody is BAT1306. In some embodiments, the anti-PD-1 antibody is LZMO09. In some embodiments, the anti-PD-1 antibody is AK105. In some embodiments, the anti-PD-1 antibody is HLX10. In some embodiments, the anti-PD-1 antibody is TSR-042. In some embodiments, the anti-PD-1 monoclonal antibody is nivolumab or pembrolizumab. In some embodiments, the anti-PD1 antibody is SHR-1210. Other anti-cancer agent(s) include antibody therapeutics such as 4-1BB (e.g., urelumab, utomilumab). In some embodiments, the inhibitor of an immune checkpoint molecule is an inhibitor of PD-L1, e.g., an anti-PD-L1 monoclonal antibody. In some embodiments, the anti-PD-L1 monoclonal antibody is atezolizumab, avelumab, durvalumab, tislelizumab, BMS-935559, MEDI4736, atezolizumab (MPDL3280A;also known as RG7446), avelumab (MSB0010718C), FAZ053, KN035, CS1001, SHR-1316, CBT-502, A167, STI-A101, CK-301, BGB-A333, MSB-2311, HLX20, or LY3300054. In some embodiments, the anti-PD-L1 antibody is atezolizumab, avelumab, durvalumab, or tislelizumab.

In some embodiments, the anti-PD-L1 antibody is atezolizumab. In some embodiments, the anti-PD-L1 antibody is avelumab. In some embodiments, the anti-PD-L1 antibody is durvalumab. In some embodiments, the anti-PD-L1 antibody is tislelizumab. In some embodiments, the anti-PD-L1 antibody is BMS-935559. In some embodiments, the anti-PD-L1 antibody is MEDI4736. In some embodiments, the anti-PD-L1 antibody is FAZ053. In some embodiments, the anti-PD-L1 antibody is KN035. In some embodiments, the anti-PD-L1 antibody is CS1001. In some embodiments, the anti-PD-L1 antibody is SHR-1316. In some embodiments, the anti-PD-L1 antibody is CBT-502. In some embodiments, the anti-PD-L1 antibody is A167. In some embodiments, the anti-PD-L1 antibody is STI-A101. In some embodiments, the anti-PD-L1 antibody is CK-301. In some embodiments, the anti-PD-L1 antibody is BGB-A333. In some embodiments, the anti-PD-L1 antibody is MSB-2311. In some embodiments, the anti-PD-L1 antibody is HLX20. In some embodiments, the anti-PD-L1 antibody is LY3300054.

In some embodiments, the inhibitor of an immune checkpoint molecule is a small molecule that binds to PD-L1, or a pharmaceutically acceptable salt thereof. In some embodiments, the inhibitor of an immune checkpoint molecule is a small molecule that binds to and internalizes PD-L1, or a pharmaceutically acceptable salt thereof. In some embodiments, the inhibitor of an immune checkpoint molecule is a compound selected from those in U.S. 2018/0179201, U.S. 2018/0179197, U.S. 2018/0179179, U.S. 2018/0179202, U.S. 2018/0177784, U.S. 2018/0177870, U.S. Ser. No. 16/369,654 (filed Mar. 29, 2019), and U.S. Ser. No. 62/688,164, or a pharmaceutically acceptable salt thereof, each of which is incorporated herein by reference in its entirety.

In some embodiments, the inhibitor of an immune checkpoint molecule is an inhibitor of KIR, TIGIT, LAIR1, CD160, 2B4 and TGFR beta.

In some embodiments, the inhibitor is MCLA-145.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, INCAGN2385, or eftilagimod alpha (IMP321).

In some embodiments, the inhibitor of an immune checkpoint molecule is an inhibitor of CD73. In some embodiments, the inhibitor of CD73 is oleclumab.

In some embodiments, the inhibitor of an immune checkpoint molecule is an inhibitor of TIGIT. In some embodiments, the inhibitor of TIGIT is OMP-31M32.

In some embodiments, the inhibitor of an immune checkpoint molecule is an inhibitor of VISTA. In some embodiments, the inhibitor of VISTA is JNJ-61610588 or CA-170.

In some embodiments, the inhibitor of an immune checkpoint molecule is an inhibitor of B7-H3. In some embodiments, the inhibitor of B7-H3 is enoblituzumab, MGD009, or 8H9.

In some embodiments, the inhibitor of an immune checkpoint molecule is an inhibitor of KIR. In some embodiments, the inhibitor of KIR is lirilumab or IPH4102.

In some embodiments, the inhibitor of an immune checkpoint molecule is an inhibitor of A2aR. In some embodiments, the inhibitor of A2aR is CPI-444.

In some embodiments, the inhibitor of an immune checkpoint molecule is an inhibitor of TGF-beta. In some embodiments, the inhibitor of TGF-beta is trabedersen, galusertinib, or M7824.

In some embodiments, the inhibitor of an immune checkpoint molecule is an inhibitor of PI3K-gamma. In some embodiments, the inhibitor of PI3K-gamma is IPI-549.

In some embodiments, the inhibitor of an immune checkpoint molecule is an inhibitor of CD47. In some embodiments, the inhibitor of CD47 is Hu5F9-G4 or TTI-621.

In some embodiments, the inhibitor of an immune checkpoint molecule is an inhibitor of CD73. In some embodiments, the inhibitor of CD73 is MEDI9447.

In some embodiments, the inhibitor of an immune checkpoint molecule is an inhibitor of CD70. In some embodiments, the inhibitor of CD70 is cusatuzumab or BMS-936561. In some embodiments, the inhibitor of an immune checkpoint molecule is an inhibitor of

TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of CD20, e.g., an anti-CD20 antibody. In some embodiments, the anti-CD20 antibody is obinutuzumab or rituximab.

In some embodiments, the agonist of an immune checkpoint molecule is an agonist of OX40, CD27, CD28, GITR, ICOS, CD40, TLR7/8, and CD137 (also known as 4-1BB).

In some embodiments, the agonist of CD137 is urelumab. In some embodiments, the agonist of CD137 is utomilumab.

In some embodiments, the agonist of an immune checkpoint molecule is an inhibitor of GITR. In some embodiments, the agonist of GITR is TRX518, MK-4166, INCAGN1876, MK-1248, AMG228, BMS-986156, GWN323, MEDI1873, or MEDI6469.In some embodiments, the agonist of an immune checkpoint molecule is an agonist of 0X40, e.g., OX40 agonist antibody or OX40L fusion protein. In some embodiments, the anti-0X40 antibody is INCAGN01949, MEDI0562 (tavolimab), MOXR-0916, PF-04518600, GSK3174998, BMS-986178, or 9B12. In some embodiments, the OX4OL fusion protein is MEDI6383.

In some embodiments, the agonist of an immune checkpoint molecule is an agonist of CD40. In some embodiments, the agonist of CD40 is CP-870893, ADC-1013, CDX-1140, SEA-CD40, R07009789, JNJ-64457107, APX-005M, or Chi Lob 7/4.

In some embodiments, the agonist of an immune checkpoint molecule is an agonist of ICOS. In some embodiments, the agonist of ICOS is GSK-3359609, JTX-2011, or MEDI-570.

In some embodiments, the agonist of an immune checkpoint molecule is an agonist of CD28. In some embodiments, the agonist of CD28 is theralizumab.

In some embodiments, the agonist of an immune checkpoint molecule is an agonist of CD27. In some embodiments, the agonist of CD27 is varlilumab.

In some embodiments, the agonist of an immune checkpoint molecule is an agonist of TLR7/8. In some embodiments, the agonist of TLR7/8 is MEDI9197.

The compounds of the present disclosure can be used in combination with bispecific antibodies. In some embodiments, one of the domains of the bispecific antibody targets PD-1, PD-L1, CTLA-4, GITR, OX40, TIM3, LAG3, CD137, ICOS, CD3 or TGFP receptor. In some embodiments, the bispecific antibody binds to PD-1 and PD-L1 . In some embodiments, the bispecific antibody that binds to PD-1 and PD-L1 is MCLA-136. In some embodiments, the bispecific antibody binds to PD-L1 and CTLA-4. In some embodiments, the bispecific antibody that binds to PD-L1 and CTLA-4 is AK104.

In some embodiments, the compounds of the disclosure can be used in combination with one or more metabolic enzyme inhibitors. In some embodiments, the metabolic enzyme inhibitor is an inhibitor of ID01, TDO, or arginase. Examples of IDO1 inhibitors include epacadostat, NLG919, BMS-986205, PF-06840003, IOM2983, RG-70099 and LY338196. Inhibitors of arginase inhibitors include INCB1158.

As provided throughout, the additional compounds, inhibitors, agents, etc. can be combined with the present compound in a single or continuous dosage form, or they can be administered simultaneously or sequentially as separate dosage forms.

### Pharmaceutical Formulations and Dosage Forms

When employed as pharmaceuticals, the compounds as described herein can be administered in the form of pharmaceutical compositions which refers to a combination of a compound as described herein, and at least one pharmaceutically acceptable carrier. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal, or intramuscular injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, an FGFR1 inhibitor and/or a KRAS inhibitor in combination with one or more pharmaceutically acceptable carriers. In making the compositions described herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions described herein can be formulated so as to provide quick, sustained or delayed release of the active ingredient after administration to the patient by employing procedures known in the art.

The compositions can be formulated in a unit dosage form, each dosage containing from about 1 to about 10 mg, or about 5 mg, of the active ingredient. In some embodiments, the unit dosage form contains about 2 mg of the active ingredient. In some embodiments, the unity dosage form contains about 1 mg of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid pre-formulation composition containing a homogeneous mixture of the active ingredient. When referring to these pre-formulation compositions as homogeneous, the active ingredient is typically dispersed evenly throughout the composition so that the composition can be readily subdivided into equally effective unit dosage forms such as tablets, pills and capsules. This solid pre-formulation is then subdivided into unit dosage forms of the type described above containing from, for example, 0.1 to about 500 mg of the active ingredient of the present disclosure.

In some embodiments, the active ingredient is pemigatinib. In some embodiments, pemigatinib is administered orally. In some embodiments, pemigatinib is administered once daily. In some embodiments, pemigatinib is administered in a daily dose of about 1 mg to about 20 mg. In some embodiments, pemigatinib is administered in a daily dose of about 1 mg to about 5 mg. In some embodiments, pemigatinib is administered in a daily dose of about 2 mg. In some embodiments, pemigatinib is administered as a tablet. In some embodiments, the tablet comprises about 0.5 mg to about 10 mg of pemigatinib. In some embodiments, the tablet comprises about 0.5 mg to about 5 mg pemigatinib. In some embodiments, the tablet comprises about 2 mg, about 4.5 mg, about 9 mg, about 13.5 mg, or about 18 mg of pemigatinib. In some embodiments, the tablet comprises about 0.5 mg of pemigatinib. In some embodiments, the tablet comprises about 2 mg of pemigatinib. In some embodiments, the tablet comprises about 4.5 mg of pemigatinib. In some embodiments, the tablet comprises about 9 mg of pemigatinib. In some embodiments, the tablet comprises about 13.5 mg of pemigatinib. In some embodiments, the tablet comprises about 18 mg of pemigatinib.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the pemigatinib, or compositions as described herein can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of pemigatinib can vary according to, for example, the particular use for which the treatment is made, the manner of administration of the compound, the health and condition of the patient, and the judgment of the prescribing physician. The proportion or concentration of pemigatinib in a pharmaceutical composition can vary depending upon a number of factors including dosage, chemical characteristics (e.g., hydrophobicity), and the route of administration. For example, pemigatinib can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

Pemigatinib can also be formulated in combination with one or more additional active ingredients which can include any pharmaceutical agent such as anti-viral agents, vaccines, antibodies, immune enhancers, immune suppressants, anti-inflammatory agents and the like.

In some embodiments, the active ingredient is sotorasib. Sotorasib as described herein can be administered in the form of pharmaceutical compositions and at least one pharmaceutically acceptable excipient. In some embodiments, the active ingredient is adagrasib. Adagrasib as described herein can be administered in the form of pharmaceutical compositions and at least one pharmaceutically acceptable excipient. These compositions can be prepared in a manner well known in the pharmaceutical art, and can be administered by a variety of routes, depending upon whether local or systemic treatment is desired and upon the area to be treated. The pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application. Typically compositions for the agents described herein are in the form of tablets.

### Labeled Compound

Another aspect of the present disclosure relates to labeled FGFR1 inhibitors, KRAS inhibitors, or both as described herein (radio-labeled, fluorescent-labeled, isotopically-labeled, etc.) that would be useful not only in imaging techniques but also in assays, both in vitro and in vivo.

The present disclosure further includes isotopically-labeled FGFR1 inhibitors, KRAS inhibitors, or both as described herein. An “isotopically” or “radio-labeled” compound is an FGFR1 inhibitor, a KRAS inhibitor, or both as described herein, where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴, ¹²⁵I and ¹³¹I. For example, one or more hydrogen atoms in a compound of the present disclosure can be replaced by deuterium atoms can be optionally substituted with deuterium atoms.

One or more constituent atoms of the FGFR1 inhibitor, the KRAS inhibitor, or both, can be replaced or substituted with isotopes of the atoms in natural or non-natural abundance. In some embodiments, the FGFR1 inhibitor, the KRAS inhibitor, or both includes at least one deuterium atom. For example, one or more hydrogen atoms in a compound presented herein can be replaced or substituted by deuterium. In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms.

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et al. J. Med. Chem. 2011, 54, 201-210; R. Xu et al. J. Label Compd. Radiopharm. 2015, 58, 308-312). In particular, substitution at one or more metabolism sites may afford one or more of the therapeutic advantages.

It is understood that a “radio-labeled” or “labeled compound” is a compound that has incorporated at least one radionuclide. In some embodiments, the radionuclide is selected from the group consisting of ³H and ¹⁴C. In some embodiments, the radionuclide is selected from the group consisting of ¹¹C, ¹⁸F, ⁷⁵Br, ⁷⁶Br, and ⁷⁷Br.

### Kits

The present disclosure also includes pharmaceutical kits useful, e.g., in the treatment of cancer, which include one or more containers containing a pharmaceutical composition comprising a therapeutically effective amount of an FGFR1 inhibitor and a KRAS inhibitor, or any of the embodiments thereof. Such kits can further include one or more of various conventional pharmaceutical kit components, such as, e.g., containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### Example A. Treatment of KRAS-G12C Inhibitor in Mesenchymal-Like Lung Cancer Cells Elevated FGFR Downstream Signaling

FGFR1 and downstream activation marker pFRS2a protein levels were examined in indicated cell lines, as well as markers for epithelial-like cells (E-cadherin) and mesenchymal-like cells (Vimentin).

Mesenchymal-like cells, indicated by low E-cadherin and high Vimentin expression, including LU99, H1792 and SW1573 displayed higher FGFR1 and pFRS2a levels compared to H358, which is a epithelial-like cell line expressing high E-cadherin and low Vimentin.

In LU99 cells, 50 nM of a KRAS G12C inhibitor (AMG510 or Compound 2) elevated the protein level of pFRS2a, and pemigatinib but not the FGFR2/3 inhibitor Compound 1, inhibited the pFRS2a level in combination with KRAS inhibitors.

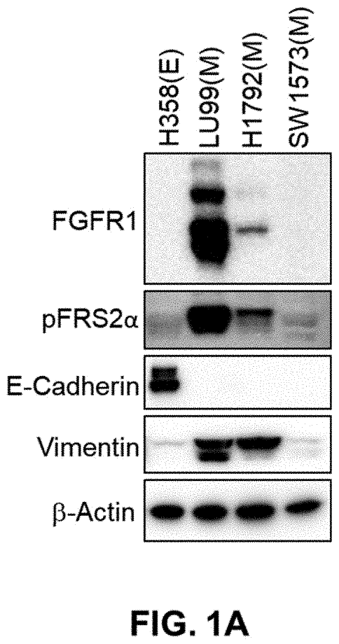

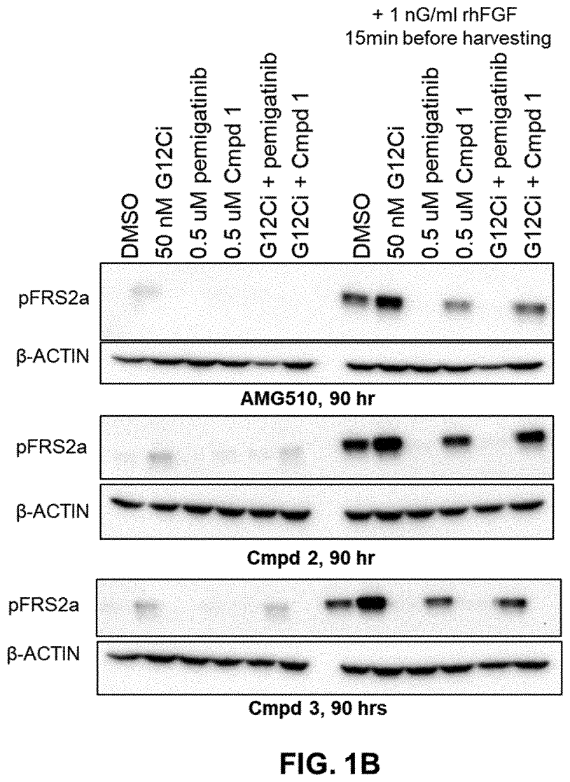

FIG. 1A depicts Western blots of FGFR1, pFRS2a, E-cadherin, Vimentin and beta-actin in four indicated cell lines.

FIG. 1B depicts Western blots of pFRS2a and beta-actin in LU99 cell lysate with indicated treatment.

Methods:

Cells were seeded in Corning 6-well tissue culture treated plates in RPMI medium with 10% FBS at 6×105 cells/well. After 48 hours, the cells were harvested for western blots (A), or treated with indicated compounds for 90 hours at 37° C., 5% CO₂. The cells were washed with PBS and lysed with lx lysis buffer (Cell Signaling #9803) with protease and phosphatase inhibitors. Twenty five μg of total protein lysates was subjected to SDS-PAGE and immunoblot analysis using antibodies from Cell Signaling Technology.

In conclusion, mesenchymal-like cancer cells displayed elevated FGFR1 signaling; and KRAS G12C inhibition alone led to an increase of the FGFR1 signaling. Combination of a KRAS G12C inhibitor with the FGFR1 inhibitor pemigatinib, but not with the FGFR2/3 inhibitor Compound 1, inhibited the KRAS G12C inhibitor induced upregulation of the FGFR1 pathway.

### Example B. Combination of Compound 2 and Pemigatinib, but Not Compound 1, Maximized the Inhibition of pERK Signaling in LU99 Cells

The MEK-ERK pathway is the main downstream signaling pathway of KRAS G12C mutated cancer cells to survive and proliferate. Therefore, the impact to phosphor-ERK (pERK) was evaluated with KRAS G12C inhibitor and FGFR inhibitor treatment.

LU99 cells were treated with 100 nM of Compound 2, pemigatinib, Compound 1, or indication combination for 24 hours, and with or without lng/ml recombinant human FGF (rhFGF) before harvesting the lysate. Both pERK and pFRS2a signaling was elevated after lng/ml hrFGF treatment. With or without rhFGF stimulation, pFRS2a was elevated after Compound 2 treatment; and combination of Compound 2 and pemigatinib fully inhibited pERK signaling.

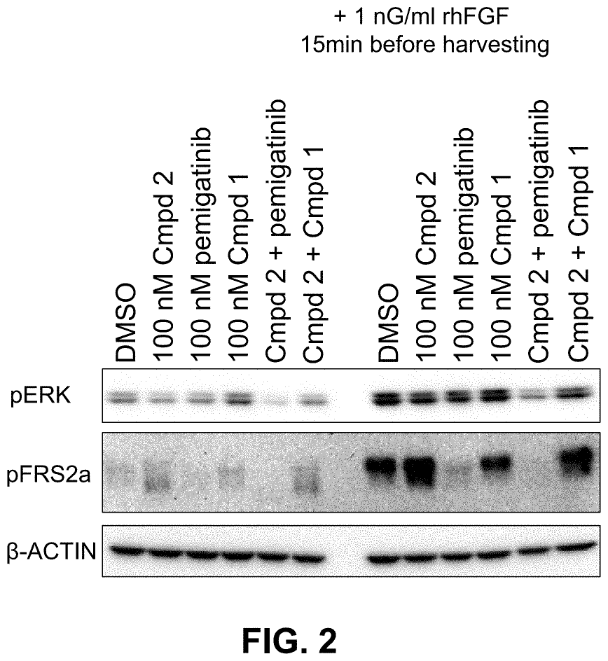

FIG. 2 showsWestern blots of pERK, pFRS2a, and beta-actin in LU99 cells treated with the KRAS G12C inhibitor Compound 2 with and without pemigatinib or Compound 1 for 24 hours.

Methods:

Cells were seeded in Corning 6-well tissue culture treated plates in RPMI medium with 10% FBS at 6×105 cells/well. After 48 hours, cells were or treated with indicated compounds for 24 hours at 37° C., 5% CO₂. rhFGF—basic (R&D systems 233-FB) was added at 1 nG/ml for 15 minutes at the end of the experiment as indicated. The cells were washed with PBS and lysed with 1x lysis buffer (Cell Signaling #9803) with protease and phosphatase inhibitors. Twenty-five μg of total protein lysates was subjected to SDS-PAGE and immunoblot analysis using the following Cell Signaling antibodies: pERK (#4370) phospho-FRS2a (#3861) and beta-Actin (#12620).

In summary, in mesenchymal-like cells like LU99, inhibiting KRAS G12C did not fully inhibit the downstream pERK signaling; however, the combination of a KRAS G12C inhibitor with an FGFR1 inhibitor could fully achieve the inhibition of the pERK pathway.

### Example C. Mesenchymal-Like Cell Lines Show an Increased Synergistic Effect Over Epithelial-Like Cell Lines with Pemigatinib+G12C Inhibition

The in-vitro effect of combining the FGFR1 inhibitor pemigatinib with a KRAS G12C inhibitor selected from AMG-510, Compound 3, or Compound 4 was assessed in various cell lines indicating a higher synergistic effect in Mesenchymal-like cell lines than was demonstrated in Epithelial-like cell lines.

TABLE 1
Table of average Bliss scores observed during in-vitro
investigating six cell lines with a combination of the
FGFR1 inhibitor pemigatinib and multiple KRAS G12C inhibitors
including AMG-510, Compound 3, and Compound 4.

| | | | KRAS | Avg. Bliss |
|---|---|---|---|---|
| Cell Line | Tissue | Type | Inhibitor | Score |
| H358 | Lung | Epithelial | AMG-510 | 3.7 |
| H358 | Lung | Epithelial | Cmpd 4 | −3 |
| H358 | Lung | Epithelial | Cmpd 3 | 3.8 |
| SW-837 | CRC | Epithelial | AMG-510 | −7.7 |
| SW-837 | CRC | Epithelial | Cmpd 4 | −11.7 |
| SW-837 | CRC | Epithelial | Cmpd 3 | 0.9 |
| Calu-1 | Lung | Mesenchymal | AMG-510 | 11.2 |
| Calu-1 | Lung | Mesenchymal | Cmpd 4 | −9.9 |
| Calu-1 | Lung | Mesenchymal | Cmpd 3 | 8.7 |
| HCC-44 | Lung | Mesenchymal | AMG-510 | 4.4 |
| HCC-44 | Lung | Mesenchymal | Cmpd 4 | 4.7 |
| HCC-44 | Lung | Mesenchymal | Cmpd 3 | 5.8 |
| SW-1573 | Lung | Mesenchymal | AMG-510 | 25.9 |
| SW-1573 | Lung | Mesenchymal | Cmpd 4 | 17 |
| SW-1573 | Lung | Mesenchymal | Cmpd 3 | 18.6 |
| H1792 | Lung | Mesenchymal | AMG-510 | 26.7 |
| H1792 | Lung | Mesenchymal | Cmpd 4 | 30 |
| H1792 | Lung | Mesenchymal | Cmpd 3 | 28.5 |

Methods:

Cells were seeded in RPMI medium with 10% HI FBS at a density of 500 cells/well in Greiner, white, clear bottom, 384-well tissue culture treated plates containing 10×10 combination matrices of compound. Plates were incubated at 37° C., 5% CO2 for 5 days. On day 5, Cell Titer Glo reagent was added to the plates and ATP luminescence was detected using a Pherastar FSX reader. The synergy score for the combination effect was calculated using a Bliss score model where the Bliss score=Yab-(Ya+Yb−(YaYb))×100, where Ya and Yb are the monotherapies. Bliss scores >20 are strongly synergistic and a higher Bliss scores indicates a higher level of synergism.

In summary, in mesenchymal-like cells, but not in epithelial-like KRAS G12C mutant cells, the combination of the FGFR1 inhibitor pemigatinib with a KRAS G12C inhibitor (i.e., AMG-510, Compound 3 or Compound 4) synergistically inhibited cell proliferation.

### Example D. Synergistic Effect Observed Across Cell Lines with FGFR and KRAS G12C Inhibitor Combination

The in-vitro effect of combining KRAS G12 inhibitor Compound 3 with FGFR inhibitors pemigatinib, Compound 1, TAS-120, Erdafitinib or BGJ398 was assessed in various cell lines indicating higher synergistic effect in Mesenchymal-like cell lines over Epithelial-like cell lines.

TABLE 2
Table of average Bliss scores observed in-vitro investigating
four cell lines with combination of KRAS G12 inhibitor
Compound 3 and multiple FGFR inhibitors including Compound
1, TAS-120, Erdafitinib or BGJ398.

| | | | | | Avg. |
|---|---|---|---|---|---|
| Cell | | | KRAS | FGFR | Bliss |
| Line | Tissue | Type | Inhibitor | Inhibitor | Score |
| H358 | Lung | Epithelial | Cmpd 3 | Pemigatinib | 1.4 |
| H358 | Lung | Epithelial | Cmpd 3 | Cmpd 1 | 3 |
| H358 | Lung | Epithelial | Cmpd 3 | TAS-120 | 3.3 |
| H358 | Lung | Epithelial | Cmpd 3 | Erdafitinib | 4.4 |
| H358 | Lung | Epithelial | Cmpd 3 | BGJ398 | −0.8 |
| H1792 | Lung | Mesenchymal | Cmpd 3 | Pemigatinib | 24.2 |
| H1792 | Lung | Mesenchymal | Cmpd 3 | Cmpd 1 | 1.9 |
| H1792 | Lung | Mesenchymal | Cmpd 3 | TAS-120 | 17.4 |
| H1792 | Lung | Mesenchymal | Cmpd 3 | Erdafitinib | 12.9 |
| H1792 | Lung | Mesenchymal | Cmpd 3 | BGJ398 | 11.2 |
| SW-1573 | Lung | Mesenchymal | Cmpd 3 | Pemigatinib | 19.3 |
| SW-1573 | Lung | Mesenchymal | Cmpd 3 | Cmpd 1 | 3.1 |
| SW-1573 | Lung | Mesenchymal | Cmpd 3 | TAS-120 | 6.6 |
| SW-1573 | Lung | Mesenchymal | Cmpd 3 | Erdafitinib | 7.9 |
| SW-1573 | Lung | Mesenchymal | Cmpd 3 | BGJ398 | 7.5 |
| LU-99 | Lung | Mesenchymal | Cmpd 3 | Pemigatinib | 44.8 |
| LU-99 | Lung | Mesenchymal | Cmpd 3 | Cmpd 1 | 13.3 |
| LU-99 | Lung | Mesenchymal | Cmpd 3 | TAS-120 | 22 |
| LU-99 | Lung | Mesenchymal | Cmpd 3 | Erdafitinib | 34.7 |
| LU-99 | Lung | Mesenchymal | Cmpd 3 | BGJ398 | 31.9 |

### Methods:

Cells were seeded in RPMI medium with 10% HI FBS at a density of 500 cells/well in Greiner, white, clear bottom, 384-well tissue culture treated plates containing 10×10 combination matrices of compound. Plates were incubated at 37° C., 5% CO2 for 5 days. On day 5, Cell Titer Glo reagent was added to the plates and ATP luminescence was detected using a Pherastar FSX reader. Synergy score for the combination effect is calculated using a Bliss score model where the Bliss score=Yab−(Ya+Yb−(YaYb))×100, where Ya and Yb are the monotherapies. Bliss scores >20 are strongly synergistic and a higher Bliss scores indicates a higher level of synergism.

In summary, in mesenchymal-like but not epithelial-like KRAS G12C mutant cells, the combination of the FGFR1 inhibitor pemigatinib with KRAS G12C inhibitors synergistically inhibited cell proliferation. Compound 1, which is an FGFR2/3 selective inhibitor, did not show synergy with a KRAS G12C inhibitor in all cell lines, indicating that pemigatinib mainly works through the FGFR1 receptor.

Example E. Combinational Effect of Pemigatinib with AMG510 Results in Increased Tumor Growth Control In Vivo

### LU99 Xenograft Model

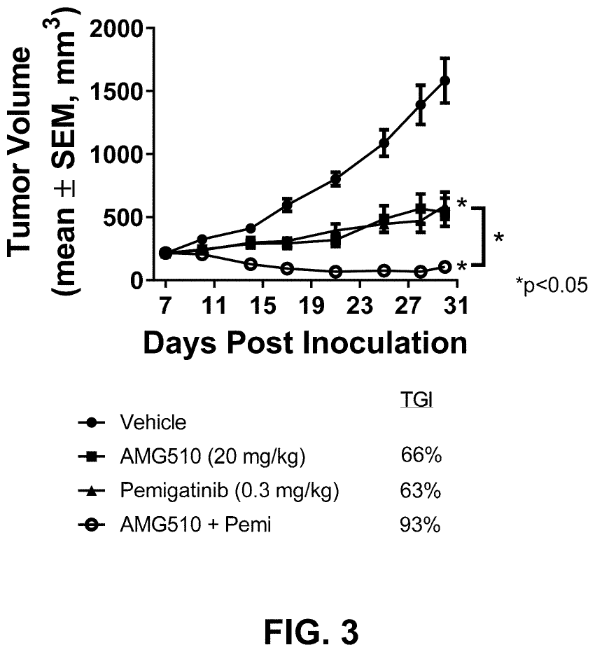

The in vivo effect of combining the FGFR1 inhibitor pemigatinib plus the KRAS G12C inhibitor AMG510 was assessed in the LU99 lung cancer model (JCRB0080, JCRB) xenograft model (FIG. 3) in 6 to 8 week old female NCr nude mice (Taconic). Pemigatinib and AMG510 were suspended in 5% N,N-dimethyl acetamide (DMAC)+50 mM Citrate buffer (pH 3.0) in 0.5% methyl cellulose for oral administration. Briefly, mice were inoculated in the left flank with 1×10⁷ LU99 cells re-suspended in a PBS and matrigel (Corning Life Sciences, Tewksbury, Mass) solution at 1:1. On Day 7, mice were randomized into 4 groups of 10 mice at approximate mean volume (˜212 mm³). Starting on Day 7 mice were dosed with (i) vehicle; (ii) 20 mg/kg of AMG510; (iii) 0.3 mg/kg of pemigatinib; or (iv) the combination of AMG510 and pemigatinib at 20 mg/kg and 0.3 mg/kg, respectively. Pemigatinib and AMG510 were administered orally once daily (QD) for the 30 day duration of the study. All treatment groups had statistically significant TGI (tumor growth inhibition) compared to Vehicle-treated mice. Tumor growth inhibition was calculated using the formula (1−(V_{T}/V_{C}))×100, where V_{T} is the tumor volume of the treatment group on the last day of treatment, and V_{c} is the tumor volume of the control group on the last day of treatment. The TGI for AMG510, pemigatinib, and the combination were 66%, 63% and 93%, respectively. The TGI of the combination group was statistically different than the single agent groups. Statistics were determined using One-Way ANOVA.

FIG. 3 is a graph depicting the tumor volume of LU99 tumor bearing mice administered (i) vehicle; (ii) 20 mg/kg of AMG510; (iii) 0.3 mg/kg of pemigatinib; or (iv) the combination of AMG510 and pemigatinib at 20 mg/kg and 0.3 mg/kg, respectively.

In summary, the combination of AMG510 and pemigatinib achieved maximal efficacy compared to each single agent treatment group in LU99 xenograft models, i.e., the combination acted synergistically.

### Example F. The Combination of Pemigatinib with AMG510 Results in Increased Phospho-ERK Inhibition in LU99 Tumors Compared to Single Agent Treatments

LU99 In Vivo pERK Inhibition

The effect of combining the combination of pemigatinib and AMG510 on pERK inhibition was assessed in vivo in the LU99 lung cancer xenograft model in 6 to 8 week old NCr nude mice (Taconic). Pemigatinib and AMG510 were suspended in 5% N,N-dimethyl acetamide (DMAC)+50 mM Citrate buffer (pH 3.0) in 0.5% methyl cellulose for oral administration. Briefly, mice were inoculated in the left flank with 1×10⁷LU99 cells re-suspended in a PBS and matrigel (Corning Life Sciences, Tewksbury, Mass) solution at 1:1. When tumors were approximately ˜564 mm³, mice were given a single dose of (i) vehicle; (ii) 20 mg/kg of AMG510; (iii) 0.3 mg/kg of pemigatinib; or (iv) 20 mg/kg AMG510 and 0.3 mg/kg of pemigatinib, and tumors were collected 2 h post dose. Tumors were then processed and levels of phospho ERK relative to total ERK were assessed on tumor lysates by MSD (Mesoscale). The data show that mice receiving the combination of AMG510 and pemigatinib had statistically higher pERK inhibition than either AMG510 alone, or pemigatinib alone. The percent inhibition was calculated relative to vehicle-treated mice, and is shown as the ratio of phospho-ERK to total ERK. Statistics were determined using One-Way ANOVA.

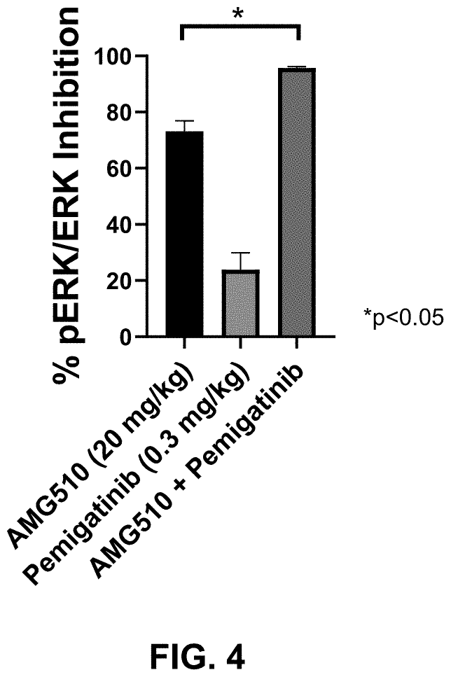

FIG. 4 is a graph depicting the inhibition of pERK in LU99 tumors from mice receiving 20 mg/kg of AMG510, 0.3 mg/kg of pemigatinib, or the combination of AMG510 and pemigatinib at 20 mg/kg and 0.3 mg/kg, respectively.

In summary, AMG510 and pemigatinib combination achieved maximal inhibition of pERK signaling in LU99 xenograft models, which is consistent with previous in vitro results.

### Example G. Treatment of Mesenchymal-Like Lung Cancer Cells with MRTX849 (adagrasib)

FGFR1 downstream activation marker pFRS2a protein levels were examined in the mesenchymal-like cell line LU99. In these cells, 50 n M of a KRAS G12C inhibitor (MRTX849) elevated the protein level of pFRS2a, and 0.5 μM pemigatinib but not the FGFR2/3 inhibitor Compound 1, inhibited the pFRS2a level in combination with KRAS inhibitors.

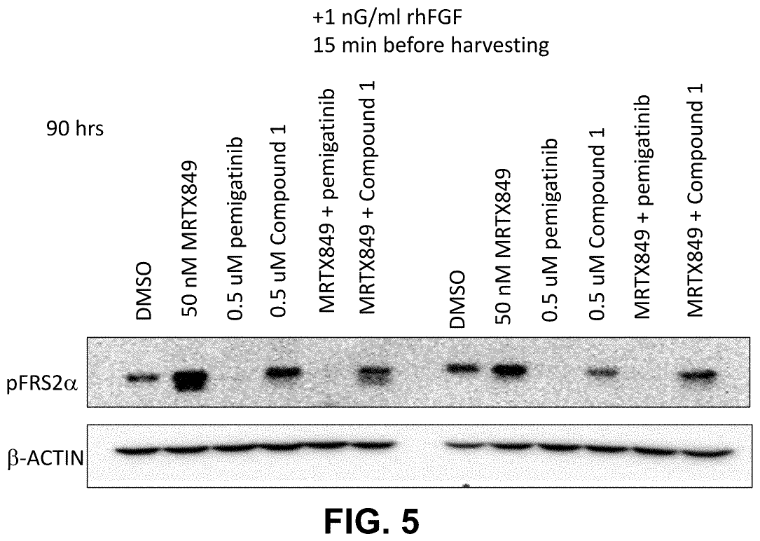

FIG. 5 depicts Western blots of pFRS2 and beta-actin in LU99 cell lysate with MRTX849 treatment.

### Methods:

Cells were seeded in Corning 6-well tissue culture treated plates in RPMI medium with 10% FBS at 6×105 cells/well. After 48 hours, the cells were harvested for western blots (A), or treated with indicated compounds for 90 hours at 37° C., 5% CO₂. The cells were washed with PBS and lysed with lx lysis buffer (Cell Signaling #9803) with protease and phosphatase inhibitors. Twenty five μg of total protein lysates was subjected to SDS-PAGE and immunoblot analysis using antibodies from Cell Signaling Technology.

In conclusion, mesenchymal-like cancer cell line Lu99 displayed elevated FGFR1 signaling; and KRAS G12C inhibition alone led to an increase of the FGFR1 signaling. Combination of a KRAS G12C inhibitor MRTX849 with the FGFR1 inhibitor pemigatinib, and to a lesser extent with the FGFR2/3 inhibitor Compound 1, inhibited the KRAS G12C inhibitor induced upregulation of the FGFR1 pathway.

### Example H. Combination of MRTX849 and pemigatinib on the Inhibition of pERK Signaling in LU99 Cells

The MEK-ERK pathway is the main downstream signaling pathway of KRAS G12C mutated cancer cells to survive and proliferate. Therefore, the impact to phospho-ERK (pERK) was evaluated with KRAS G12C inhibitor and FGFR inhibitor treatment.

LU99 cells were treated with 100 nM of MRTX849, 100 nM pemigatinib, Compound 1, or indicated combination for 24 hours, and with or without 1 ng/ml recombinant human FGF (rhFGF) before harvesting the lysate. Both pERK and pFRS2a signaling was elevated after 1 ng/ml hrFGF treatment. With or without rhFGF stimulation, pFRS2a was elevated after MRTX849 treatment; and combination of MRTX849 and pemigatinib fully inhibited pERK signaling.

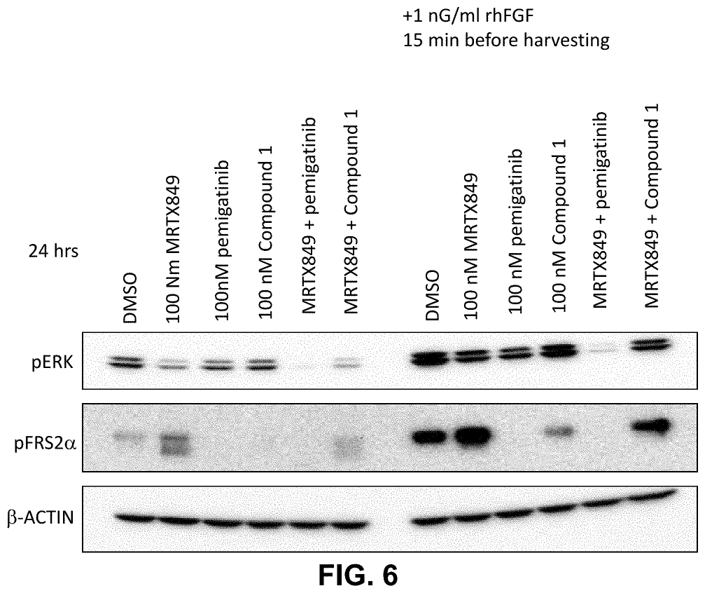

FIG. 6 shows Western blots of pERK, pFRS2a, and beta-actin in LU99 cells treated with the KRAS G12C inhibitor, MRTX849, with and without pemigatinib or Compound 1 for 24 hours.

Methods:

Cells were seeded in Corning 6-well tissue culture treated plates in RPMI medium with 10% FBS at 6x105 cells/well. After 48 hours, cells were or treated with indicated compounds for 24 hours at 37° C., 5% CO₂. rhFGF—basic (R&D systems 233-FB) was added at 1 nG/ml for 15 minutes at the end of the experiment as indicated. The cells were washed with PBS and lysed with 1× lysis buffer (Cell Signaling #9803) with protease and phosphatase inhibitors. Twenty-five μg of total protein lysates was subjected to SDS-PAGE and immunoblot analysis using the following Cell Signaling antibodies: pERK (#4370) phospho-FRS2a (#3861) and beta-Actin (#12620).

In summary, in mesenchymal-like cells like LU99, inhibiting KRAS G12C did not fully inhibit the downstream pERK signaling; however, the combination of a KRAS G12C with an FGFR1 inhibitor could fully achieve the inhibition of the pERK pathway.

### Example I. Combination of MRTX849 and FGFR Inhibitors on Epithelial and Mesenchymal-Like Cell Lines

The in-vitro effect of combining the FGFR1 inhibitor pemigatinib and the FGFR2/3 inhibitor Compound 1 with the KRAS G12C inhibitor MRTX849 was assessed in various cell lines.

TABLE 3
Table of average Bliss scores observed in-vitro investigating
seven cell lines with combination of FGFR1 inhibitor pemigatinib,
FGFR2/3 inhibitor compound1 and MRTX849.

| | | | | | Avg. |
|---|---|---|---|---|---|
| Cell | | | KRAS | FGFR | Bliss |
| Line | Tissue | Type | Inhibitor | Inhibitor | Score |
| H358 | Lung | Epithelial | MRTX849 | Pemigatinib | 3.7 |
| H358 | Lung | Epithelial | MRTX849 | Compound 1 | 1.3 |
| SW837 | CRC | Epithelial | MRTX849 | Pemigatinib | 6.1 |
| SW837 | CRC | Epithelial | MRTX849 | Compound 1 | 4.5 |
| H2122 | Lung | Epithelial | MRTX849 | Pemigatinib | 4.5 |
| H2122 | Lung | Epithelial | MRTX849 | Compound 1 | 4.5 |
| HCC44 | Lung | Mesenchymal | MRTX849 | Pemigatinib | 11.6 |
| HCC44 | Lung | Mesenchymal | MRTX849 | Compound 1 | 4 |
| H1792 | Lung | Mesenchymal | MRTX849 | Pemigatinib | 19.9 |
| H1792 | Lung | Mesenchymal | MRTX849 | Compound 1 | 1 |
| SW1573 | Lung | Mesenchymal | MRTX849 | Pemigatinib | 29.5 |
| SW1573 | Lung | Mesenchymal | MRTX849 | Compound 1 | 14.9 |
| Lu99 | Lung | Mesenchymal | MRTX849 | Pemigatinib | 37.4 |
| Lu99 | Lung | Mesenchymal | MRTX849 | Compound 1 | 7.5 |

### Methods:

Cells were seeded in RPMI medium with 10% HI FBS at a density of 500 cells/well in Greiner, white, clear bottom, 384-well tissue culture treated plates containing 10×10 combination matrices of compound. Plates were incubated at 37° C., 5% CO₂ for 5 days. On day 5, Cell Titer Glo reagent was added to the plates and ATP luminescence was detected using a Pherastar FSX reader. The synergy score for the combination effect was calculated using a Bliss score model where the Bliss score=Yab−(Ya+Yb−(YaYb))×100, where Ya and Yb are the monotherapies. Bliss scores >20 are strongly synergistic and a higher Bliss scores indicates a higher level of synergism.

In summary, in mesenchymal-like cells, but not in epithelial-like KRAS G12C mutant cells, the combination of the FGFR1 inhibitor pemigatinib with KRAS G12C inhibitor MRTX849 synergistically inhibited cell proliferation.

### Example J. Testing the Combinational Effects of pemigatinib with MRTX849 on Tumor Growth Control In Vivo

### LU99 Xenograft Model

The in vivo effect of combining the FGFR1 inhibitor pemigatinib plus the KRAS G12C inhibitor MRTX849 (adagrasib) was assessed in the LU99 lung xenograft model (JCRB0080, JCRB) in 6 to 8 week old female NCr nude mice (Taconic). Pemigatinib was suspended in 5% N,N-dimethyl acetamide (DMAC)+50 mM Citrate buffer (pH 3.0) in 0.5% methyl cellulose and MRTX849 was suspended in 10% Captisol+50 mM Citrate buffer (pH 2.5), both for oral administration. Briefly, mice were inoculated in the left flank with 1×10⁷LU99 cells re-suspended in a PBS and matrigel (Corning Life Sciences, Tewksbury, Mass) solution at 1:1. When tumors reached suitable size (on approximately Day 7), mice were randomized into groups of 10 mice based on tumor volume. From there, mice were dosed with (i) vehicle; (ii) 10 mg/kg of MRTX849; (ii) 30 mg/kg of MRTX849; (iii) 0.3 mg/kg of pemigatinib; (iv) the combination of 10 mg/kg of MRTX849 and 0.3 mg/kg of pemigatinib; (v) the combination of 30 mg/kg of MRTX849 and 0.3 mg/kg of pemigatinib. Pemigatinib and MRTX849 were administered orally once daily (QD) for the duration of the study. The primary endpoint of this study was tumor growth inhibition (TGI). TGI was calculated using the formula (1−(V_{T}/V_{C}))×100, where V_{T} is the tumor volume of the treatment group on the last day of treatment, and V_{C} is the tumor volume of the control group on the last day of treatment. Statistical relationships were tested using One-Way ANOVA.

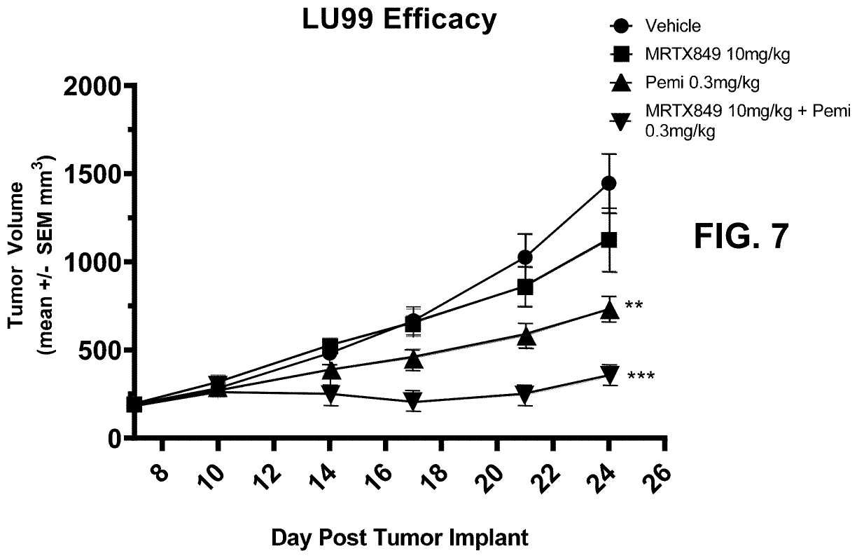

FIG. 7 is a graph depicting the tumor volume of LU99 tumor bearing mice administered (i) vehicle; (ii) 10 mg/kg of MRTX849; (iii) 0.3 mg/kg of pemigatinib; or (iv) the combination of MRTX849 and pemigatinib at 10 mg/kg and 0.3 mg/kg, respectively.

In summary, the combination of MRTX849 and pemigatinib achieved maximal efficacy compared to each single agent treatment group in LU99 xenograft models.

### Example K. Testing the Combinational Effects of pemigatinib with MRTX849 on Phospho-ERK Inhibition in LU99 Tumors Compared to Single Agent Treatments

LU99 In Vivo pERK Inhibition

The effect of combining pemigatinib and MRTX849 (adagrasib) on pERK inhibition was assessed in vivo in the LU99 lung cancer xenograft model in 6 to 8 week old NCr nude mice (Taconic). Pemigatinib was suspended in 5% N,N-dimethyl acetamide (DMAC)+50 mM Citrate buffer (pH 3.0) in 0.5% methyl cellulose and MRTX849 was suspended in 10% Captisol+50 mM Citrate buffer (pH 2.5), both for oral administration. Briefly, mice were inoculated in the left flank with 1×10⁷ LU99 cells re-suspended in a PBS and matrigel (Corning Life Sciences, Tewksbury, Mass) solution at 1:1. When tumors were approximately ˜500mm³, mice were given a single dose of (i) vehicle; (ii) 10 mg/kg of MRTX849; (ii) 0.3 mg/kg of pemigatinib; (iv) the combination of 10 mg/kg of MRTX849 and 0.3 mg/kg of pemigatinib. Tumors were collected 2 h post dose. Tumors were then processed and levels of phospho ERK relative to total ERK assessed on tumor lysates using MSD (Mesoscale). The percent inhibition was calculated relative to vehicle-treated mice. Data are shown as the ratio of phospho-ERK to total ERK. Statistical relationships were tested using One-Way ANOVA.

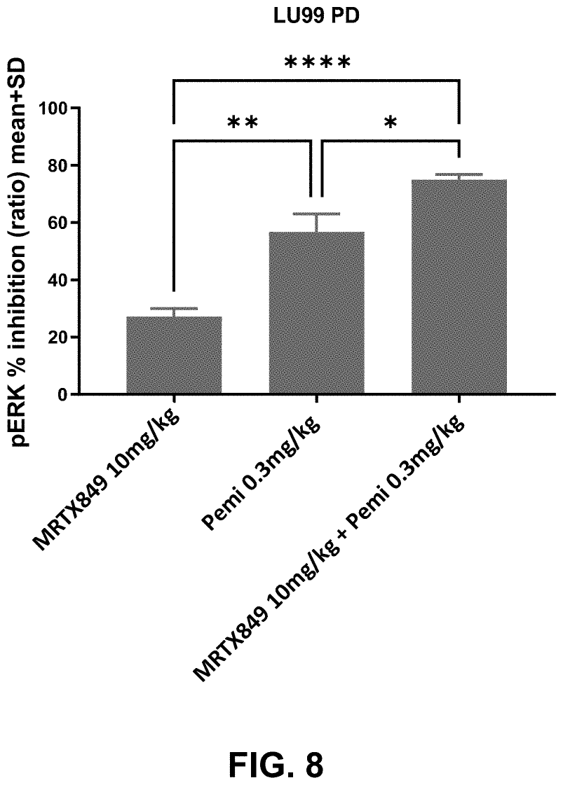

FIG. 8 is a graph depicting the inhibition of pERK in LU99 tumors from mice receiving 10 mg/kg of MRTX849, 0.3 mg/kg of pemigatinib, or the combination of MRTX849 and pemigatinib at 10 mg/kg and 0.3 mg/kg, respectively. In summary, MRTX849 and pemigatinib combination achieved maximal inhibition of pERK signaling in LU99 xenograft models, which is consistent with previous in vitro results.

### Example L. siRNA FGFR Isoforms Knockdown in Combination with KRAS G12C Inhibitors Decreased In-Vitro Cell Proliferation in LU99 Cells

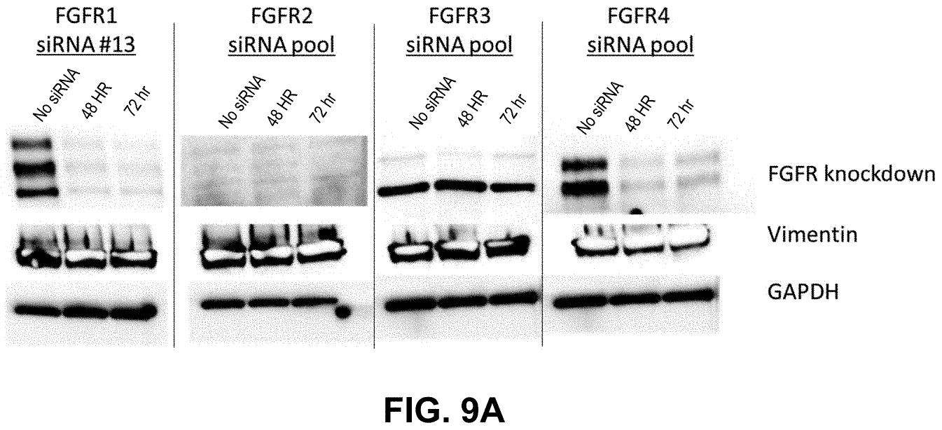

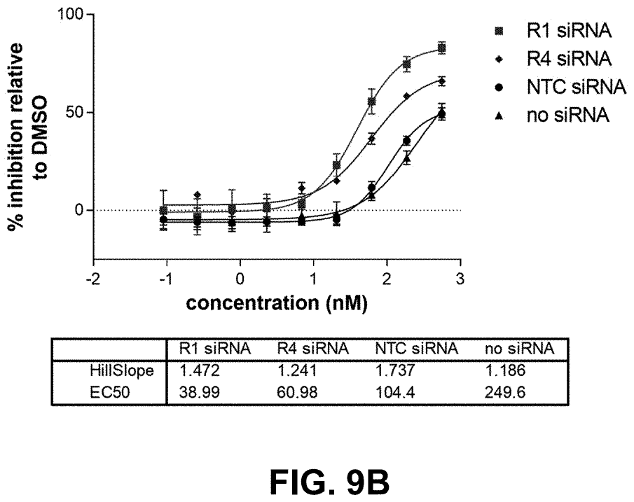

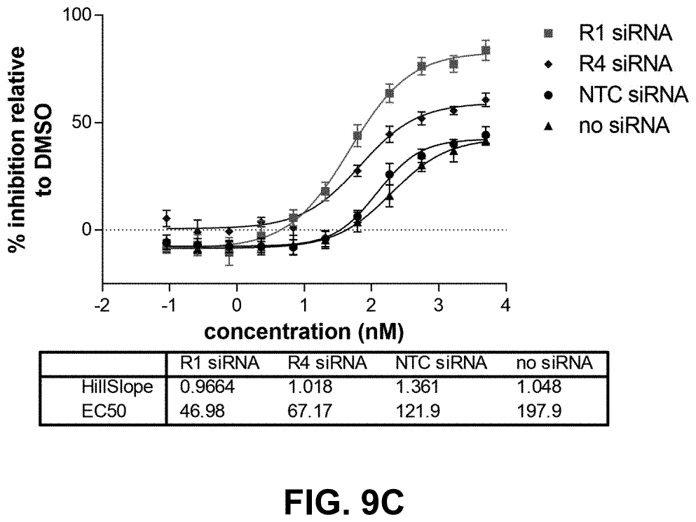

To further evaluate the KRAS/FGFR combination mechanism of action, multiple siRNA FGFR knockdown experiments were conducted in LU99 cells, followed by treatment with KRAS G12C inhibitor Compound 2, Compound 3, or AMG510. Knockdown efficiency was assessed by Western blot and its effect was measured by cell proliferation inhibition. In a first experiment (FIG. 9), FGFR1 and FGFR4 knockdown was achieved (FIG. 9A). The addition of either compound 3 (FIG. 9B) or AMG510 (FIG. 9C) resulted in a higher inhibition in proliferation in the absence of FGFR1 or FGFR4.

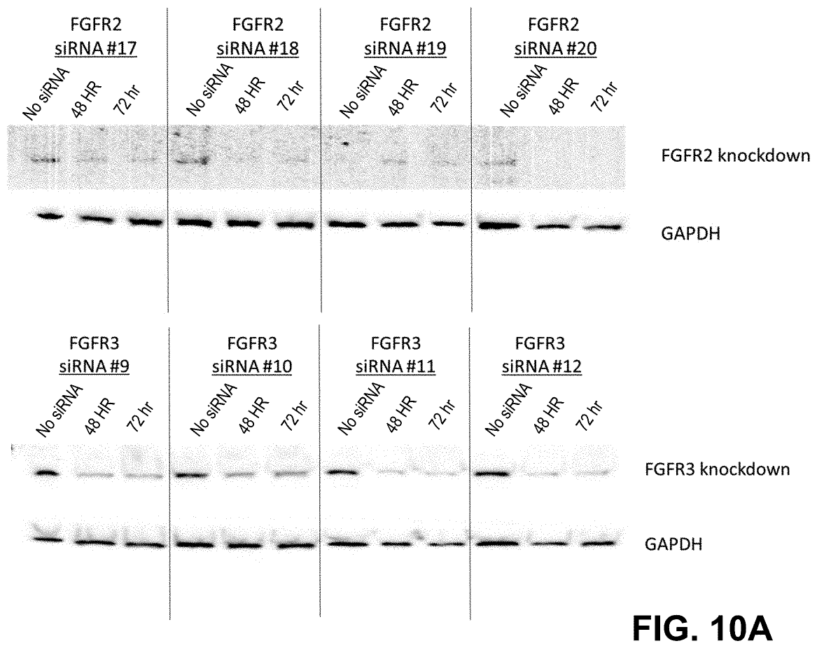

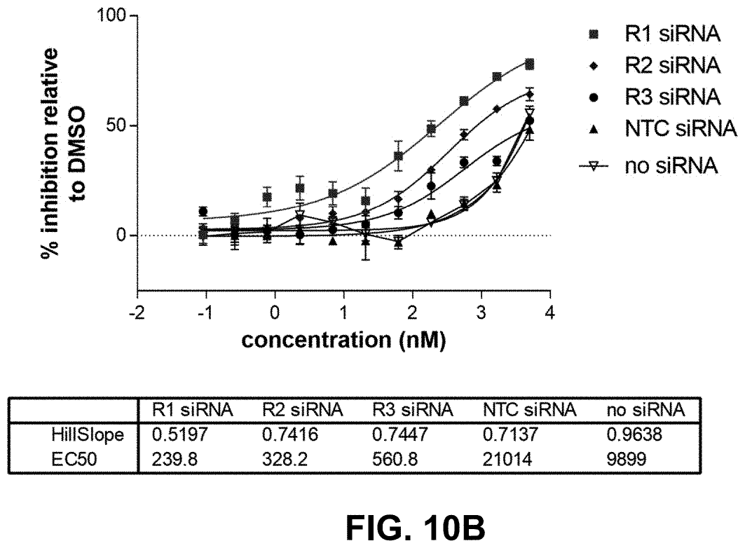

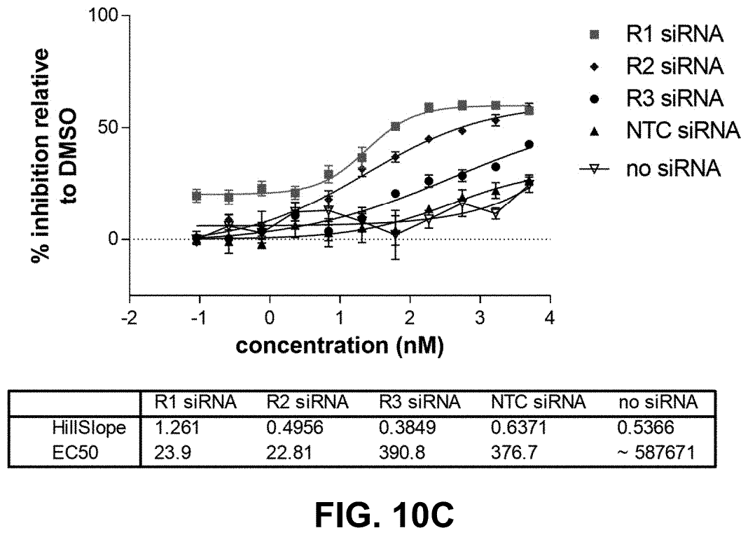

In a follow up experiment, FGFR2 and FGFR3 knockdown was achieved (FIG. 10A) in addition to FGFR1 knockdown. As in the previous example, the addition of either compound 2 (FIG. 10B) or AMG-510 (FIG. 10C) resulted in a higher inhibition in proliferation in the absence of FGFR1, FGFR2 or FGFR3.

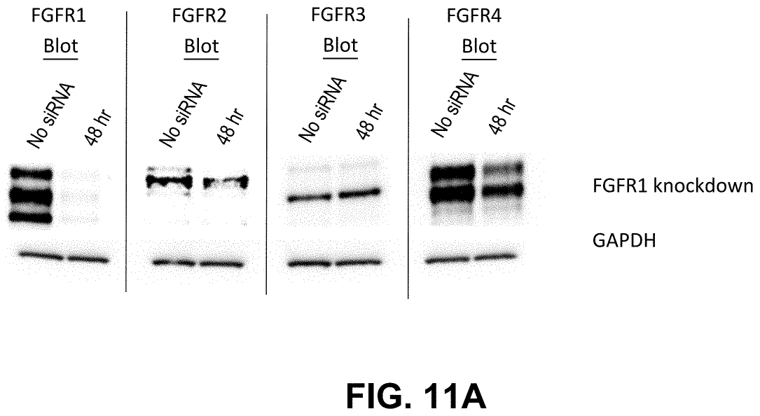

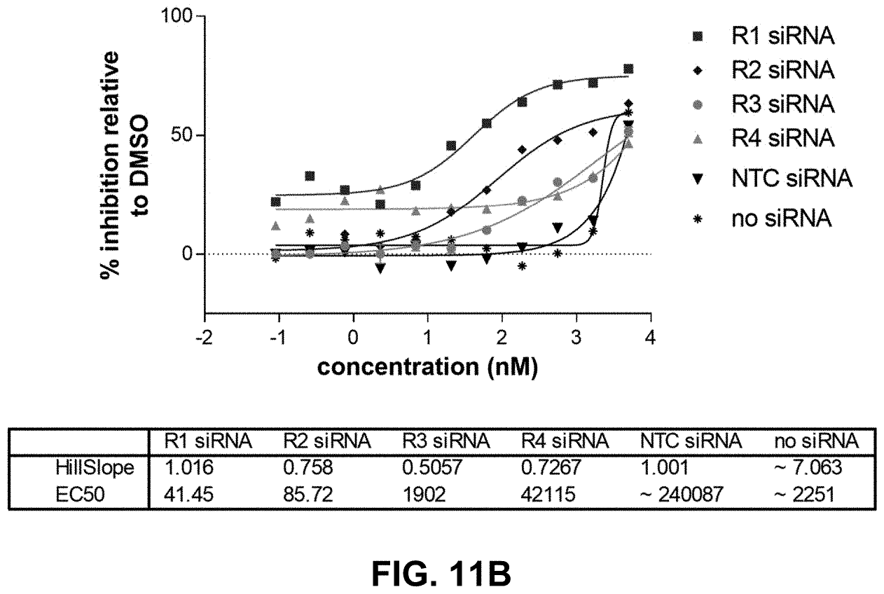

Finally, all isoforms of FGFR were knocked down to some degree to evaluate their effect in proliferation (FIG. 11A). As previously observed, the addition of compound 2 (FIG. 11B) resulted in a higher inhibition in proliferation, in particular in the absence of FGFR1 and FGFR2.

In summary, the combination of a KRAS G12C inhibitor after knockdown of FGFR isoforms resulted in a higher inhibition of LU99 in vitro proliferation, closely resembling the effects of combining KRAS G12C inhibitors with FGFR inhibitors.

### Methods:

The lyophilized siRNA pools or individuals (Dharmacon ON-TARGET plus siRNA from Horizon Discovery) were reconstituted by combining 1× siRNA buffer (200 μL) with lyophilized siRNA (20 nmol); or combining 1× siRNA buffer (100 μL) with lyophilized siRNA (10 nmol). Dharmafect Reagent 1 was prepared at a 1:200 dilution in 1X siRNA buffer. One tube of diluted reagent was prepared for each siRNA dilution. To each tube, the appropriate siRNA was added to Dharmafect Reagent 1 at a 1:400 dilution. The prepared siRNA reagent (25 per well) was added to the appropriate number of wells on a 96-well clear bottom Greiner plate and incubated at room temperature for 30 min.

For plating in 96-well pates, 2000 LU99 cells per well in 100 μL of medium were prepared. The prepared cells (100 μL per well) were added to each well of the 96-well plate and incubated at 48 h.

Compound plates were prepared with a compound starting concentration of 5 mM in a 3-fold dilution. Following 48 h transfection incubation, the appropriate compounds were added to the 96-well plate at a final starting concentration of 5 μM in 3-fold dilution (11 point dose response curve). After addition of the compounds, the 96-well plates were incubated at 37° C. for 120 h. After this time, Cell Titer Glo reagent is reconstituted and 100 μL of reagent was added per well. The plates were tapped gently and luminescence was read on a Pherastar microplate reader.

For Western blot analysis, LU99 cells (5×10⁵ cells) were seeded in plates containing siRNA and incubated for 48 h. Cells were trypsinized and washed and the appropriate volumes of 1× Cell Signaling Technology Lysis Buffer supplemented with 1× protease/phosphatase inhibitor for lysis were prepared. Lysates were subjected to Western blot analysis using antibodies against FGFR1 (CST #9740S), FGFR2 (Abcam #ab109372), FGFR3 (Abcam #ab133644), FGFR4 (CST #8562S), vimentin (CST #5741S), and GAPDH (CST #5174S).

FIG. 9A depicts Western blots for FGFR1, FGFR2, FGFR3, and FGFR4 knockdown experiments.

FIG. 9B shows the inhibitory effect of FGFR1 and FGFR4 siRNA knockdown in combination with Compound 3 on LU99 cell proliferation after 120 h.

FIG. 9C shows the inhibitory effect of FGFR1 and FGFR4 siRNA knockdown in combination with AMG510 on LU99 cell proliferation after 120 h.

FIG. 10A depicts Western blots for FGFR2 and FGFR3 knockdown experiments.

FIG. 10B shows the inhibitory effect of FGFR1, FGFR2 and FGFR3 siRNA knockdown in combination with Compound 2 on LU99 cell proliferation after 120 h.

FIG. 10C shows the inhibitory effect of FGFR1, FGFR2 and FGFR3 siRNA knockdown in combination with AMG510 on LU99 cell proliferation after 120 h.

FIG. 11A depicts Western blots for FGFR1, FGFR2, FGFR3, and FGFR4 after knockdown of FGR1 in order to evaluate possible compensation of FGFR2, FGFR3, and FGFR4.

FIG. 11B shows the inhibitory effect of FGFR1, FGFR2, FGFR3, and FGFR4 siRNA knockdown in combination with Compound 2 on LU99 cell proliferation after 120 h.

### Example M. Combination of KRAS G12C Inhibition with pemigatinib Overcomes Resistance in MiaPaca2 KRAS G12C Resistant Clones

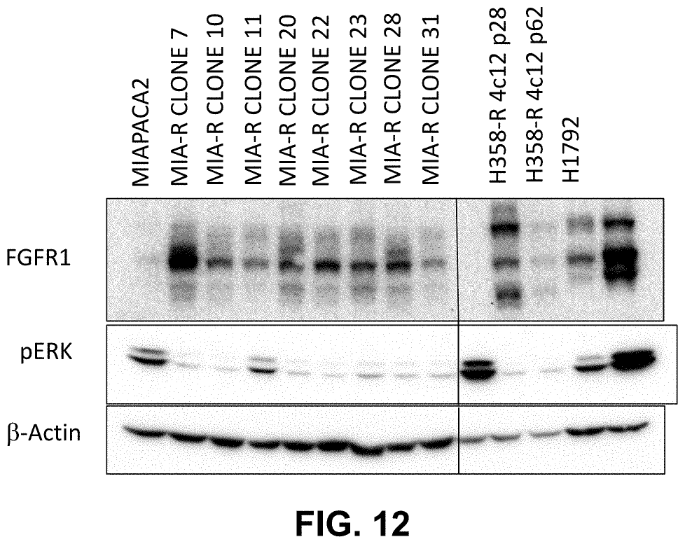

The in-vitro effect of combining KRAS G12 inhibitor AMG510 with FGFR inhibitors pemigatinib and Compound 1 was assessed in MiaPaca2 KRAS G12C resistant clones generated by culturing cells in the presence of increasing concentrations of AMG510 over time followed by limiting dilution. KRAS G12C resistant clones showed increase expression of FGFR1 and activation of its signaling pathway compared to control MiaPaca2 cells (FIG. 12). MiaPaca2 KRAS G12C resistant clones showed greater synergy (Bliss score) when a KRAS G12C inhibitor was added with the FGFR1 inhibitor, pemigatinib, compared with compound 1 in a 5 day Cell Titer Glo Assay (Table 4). In addition, KRAS G12C resistance sensitized MiaPaca2 cells to combination as noted by greater synergy (Bliss score) at low pemigatinib concentrations (Table 5).

TABLE 4
shows the 5-day CTG- Bliss scores of MiaPaca2 KRAS G12C resistance clones.

| | | | | | | Compound | | |
|---|---|---|---|---|---|---|---|---|
| | G12C i | Pemi | | BLISS | G12C i | 1 | | BLISS |
| % viability | 111 nM | 1 μM | Together | Score | 111 nM | 1 μM | Together | Score |
| MIAPACA2 | 21 | 102 | 11 | 8.8 | 20 | 100 | 14 | 4.8 |
| | 12 | 100 | 7 | 5.6 | 12 | 97.6 | 10.7 | 0.9 |
| | 11.7 | 89.1 | 5.8 | 4.6 | 10.4 | 93.1 | 8.8 | 0.94 |
| Average | 14.9 | 97.0 | 7.9 | 6.3 | 14.1 | 96.9 | 11.2 | 2.2 |
| CLONE 7 | 69.6 | 104 | 63 | 8.9 | 67.1 | 92.6 | 64 | −2.4 |
| | 90 | 106 | 53 | 21 | 88 | 108 | 82 | 11.2 |
| | 73.7 | 93.4 | 47.7 | 21 | | | | |
| Average | 77.8 | 101.1 | 54.6 | 15.0 | 77.6 | 100.3 | 73.0 | 4.4 |
| CLONE 11 | 67 | 87.6 | 33.8 | 24.9 | 63 | 94 | 57 | 1.8 |
| CLONE 23 | 90.9 | 100.1 | 74.9 | 16.1 | 83.9 | 90.7 | 80.5 | −4.4 |
| CLONE 28 | 69.6 | 78 | 32.4 | 21.8 | 77.5 | 87.2 | 58 | 9.4 |

TABLE 5
shows the 5 day CTG-representative dose response heat maps
of Bliss scores of MiaPaca2 KRAS G12C resistance clones.

| BLISS | | KRAS G12C i | | | | | | | | |
|---|---|---|---|---|---|---|---|---|---|---|
| Score | nM | 1000.00 | 333.33 | 111.11 | 37.04 | 12.35 | 4.12 | 1.37 | 0.46 | 0.15 |
| MIA | | | | | | | | | | |
| PaCa2 | | | | | | | | | | |
| Pemi | 1000 | 4.32 | 3.71 | 4.62 | 4.32 | 2.84 | 6.59 | 3.46 | −5.74 | −10.95 |
| Pemi | 333 | 2.38 | 3.97 | 4.88 | 6.27 | 3.76 | 7.47 | 11.10 | −5.77 | −12.96 |
| Pemi | 111 | 4.56 | 3.66 | 4.31 | 6.56 | 4.83 | 4.70 | 9.38 | −7.15 | −10.62 |
| Pemi | 37 | 2.69 | 2.88 | 3.10 | 5.01 | 3.06 | 4.25 | 6.72 | −5.08 | −13.53 |
| Pemi | 12 | 0.29 | 0.73 | 0.06 | 1.33 | −1.91 | −1.78 | −4.50 | −9.92 | −16.39 |
| MIA-R | | | | | | | | | | |
| clone 11 | | | | | | | | | | |
| Pemi | 1000 | 21.43 | 20.06 | 24.91 | 21.52 | 29.47 | 31.01 | 23.94 | 11.01 | −0.21 |
| Pemi | 333 | 13.90 | 14.69 | 25.28 | 24.19 | 20.46 | 34.95 | 17.83 | 20.56 | 6.68 |
| Pemi | 111 | 3.15 | 6.86 | 9.80 | 13.58 | 15.70 | 13.15 | 8.76 | 6.04 | −3.03 |
| Pemi | 37 | 4.52 | −5.78 | 8.39 | 10.60 | 15.07 | 20.01 | 6.05 | 6.20 | 14.47 |
| Pemi | 12 | 1.05 | −7.24 | 11.57 | 5.62 | 1.32 | 11.81 | 6.90 | 11.42 | 2.35 |

In summary, the combination of a KRAS G12C inhibitor plus pemigatinib was effective to overcome resistance to KRAS G12C inhibitors.

### Methods:

For Western blots analysis, cells were seeded in Corning 6-well tissue culture treated plates in RPMI medium with 10% FBS at 6×105 cells/well. After 48 hours, the cells were harvested for Western blots (A), or treated with indicated compounds for 90 hours at 37° C., 5% CO2. The cells were washed with PBS and lysed with lx lysis buffer (Cell Signaling #9803) with protease and phosphatase inhibitors. A portion of the total protein lysates (25 pg) was subjected to SDS-PAGE and immunoblot analysis using antibodies from Cell Signaling Technology.

For G12C resistant clone generation, MiaPaca2 parent cells were cultured with increasing concentrations of AMG510 (up to 1μM) over time. The viable cells were re-fed fresh media containing AMG510 weekly. Once the AMG510 resistant cells were growing at normal rates in the presence of 1μM AMG510, they were cloned by limiting dilution. Eight clones were selected for continued passage and further experiments.

For synergy experiments, cells were seeded in RPMI medium with 10% HI FBS at a density of 500 cells/well in Greiner, white, clear bottom, 384-well tissue culture treated plates containing 10x10 combination matrices of compound. Plates were incubated at 37° C., 5% CO₂ for 5 days. On day 5, Cell Titer Glo reagent was added to the plates and ATP luminescence was detected using a Pherastar FSX reader. The synergy score for the combination effect was calculated using a Bliss score model where the Bliss score=Yab−(Ya+Yb−(YaYb))×100, where Ya and Yb are the monotherapies. Bliss scores >20 are strongly synergistic and a higher Bliss scores indicates a higher level of synergism.

FIG. 12 shows the Western blot analysis of FGFR1, pERK, and B-Actin in MiaPaca2 KRAS G12C resistance clones.

### Example N. siRNA FGFR isoforms knockdown in combination with KRAS G12D inhibitors decreased in-vitro cell proliferation in A427 cells

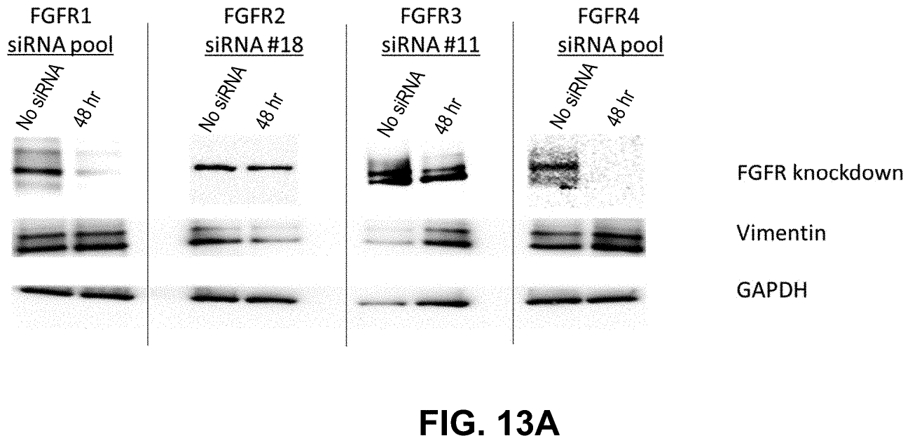

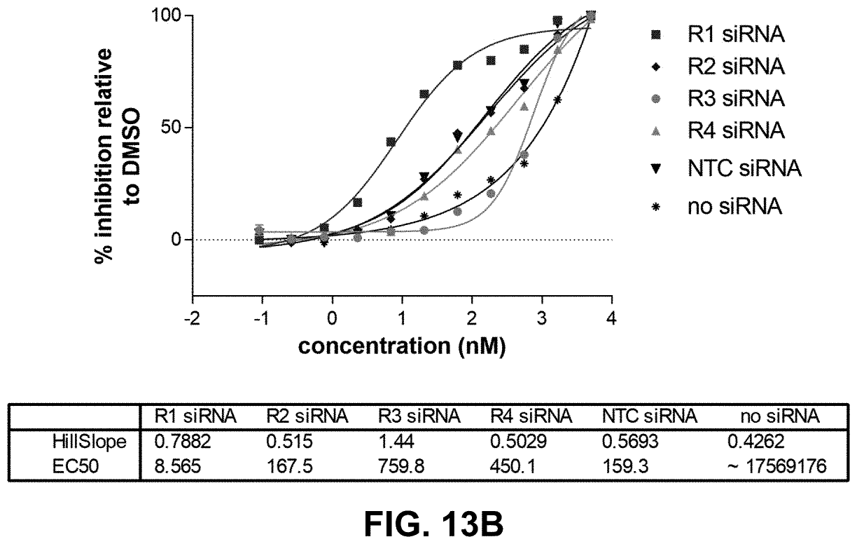

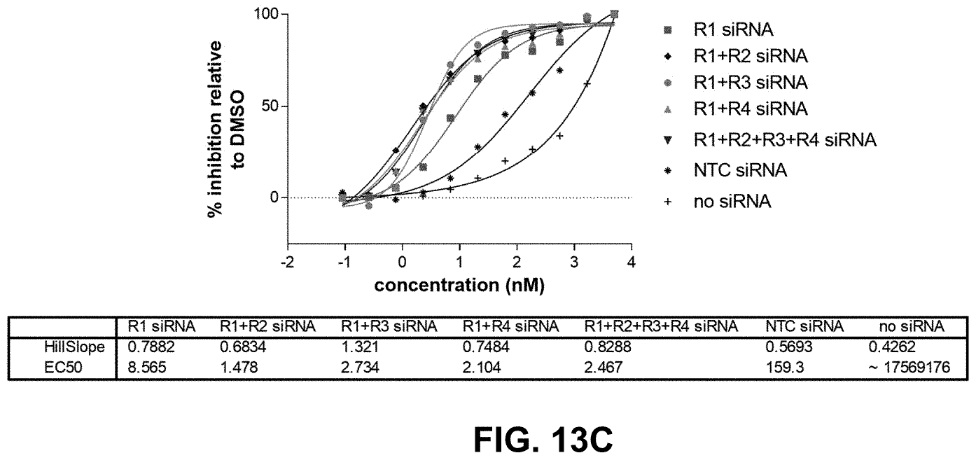

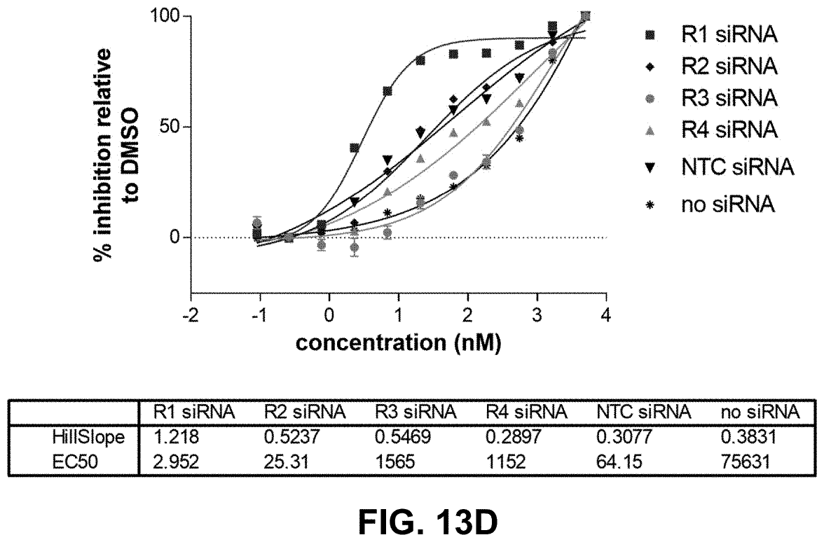

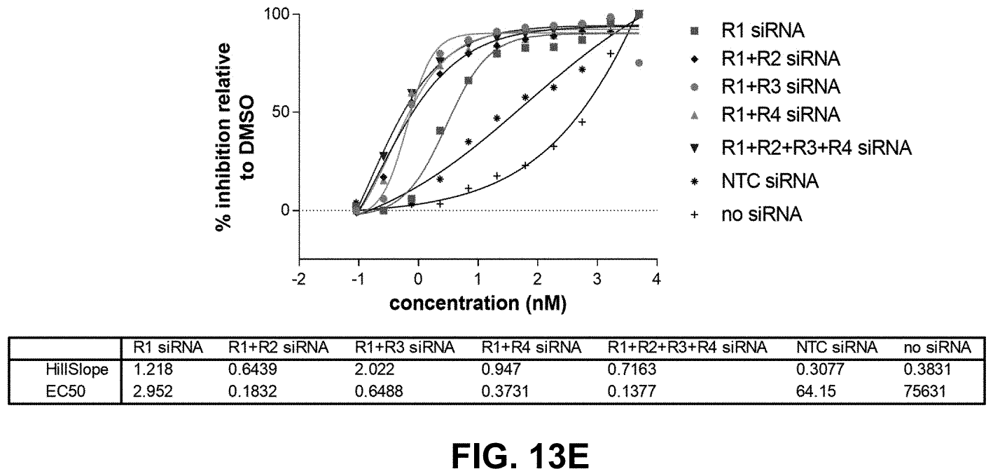

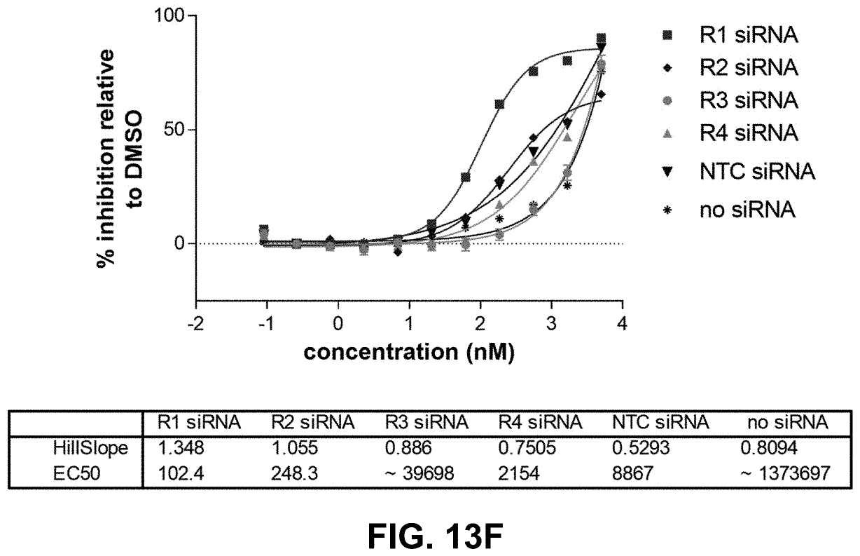

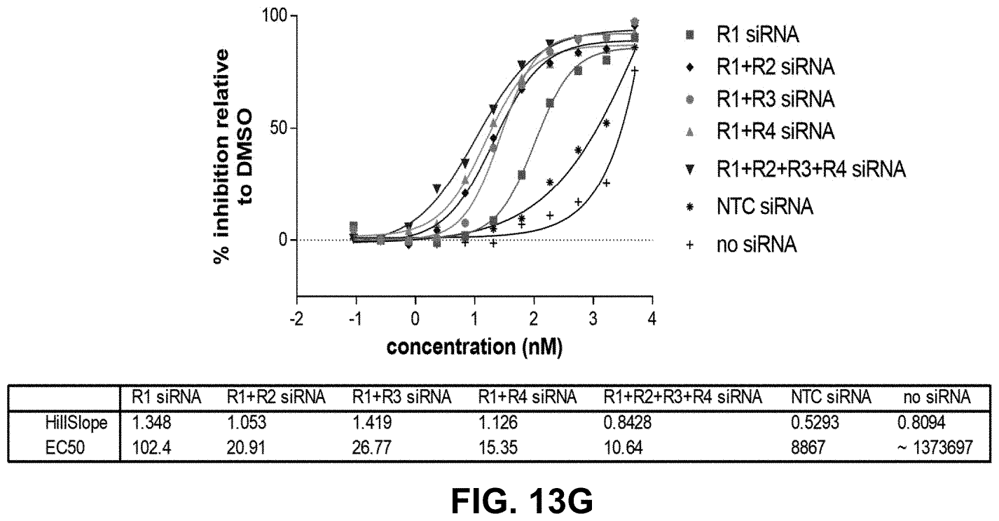

To further evaluate the KRAS/FGFR combination mechanism of action, siRNA FGFR knockdown experiments were conducted in A427 cells, followed by treatment with KRAS G12D inhibitors Compound 5, Compound 6, and Compound 7. Knockdown efficiency was assessed by Western blot (FIG. 13A) and its effect was measured by cell proliferation inhibition (FIGS. 13B-G). Initially, the effect of single FGFR isoforms knockdown in combination with KRAS G12D inhibitors Compound 5 (FIG. 13B), Compound 6 (FIG. 13D) and Compound 7 (FIG. 13F) was assessed. The data shows that the best combinatorial effect was achieved when FGFR1 isoform was knocked down from A427 cells, showing higher inhibition in proliferation compared to controls and knockdown of the rest of the isoforms. In addition, combining FGFR1 knockdown with knockdown of additional FGFR isoforms further increased the effect of KRAS G12D inhibitors (FIGS. 13C, E and G)

In summary, the combination of a KRAS G12D inhibitors after knockdown of FGFR isoforms resulted in a higher inhibition of A427 in vitro proliferation.

### Methods:

The lyophilized siRNA pools or individuals (Dharmacon ON-TARGET plus siRNA from Horizon Discovery) were reconstituted by combining 1× siRNA buffer (200 μL) with lyophilized siRNA (20 nmol); or combining lx siRNA buffer (100 μL) with lyophilized siRNA (10 nmol). Dharmafect Reagent 1 was prepared at a 1:200 dilution in 1× siRNA buffer. One tube of diluted reagent was prepared for each siRNA dilution. To each tube, the appropriate siRNA was added to Dharmafect Reagent 1 at a 1:400 dilution. The prepared siRNA reagent (25 μL per well) was added to the appropriate number of wells on a 96-well clear bottom Greiner plate and incubated at room temperature for 30 min.

For plating in 96-well pates, 2000 A427 cells per well in 100 !IL of medium were prepared. The prepared cells (100 !IL per well) were added to each well of the 96-well plate and incubated at 48 h.

Compound plates were prepared with a compound starting concentration of 5 mM in a 3-fold dilution. Following 48 h transfection incubation, the appropriate compounds were added to the 96-well plate at a final starting concentration of 5μM in 3-fold dilution (11 point dose response curve). After addition of the compounds, the 96-well plates were incubated at 37° C. for 120 h. After this time, Cell Titer Glo reagent is reconstituted and 100 μL of reagent was added per well. The plates were tapped gently and luminescence was read on a Pherastar microplate reader.

For Western blot analysis, A427 cells (5×10⁵ cells) were seeded in plates containing siRNA and incubated for 48 h. Cells were trypsinized and washed and the appropriate volumes of 1× Cell Signaling Technology Lysis Buffer supplemented with 1× protease/phosphatase inhibitor for lysis were prepared. Lysates were subjected to Western blot analysis using antibodies against FGFR1 (CST #9740S), FGFR2 (Abcam #ab109372), FGFR3 (Abcam #ab133644), FGFR4 (CST #8562S), vimentin (CST #5741S), and GAPDH (CST #5174S).

FIG. 13A depicts Western blots for FGFR1, FGFR2, FGFR3, and FGFR4 knockdown experiments.

FIG. 13B shows the inhibitory effect of siRNA knockdown of single FGFR isoforms in combination with Compound 5 on A427 cell proliferation after 120 h.

FIG. 13C shows the inhibitory effect of siRNA knockdown of multiple FGFR isoforms in combination with Compound 5 on A427 cell proliferation after 120 h.

FIG. 13D shows the inhibitory effect of siRNA knockdown of single FGFR isoforms in combination with Compound 6 on A427 cell proliferation after 120 h.

FIG. 13E shows the inhibitory effect of siRNA knockdown of multiple FGFR isoforms in combination with Compound 6 on A427 cell proliferation after 120 h.

FIG. 13F shows the inhibitory effect of siRNA knockdown of single FGFR isoforms in combination with Compound 7 on A427 cell proliferation after 120 h.

FIG. 13G shows the inhibitory effect of siRNA knockdown of multiple FGFR isoforms in combination with Compound 7 on A427 cell proliferation after 120 h.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A method of treating cancer in a patient, comprising administering to said patient:
(i) an FGFR1 inhibitor; and
(ii) a KRAS inhibitor.

2. The method of claim 1, wherein the FGFR1 inhibitor is selected from pemigatinib, futibatinib, erdafitinib and infigratinib, or a pharmaceutically acceptable salt thereof.

3. The method of claim 1, wherein the FGFR1 inhibitor is pemigatinib, or a pharmaceutically acceptable salt thereof.

4. The method of claim 1, wherein the FGFR1 inhibitor is futibatinib, or a pharmaceutically acceptable salt thereof.

5. The method of claim 1, wherein the FGFR1 inhibitor is erdafitinib, or a pharmaceutically acceptable salt thereof.

6. The method of claim 1, wherein the FGFR1 inhibitor is infigratinib, or a pharmaceutically acceptable salt thereof.

7. The method of claim 1, wherein the FGFR1 inhibitor further inhibits FGFR2, FGFR3, or a combination thereof.

8. The method of claim 1, wherein the KRAS inhibitor is a compound of Formula (I): 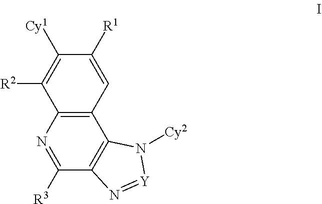
or a pharmaceutically acceptable salt thereof, wherein:
Y is N or CH;
R¹ is selected from Cl, CH₃, CH₂F, CHF₂, and CF₃;
Cy¹ is selected from 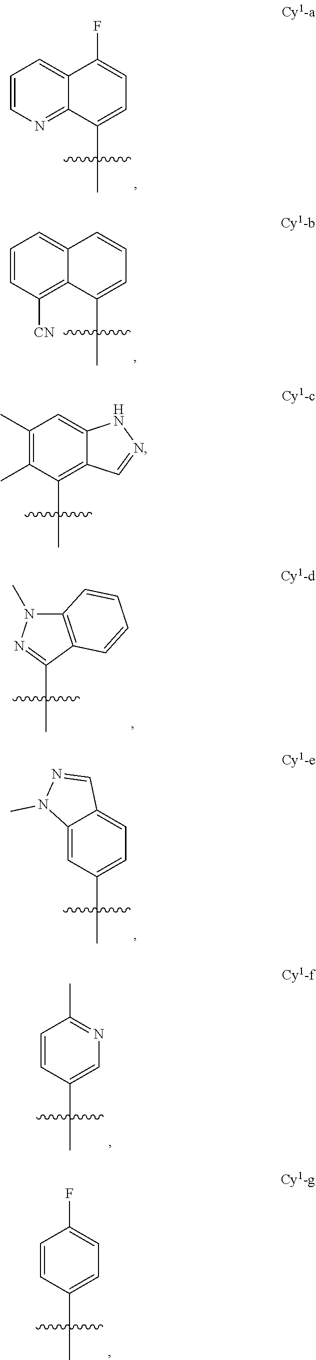 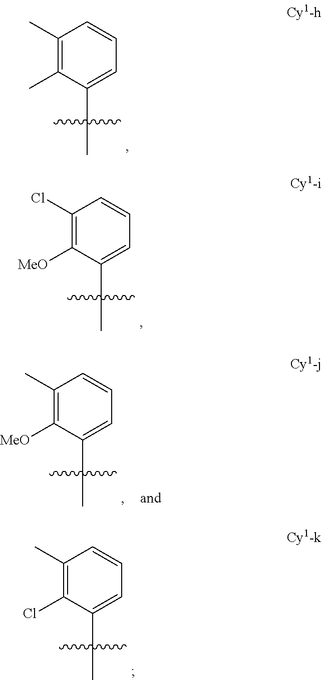
R² is selected from F and Cl;
R³ is selected from 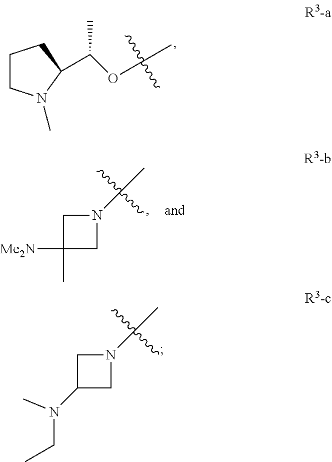
and,
Cy² is selected from 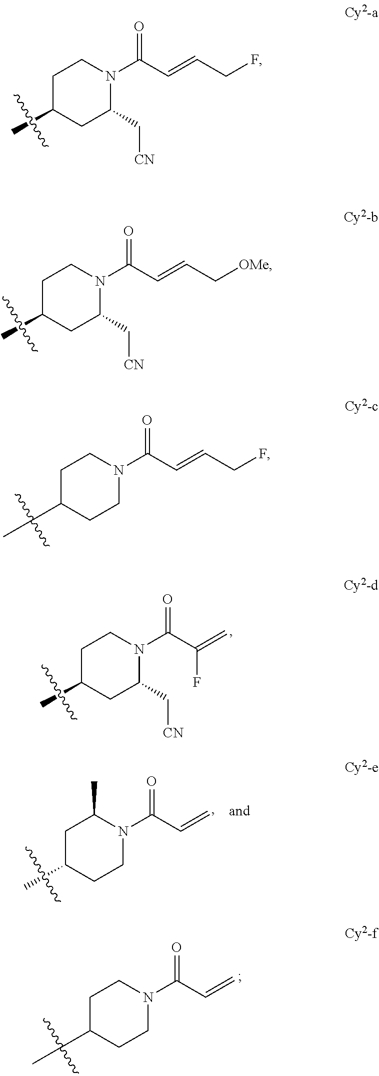
provided that the compound of Formula (I) is other than
2-((2S,4S)-4-(7-(2-chloro-3-methylphenyl)-4-(3-(dimethylamino)-3-
methylazetidin-1-yl)-6-fluoro-8-methyl-1H-[1,2,3]triazolo[4,5-c]quinolin-
1-yl)-1-((E)-4-fluorobut-2-enoyl)piperidin-2-yl)acetonitrile and
2-((2S,4S)-4-(4-(3-(dimethylamino)-3-methylazetidin-1-yl)-7-(2,3-
dimethylphenyl)-6-fluoro-8-methyl-1H-[1,2,3]triazolo[4,5-c]quinolin-
1-yl)-1-((E)-4-fluorobut-2-enoyl)piperidin-2-yl)acetonitrile.

9. The method of claim 8, wherein the compound of Formula (I) or the pharmaceutically acceptable salt thereof is selected from:
2-((2S,4S)-4-(4-(3-(dimethylamino)-3-methylazetidin-1-yl)-6-fluoro-7-(5-fluoroquinolin-8-yl)-8-(trifluoromethyl)-1H-[1,2,3]triazolo[4,5-c]quinolin-1-yl)-1-((E)-4-fluorobut-2-enoyl)piperidin-2-yl)acetonitrile;
2-((2S,4S)-4-(4-(3-(dimethylamino)-3-methylazetidin-1-yl)-6-fluoro-7-(2-methoxy-3-methylphenyl)-8-(trifluoromethyl)-1H-[1,2,3]triazolo[4,5-c]quinolin-1-yl)-((E)-4-fluorobut-2-enoyl)piperidin-2-yl)acetonitrile;
2-((2S,4S)-4-(7-(3-chloro-2-methoxyphenyl)-4-(3-(dimethylamino)-3-methylazetidin-1-yl)-6-fluoro-8-(trifluoromethyl)-1H-[1,2,3]triazolo[4,5-c]quinolin-1-yl)-1-((E)-4-fluorobut enoyl)piperidin-2-yl)acetonitrile;
2-((2S,4S)-4-(6-fluoro-7-(5-fluoroquinolin-8-yl)-4-(S)-1-(S)-1-methylpyrrolidin yl)ethoxy)-8-(trifluoromethyl)-1H-[1,2,3]triazolo[4,5-c]quinolin-1-yl)-1-((E)-4-fluorobut enoyl)piperidin-2-yl)acetonitrile;
1-(4-(6-fluoro-7-(5-fluoroquinolin-8-yl)-4-((S)-1-((S)-1-methylpyrrolidin-2-yl)ethoxy)-8-(trifluoromethyl)-1H-[1,2,3]triazolo[4,5-c]quinolin-1-yl)piperidin-1-yl)prop-2-en-1-one;
2-((2S,4S)-4-(4-(3-(dimethylamino)-3-methylazetidin-1-yl)-'7-(2,3-dimethylphenyl)-6-fluoro-8-methyl-1H-[1,2,3]triazolo[4,5-c]quinolin-1-yl)-1-((E)-4-methoxybut-2-enoyl)piperidin-2-yl)acetonitrile;
2-((2S,4S)-4-(6-fluoro-8-methyl-7-(1-methyl-1H-indazol-6-yl)-4-((S)-1-(S)-1-methylpyrrolidin-2-yl)ethoxy)-1H-[1,2,3]triazolo[4,5-c]quinolin-1-yl)-1-((E)-4-fluorobut-2-enoyl)piperidin-2-yl)acetonitrile;
2-((2S,4S)-4-(6-fluoro-8-methyl-7-(6-methylpyri din-3-yl)-4-((S)-1-((S)-1-methylpyrrolidin-2-yl)ethoxy)-1H-[1,2,3]triazolo[4,5-c]quinolin-1-yl)-1-((E)-4-fluorobut-2-enoyl)piperidin-2-yl)acetonitrile;
2-((2S,4S)-4-(6-fluoro-8-methyl-7-(1-methyl-1H-indazol-3-yl)-4-((S)-1-((S)-1-methylpyrrolidin-2-yl)ethoxy)-1H-[1,2,3]triazolo[4,5-c]quinolin-1-yl)-1-((E)-4-methoxybut-2-enoyl)piperidin-2-yl)acetonitrile;
2-((2S,4S)-4-(6-fluoro-7-(4-fluorophenyl)-8-methyl-4-((S)-1-((S)-1-methylpyrrolidin-2-yl)ethoxy)-1H-[1,2,3]triazolo[4,5-c]quinolin-1-yl)-1-((E)-4-methoxybut-2-enoyl)piperidin-2-yl)acetonitrile;
8-(1-(1-acryloylpiperidin-4-yl)-6-fluoro-8-methyl-4-(S)-1-(S)-1-methylpyrrolidin-2-yl)ethoxy)-1H-[1,2,3]triazolo[4,5-c]quinolin-7-yl)-1-naphthonitrile;
2-((2S,4S)-4-(7-(2-chloro-3-methylphenyl)-4-(3-(dimethylamino)-3-methylazetidin-1-yl)-6-fluoro-8-methyl-1H-imidazo[4,5-c]quinolin-1-yl)-1-((E)-4-fluorobut-2-enoyl)piperidin-2-yl)acetonitrile;
2-((2S,4S)-4-(7-(2-chloro-3-methylphenyl)-6-fluoro-8-methyl-4-(S)-1-((S)-1-methylpyrrolidin-2-yl)ethoxy)-1H-imidazo[4,5-c]quinolin-1-yl)-1-(2-fluoroacryloyl)piperidin-2-yl)acetonitrile;
8-(1-(2R,4S)-1-acryloyl-2-methylpiperidin-4-yl)-6-fluoro-8-methyl-4-(S)-1-(S) methylpyrrolidin-2-yl)ethoxy)-1H-[1,2,3]triazolo[4,5-c]quinolin-7-yl)-1-naphthonitrile;
2-((2S,4S)-4-(7-(5,6-dimethyl-1H-indazol-4-yl)-4-(3-(ethyl(methyl)amino)azetidin-1-yl)-6-fluoro-8-methyl-1H-[1,2,3]triazolo[4,5-c]quinolin-1-yl)-1-(2-fluoroacryloyl)piperidin yl)acetonitrile;
8-(6-fluoro-1-(4-(E)-4-fluorobut-2-enoyl)piperidin-4-yl)-8-methyl-4-(S)-1-((S)-1-methylpyrrolidin-2-yl)ethoxy)-1H-[1,2,3]triazolo[4,5-c]quinolin-7-yl)-1-naphthonitrile;
8-(1-(2S,4S)-2-(cyanomethyl)-1-(2-fluoroacryloyl)piperidin-4-yl)-4-(3-(dimethylamino)-3-methylazetidin-1-yl)-6-fluoro-8-methyl-1H-[1,2,3]triazolo[4,5-c]quinolin-7-yl)-1-naphthonitrile;
2-((2S,4S)-4-(6, 8-dichloro-4-(3-(dimethylamino)-3-methylazetidin-1-yl)-7-(5-fluoroquinolin-8-yl)-1H-[1,2,3 ]triazolo[4, 5-c]quinolin-1-yl)-1-((E)-4-methoxybut-2-enoyl)piperidin-2-yl)acetonitrile;
2-((2S,4S)-4-(6, 8-dichloro-4-(3-(dimethylamino)-3-methylazetidin-1-yl)-7-(5-fluoroquinolin-8-yl)-1H-[1,2,3 ]triazolo[4, 5-c]quinolin-1-yl)-1-((E)-4-fluorobut-2-enoyl)piperidin-2-yl)acetonitrile; and
2-((2S,4S)-4-(4-(3-(dimethylamino)-3-methylazetidin-1-yl)-6-fluoro-7-(5-fluoroquinolin-8-yl)-8-(trifluoromethyl)-1H-[1,2,3]triazolo[4,5-c]quinolin-1-yl)-1-((E)-4-methoxybut-2-enoyl)piperidin-2-yl)acetonitrile;
or a pharmaceutically acceptable salt thereof.

10. The method of claim 1, wherein the KRAS inhibitor is a compound of Formula (II): 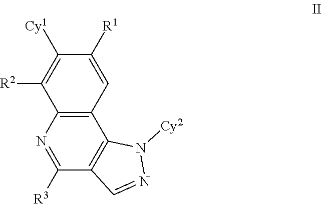
or a pharmaceutically acceptable salt thereof, wherein:
R¹ is selected from Cl, CH₃, CH₂F, CHF₂, and CF₃;
Cy¹ is selected from 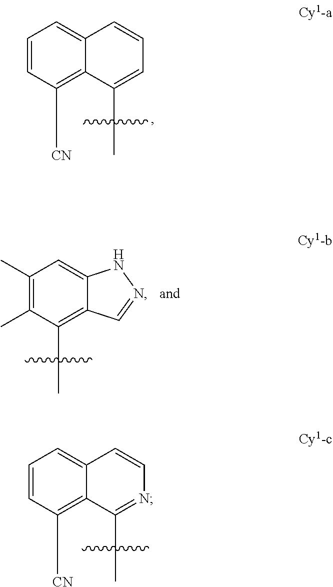 [SMILES: *c1cccc2cccc(CC#N)c12 ; *c1c(C)c(C)cc2[nH]ncc12 ; *c1nccc2cccc(CC#N)c12]
R² is selected from F and Cl;
R³ is selected from 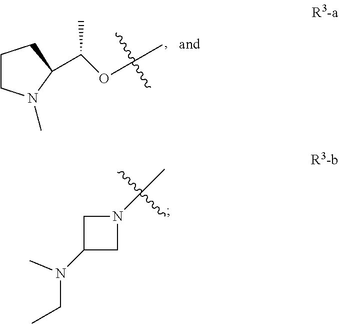
and,
Cy² is selected from 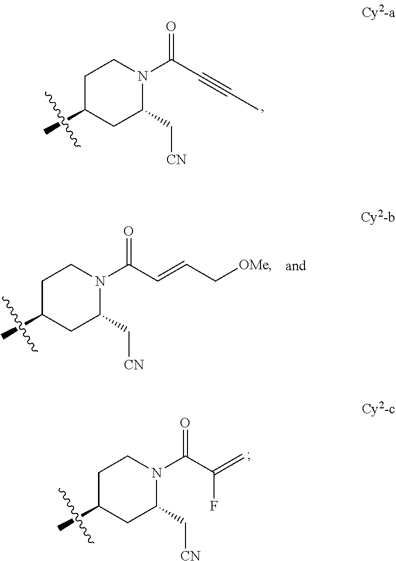
provided that the compound of Formula (II) is other than,
8-(1-((2S,4S)-2-(cyanomethyl)-1-(2-fluoroacryloyl)piperidin-4-yl)-6-fluoro-8-methyl-4-((S)-
1-((S)-1-methylpyrrolidin-2-yl)ethoxy)-1H-pyrazolo[4,3-c]quinolin-7-yl)-1-naphthonitrile,
8-(1-((2S,4S)-2-(cyanomethyl)-1-((E)-4-methoxybut-2-enoyl)piperidin-4-yl)-6-fluoro-8-
methyl-4-((S)-1-((S)-1-methylpyrrolidin-2-yl)ethoxy)-1H-pyrazolo[4,3-c]quinolin-7-yl)-1-
naphthonitrile,
2-((2S,4S)-4-(7-(5,6-dimethyl-1H-indazol-4-yl)-6-fluoro-8-methyl-4-((S)-1-((S)-1-
methylpyrrolidin-2-yl)ethoxy)-1H-pyrazolo[4,3-c]quinolin-1-yl)-1-(2-
fluoroacryloyl)piperidin-2-yl)acetonitrile,
2-((2S,4S)-1-(but-2-ynoyl)-4-(7-(5,6-dimethyl-1H-indazol-4-yl)-6-fluoro-8-methyl-4-((S)-1-
((S)-1-methylpyrrolidin-2-yl)ethoxy)-1H-pyrazolo[4,3-c]quinolin-1-yl)piperidin-2-
yl)acetonitrile, and
2-((2S,4S)-4-(7-(5,6-dimethyl-1H-indazol-4-yl)-6-fluoro-8-methyl-4-((S)-1-((S)-1-
methylpyrrolidin-2-yl)ethoxy)-1H-pyrazolo[4,3-c]quinolin-1-yl)-1-((E)-4-methoxybut-2-
enoyl)piperidin-2-yl)acetonitrile.

11. The method of claim 10, wherein the compound of Formula (II) or the pharmaceutically acceptable salt thereof is selected from:
1-(1-(2S,4S)-1-(but-2-ynoyl)-2-(cyanomethyl)piperidin-4-yl)-8-chloro-6-fluoro-4-(S)-1-((S)-1-methylpyrrolidin-2-yl)ethoxy)-1H-pyrazolo[4,3-c]quinolin-7-yl)isoquinoline-8-carbonitrile;
1-(8-chloro-1-(2S,4S)-2-(cyanomethyl)-1-(E)-4-methoxybut-2-enoyl)piperidin-4-yl)-6-fluoro-4-(S)-1-((S)-1-methylpyrrolidin-2-yl)ethoxy)-1H-pyrazolo[4,3-c]quinolin-7-yl)isoquinoline-8-carbonitrile;
1-(8-chloro-1-(2S,4S)-2-(cyanomethyl)-1-(2-fluoroacryloyl)piperidin-4-yl)-6-fluoro-4-((S)-1-(S)-1-methylpyrrolidin-2-yl)ethoxy)-1H-pyrazolo[4,3-c]quinolin-7-yl)isoquinoline-8-carbonitrile;
2-((2S,4S)-4-(8-chloro-7-(5,6-dimethyl-1H-indazol-4-yl)-4-(3-(ethyl(methyl)amino)azetidin-1-yl)-6-fluoro-1H-pyrazolo[4,3-c]quinolin-1-yl)-1-(2-fluoroacryloyl)piperidin-2-yl)acetonitrile;
8-(8-chloro-1-(2S,4S)-2-(cyanomethyl)-1-(2-fluoroacryloyl)piperidin-4-yl)-6-fluoro-4-((S)-1-(S)-1-methylpyrrolidin-2-yl)ethoxy)-1H-pyrazolo[4,3-c]quinolin-7-yl)-1-naphthonitrile;
8-(8-chloro-1-(2S,4S)-2-(cyanomethyl)-1-(E)-4-methoxybut-2-enoyl)piperidin-4-yl)-6-fluoro-4-(S)-1-((S)-1-methylpyrrolidin-2-yl)ethoxy)-1H-pyrazolo[4,3-c]quinolin-7-yl)-1-naphthonitrile; and
8-(1-(2S,4S)-1-(but-2-ynoyl)-2-(cyanomethyl)piperidin-4-yl)-6-fluoro-8-methyl-4-(S)-1-(S)-1-methylpyrrolidin-2-yl)ethoxy)-1H-pyrazolo[4,3-c]quinolin-7-yl)-1-naphthonitrile;
or a pharmaceutically acceptable salt thereof.

12. The method of claim 1, wherein the KRAS inhibitor a compound of Formula (III): 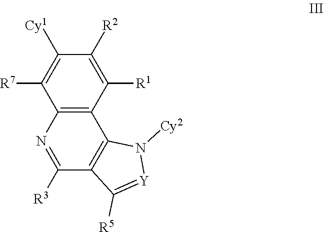
or a pharmaceutically acceptable salt thereof, wherein:
Y is N or CR⁶;
R¹ is selected from H, C₁₋₃ alkyl, C₁₋₃ haloalkyl, cyclopropyl, halo, D, CN, and OR^{a1};
wherein said C₁₋₃ alkyl and cyclopropyl are each optionally substituted with 1 or 2 substituents independently selected from R^{g};
R² is selected from H, C₁₋₃ alkyl, C₁₋₃ haloalkyl, 4-6 membered heterocycloalkyl, phenyl, 5-6 membered heteroaryl, 4-6 membered heterocycloalkyl-C₁₋₃ alkylene, phenyl-C₁₋₃ alkylene, 5-6 membered heteroaryl-C₁₋₃ alkylene, halo, D, CN, and OR^{a2}; wherein said C₁₋₃ alkyl, 4-6 membered heterocycloalkyl, phenyl, 5-6 membered heteroaryl, 4-6 membered heterocycloalkyl-C₁₋₃ alkylene, phenyl-C₁₋₃ alkylene, 5-6 membered heteroaryl-C₁₋₃ alkylene are each optionally substituted with 1 or 2 substituents independently selected from R^{g};
Cy¹ is selected from C₃₋₁₀ cycloalkyl, 4-10 membered heterocycloalkyl, C₆₋₁₀ aryl and 6-10 membered heteroaryl; wherein the 4-10 membered heterocycloalkyl and 6-10 membered heteroaryl each has at least one ring-forming carbon atom and 1, 2, 3, or 4 ring-forming heteroatoms independently selected from N, O, and S; wherein a ring-forming carbon atom of 6-10 membered heteroaryl and 4-10 membered heterocycloalkyl is optionally substituted by oxo to form a carbonyl group; and wherein the C3-io cycloalkyl, 4-10 membered heterocycloalkyl, C₆₋₁₀ aryl and 6-10 membered heteroaryl are each optionally substituted with 1, 2, 3, or 4 substituents independently selected from R¹⁰;
R³ is selected from H, C₁₋₃ alkyl, C₁₋₃ haloalkyl, C₃₋₆ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, halo, D, CN, OR^{f3}, C(O)NR^{c3}R^{d3}, NR^{c3}R^{j3}, and NR^{c3}C(O)R^{b3}; wherein said C₁₋₃ alkyl, C₃₋₆ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene are each optionally substituted with 1, 2, or 3 substituents independently selected from R³⁰;
R⁵ is selected from H, C₁₋₃ alkyl, C₁₋₃ haloalkyl, cyclopropyl, halo, D, CN, and OR^{a5}; wherein said C₁₋₃ alkyl and cyclopropyl are each optionally substituted with 1 or 2 substituents independently selected from R^{g};
R⁶ is selected from H, C₁₋₃ alkyl, C₁₋₃ haloalkyl, C₃₋₆ cycloalkyl, 4-9 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, halo, D, CN, OR^{a6}, and C(O)NR^{c6}R^{d6}; wherein said C₁₋₃ alkyl, C₃₋₆ cycloalkyl, 4-9 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene are each optionally substituted with 1 or 2 substituents independently selected from R⁶⁰;
R⁷ is selected from H, C₁₋₃ alkyl, C₁₋₃ haloalkyl, cyclopropyl, halo, D, CN, and OR^{a7};
wherein said C₁₋₃ alkyl and cyclopropyl are each optionally substituted with 1 or 2 substituents independently selected from R^{g};
Cy² is selected from 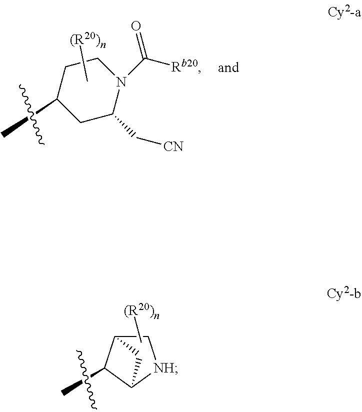
wherein n is 0, 1, or 2;
each R¹⁰ is independently selected from C₁₋₃ alkyl, C₁₋₃ haloalkyl, halo, D, CN, OR^{a10}, C(O)R_{b10}, C(O)NR^{c10}R^{d10}, C(O)OR^{a10}R^{d10}, and S(O)₂R_{b10};
each R²⁰ is independently selected from C₁₋₃ alkyl, C₁₋₃ haloalkyl, halo, D, CN, and OR^{a20};
each R³⁰ is independently selected from C₁₋₃ alkyl, C₁₋₃ haloalkyl, C₃₋₆ cycloalkyl, 4-6 membered heterocycloalkyl, phenyl, 5-6 membered heteroaryl, halo, D, CN, OR^{a30}, C(O)R^{b30}, C(O)NR^{c30}, R^{d30}, C(O)OR^{a30}, NR^{c30}R^{d30} and S(O)₂R^{b30}; wherein said C₁₋₃ alkyl, C₃₋₆ cycloalkyl, 4-6 membered heterocycloalkyl, phenyl, and 5-6 membered heteroaryl are each optionally substituted with 1 or 2 substituents independently selected from R³¹;
each R³¹ is independently selected from C₁₋₃ alkyl, C₁₋₃ haloalkyl, halo, D, CN, OR^{a31}, C(O)R^{b31}, C(O)NR^{c31}R^{d31}, C(O)OR^{a31}, NR^{c31}R^{d31}, and S(O)₂R^{b31};
each R³³ is independently selected from C₁₋₃ alkyl, C₁₋₃ haloalkyl, C₃₋₆ cycloalkyl, 4-membered heterocycloalkyl, 6-membered heterocycloalkyl, phenyl, 5-6 membered heteroaryl, halo, D, CN, OR^{a30}, C(O)NR^{c31}OR^{d30}, and NR^{c30}R^{d30}; wherein said C₁₋₃ alkyl, C₃₋₆ cycloalkyl, 4-membered heterocycloalkyl, 6-membered heterocycloalkyl, phenyl, and 5-6 membered heteroaryl are each optionally substituted with 1 or 2 substituents independently selected from R³¹;
each R⁶⁰ is independently selected from C₁₋₃ alkyl, C₁₋₃ haloalkyl, 4-6 membered heterocycloalkyl, 5-6 membered heteroaryl, halo, D, CN, OR^{a60}, C(O)R^{b60}; C(O)NR^{c60}R^{d60}, NR^{c60}C(O)R^{b60}, C(O)R^{b60}; NR^{c60}C(O)OR^{a60}, NR^{c60}R^{d60}, NR^{c60}S(O)₂R^{b60}, and S(O)₂R^{b60}; wherein said C₁₋₃ alkyl, 4-6 membered heterocycloalkyl, and 5-6 membered heteroaryl are each optionally substituted with 1 or 2 substituents independently selected from R⁶¹;
each R⁶¹ is independently selected from C₁₋₃ alkyl, C₁₋₃ haloalkyl, halo, D, CN, OR^{a61}, and NR^{c61}R^{d61};
R^{a1} is selected from H, C₁₋₃ alkyl, and C₁₋₃ haloalkyl;
each R^{a2} is independently selected from H, C₁₋₃ alkyl, and C₁₋₃ haloalkyl;
each R^{b3}, R^{c3} and R^{d3} is independently selected from H, C₁₋₃ alkyl, C₁₋₃ haloalkyl, C₃₋₆ cycloalkyl, 4-6 membered heterocycloalkyl, phenyl and 5-6 membered heteroaryl; wherein said, C₁₋₃ alkyl, C₃₋₆ cycloalkyl, 4-6 membered heterocycloalkyl, phenyl and 5-6 membered heteroaryl are each optionally substituted with 1, 2, or 3 substituents independently selected from R³⁰;
or R^{c3} and R^{d3} attached to the same N atom, together with the N atom to which they are attached, form a 4-, 5-, or 6-membered heterocycloalkyl group optionally substituted with 1, 2, or 3 substituents independently selected from R³⁰;
R^{j3} is selected from C₁₋₃ alkyl, C₁₋₃ haloalkyl, C₃₋₆ cycloalkyl, 4-6 membered heterocycloalkyl, phenyl and 5-6 membered heteroaryl; wherein said , C₁₋₃ alkyl, C₃₋₆ cycloalkyl, 4-6 membered heterocycloalkyl, phenyl and 5-6 membered heteroaryl are each optionally substituted with 1, 2, or 3 substituents independently selected from R³⁰;
or R^{c3} and R^{j3} attached to the same N atom, together with the N atom to which they are attached, form a 4-, 5-, or 6-membered heterocycloalkyl group optionally substituted with 1, 2, or 3 substituents independently selected from R³⁰;
R^{f3} is selected from C₁₋₃ haloalkyl, C₃₋₆ cycloalkyl, 4-6 membered heterocycloalkyl, phenyl and 5-6 membered heteroaryl; wherein said C₁₋₃ haloalkyl, C₃₋₆ cycloalkyl, 4-6 membered heterocycloalkyl, phenyl, and 5-6 membered heteroaryl are each optionally substituted with 1, 2, or 3 substituents independently selected from R³⁰; or
R^{f3} is selected from 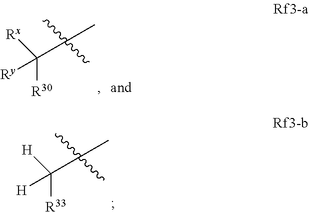
wherein R^{x} is H or C₁₋₂ alkyl and R^{y} is C₁₋₂ alkyl;
or R^{x} and R^{y}, together with the C atom to which they are attached, form a 3-, or 4-membered cycloalkyl group;
R^{a5} is selected from H, C₁₋₃ alkyl, and C₁₋₃ haloalkyl;
each R^{a6}, R^{c6} and R^{d6} is independently selected from H, C₁₋₃ alkyl, C₁₋₃ haloalkyl, C₃₋₆ cycloalkyl, 4-6 membered heterocycloalkyl, phenyl and 5-6 membered heteroaryl; wherein said C₁₋₃ alkyl, C₃₋₆ cycloalkyl, 4-6 membered heterocycloalkyl, phenyl and 5-6 membered heteroaryl are each optionally substituted with 1 or 2 substituents independently selected from R⁶⁰;
R^{a7} is selected from H, C₁₋₃ alkyl, and C₁₋₃ haloalkyl;
each R^{a10}, R^{b10}, R^{c10} and R^{d30} selected from H, C₁₋₃ alkyl, and C₁₋₃ haloalkyl;
each R^{a5} is independently selected from H, C₁₋₃ alkyl, and C₁₋₃ haloalkyl;
R^{b20} is selected from NH2, C₁₋₃ alkyl, and C₁₋₃ haloalkyl;
each R^{a30}, R^{b30}, R^{c30} and R^{d30} is independently selected from H, C₁₋₃ alkyl, and C₁₋₃ haloalkyl;
each R^{a31}, R^{b31}, R^{c31} and R^{d31} is independently selected from H, C₁₋₃ alkyl, and C₁₋₃ haloalkyl;
each R^{a60}, R^{b60}, R^{c60} and R^{d60} is independently selected from H, C₁₋₃ alkyl, C₁₋₃ haloalkyl, C₃₋₆ cycloalkyl, 4-6 membered heterocycloalkyl, and 5-6 membered heteroaryl; wherein said C₁₋₃ alkyl, C₃₋₆ cycloalkyl, 4-6 membered heterocycloalkyl, and 5-6 membered heteroaryl are each optionally substituted with 1 or 2 substituents independently selected from R⁶¹;
or any R^{c60} and R^{d60} attached to the same N atom, together with the N atom to which they are attached, form a 4-, 5-, or 6-membered heterocycloalkyl group optionally substituted with 1 or 2 substituents independently selected from R⁶¹; and
each R^{a61}, R^{c61}, and R^{d61}, is independently selected from H, C₁₋₃ alkyl, and C₁₋₃ haloalkyl; and
each R^{g} is independently selected from D, OH, CN, halo, C₁₋₃ alkyl, C₁₋₃ haloalkyl, C₁₋₃ alkoxy, C₁₋₃ haloalkoxy, amino, C₁₋₃ alkylamino, and di(C₁₋₃ alkyl)amino;
provided that the compound of Formula (III) is other than,
3-(1-((1R,4R,5S)-2-azabicyclo[2.1.1]hexan-5-yl)-8-(2-cyanoethyl)-4-ethoxy-6-fluoro (3-hydroxynaphthalen-1-yl)-1H-pyrrolo[3,2-c]quinolin-2-yl)-N,N-dimethylpropanamide.

13. The method of claim 12, wherein the KRAS inhibitor is selected from:
3-(1-((1R,4R,5S)-2-Azabicyclo[2.1.1]hexan-5-yl)-7-(7-chloro-3-hydroxynaphthalen-1-yl)-6-fluoro-2-methyl-4-(1H-1,2,4-triazol-1-yl)-1H-pyrrolo[3,2-c]quinolin-8-yl)propanenitrile;
3-(1-((1R,4R,5S)-2-Azabicyclo[2.1.1]hexan-5-yl)-7-(5,7-difluoro-1H-indol-3-yl)-6-fluoro-2-methyl-4-(S)-1-((S)-1-methylpyrrolidin-2-yl)ethoxy)-1H-pyrrolo[3,2-c]quinolin-8-yl)propanenitrile;
3-(1-((1R,4R,5S)-2-Azabicyclo[2.1.1]hexan-5-yl)-6-fluoro-7-(6-fluoro-5-methyl-1H-indol-3-yl)-2-methyl-4-(S)-1-((S)-1-methylpyrrolidin-2-yl)ethoxy)-1H-pyrrolo[3,2-c]quinolin-8-yl)propanenitrile;
3-(2-(3-(Azetidin-1-yl)-3-oxopropyl)-1-((1R, 4R, 5S)-2-azabicyclo[2.1.1]hexan-5-yl)-7-(2,3-dichlorophenyl)-6-fluoro-4-((S)-1-((S)-1-methylpyrrolidin-2-yl)ethoxy)-1H-pyrrolo[3,2-c]quinolin-8-yl)propanenitrile;
3-((1-(1R,4R,5S)-2-Azabicyclo[2.1.1]hexan-5-yl)-6-fluoro-7-(3-hydroxynaphthalen-1-yl)-8-methyl-4-(S)-1-((S)-1-methylpyrrolidin-2-yl)ethoxy)-1H-pyrrolo[3,2-c]quinolin-2-yl)methyl)oxazolidin-2-one;
8-(1-((1R,4R,5S)-2-Azabicyclo[2.1.1]hexan-5-yl)-6-fluoro-2,8-dimethyl-4-((S)-1-((S)-1-methylpyrrolidin-2-yl)ethoxy)-1H-pyrrolo[3,2-c]quinolin-7-yl)-1-naphthonitrile;
1-((2S,4S)-1-Acetyl-2-(cyanomethyl)piperidin-4-yl)-7-(8-cyanonaphthalen-1-yl)-6-fluoro-4-((S)-1-((S)-1-methylpyrrolidin-2-yl)ethoxy)-1H-pyrazolo[4,3-c]quinoline-8-carbonitrile;
8-(1-((1R,4R,5S)-2-Azabicyclo[2.1.1]hexan-5-yl)-6-fluoro-8-methyl-4-((S)-1-((S)-1-methylpyrrolidin-2-yl)ethoxy)-2-((3-oxomorpholino)methyl)-1H-pyrrolo[3,2-c]quinolin-7-yl)-1-naphthonitrile;
3-(7-(Benzo[b]thiophen-3-yl)-1-((1R,4R,5S)-2-azabicyclo[2.1.1]hexan-5-yl)-6-fluoro-4-((S)-1-((S)-1-methylpyrrolidin-2-yl)ethoxy)-2-((2-oxopyrrolidin-1-yl)methyl)-1H-pyrrolo[3,2-c]quinolin-8-yl)propanenitrile;
3-(1-((1R,4R,5S)-2-Azabicyclo[2.1.1]hexan-5-yl)-4-(S)-1-(dimethylamino)propan yl)oxy)-6-fluoro-7-(7-fluoronaphthalen-1-yl)-2-((2-oxopyrrolidin-1-yl)methyl)-1H-pyrrolo[3,2-c]quinolin-8-yl)propanenitrile;
8-(1-((1R,4R,5S)-2-Azabicyclo[2.1.1]hexan-5-yl)-8-(2-cyanoethyl)-6-fluoro-2-methyl-4-((S)-1-((S)-1-methylpyrrolidin-2-yl)ethoxy)-1H-pyrrolo[3,2-c]quinolin-7-yl)-1-naphthonitrile;
3-(1-((1R,4R,5S)-2-Azabicyclo[2.1.1]hexan-5-yl)-7-(2,3-dichloro-5-hydroxyphenyl) fluoro-2-methyl-4-((S)-1-((S)-1-methylpyrrolidin-2-yl)ethoxy)-1H-pyrrolo[3,2-c]quinolin-8-yl)propanenitrile;
3-(1-((1R,4R,5S)-2-Azabicyclo[2.1.1]hexan-5-yl)-8-(2-cyanoethyl)-6-fluoro-4-((3-fluoro-1-methylazetidin-3-yl)methoxy)-7-(3-hydroxynaphthalen-1-yl)-1H-pyrrolo[3,2-c]quinolin-2-yl)-N,N-dimethylpropanamide;
3-(1-((1R,4R,5S)-2-Azabicyclo[2.1.1]hexan-5-yl)-6-fluoro-7-(3-hydroxynaphthalen-1-yl)-2-methyl-4-(5-methylpyrazin-2-yl)-1H-pyrrolo[3,2-c]quinolin-8-yl)propanenitrile;
3-(1-((1R,4R,5S)-2-Azabicyclo[2.1.1]hexan-5-yl)-6-fluoro-7-(7-fluoronaphthalen-1-yl)-4-methyl-2-((4-methyl-2-oxopiperazin-1-yl)methyl)-1H-pyrrolo[3,2-c]quinolin-8-yl)propanenitrile;
3-(1-((1R,4R,5S)-2-azabicyclo[2.1.1]hexan-5-yl)-7-(2,3-dichloro-5-hydroxyphenyl)-4-ethoxy-6-fluoro-24(4-isopropyl-2-oxopiperazin-1-yl)methyl)-1H-pyrrolo[3,2-c]quinolin-8-yl)propanenitrile;
3-(1-((1R,4R,5S)-2-Azabicyclo[2.1.1]hexan-5-yl)-4-(3-(dimethylamino)-3-methylazetidin-1-yl)-6-fluoro-7-(7-fluoronaphthalen-1-yl)-2-((3-oxomorpholino)methyl)-1H-pyrrolo[3,2-c]quinolin-8-yl)propanenitrile;
3-(1-((1R,4R,5S)-2-azabicyclo[2.1.1]hexan-5-yl)-4-ethoxy-6-fluoro-7-(3-hydroxynaphthalen-1-yl)-2-(1-(3-oxomorpholino)ethyl)-1H-pyrrolo[3,2-c]quinolin-8-yl)propanenitrile;
3-(1-((endo)-2-Azabicyclo[2.1.1]hexan-5-yl)-6-fluoro-7-(3-hydroxynaphthalen-1-yl)-4-((S)-1-((S)-1-methylpyrrolidin-2-yl)ethoxy)-2-(pyridin-3-yl)-1H-pyrrolo[3,2-c]quinolin-8-yl)propanenitrile;
3-(2-(3-(azetidin-1-yl)-3-oxopropyl)-1-(1R,4R,5S)-2-azabicyclo[2.1.1]hexan-5-yl)-7-(7,8-difluoronaphthalen-1-yl)-6-fluoro-4-((S)-1-((S)-1-methylpyrrolidin-2-yl)ethoxy)-1H-pyrrolo[3,2-c]quinolin-8-yl)propanenitrile;
3-(2-(3-(azetidin-1-yl)-3-oxopropyl)-1-(1R,4R,5S)-2-azabicyclo[2.1.1]hexan-5-yl) (6,7-difluoronaphthalen-1-yl)-6-fluoro-4-((S)-1-((S)-1-methylpyrrolidin-2-yl)ethoxy)-1H-pyrrolo[3,2-c]quinolin-8-yl)propanenitrile;
3-(1-((1R,4R,5S)-2-azabicyclo[2.1.1]hexan-5-yl)-6-fluoro-7-(7-fluoro hydroxynaphthalen-1-yl)-2-methyl-4-((S)-1-((S)-1-methylpyrrolidin-2-yl)ethoxy)-1H-pyrrolo[3,2-c]quinolin-8-yl)propanenitrile;
1-(1-((2S,4S)-1-Acetyl-2-(cyanomethyl)piperidin-4-yl)-8-chloro-6-fluoro-4-((S)-1-((S)-1-methylpyrrolidin-2-yl)ethoxy)-1H-pyrazolo[4,3-c]quinolin-7-yl)isoquinoline-8-carbonitrile;
8-(1-((2S,4S)-1-acetyl-2-(cyanomethyl)piperidin-4-yl)-8-chloro-6-fluoro-4-((S)-1-((S)-1-methylpyrrolidin-2-yl)ethoxy)-1H-pyrrolo[3,2-c]quinolin-7-yl)-1-naphthonitrile;
8-(1-((2S,4S)-1-acetyl-2-(cyanomethyl)piperidin-4-yl)-8-chloro-6-fluoro-4-((S)-1-((S)-1-methylpyrrolidin-2-yl)ethoxy)-1H-pyrazolo[4,3-c]quinolin-7-yl)-1-naphthonitrile;
3-(1-((1R,4R,5S)-2-Azabicyclo[2.1.1]hexan-5-yl)-6-fluoro-7-(7-fluoro-3-hydroxynaphthalen-1-yl)-2-methyl-4-(1H-1,2,4-triazol-1-yl)-1H-pyrrolo[3,2-c]quinolin-8-yl)propanenitrile;
3-(1-((1R,4R,5S)-2-Azabicyclo[2.1.1]hexan-5-yl)-6-fluoro-7-(7-fluoronaphthalen-1-yl)-2-methyl-4-(S)-1-((S)-1-methylpyrrolidin-2-yl)ethoxy)-1H-pyrrolo[3,2-c]quinolin-8-yl)propanenitrile;
(2R)-2-(1-((1R,4R,5S)-2-azabicyclo[2.1.1]hexan-5-yl)-8-(2-cyanoethyl)-7-(2,3-dichlorophenyl)-6-fluoro-4-(1H-1,2,4-triazol-1-yl)-1H-pyrrolo[3,2-c]quinolin-2-yl)-N,N-dimethylpyrrolidine-1-carboxamide;
methyl (2R)-2-(1-((1R,4R,5S)-2-azabicyclo[2.1.1]hexan-5-yl)-7-(2-chloro-3-methylphenyl)-8-(2-cyanoethyl)-6-fluoro-4-(1H-1,2,4-triazol-1-yl)-1H-pyrrolo[3,2-c]quinolin-2-yl)pyrrolidine-1-carboxylate;
Methyl (1S,3R,5S)-3-(1-((1R,4R,5S)-2-azabicyclo[2.1.1]hexan-5-yl)-8-(2-cyanoethyl)-7-(2,3-dichlorophenyl)-4-(6-(dimethylcarbamoyl)pyridin-3-yl)-6-fluoro-1H-pyrrolo[3,2-c]quinolin-2-yl)-2-azabicyclo[3.1.0]hexane-2-carboxylate;
3-(1-((1R,4R,5S)-2-Azabicyclo[2.1.1]hexan-5-yl)-7-(2,3-dichlorophenyl)-6-fluoro-4-methyl-2-(5-oxo-1,2,3,5-tetrahydroindolizin-3-yl)-1H-pyrrolo[3,2-c]quinolin-8-yl)propanenitrile;
Methyl (2R)-2-(1-((1R,4R,5S)-2-azabicyclo[2.1.1]hexan-5-yl)-8-(2-cyanoethyl)-7-(2,3-dichlorophenyl)-4-(6-(dimethylcarbamoyl)pyridin-3-yl)-6-fluoro-1H-pyrrolo[3,2-c]quinolin-2-yl)pyrrolidine-1-carboxylate;
Methyl (2R)-2-(1-((1R,4R,5S)-2-azabicyclo[2.1.1]hexan-5-yl)-8-(2-cyanoethyl)-7-(2,3-dichlorophenyl)-6-fluoro-4-(6-(methylcarbamoyl)pyridin-3-yl)-1H-pyrrolo[3,2-c]quinolin yl)pyrrolidine-1-carboxylate;
3-(1-((1R,4R,5S)-2-Azabicyclo[2.1.1]hexan-5-yl)-7-(2-chloro-3-fluorophenyl)-2-((R)-1-(cyclopropanecarbonyl)pyrrolidin-2-yl)-6-fluoro-4-methyl-1H-pyrrolo[3,2-c]quinolin-8-yl)propanenitrile;
8-(2-((R)-1-Acetylpyrrolidin-2-yl)-1-(1R,4R,5S)-2-azabicyclo[2.1.1]hexan-5-yl)-6-fluoro-8-methyl-4-(2-methylpyridin-4-yl)-1H-pyrrolo[3,2-c]quinolin-7-yl)-1,2,3,4-tetrahydronaphthalene-1-carbonitrile;
5-(1-((1R,4R,5S)-2-Azabicyclo[2.1.1]hexan-5-yl)-7-(3-chloro-2-methylphenyl)-8-(2-cyanoethyl)-6-fluoro-2-((R)-1-(2-oxopyrazin-1(2H)-yl)ethyl)-1H-pyrrolo[3,2-c]quinolin-4-yl)-N-methylpicolinamide;
3-(1-((1R,4R,5S)-2-Azabicyclo[2.1.1]hexan-5-yl)-6-fluoro-7-(7-fluoronaphthalen-1-yl)-4-(6-(2-hydroxypropan-2-yl)pyridin-3-yl)-2-((R)-1-(2-oxopyrazin-1(2H)-yl)ethyl)-1H-pyrrolo[3,2-c]quinolin-8-yl)propanenitrile;
3-(1-((1R,4R,5S)-2-Azabicyclo[2.1.1]hexan-5-yl)-7-(3-chloro-2-methylphenyl)-6-fluoro-4-(5-methylpyrazin-2-yl)-2-((R)-1-(2-oxopyrazin-1(2H)-yl)ethyl)-1H-pyrrolo[3,2-c]quinolin-8-yl)propanenitrile;
Methyl (2R)-2-(1-((1R,4R,5S)-2-azabicyclo[2.1.1]hexan-5-yl)-8-(2-cyanoethyl)-7-(2,3-dichlorophenyl)-6-fluoro-4-(5-fluoro-6-(methylcarbamoyl)pyridin-3-yl)-1H-pyrrolo[3,2-c]quinolin-2-yl)pyrrolidine-1-carboxylate;
Methyl (2R)-2-(1-((1R,4R,5S)-2-azabicyclo[2.1.1]hexan-5-yl)-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)pyrrolidine-1-carboxylate;
Ethyl (2R)-2-(1-((1R,4R,5S)-2-azabicyclo[2.1.1]hexan-5-yl)-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)pyrrolidine-1-carboxylate;
3-(1-((1R,4R,5S)-2-Azabicyclo[2.1.1]hexan-5-yl)-7-(2,3-dichlorophenyl)-2-((R)-1-(3,3-difluoroazetidine-1-carbonyl)pyrrolidin-2-yl)-6-fluoro-4-(methyl-d3)-1H-pyrrolo[3,2-c]quinolin-8-yl)propanenitrile;
3-(1-((1R,4R,5S)-2-Azabicyclo[2.1.1]hexan-5-yl)-7-(2,3-dichlorophenyl)-2-((R)-1-(3,3-difluoroazetidine-1-carbonyl)pyrrolidin-2-yl)-6-fluoro-1H-pyrrolo[3,2-c]quinolin yl)propanenitrile;
3-(1-((1R,4R,5S)-2-Azabicyclo[2.1.1]hexan-5-yl)-7-(3-chloro-2-methylphenyl)-6-fluoro-4-(5-methylpyrazin-2-yl)-2-((R)-1-(3-oxomorpholino)ethyl)-1H-pyrrolo[3,2-c]quinolin-8-yl)propanenitrile;
5-(1-((1R,4R,5S)-2-Azabicyclo[2.1.1]hexan-5-yl)-8-(2-cyanoethyl)-6-fluoro-7-(7-fluoronaphthalen-1-yl)-24(R)-1-(3-oxomorpholino)ethyl)-1H-pyrrolo[3,2-c]quinolin-4-yl)-N-methylpicolinamide;
3-(1-((1R,4R,5S)-2-Azabicyclo[2.1.1]hexan-5-yl)-6-fluoro-7-(7-fluoronaphthalen-1-yl)-4-(6-(2-hydroxypropan-2-yl)pyridin-3-yl)-24(R)-1-(3-oxomorpholino)ethyl)-1H-pyrrolo[3,2-c]quinolin-8-yl)propanenitrile;
3-(1-((1R,4R,5S)-2-Azabicyclo[2.1.1]hexan-5-yl)-6-fluoro-7-(7-fluoronaphthalen-1-yl)-4-(5-methylpyrazin-2-yl)-2-((R)-1-(3-oxomorpholino)ethyl)-1H-pyrrolo[3,2-c]quinolin-8-yl)propanenitrile;
Methyl (1R,3R,5R)-3-(1-((1R,4R,5S)-2-azabicyclo[2.1.1]hexan-5-yl)-8-(2-cyanoethyl)-7-(2,3-dichlorophenyl)-4-(6-(dimethylcarbamoyl)pyridin-3-yl)-6-fluoro-1H-pyrrolo[3,2-c]quinolin-2-yl)-2-azabicyclo[3.1.0]hexane-2-carboxylate;
3-(1-((1R,4R,5S)-2-Azabicyclo[2.1.1]hexan-5-yl)-24(1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile;
3-(1-((1R,4R,5S)-2-Azabicyclo[2.1.1]hexan-5-yl)-7-(2,3-dichlorophenyl)-6-fluoro-4-(6-(2-hydroxypropan-2-yl)pyridin-3-yl)-2-((R)-1-(2-oxopyrazin-1(2H)-yl)ethyl)-1H-pyrrolo[3,2-c]quinolin-8-yl)propanenitrile;
Methyl (2R,4S)-2-(1-((1R,4R,5S)-2-azabicyclo[2.1.1]hexan-5-yl)-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)-4-fluoropyrrolidine-1-carboxylate;
Methyl (2R,5R)-2-(1-((1R,4R,5S)-2-azabicyclo[2.1.1]hexan-5-yl)-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)-5-methylpyrrolidine-1-carboxylate;
Methyl (2R)-2-(1-((1R,4R,5S)-2-azabicyclo[2.1.1]hexan-5-yl)-3-chloro-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)pyrrolidine-1-carboxylate;
4-(1-((1R,4R,5S)-2-Azabicyclo[2.1.1]hexan-5-yl)-8-(2-cyanoethyl)-7-(2,3-dichlorophenyl)-6-fluoro-2-((R)-1-(2-oxopyrazin-1(21/)-yl)ethyl)-1H-pyrrolo[3,2-c]quinolin-4-yl)-2-fluoro-N-methylbenzamide;
Methyl ((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)carbamate;
N-((1R)-1-(1-((1R,4R,5S)-2-Azabicyclo[2.1.1]hexan-5-yl)-8-(2-cyanoethyl)-7-(2,3-dichlorophenyl)-6-fluoro-4-methyl-1H-pyrrolo[3 ,2-c]quinolin-2-yl)ethyl)-2,2-difluoroacetamide;
N-((1R)-1-(1-((1R,4R,5S)-2-Azabicyclo[2.1.1]hexan-5-yl)-8-(2-cyanoethyl)-7-(2,3-dichlorophenyl)-6-fluoro-4-methyl-1H-pyrrolo[3 ,2-c]quinolin-2-yl)ethyl)-2,2-difluoroacetamide;
(2S)-N-((1R)-1-(1-((1R,4R,5S)-2-Azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)tetrahydrofuran-2-carboxamide;
N-((1R)-1-(1-((1R,4R,5S)-2-Azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)cyclopropanesulfonamide;
N-((1R)-1-(1-((1R,4R,5S)-2-Azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)thiazole-4-carboxamide;
N-((1R)-1-(1-((1R,4R,5S)-2-Azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)-N-methylcyclopropanecarboxamide;
N-((1R)-1-(1-((1R,4R,5S)-2-Azabicyclo[2.1.1]hexan-5-yl)-8-(2-cyanoethyl)-7-(2,3-dichlorophenyl)-6-fluoro-4-(1-hydroxyethyl)-1H-pyrrolo[3,2-c]quinolin-2-yl)ethyl)-1-methylcyclopropane-1-carboxamide;
3-(1-((1R,4R,5S)-2-Azabicyclo[2.1.1]hexan-5-yl)-7-(2,3-dichlorophenyl)-6-fluoro-4-(1-hydroxyethyl)-2-((1R,3R,5R)-2-(1-methylcyclopropane-1-carbonyl)-2-azabicyclo[3.1.0]hexan yl)-1H-pyrrolo[3,2-c]quinolin-8-yl)propanenitrile;
3-(1-((1R,4R,5S)-2-Azabicyclo[2.1.1]hexan-5-yl)-7-(2,3-dichlorophenyl)-6-fluoro ((1R,3R,5R)-2-(1-fluorocyclopropane-1-carbonyl)-2-azabicyclo[3.1.0]hexan-3-yl)-4-(1-hydroxyethyl)-1H-pyrrolo[3,2-c]quinolin-8-yl)propanenitrile;
3-(1-((1R,4R,5S)-2-Azabicyclo[2.1.1]hexan-5-yl)-7-(2,3-dichlorophenyl)-6-fluoro-2-((1R,3R,5R)-2-(1-fluorocyclopropane-1-carbonyl)-2-azabicyclo[3.1.0]hexan-3-yl)-4-methyl-1H-pyrrolo[3,2-c]quinolin-8-yl)propanenitrile;
N-((1R)-1-(1-((1R,4R,5S)-2-Azabicyclo[2.1.1]hexan-5-yl)-8-(2-cyanoethyl)-7-(2,3-dichlorophenyl)-6-fluoro-4-(1-hydroxyethyl)-1H-pyrrolo[3,2-c]quinolin-2-yl)ethyl)-1-fluorocyclopropane-1-carboxamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-(1-hydroxyethyl)-1H-pyrrolo[3,2-c]quinolin-2-yl)ethyl)-1-fluorocyclobutane-1-carboxamide;
3-(1-((1R,4R,5S)-2-azabicyclo[2.1.1]hexan-5-yl)-7-(3-chloro-2-methylphenyl)-2-(1-(2,6-dimethyl-3-oxo-2,3-dihydropyridazin-4-yl)ethyl)-6-fluoro-4-methyl-1H-pyrrolo[3,2-c]quinolin-8-yl)propanenitrile;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)pyrimidine-4-carboxamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)pyridazine-3-carboxamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)-3,3-difluoroazetidine-1-carboxamide;
3-(1-((1R,4R,5S)-2-azabicyclo[2.1.1]hexan-5-yl)-7-(2,3-dichlorophenyl)-6-fluoro-4-methyl-2-((R)-1-(1-methyl-1H-pyrazol-4-yl)amino)ethyl)-1H-pyrrolo[3,2-c]quinolin-8-yl)propanenitrile;
5-(1-((1R,4R,5S)-2-Azabicyclo[2.1.1]hexan-5-yl)-8-(2-cyanoethyl)-7-(2,3-dichlorophenyl)-6-fluoro-2-((R)-1-(1-fluorocyclopropane-1-carbonyl)pyrrolidin-2-yl)-1H-pyrrolo[3,2-c]quinolin-4-yl)-N,N-dimethylpicolinamide; and
methyl (2R)-2-(1-((1R,4R,5S)-2-Azabicyclo[2.1.1]hexan-5-yl)-8-(2-cyanoethyl)-7-(2,3-dichlorophenyl)-4-(4-((dimethylamino)methyl)-2,3-difluorophenyl)-6-fluoro-1H-pyrrolo[3,2-c]quinolin-2-yl)pyrrolidine-1-carboxylate,
or a pharmaceutically acceptable salt thereof.

14. The method of claim 1, wherein the KRAS inhibitor is selected from sotorasib, adagrasib, Compound 2, Compound 3 and Compound 4, or a pharmaceutically acceptable salt thereof.

15. The method of claim 1, wherein the KRAS inhibitor is selected from sotorasib, Compound 2, Compound 3 and Compound 4, or a pharmaceutically acceptable salt thereof.

16. The method of claim 1, wherein the KRAS inhibitor is selected from Compound 5, Compound 6, and Compound 7.

17. The method of claim 1, wherein the KRAS inhibitor is sotorasib, or a pharmaceutically acceptable salt thereof.

18. The method of claim 1, wherein the KRAS inhibitor is adagrasib, or a pharmaceutically acceptable salt thereof.

19. The method of claim 1, wherein the KRAS inhibitor is Compound 2, or a pharmaceutically acceptable salt thereof.

20. The method of claim 1, wherein the KRAS inhibitor is Compound 3, or a pharmaceutically acceptable salt thereof.

21. The method of claim 1, wherein the KRAS inhibitor is Compound 4, or a pharmaceutically acceptable salt thereof.

22. The method of claim 1, wherein the KRAS inhibitor is a KRAS G12C inhibitor.

23. The method of claim 1, wherein the KRAS inhibitor is a KRAS G12D inhibitor.

24. A method of treating cancer in a patient, comprising administering to said patient:
(i) pemigatinib, or a pharmaceutically acceptable salt thereof; and
(ii) a KRAS inhibitor, which is a compound of Formula (I): 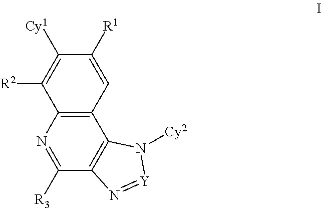
or a pharmaceutically acceptable salt thereof, wherein:
Y is N or CH;
R¹ is selected from Cl, CH₃, CH₂F, CHF₂, and CF₃;
Cy¹ is selected from 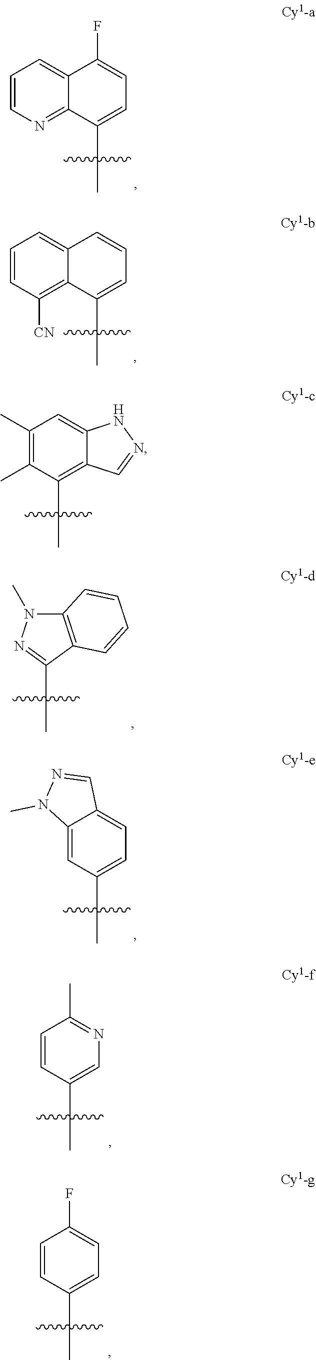 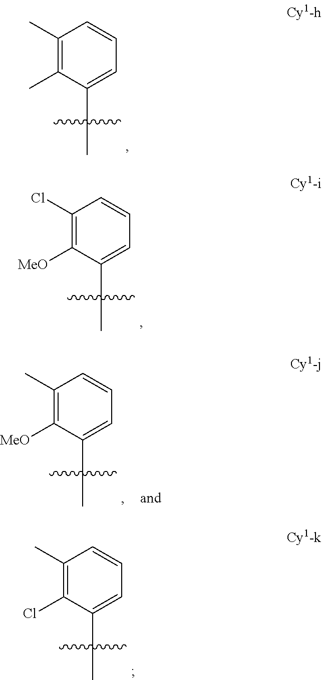
R² is selected from F and Cl;
R³ is selected from 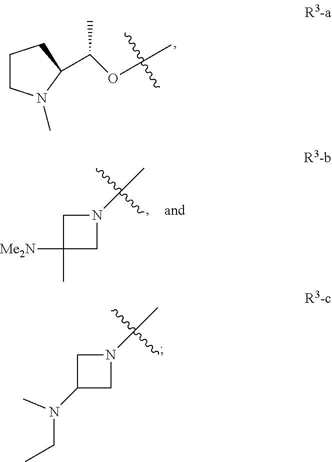
and,
Cy² is selected from 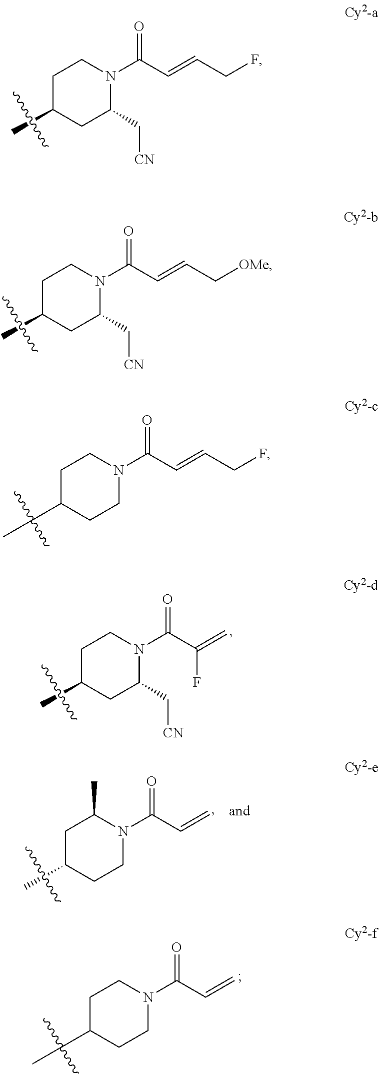
provided that the compound of Formula (I) is other than
2-((2S,4S)-4-(7-(2-chloro-3-methylphenyl)-4-(3-(dimethylamino)-3-
methylazetidin-1-yl)-6-fluoro-8-methyl-1H-[1,2,3]triazolo[4,5-c]quinolin-
1-yl)-1-((E)-4-fluorobut-2-enoyl)piperidin-2-yl)acetonitrile and
2-((2S,4S)-4-(4-(3-(dimethylamino)-3-methylazetidin-1-yl)-7-(2,3-
dimethylphenyl)-6-fluoro-8-methyl-1H-[1,2,3]triazolo[4,5-c]quinolin-
1-yl)-1-((E)-4-fluorobut-2-enoyl)piperidin-2-yl)acetonitrile.

25. The method of claim 24, wherein the compound of Formula (I) or the pharmaceutically acceptable salt thereof is selected from:
2-((2S,4S)-4-(4-(3-(dimethylamino)-3-methylazetidin-1-yl)-6-fluoro-7-(5-fluoroquinolin-8-yl)-8-(trifluoromethyl)-1H-[1,2,3]triazolo[4,5-c]quinolin-1-yl)-1-((E)-4-fluorobut enoyl)piperidin-2-yl)acetonitrile;
2-((2S,4S)-4-(4-(3-(dimethylamino)-3-methylazetidin-1-yl)-6-fluoro-7-(2-methoxy-3-methylphenyl)-8-(trifluoromethyl)-1H-[1,2,3]triazolo[4,5-c]quinolin-1-yl)-1-(E)-4-fluorobut-2-enoyl)piperidin-2-yl)acetonitrile;
2-((2S,4S)-4-(7-(3-chloro-2-methoxyphenyl)-4-(3-(dimethylamino)-3-methylazetidin-1-yl)-6-fluoro-8-(trifluoromethyl)-1H-[1,2,3]triazolo[4,5-c]quinolin-1-yl)-1-((E)-4-fluorobut-2-enoyl)piperidin-2-yl)acetonitrile;
2-((2S,4S)-4-(6-fluoro-7-(5-fluoroquinolin-8-yl)-4-(S)-1-(S)-1-methylpyrrolidin-2-yl)ethoxy)-8-(trifluoromethyl)-1H-[1,2,3]triazolo[4,5-c]quinolin-1-yl)-1-((E)-4-fluorobut-2-enoyl)piperidin-2-yl)acetonitrile;
1-(4-(6-fluoro-7-(5-fluoroquinolin-8-yl)-4-((S)-1-((S)-1-methylpyrrolidin-2-yl)ethoxy)-8-(trifluoromethyl)-1H-[1,2,3]triazolo[4,5-c]quinolin-1-yl)piperidin-1-yl)prop-2-en-1-one;
2-((2S,4S)-4-(4-(3-(dimethylamino)-3-methylazetidin-1-yl)-7-(2,3-dimethylphenyl)-6-fluoro-8-methyl-1H-[1,2,3]triazolo[4,5-c]quinolin-1-yl)-1-((E)-4-methoxybut-2-enoyl)piperidin-2-yl)acetonitrile;
2-((2S,4S)-4-(6-fluoro-8-methyl-7-(1-methyl-1H-indazol-6-yl)-4-((S)-1-(S)-1-methylpyrrolidin-2-yl)ethoxy)-1H-[1,2,3]triazolo[4,5-c]quinolin-1-yl)-1-((E)-4-fluorobut-2-enoyl)piperidin-2-yl)acetonitrile;
2-((2S,4S)-4-(6-fluoro-8-methyl-7-(6-methylpyridin-3-yl)-4-((S)-1-((S)-1-methylpyrrolidin-2-yl)ethoxy)-1H-[1,2,3]triazolo[4,5-c]quinolin-1-yl)-1-((E)-4-fluorobut-2-enoyl)piperidin-2-yl)acetonitrile;
2-((2S,4S)-4-(6-fluoro-8-methyl-7-(1-methyl-1H-indazol-3-yl)-4-((S)-1-((S)-1-methylpyrrolidin-2-yl)ethoxy)-1H-[1,2,3]triazolo[4,5-c]quinolin-1-yl)-1-((E)-4-methoxybut-2-enoyl)piperidin-2-yl)acetonitrile;
2-((2S,4S)-4-(6-fluoro-7-(4-fluorophenyl)-8-methyl-4-((S)-1-((S)-1-methylpyrrolidin-2-yl)ethoxy)-1H-[1,2,3]triazolo[4,5-c]quinolin-1-yl)-1-((E)-4-methoxybut-2-enoyl)piperidin-2-yl)acetonitrile;
8-(1-(1-acryloylpiperidin-4-yl)-6-fluoro-8-methyl-4-(S)-1-(5)-1-methylpyrrolidin yl)ethoxy)-1H-[1,2,3]triazolo[4,5-c]quinolin-7-yl)-1-naphthonitrile;
2-((2S,4S)-4-(7-(2-chloro-3-methylphenyl)-4-(3-(dimethylamino)-3-methylazetidin-1-yl)-6-fluoro-8-methyl-1H-imidazo[4,5-c]quinolin-1-yl)-1-((E)-4-fluorobut-2-enoyl)piperidin yl)acetonitrile;
2-((2S,4S)-4-(7-(2-chloro-3-methylphenyl)-6-fluoro-8-methyl-4-((S)-1-((S)-1-methylpyrrolidin-2-yl)ethoxy)-1H-imidazo[4,5-c]quinolin-1-yl)-1-(2-fluoroacryloyl)piperidin-2-yl)acetonitrile;
8-(1-(2R,4S)-1-acryloyl-2-methylpiperidin-4-yl)-6-fluoro-8-methyl-4-(S)-1-(S)-1-methylpyrrolidin-2-yl)ethoxy)-1H-[1,2,3]triazolo[4,5-c]quinolin-7-yl)-1-naphthonitrile;
2-((2S,4S)-4-(7-(5,6-dimethyl-1H-indazol-4-yl)-4-(3-(ethyl(methyl)amino)azetidin-1-yl)-6-fluoro-8-methyl-1H-[1,2,3]triazolo[4,5-c]quinolin-1-yl)-1-(2-fluoroacryloyl)piperidin-2-yl)acetonitrile;
8-(6-fluoro-1-(14(E)-4-fluorobut-2-enoyl)piperidin-4-yl)-8-methyl-4-(S)-1-((S)-1-methylpyrrolidin-2-yl)ethoxy)-1H-[1,2,3]triazolo[4,5-c]quinolin-7-yl)-1-naphthonitrile;
8-(1-(2S,4S)-2-(cyanomethyl)-1-(2-fluoroacryloyl)piperidin-4-yl)-4-(3-(dimethylamino)-3-methylazetidin-1-yl)-6-fluoro-8-methyl-1H-[1,2,3]triazolo[4,5-c]quinolin-7-yl)-1-naphthonitrile;
2-((2S,4S)-4-(6, 8-dichloro-4-(3-(dimethylamino)-3-methylazetidin-1-yl)-7-(5-fluoroquinolin-8-yl)-1H-[1,2,3 ]triazolo[4, 5-c]quinolin-1-yl)-1-((E)-4-methoxybut-2-enoyl)piperidin-2-yl)acetonitrile;
2-((2S,4S)-4-(6, 8-dichloro-4-(3-(dimethylamino)-3-methylazetidin-1-yl)-7-(5-fluoroquinolin-8-yl)-1H-[1,2,3 ]triazolo[4, 5-c]quinolin-1-yl)-1-((E)-4-fluorobut-2-enoyl)piperidin-2-yl)acetonitrile; and
2-((2S,4S)-4-(4-(3-(dimethylamino)-3-methylazetidin-1-yl)-6-fluoro-7-(5-fluoroquinolin-8-yl)-8-(trifluoromethyl)-1H-[1,2,3]triazolo[4,5-c]quinolin-1-yl)-1-((E)-4-methoxybut-2-enoyl)piperidin-2-yl)acetonitrile;
or a pharmaceutically acceptable salt thereof.

26. A method of treating cancer in a patient, comprising administering to said patient:
(i) pemigatinib, or a pharmaceutically acceptable salt thereof; and
(ii) a KRAS inhibitor, which is a compound of Formula (II): 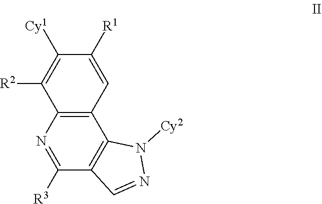
or a pharmaceutically acceptable salt thereof, wherein:
R¹ is selected from Cl, CH₃, CH₂F, CHF₂, and CF₃;
Cy¹ is selected from 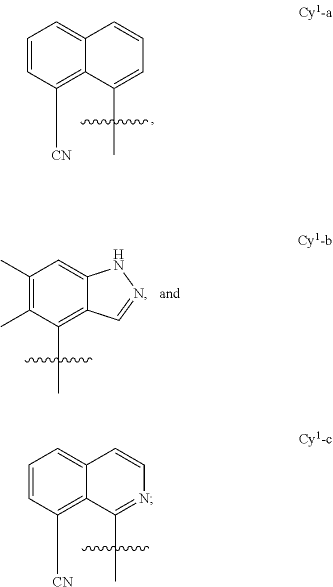 [SMILES: *c1cccc2cccc(CC#N)c12 ; *c1c(C)c(C)cc2[nH]ncc12 ; *c1nccc2cccc(CC#N)c12]
R² is selected from F and Cl;
R³ is selected from 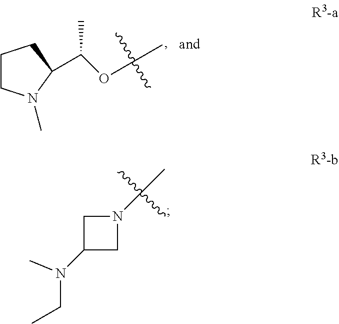
and,
Cy² is selected from 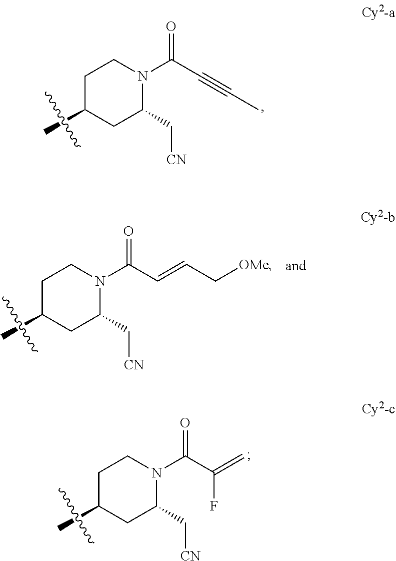
provided that the compound of Formula (II) is other than,
8-(1-((2S,4S)-2-(cyanomethyl)-1-(2-fluoroacryloyl)piperidin-4-yl)-6-fluoro-8-methyl-4-((S)-
1-((S)-1-methylpyrrolidin-2-yl)ethoxy)-1H-pyrazolo[4,3-c]quinolin-7-yl)-1-naphthonitrile,
8-(1-((2S,4S)-2-(cyanomethyl)-1-((E)-4-methoxybut-2-enoyl)piperidin-4-yl)-6-fluoro-8-
methyl-4-((S)-1-((S)-1-methylpyrrolidin-2-yl)ethoxy)-1H-pyrazolo[4,3-c]quinolin-7-yl)-1-
naphthonitrile,
2-((2S,4S)-4-(7-(5,6-dimethyl-1H-indazol-4-yl)-6-fluoro-8-methyl-4-((S)-1-((S)-1-
methylpyrrolidin-2-yl)ethoxy)-1H-pyrazolo[4,3-c]quinolin-1-yl)-1-(2-
fluoroacryloyl)piperidin-2-yl)acetonitrile,
2-((2S,4S)-1-(but-2-ynoyl)-4-(7-(5,6-dimethyl-1H-indazol-4-yl)-6-fluoro-8-methyl-4-((S)-1-
((S)-1-methylpyrrolidin-2-yl)ethoxy)-1H-pyrazolo[4,3-c]quinolin-1-yl)piperidin-2-
yl)acetonitrile, and
2-((2S,4S)-4-(7-(5,6-dimethyl-1H-indazol-4-yl)-6-fluoro-8-methyl-4-((S)-1-((S)-1-
methylpyrrolidin-2-yl)ethoxy)-1H-pyrazolo[4,3-c]quinolin-1-yl)-1-((E)-4-methoxybut-2-
enoyl)piperidin-2-yl)acetonitrile.

27. The method of claim 26, wherein the compound of Formula (II) or the pharmaceutically acceptable salt thereof is selected from:
1-(1-(2S,4S)-1-(but-2-ynoyl)-2-(cyanomethyl)piperidin-4-yl)-8-chloro-6-fluoro-4-(S)-1-((S)-1-methylpyrrolidin-2-yl)ethoxy)-1H-pyrazolo[4,3-c]quinolin-7-yl)isoquinoline-8-carbonitrile;
1-(8-chloro-1-(2S,4S)-2-(cyanomethyl)-1-(E)-4-methoxybut-2-enoyl)piperidin-4-yl)-6-fluoro-4-(S)-1-((S)-1-methylpyrrolidin-2-yl)ethoxy)-1H-pyrazolo[4,3-c]quinolin-7-yl)isoquinoline-8-carbonitrile;
1-(8-chloro-1-(2S,4S)-2-(cyanomethyl)-1-(2-fluoroacryloyl)piperidin-4-yl)-6-fluoro-4-((S)-1-(S)-1-methylpyrrolidin-2-yl)ethoxy)-1H-pyrazolo[4,3-c]quinolin-7-yl)isoquinoline-8-carbonitrile;
2-((2S,4S)-4-(8-chloro-7-(5,6-dimethyl-1H-indazol-4-yl)-4-(3-(ethyl(methyl)amino)azetidin-1-yl)-6-fluoro-1H-pyrazolo[4,3-c]quinolin-1-yl)-1-(2-fluoroacryloyl)piperidin-2-yl)acetonitrile;
8-(8-chloro-1-(2S,4S)-2-(cyanomethyl)-1-(2-fluoroacryloyl)piperidin-4-yl)-6-fluoro-4-((S)-1-(S)-1-methylpyrrolidin-2-yl)ethoxy)-1H-pyrazolo[4,3-c]quinolin-7-yl)-1-naphthonitrile;
8-(8-chloro-1-(2S,4S)-2-(cyanomethyl)-1-((E)-4-methoxybut-2-enoyl)piperidin-4-yl)-6-fluoro-4-((S)-1-((S)-1-methylpyrrolidin-2-yl)ethoxy)-1H-pyrazolo[4,3-c]quinolin-7-yl)-1-naphthonitrile; and
8-(1-(2S,4S)-1-(but-2-ynoyl)-2-(cyanomethyl)piperidin-4-yl)-6-fluoro-8-methyl-4-(S)-1-((S)-1-methylpyrrolidin-2-yl)ethoxy)-1H-pyrazolo[4,3-c]quinolin-7-yl)-1-naphthonitrile;
or a pharmaceutically acceptable salt thereof.

28. A method of treating cancer in a patient, comprising administering to said patient:
(i) an FGFR1 inhibitor selected from pemigatinib, futibatinib, erdafitinib and infigratinib, or a pharmaceutically acceptable salt thereof; and
(ii) a KRAS inhibitor selected from sotorasib, adagrasib, Compound 2, Compound 3 and Compound 4, or a pharmaceutically acceptable salt thereof.

29. The method of claim 28, wherein the FGFR1 inhibitor is pemigatinib, or a pharmaceutically acceptable salt thereof.

30. The method of claim 28, wherein the FGFR1 inhibitor is futibatinib, or a pharmaceutically acceptable salt thereof.

31. The method of claim 28, wherein the FGFR1 inhibitor is erdafitinib, or a pharmaceutically acceptable salt thereof.

32. The method of claim 28, wherein the FGFR1 inhibitor is infigratinib, or a pharmaceutically acceptable salt thereof.

33. The method of claim 28, wherein the KRAS inhibitor is sotorasib, or a pharmaceutically acceptable salt thereof.

34. The method of claim 28, wherein the KRAS inhibitor is adagrasib, or a pharmaceutically acceptable salt thereof.

35. The method of claim 28, wherein the KRAS inhibitor is Compound 2, or a pharmaceutically acceptable salt thereof.

36. The method of claim 28, wherein the KRAS inhibitor is Compound 3, or a pharmaceutically acceptable salt thereof.

37. The method of claim 28, wherein the KRAS inhibitor is Compound 4, or a pharmaceutically acceptable salt thereof.

38. A method of treating cancer in a patient, comprising administering to said patient:
(i) pemigatinib, or a pharmaceutically acceptable salt thereof; and
(ii) a KRAS inhibitor selected from sotorasib, adagrasib, Compound 2, Compound 3 and Compound 4, or a pharmaceutically acceptable salt thereof.

39. The method of claim 38, wherein the KRAS inhibitor is sotorasib, or a pharmaceutically acceptable salt thereof.

40. The method of claim 38, wherein the KRAS inhibitor is adagrasib, or a pharmaceutically acceptable salt thereof.

41. The method of claim 38, wherein the KRAS inhibitor is Compound 2, or a pharmaceutically acceptable salt thereof.

42. The method of claim 38, wherein the KRAS inhibitor is Compound 3, or a pharmaceutically acceptable salt thereof.

43. The method of claim 38, wherein the KRAS inhibitor is Compound 4, or a pharmaceutically acceptable salt thereof.

44. A method of treating cancer in a patient, comprising administering to said patient:
(i) pemigatinib, or a pharmaceutically acceptable salt thereof; and
(ii) sotorasib, or a pharmaceutically acceptable salt thereof.

45. The method of claim 44, wherein pemigatinib, or a pharmaceutically acceptable salt thereof, and sotorasib or a pharmaceutically acceptable salt thereof, are administered simultaneously.

46. The method of claim 44, wherein pemigatinib, or a pharmaceutically acceptable salt thereof, and sotorasib or a pharmaceutically acceptable salt thereof, are administered sequentially.

47. The method of claim 44, wherein pemigatinib, or a pharmaceutically acceptable salt thereof, is administered orally.

48. The method of claim 47, wherein pemigatinib, or a pharmaceutically acceptable salt thereof, is administered in the form of a tablet.

49. The method of claim 44, wherein pemigatinib, or a pharmaceutically acceptable salt thereof, is administered in a daily dose of about 1 mg to about 10 mg.

50. The method of claim 44, wherein pemigatinib, or a pharmaceutically acceptable salt thereof, is administered in a daily dose of about 1 mg to about 5 mg.

51. The method of claim 44, wherein pemigatinib, or a pharmaceutically acceptable salt thereof, is administered in a daily dose of about 2 mg.

52. The method of claim 44, wherein sotorasib, or a pharmaceutically acceptable salt thereof, is administered orally.

53. The method of claim 52, wherein sotorasib, or a pharmaceutically acceptable salt thereof, is administered in the form of a tablet.

54. The method of claim 44, wherein sotorasib, or a pharmaceutically acceptable salt thereof, is administered in a daily dose of about 50 mg to about 300 mg.

55. The method of claim 44, wherein sotorasib, or a pharmaceutically acceptable salt thereof, is administered in a daily dose of about 100 mg to about 200 mg.

56. The method of claim 44, wherein sotorasib, or a pharmaceutically acceptable salt thereof, is administered in a daily dose of about 133 mg.

57. The method of claim 44, wherein sotorasib, or a pharmaceutically acceptable salt thereof, is administered in a daily dose of about 120 mg.

58. The method of claim 44, wherein pemigatinib, or a pharmaceutically acceptable salt thereof, and sotorasib, or a pharmaceutically acceptable salt thereof, are orally administered simultaneously in a daily dose of about 2 mg and 120 mg, respectively.

59. The method of claim 44, wherein pemigatinib, or a pharmaceutically acceptable salt thereof, and sotorasib, or a pharmaceutically acceptable salt thereof, are orally administered sequentially in a daily dose of about 2 mg and 120 mg, respectively.

60. The method of claim 58, wherein pemigatinib, or a pharmaceutically acceptable salt thereof, and sotorasib, or a pharmaceutically acceptable salt thereof, are each administered in the form of a tablet.

61. The method of claim 1, further comprising administering one or more additional therapeutic agents.

62. The method of claim 61, comprising administering one additional therapeutic agent.

63. The method of claim 62, wherein the additional therapeutic agent in an inhibitor of PD-1 or PD-L1.

64. The method of claim 1, wherein the cancer comprises one or more KRAS mutations.

65. The method of claim 64, wherein the one or more KRAS mutations comprise mutations selected from G12C, G12D, C12V and combinations thereof.

66. The method of claim 64, wherein the cancer further comprises high FGFR1 expression.

67. The method of claim 1, wherein the cancer is selected from carcinomas, pancreatic cancer, colorectal cancer, lung cancer, non-small cell lung cancer, ovarian cancer, bladder cancer, gastric cancer, esophageal cancer, breast cancer, head and neck cancer, cervical cancer, skin cancer, thyroid cancer, hematopoietic malignancies, multiple myeloma, acute myelogenous leukemia, myeloproliferative neoplasms, neoplasms, glioblastoma and sarcomas.

68. The method of claim 1, wherein the cancer is lung cancer.

69. The method of claim 68, wherein the lung cancer is non-small cell lung cancer.

70. The method of claim 1, wherein the cancer is colorectal cancer.

71. The method of claim 1, wherein the cancer is pancreatic cancer.

72. The method of claim 1, wherein the cancer is ovarian cancer.
